SEQUENCE LISTING (49 sequences)

SEQ ID NO: 1 - DNA, 849 bp, Artificial Sequence
  Artificially synthesized sequence
ggtctcacacaccacaattcagcaaattgtgaacatcatcacgttcatctttccctggttgccaatggcccattttcctgtcagtaacgagaaggtcgcgaattcaggcgctttttagactggtcgtaatgaaattctttttaagaaggagatatacatatgattaaattaaaatttggtgttttttttacagttttactatcttcagcatatgcacatggaacacctcaaaatattactcacggtgtgttaaccctgtcttctcgtggtctggactatccatacgatgtaccggattacgcgcgttatggaagacgagtgttgaacaagggatggtatttaaccgtgttaacaatggagatcttcgaaaatgtggctaatggcgatatttctgccacttccaccgatgcgattaacggaagtcagttgtatgctgtggcaaaaggggtaacaaaccttgctggacaagtgaataatcttgagggcaaagtgaataaagtgggcaaacgtgcagatgcaggtacagcaagtgcattagcggcttcacagttaccacaagccactatgccaggtaaatcaatggttgctattgcgggaagtagttatcaaggtcaaaatggtttagctatcggggtatcaagaatttccgataatggcaaagtgattattcgcttgtcaggcacaaccaatagtcaaggtaaaacaggcgttgcagcaggtgttggttaccagtggtaagttagagcggccgccaccgctgagcaataactagcataaccccttggggcctctaaacgggtcttgaggggttttttgctgaaaggaggaactatatccgggtaacccggg

SEQ ID NO: 2 - DNA, 3475 bp, Artificial Sequence
  Artificially synthesized sequence
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggtctcacacaccacaattcagcaaattgtgaacatcatcacgttcatctttccctggttgccaatggcccattttcctgtcagtaacgagaaggtcgcgaattcaggcgctttttagactggtcgtaatgaaattctttttaagaaggagatatacatatgattaaattaaaatttggtgttttttttacagttttactatcttcagcatatgcacatggaacacctcaaaatattactcacggtgtgttaaccctgtcttctcgtggtctggactatccatacgatgtaccggattacgcgcgttatggaagacgagtgttgaacaagggatggtatttaaccgtgttaacaatggagatcttcgaaaatgtggctaatggcgatatttctgccacttccaccgatgcgattaacggaagtcagttgtatgctgtggcaaaaggggtaacaaaccttgctggacaagtgaataatcttgagggcaaagtgaataaagtgggcaaacgtgcagatgcaggtacagcaagtgcattagcggcttcacagttaccacaagccactatgccaggtaaatcaatggttgctattgcgggaagtagttatcaaggtcaaaatggtttagctatcggggtatcaagaatttccgataatggcaaagtgattattcgcttgtcaggcacaaccaatagtcaaggtaaaacaggcgttgcagcaggtgttggttaccagtggtaagttagagcggccgccaccgctgagcaataactagcataaccccttggggcctctaaacgggtcttgaggggttttttgctgaaaggaggaactatatccgggtaacccgggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 3 - DNA, 675 bp, Artificial Sequence
  Artificially synthesized sequence
ggtctcacacaccacaattcagcaaattgtgaacatcatcacgttcatctttccctggttgccaatggcccattttcctgtcagtaacgagaaggtcgcgaattcaggcgctttttagactggtcgtaatgaaattctttttaagaaggagatatacatatgattaaattaaaatttggtgttttttttacagttttactatcttcagcatatgcacatggaacacctcaaaatattactcacggtgtgttaaccctgtcttctcgtggtctggactatccatacgatgtaccggattacgcgcgttatggaagacgagtgttgaacaagggatggtatttaaccgtgttaacaatggagatcttcgaaagccagctgccgcaggcgaccatgccgggcaaaagcatggtggcgattgcgggcagcagctatcagggccagaacggcctggcgattggcgtgagccgcattagcgataacggcaaagtgattattcgcctgagcggcaccaccaacagccagggcaaaaccggcgtggcggcgggcgtgggctatcagtggtaagttagagcggccgccaccgctgagcaataactagcataaccccttggggcctctaaacgggtcttgaggggttttttgctgaaaggaggaactatatccgggtaacccggg

SEQ ID NO: 4 - DNA, 782 bp, SARS-CoV-2
aaccacgtcttcaaaagtggaagccgaagttcaaatcgaccgtctgatcaccggtcgtctgcagagtctgcagacctatgtgacccagcagctcatccgcgccgcggaaattcgtgccagcgcgaatctggccgcgacgaaaatgagcgagtgcgtgctgggccagagcaaacgcgtggatttctgcggcaagggctaccatctgatgagcttcccacagagcgcgccgcacggtgtggtttttctgcatgttacctacgtgccagcccaagaaaaaaacttcacgaccgcgccagcgatctgtcacgacggcaaggcccacttcccacgcgaaggtgtgttcgtgagcaatggtacccactggtttgtgacgcagcgcaacttctacgaaccgcagatcatcaccacggacaacacgttcgtgagcggtaactgcgacgtggtgattggcatcgtgaacaacacggtgtacgatccgctccagccagaactggacagcttcaaggaggagctggacaagtacttcaaaaaccacaccagcccggacgttgatctgggcgacatcagcggcatcaacgcgagcgtggtgaacatccagaaagagatcgatcgtctgaacgaagtggcgaaaaacctcaacgagagtctgatcgatctgcaagaactgggcaaatacgagcagtacatcaagtggccgtggtacatttggctgggtttcattgccggtctgatcgccatcgtgatggtgaccatcatgctgtgctgcatgagaagacttgtgt

SEQ ID NO: 5 - DNA, 746 bp, SARS-CoV-2
aaccacgtcttcaaaagtggaagccgaagttcaaatcgaccgtctgatcaccggtcgtctgcagagtctgcagacctatgtgacccagcagctcatccgcgccgcggaaattcgtgccagcgcgaatctggccgcgacgaaaatgagcgagtgcgtgctgggccagagcaaacgcgtggatttctgcggcaagggctaccatctgatgagcttcccacagagcgcgccgcacggtgtggtttttctgcatgttacctacgtgccagcccaagaaaaaaacttcacgaccgcgccagcgatctgtcacgacggcaaggcccacttcccacgcgaaggtgtgttcgtgagcaatggtacccactggtttgtgacgcagcgcaacttctacgaaccgcagatcatcaccacggacaacacgttcgtgagcggtaactgcgacgtggtgattggcatcgtgaacaacacggtgtacgatccgctccagccagaactggacagcttcaaggaggagctggacaagtacttcaaaaaccacaccagcccggacgttgatctgggcgacatcagcggcatcaacgcgagcgtggtgaacatccagaaagagatcgatcgtctgaacgaagtggcgaaaaacctcaacgagagtctgatcgatctgcaagaactgggcaaatacgagcagtacatcaagtggccgtggtacatttggctgggtttcattgccggtctgatcagaagacttgtgt

SEQ ID NO: 6 - DNA, 710 bp, SARS-CoV-2
aaccacgtcttcaaaagtggaagccgaagttcaaatcgaccgtctgatcaccggtcgtctgcagagtctgcagacctatgtgacccagcagctcatccgcgccgcggaaattcgtgccagcgcgaatctggccgcgacgaaaatgagcgagtgcgtgctgggccagagcaaacgcgtggatttctgcggcaagggctaccatctgatgagcttcccacagagcgcgccgcacggtgtggtttttctgcatgttacctacgtgccagcccaagaaaaaaacttcacgaccgcgccagcgatctgtcacgacggcaaggcccacttcccacgcgaaggtgtgttcgtgagcaatggtacccactggtttgtgacgcagcgcaacttctacgaaccgcagatcatcaccacggacaacacgttcgtgagcggtaactgcgacgtggtgattggcatcgtgaacaacacggtgtacgatccgctccagccagaactggacagcttcaaggaggagctggacaagtacttcaaaaaccacaccagcccggacgttgatctgggcgacatcagcggcatcaacgcgagcgtggtgaacatccagaaagagatcgatcgtctgaacgaagtggcgaaaaacctcaacgagagtctgatcgatctgcaagaactgggcaaatacgagcagtacatcaagtggccgagaagacttgtgt

SEQ ID NO: 7 - DNA, 512 bp, SARS-CoV-2
aaccacgtcttcaacgaccgcgccagcgatctgtcacgacggcaaggcccacttcccacgcgaaggtgtgttcgtgagcaatggtacccactggtttgtgacgcagcgcaacttctacgaaccgcagatcatcaccacggacaacacgttcgtgagcggtaactgcgacgtggtgattggcatcgtgaacaacacggtgtacgatccgctccagccagaactggacagcttcaaggaggagctggacaagtacttcaaaaaccacaccagcccggacgttgatctgggcgacatcagcggcatcaacgcgagcgtggtgaacatccagaaagagatcgatcgtctgaacgaagtggcgaaaaacctcaacgagagtctgatcgatctgcaagaactgggcaaatacgagcagtacatcaagtggccgtggtacatttggctgggtttcattgccggtctgatcgccatcgtgatggtgaccatcatgctgtgctgcatgagaagacttgtgt

SEQ ID NO: 8 - DNA, 476 bp, SARS-CoV-2
aaccacgtcttcaacgaccgcgccagcgatctgtcacgacggcaaggcccacttcccacgcgaaggtgtgttcgtgagcaatggtacccactggtttgtgacgcagcgcaacttctacgaaccgcagatcatcaccacggacaacacgttcgtgagcggtaactgcgacgtggtgattggcatcgtgaacaacacggtgtacgatccgctccagccagaactggacagcttcaaggaggagctggacaagtacttcaaaaaccacaccagcccggacgttgatctgggcgacatcagcggcatcaacgcgagcgtggtgaacatccagaaagagatcgatcgtctgaacgaagtggcgaaaaacctcaacgagagtctgatcgatctgcaagaactgggcaaatacgagcagtacatcaagtggccgtggtacatttggctgggtttcattgccggtctgatcagaagacttgtgt

SEQ ID NO: 9 - DNA, 440 bp, SARS-CoV-2
aaccacgtcttcaacgaccgcgccagcgatctgtcacgacggcaaggcccacttcccacgcgaaggtgtgttcgtgagcaatggtacccactggtttgtgacgcagcgcaacttctacgaaccgcagatcatcaccacggacaacacgttcgtgagcggtaactgcgacgtggtgattggcatcgtgaacaacacggtgtacgatccgctccagccagaactggacagcttcaaggaggagctggacaagtacttcaaaaaccacaccagcccggacgttgatctgggcgacatcagcggcatcaacgcgagcgtggtgaacatccagaaagagatcgatcgtctgaacgaagtggcgaaaaacctcaacgagagtctgatcgatctgcaagaactgggcaaatacgagcagtacatcaagtggccgagaagacttgtgt

SEQ ID NO: 10 - DNA, 251 bp, SARS-CoV-2
aaccacgtcttcagacgttgatctgggcgacatcagcggcatcaacgcgagcgtggtgaacatccagaaagagatcgatcgtctgaacgaagtggcgaaaaacctcaacgagagtctgatcgatctgcaagaactgggcaaatacgagcagtacatcaagtggccgtggtacatttggctgggtttcattgccggtctgatcgccatcgtgatggtgaccatcatgctgtgctgcatgagaagacttgtgt

SEQ ID NO: 11 - DNA, 215 bp, SARS-CoV-2
aaccacgtcttcagacgttgatctgggcgacatcagcggcatcaacgcgagcgtggtgaacatccagaaagagatcgatcgtctgaacgaagtggcgaaaaacctcaacgagagtctgatcgatctgcaagaactgggcaaatacgagcagtacatcaagtggccgtggtacatttggctgggtttcattgccggtctgatcagaagacttgtgt

SEQ ID NO: 12 - DNA, 179 bp, SARS-CoV-2
aaccacgtcttcagacgttgatctgggcgacatcagcggcatcaacgcgagcgtggtgaacatccagaaagagatcgatcgtctgaacgaagtggcgaaaaacctcaacgagagtctgatcgatctgcaagaactgggcaaatacgagcagtacatcaagtggccgagaagacttgtgt

SEQ ID NO: 13 - DNA, 176 bp, SARS-CoV-2
aaccacgtcttcagaagtggcgaaaaacctcaacgagagtctgatcgatctgcaagaactgggcaaatacgagcagtacatcaagtggccgtggtacatttggctgggtttcattgccggtctgatcgccatcgtgatggtgaccatcatgctgtgctgcatgagaagacttgtgt

SEQ ID NO: 14 - DNA, 140 bp, SARS-CoV-2
aaccacgtcttcagaagtggcgaaaaacctcaacgagagtctgatcgatctgcaagaactgggcaaatacgagcagtacatcaagtggccgtggtacatttggctgggtttcattgccggtctgatcagaagacttgtgt

SEQ ID NO: 15 - DNA, 104 bp, SARS-CoV-2
aaccacgtcttcagaagtggcgaaaaacctcaacgagagtctgatcgatctgcaagaactgggcaaatacgagcagtacatcaagtggccgagaagacttgtgt

SEQ ID NO: 16 - protein, 252 aa, SARS-CoV-2
KVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCM

SEQ ID NO: 17 - protein, 9 aa, SARS-CoV-2
LITGRLQSL

SEQ ID NO: 18 - protein, 18 aa, SARS-CoV-2
QLIRAAEIRASANLAATK

SEQ ID NO: 19 - protein, 9 aa, SARS-CoV-2
VVFLHVTYV

SEQ ID NO: 20 - protein, 15 aa, SARS-CoV-2
HWFVTQRNFYEPQII

SEQ ID NO: 21 - protein, 12 aa, SARS-CoV-2
NNTVYDPLQPEL

SEQ ID NO: 22 - protein, 9 aa, SARS-CoV-2
RLNEVAKNL

SEQ ID NO: 23 - protein, 9 aa, SARS-CoV-2
NLNESLIDL

SEQ ID NO: 24 - protein, 9 aa, SARS-CoV-2
FIAGLIAIV

SEQ ID NO: 25 - protein, 22 aa, SARS-CoV-2
FGAGAALQIPFAMQMAYRFNGI

SEQ ID NO: 26 - protein, 14 aa, SARS-CoV-2
IPFAMQMAYRFNGI

SEQ ID NO: 27 - protein, 9 aa, SARS-CoV-2
IPFAMQMAY

SEQ ID NO: 28 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 29 - protein, 18 aa, SARS-CoV-2
SFIEDLLFNKVTLADAGF

SEQ ID NO: 30 - protein, 15 aa, SARS-CoV-2
PLKPTKRSFIEDLLF

SEQ ID NO: 31 - protein, 25 aa, SARS-CoV-2
PSKPSKRSFIEDLLFNKVTLADAGF

SEQ ID NO: 32 - DNA, 110 bp, Artificial Sequence
  Artificially synthesized sequence
aaccacgtcttcaccgagcaaaccgagcaaacgcagcttcatcgaggatctgctgttcaacaaggtgacgctggccgatgccggttttggtggcggcagaagacttgtgt

SEQ ID NO: 33 - DNA, 3432 bp, Artificial Sequence
  Artificially synthesized sequence
ggccgccaccgctgagcaataactagcataaccccttggggcctctaaacgggtcttgaggggttttttgctgaaaggaggaactatatccgggtaacgaattcaagcttgatatcattcaggacgagcctcagactccagcgtaactggactgcaatcaactcactggctcaccttcacgggtgggcctttcttcggtagaagtcatctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgcgcagaaaggcccacccgaaggtgagccaggtgattacatttgggccctcattagaaaaactcatcgagcatcaaatgaaattgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatagctcctgaaaatctcgataactcaaaaaatacgcccggtagtgatcttatttcattatggtgaaagttggaacctcttacgtgccgatcaaaaagacggtcaaaagcctccggtcggaggcttttgactttctgctatggaggtcaggtatgatttaaatggtcagtattgagcgatatctagagaattcgtccaccacaattcagcaaattgtgaacatcatcacgttcatctttccctggttgccaatggcccattttcctgtcagtaacgagaaggtcgcgaattcaggcgctttttagactggtcgtaatgaaattctttttaagaggagatatacataggtctcacatgattaaattaaaatttggtgttttttttacagttttactatcttcagcatatgcacatggaacacctcaaaatattactcacggtgtgttaaccacgtcttcaccgagcaaaccgagcaaacgcagcttcatcgaggatctgctgttcaacaaggtgacgctggccgatgccggttttggtggcggcagaagacttgtgttaacaatggagatcttcgaacaatacagaccggagaacggaagttatgctaccaatatggcactggctaactcactgttcctcatggatttgaatgagcgtaagcaattcagggccatgagtgataatacacagcctgagtctgcatccgtgtggatgaagatcactggaggaataagctctggtaagcttaatgacgggcaaaataaaacaacaaccaatcagtttatcaatcagctcgggggggatatttataaattccatgctgaacaactgggtgattttaccttagggattatgggaggatacgcgaatgcaaaaggtaaaacgataaattacacgagcaacaaagctgccagaaacacactggatggttattctgtcggggtatacggtacgtggtatcagaatggggaaaatgcaacagggctctttgctgaaacttggatgcaatataactggtttaatgcatcagtgaaaggtgacggactggaagaagaaaaatataatctgaatggtttaaccgcttctgcaggtgggggatataacctgaatgtgcacacatggacatcacctgaaggaataacaggtgaattctggttacagcctcatttgcaggctgtctggatgggggttacaccggatacacatcaggaggataacggaacggtggtgcagggagcagggaaaaataatattcagacaaaagcaggtattcgtgcatcctggaaggtgaaaagcaccctggataaggataccgggcggaggttccgtccgtatatagaggcaaactggatccataacactcatgaatttggtgttaaaatgagtgatgacagccagttgttgtcaggtagccgaaatcagggagagataaagacaggtattgaaggggtgattactcaaaacttgtcagtgaatggcggagtcgcatatcaggcaggaggtcacgggagcaatgccatctccggagcactggggataaaatacagcttcggttagagc

SEQ ID NO: 34 - DNA, 446 bp, Artificial Sequence
  Artificially synthesized sequence
aaccacgtcttcaccgagtaaaccgagtaaacgcagctttattgaggatctgctcttcaacaaggtgacgctggccgacgccggttttggcggcggcccgagcaagccgagcaaacgtagctttatcgaggatctgctctttaataaagtgacgctggcggatgcgggtttcggcggtggcccgagcaaaccaagcaaacgtagtttcatcgaggatctgctgttcaataaagttacgctggccgatgcgggttttggtggtggtccaagcaaaccaagtaagcgtagttttattgaggatctgctgtttaacaaggttaccctcgccgatgccggttttggtggcggtccgagtaagccgagtaagcgtagcttcattgaagatctgctgttcaacaaagtgacgctggccgatgccggctttggcggtggtagaagacttgtgt

SEQ ID NO: 35 - DNA, 3768 bp, Artificial Sequence
  Artificially synthesized sequence
ggccgccaccgctgagcaataactagcataaccccttggggcctctaaacgggtcttgaggggttttttgctgaaaggaggaactatatccgggtaacgaattcaagcttgatatcattcaggacgagcctcagactccagcgtaactggactgcaatcaactcactggctcaccttcacgggtgggcctttcttcggtagaagtcatctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgcgcagaaaggcccacccgaaggtgagccaggtgattacatttgggccctcattagaaaaactcatcgagcatcaaatgaaattgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatagctcctgaaaatctcgataactcaaaaaatacgcccggtagtgatcttatttcattatggtgaaagttggaacctcttacgtgccgatcaaaaagacggtcaaaagcctccggtcggaggcttttgactttctgctatggaggtcaggtatgatttaaatggtcagtattgagcgatatctagagaattcgtccaccacaattcagcaaattgtgaacatcatcacgttcatctttccctggttgccaatggcccattttcctgtcagtaacgagaaggtcgcgaattcaggcgctttttagactggtcgtaatgaaattctttttaagaggagatatacataggtctcacatgattaaattaaaatttggtgttttttttacagttttactatcttcagcatatgcacatggaacacctcaaaatattactcacggtgtgttaaccacgtcttcaccgagtaaaccgagtaaacgcagctttattgaggatctgctcttcaacaaggtgacgctggccgacgccggttttggcggcggcccgagcaagccgagcaaacgtagctttatcgaggatctgctctttaataaagtgacgctggcggatgcgggtttcggcggtggcccgagcaaaccaagcaaacgtagtttcatcgaggatctgctgttcaataaagttacgctggccgatgcgggttttggtggtggtccaagcaaaccaagtaagcgtagttttattgaggatctgctgtttaacaaggttaccctcgccgatgccggttttggtggcggtccgagtaagccgagtaagcgtagcttcattgaagatctgctgttcaacaaagtgacgctggccgatgccggctttggcggtggtagaagacttgtgttaacaatggagatcttcgaacaatacagaccggagaacggaagttatgctaccaatatggcactggctaactcactgttcctcatggatttgaatgagcgtaagcaattcagggccatgagtgataatacacagcctgagtctgcatccgtgtggatgaagatcactggaggaataagctctggtaagcttaatgacgggcaaaataaaacaacaaccaatcagtttatcaatcagctcgggggggatatttataaattccatgctgaacaactgggtgattttaccttagggattatgggaggatacgcgaatgcaaaaggtaaaacgataaattacacgagcaacaaagctgccagaaacacactggatggttattctgtcggggtatacggtacgtggtatcagaatggggaaaatgcaacagggctctttgctgaaacttggatgcaatataactggtttaatgcatcagtgaaaggtgacggactggaagaagaaaaatataatctgaatggtttaaccgcttctgcaggtgggggatataacctgaatgtgcacacatggacatcacctgaaggaataacaggtgaattctggttacagcctcatttgcaggctgtctggatgggggttacaccggatacacatcaggaggataacggaacggtggtgcagggagcagggaaaaataatattcagacaaaagcaggtattcgtgcatcctggaaggtgaaaagcaccctggataaggataccgggcggaggttccgtccgtatatagaggcaaactggatccataacactcatgaatttggtgttaaaatgagtgatgacagccagttgttgtcaggtagccgaaatcagggagagataaagacaggtattgaaggggtgattactcaaaacttgtcagtgaatggcggagtcgcatatcaggcaggaggtcacgggagcaatgccatctccggagcactggggataaaatacagcttcggttagagc

SEQ ID NO: 36 - DNA, 3393 bp, Artificial Sequence
  Artificially synthesized sequence
ggccgccaccgctgagcaataactagcataaccccttggggcctctaaacgggtcttgaggggttttttgctgaaaggaggaactatatccgggtaacgaattcaagcttgatatcattcaggacgagcctcagactccagcgtaactggactgcaatcaactcactggctcaccttcacgggtgggcctttcttcggtagaagtcatctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgcgcagaaaggcccacccgaaggtgagccaggtgattacatttgggccctcattagaaaaactcatcgagcatcaaatgaaattgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatagctcctgaaaatctcgataactcaaaaaatacgcccggtagtgatcttatttcattatggtgaaagttggaacctcttacgtgccgatcaaaaagacggtcaaaagcctccggtcggaggcttttgactttctgctatggaggtcaggtatgatttaaatggtcagtattgagcgatatctagagaattcgtccaccacaattcagcaaattgtgaacatcatcacgttcatctttccctggttgccaatggcccattttcctgtcagtaacgagaaggtcgcgaattcaggcgctttttagactggtcgtaatgaaattctttttaagaggagatatacataggtctcacatgattaaattaaaatttggtgttttttttacagttttactatcttcagcatatgcacatggaacacctcaaaatattactcacggtgtgttaaccctgtcttctcgtggtctggactatccatacgatgtaccggattacgcgcgttatggaagacgagtgttaacaatggagatcttcgaacaatacagaccggagaacggaagttatgctaccaatatggcactggctaactcactgttcctcatggatttgaatgagcgtaagcaattcagggccatgagtgataatacacagcctgagtctgcatccgtgtggatgaagatcactggaggaataagctctggtaagcttaatgacgggcaaaataaaacaacaaccaatcagtttatcaatcagctcgggggggatatttataaattccatgctgaacaactgggtgattttaccttagggattatgggaggatacgcgaatgcaaaaggtaaaacgataaattacacgagcaacaaagctgccagaaacacactggatggttattctgtcggggtatacggtacgtggtatcagaatggggaaaatgcaacagggctctttgctgaaacttggatgcaatataactggtttaatgcatcagtgaaaggtgacggactggaagaagaaaaatataatctgaatggtttaaccgcttctgcaggtgggggatataacctgaatgtgcacacatggacatcacctgaaggaataacaggtgaattctggttacagcctcatttgcaggctgtctggatgggggttacaccggatacacatcaggaggataacggaacggtggtgcagggagcagggaaaaataatattcagacaaaagcaggtattcgtgcatcctggaaggtgaaaagcaccctggataaggataccgggcggaggttccgtccgtatatagaggcaaactggatccataacactcatgaatttggtgttaaaatgagtgatgacagccagttgttgtcaggtagccgaaatcagggagagataaagacaggtattgaaggggtgattactcaaaacttgtcagtgaatggcggagtcgcatatcaggcaggaggtcacgggagcaatgccatctccggagcactggggataaaatacagcttcggttagagc

SEQ ID NO: 37 - protein, 6 aa, SARS-CoV-2
IEDLLF

SEQ ID NO: 38 - protein, 8 aa, Human immunodeficiency virus
AVGIGAVF

SEQ ID NO: 39 - DNA, 29903 bp, SARS-CoV-2
  CDS (21563)..(25384)
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 40 - protein, 1273 aa, SARS-CoV-2; identical to SEQ ID NO: 28

SEQ ID NO: 41 - protein, 24 aa, porcine epidemic diarrhea virus
GRVVQKRSFIEDLLFNKVVTNGLG

SEQ ID NO: 42 - protein, 24 aa, SARS-CoV-2
PSKPSKRSFIEDLLFNVKTLADAG

SEQ ID NO: 43 - protein, 8 aa, SARS-CoV-2
SFIEDLLF

SEQ ID NO: 44 - protein, 9 aa, Influenza virus
YPYDVPDYA

SEQ ID NO: 45 - protein, 8 aa, Homo sapiens
YSNIGVCK

SEQ ID NO: 46 - DNA, 110 bp, Artificial Sequence
  Artificially synthesized oligonucleotide sequence
aaccacgtcttcaggccgcgttgttcagaaacgcagcttcatcgaggatctgctgttcaacaaggtggtgaccaatggtctgggcaccggtggcggcagaagacttgtgt

SEQ ID NO: 47 - protein, 8 aa, Human immunodeficiency virus type 1
ALGIGAAF

SEQ ID NO: 48 - protein, 8 aa, Human immunodeficiency virus type 1
AVGFGAAF

SEQ ID NO: 49 - protein, 8 aa, Human immunodeficiency virus type 1
AAGFGAMF

(19) United States
(12) Patent Application Publication
(10) Pub. No.: US 2024/0261394 A1
(43) Pub. Date: Aug. 8, 2024

(54) COMPOSITIONS AND METHODS FOR INDUCING IMMUNE RESPONSES AGAINST CLASS I FUSION PROTEIN VIRUSES

(71) Applicants: University of Virginia Patent Foundation, Charlottesville, VA (US); Virginia Tech Intellectual Properties, Inc., Blacksburg, VA (US)

(72) Inventors: Steven L. Zeichner; Xiang-Jin Meng; Debin Tian

(21) Appl. No.: 17/924,963

(22) PCT Filed: May 11, 2021
(86) PCT No.: PCT/US2021/031798

§ 371 Date: Nov. 11, 2022

Related U.S. Application Data
(60) Provisional application No. 63/127,712, filed on Dec. 18, 2020.
(60) Provisional application No. 63/022,746, filed on May 11, 2020.

(51) Int. Cl. A61K 39/225; A61K 39/00; A61P 31/14; C12N 1/20; C12N 7/00; C12R 1/19

(52) U.S. Cl. CPC A61K 39/225; A61P 31/14; C12N 1/205; C12N 7/00; A61K 2039/522; A61K 2039/523; C12N 2770/00034; C12R 2001/19

(57) ABSTRACT

Provided are modified bacteria and derivatives thereof that express nucleotide sequence encoding an antigen of a viral family selected from the group comprising Retroviridae (e.g., HIV, including a HIV Fusion Peptide antigen), Orthomyxoviridae, Paramyxoviridae, Arenaviridae, 5 Filoviridae, and/or Coronaviridae (e.g., an SARS-CoV, SARS-CoV-2 Fusion Peptide, and/or PEDV). In some embodiments, the bacterium has a reduced genome and induces an enhanced immune response against the viral antigen of interest when administered to a subject. In some embodiments, the viral (e.g., SARS-CoV, 10 SARS-CoV-2, PEDV, and/or HIV) antigen is expressed on a surface of a bacterium. Also provided are method for producing antibodies against viral antigens, vaccine compositions, methods for vaccinating subjects, methods for treating viral infections in subjects, and expression vectors for expressing viral antigens including but not limited to coronavirus (e.g., SARS-CoV, SARS-CoV-2, and/or PEDV) antigens and/or HIV antigens on the surface of reduced 15 genome bacteria.

### CROSS REFERENCE TO RELATED APPLICATIONS

The present application claims the benefit of U.S. Provisional Patent Application Ser. No. 63/022,746, filed May 11, 2020, and the benefit of U.S. Provisional Patent Application Ser. No. 63/127,712, filed Dec. 18, 2020, the disclosures of each of which are incorporated herein by reference in their entireties.

### REFERENCE TO SEQUENCE LISTING SUBMITTED ELECTRONICALLY

The content of the electronically submitted sequence listing in ASCII text file (Name: 3062_129_PCT_ST25.txt; Size: 110 kilobytes; and Date of Creation: May 9, 2021) filed with the application is incorporated herein by reference in its entirety.

### TECHNICAL FIELD

The presently disclosed subject matter relates to compositions and methods useful for inducing cellular and humoral immune responses, such as but not limited to compositions and methods employed in the context of vaccine development and antibody production. In representative embodiments, the presently disclosed subject matter relates to bacteria modified to have reduced expression of genes, such as by having a reduction of the bacterial genomes, and using those bacteria to express viral (e.g., coronavirus and/or HIV) antigens of interest. The presently disclosed subject matter also relates in some embodiments to vaccine compositions and materials to elicit useful antibody responses from humans and animals comprising modified bacteria expressing antigens that induce immune responses against viruses with class I fusion proteins, including Retroviridae (e.g., HIV), Orthomyxoviridae, Paramyxoviridae, Arenaviridae, Filoviridae, and Coronaviridae, including specifically, but not limited to SARS-CoV-2. The presently disclosed subject matter also relates in some embodiments to vaccine compositions and materials to elicit useful antibody responses from humans and animals comprising modified bacteria expressing antigens including, but not limited to SARS-CoV-2 FP and PEDV FP, that induce immune responses against viruses with class I fusion proteins, including Coronaviridae.

### BACKGROUND

Given their importance for human health, much attention has been focused on the development and optimization of vaccines. Platforms that enable rapid production of new vaccines include many viral vectors-, DNA-, and mRNA-based vaccines (reviewed in Hasbold et al., 1998; Onodera et al., 2008; Stavnezer et al., 2008). These platforms can produce promising vaccines, but can require new and expensive dedicated production facilities, employ expensive and sometimes scarce materials, and can demand logistically challenging cold chains.

Severe acute respiratory syndrome coronavirus 2 (SARS-CoV-2), the causative agent of novel coronavirus disease (COVID-19), emerged in Wuhan, China in December 2019, causing respiratory disease and diarrhea in some patients with a high mortality rate of ˜2%. SARS-CoV-2 infects host cells via its spike protein (S), a class I viral fusion protein, structurally and functionally similar to other class I fusion proteins (e.g., Retroviridae, the HIV Env, and Orthomyxoviridae, the influenza HA), and the analogous fusion proteins of other virus families, other Orthomyxoviridae, Paramyxoviridae, Arenaviridae, and Filoviridae (Newman et al., 1992; Roost et al., 1995; Hasbold et al., 1998; Hangartner et al., 2006; Onodera et al., 2008; Stavnezer et al., 2008; White et al., 2008; Harrison, 2015; Rey & Lok, 2018). The HIV and influenza stalks are targets of broadly neutralizing (BN) monoclonal antibodies (mAbs) and the subject of active vaccine development work. mAbs against certain regions of the spike protein of the original SARS-CoV have been shown to prevent disease in animal models. Formalin fixed killed whole cell (KWC) bacterial vaccines are a >100 y old technology. Many licensed, WHO-prequalified KWC vaccines are available, are highly scalable, inexpensive, and highly appropriate for global use. For example, 6 million doses of the WHO-prequalified Euvichol oral cholera vaccine were produced in 1 year using a single 100 L fermenter for ˜$1/dose (Thomsen et al., 1997).

A globally appropriate vaccine for COVID-19 is needed to control the current pandemic. Indeed, rapid development of an effective, inexpensive, globally appropriate vaccine for SARS-CoV-2, as well as other viral agents, represents a long-felt and continuing need in the art.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

The presently disclosed subject matter provides in some embodiments modified bacteria or derivatives thereof having a reduced number of expressed genes and that express one or more antigens from viruses with class I fusion proteins, optionally coronavirus antigens, optionally one or more SARS-CoV and/or SARS-CoV-2 and/or PEDV antigen, further optionally wherein the one or more coronavirus antigens is/are expressed on a surface of a membrane or derivative thereof, wherein the bacterium induces an enhanced immune response against at least one of the one or more coronavirus (e.g., SARS-CoV and/or SARS-CoV-2 and/or PEDV) antigens when administered to a subject as compared to an immune response that would have been induced in the subject by a bacterium of the same strain that has a full complement of expressed genes. In some embodiments, the fusion peptides are those from other viruses with class I fusion proteins, including Retroviridae (e.g., HIV), Orthomyxoviridae (e.g., influenza), Paramyxoviridae, Arenaviridae, and Filoviridae.

The presently disclosed subject matter provides in some embodiments a modified bacterium or derivative thereof having a reduced number of expressed genes and comprising a viral antigen, optionally an antigen from a virus with a class I fusion protein, further optionally a Coronaviridae, which in some embodiments can be a coronavirus (e.g., SARS-CoV, SARS-CoV-2, and/or porcine epidemic diarrhea virus (PEDV)) antigen, optionally wherein the viral antigen is expressed on a surface of a membrane or derivative thereof, wherein the bacterium induces an enhanced immune response against the viral antigen when administered to a subject as compared to an immune response that would have been induced in the subject by a bacterium of the same strain that has a full complement of expressed genes.

In some embodiments, reducing and/or eliminating expression of one or more gene in the bacterium yields the enhanced immunogenicity to the one or more antigens (e.g., SARS-CoV and/or SARS-CoV-2 and/or PEDV, HIV, or other antigens). In some embodiments, the bacterium is a Gram-negative bacterium, optionally a member of the Enterobacteriaceae. In some embodiments, the bacterium is an E. coli. In some embodiments, the reduced number of expressed genes comprises a reduction of at least about 1.0%, 2.0%, 3.0%, 4.0%, 5.0%, 6.0%, 7.0%, 8.0%, 9.0%, 10%, 11%, 12%, 13%, 14%, 15%, or greater than 15% of genes. In some embodiments, the reduced number of expressed genes comprises a reduction of expressed genes selected from the group consisting of at least about 2.4%, at least about 15.9%, and at least about 29.7%. In some embodiments, the coronavirus (e.g., SARS-CoV and/or SARS-CoV-2 and/or PEDV) antigen(s) is/are put on the surface of the bacterium by an approach selected from the group consisting of expression by the cell itself, covalent or non-covalent association with the outer membrane, and combinations thereof.

In some embodiments, the modified bacterium comprises an autotransporter (AT) expression vector encoding the antigen, wherein the expression on the surface is provided by the AT expression vector. In some embodiments, the autotransporter expression vector comprises a codon optimized sequence encoding the antigen. In some embodiments, the AT expression vector comprises a monomeric autotransporter vector or a trimeric autotransporter vector.

In some embodiments, the coronavirus (e.g., SARS-CoV and/or SARS-CoV-2) antigen is derived from the SARS-CoV-2 spike (S) polypeptide. In some embodiments, the coronavirus (e.g., SARS-CoV and/or SARS-CoV-2) antigen comprises, consists essentially of, or consists of amino acid sequences derived from a coronavirus (e.g., SARS-CoV and/or SARS-CoV-2) spike protein, which in some embodiments can be an amino acid sequence as set forth herein. In some embodiments, the amino acid sequence derived from a coronavirus (e.g., SARS-CoV and/or SARS-CoV-2) spike protein is any of SEQ ID NOs: 16-31 and 37, and/or an immunogenic subsequence thereof, and/or a derivative thereof that is capable of inducing an immune response against a coronavirus (e.g., SARS-CoV and/or SARS-CoV-2) spike protein, and/or is encoded by a nucleotide sequence comprising, consisting essentially of, or consisting of any of SEQ ID NOs: 1-15 and 32-35, or any subsequence thereof. In some embodiments the antigen comprises, consists essentially of, or consists of an amino acid sequence as set forth in any of SEQ ID NOs: 16-31 and 37.

In some embodiments, the coronavirus (e.g., SARS-CoV, SARS-CoV-2, and/or porcine epidemic diarrhea virus (PEDV)) antigen comprises, consists essentially of, and/or consists of an amino acid sequence selected from the group consisting of PSKPSKRSFIEDLLFNKVTLADAGF (SEQ ID NO: 31); PSKPSKRSFIEDLLFNVKTLADAG (SEQ ID NO: 42), SFIEDLLF (SEQ ID NO: 43), SFIEDLLFNKVTLADAGF (SEQ ID NO: 29), and GRVVQKRSFIEDLLFNKVVTNGLG (SEQ ID NO: 41). In some embodiments, the coronavirus antigen comprises, consists essentially of, or consists of the amino acid sequence

(SEQ ID NO: 42)
PSKPSKRSFIEDLLFNVKTLADAG
or
(SEQ ID NO: 41)
GRVVQKRSFIEDLLFNKVVINGLG.

The presently disclosed subject matter also relates in some embodiments to methods for producing an antibody in a subject. In some embodiments, the methods comprise providing a modified bacterium as set forth herein and administering the modified bacterium to a subject in an amount and via a route sufficient to produce an antibody in the subject against a coronavirus (e.g., SARS-CoV and/or SARS-CoV-2 and/or PEDV) antigen expressed by the modified bacterium. In some embodiments, the production of the antibody is enhanced in the subject as compared to an immune response produced in a subject by a bacterium of the same strain that has a full complement of expressed genes and that expresses the coronavirus (e.g., SARS-CoV and/or SARS-CoV-2 and/or PEDV) antigen on its surface. In some embodiments, the presently disclosed methods comprise administering the modified bacterium to the subject intranasally, transmucosally, including but not limited to orally, rectally, and vaginally; subcutaneously, intradermally, intramuscularly, other parenteral routes, or any combination thereof.

The presently disclosed subject matter also relates in some embodiments to vaccine compositions comprising one or more modified bacteria as set forth herein and a pharmaceutically acceptable carrier. In some embodiments, the vaccine compositions further comprise one or more adjuvants. In some embodiments, the modified bacterium is a live attenuated bacterium or a killed whole cell bacterium, or derivatives or fragments thereof. In some embodiments, the vaccine composition is adapted to be administered orally, rectally, vaginally, intra-nasally, parenterally, intradermally, subcutaneously, or intramuscularly. In some embodiments, the vaccine composition further comprises an adjuvant.

The presently disclosed subject matter also relates in some embodiments to methods for vaccinating subjects in need thereof against a virus, such as a coronavirus. In some embodiments, the methods comprise providing a vaccine composition as set forth herein and administering the vaccine composition to the subject. In some embodiments, the vaccine composition is administered orally, rectally, vaginally, intra-nasally, parenterally, intradermally, subcutaneously, or intramuscularly. In some embodiments, the method comprises vaccinating a subject in need thereof against a viral class I fusion protein, optionally wherein the viral class I fusion protein is a fusion protein from a virus of a Coronaviridae (e.g., SARS-CoV, SARS-CoV-2, and/or porcine epidemic diarrhea virus (PEDV), optionally a SARS-CoV-2 FP and/or a PEDV FP),

The presently disclosed subject matter also relates in some embodiments to methods for treating viral infections, such as coronavirus infections, in subjects in need thereof. In some embodiments, the methods comprise providing a vaccine composition as set forth herein and administering the vaccine to the subject. In some embodiments, the vaccine composition is administered orally, rectally, vaginally, intra-nasally, parenterally, intradermally, subcutaneously, or intramuscularly. In some embodiments, the method comprises treating an infection of a virus, optionally a virus of a Coronaviridae (e.g., SARS-CoV, SARS-CoV-2, and/or porcine epidemic diarrhea virus (PEDV), optionally a SARS-CoV-2 FP and/or a PEDV FP) in a subject in need thereof.

The presently disclosed subject matter also relates in some embodiments to expression vectors comprising nucleotide sequences encoding coronavirus (e.g., SARS-CoV and/or SARS-CoV-2 and/or PEDV) antigens. In some embodiments, the expression vectors are configured to express one or more coronavirus (e.g., SARS-CoV and/or SARS-CoV-2 and/or PEDV) antigens in a modified bacterium or derivative thereof having a reduced number of expressed genes, optionally on the surface of the modified bacterium or derivative thereof. In some embodiments, the expression vector comprises an autotransporter (AT) expression vector. The presently disclosed subject matter also relates in some embodiments to expression vectors comprising a nucleotide sequence encoding a viral antigen, optionally a viral antigen from a virus of a Coronaviridae (e.g., SARS-CoV, SARS-CoV-2, and/or porcine epidemic diarrhea virus (PEDV)), wherein the expression vector is configured to express the viral antigen in a modified bacterium or derivative thereof having a reduced number of expressed genes, optionally on the surface of the modified bacterium or derivative thereof.

In some embodiments, the vector comprises a codon optimized sequence encoding the antigen. In some embodiments, the AT expression vector comprises a monomeric autotransporter vector or a trimeric autotransporter vector. In some embodiments, the nucleotide sequence encoding the coronavirus (e.g., SARS-CoV and/or SARS-CoV-2 and/or PEDV) antigen or the nucleotide sequence encoding the viral, optionally coronavirus (e.g., SARS-CoV, SARS-CoV-2, and/or porcine epidemic diarrhea virus (PEDV), antigen, optionally comprising, consisting essentially of, or consisting of a SARS-CoV-2 FP and/or a PEDV FP, is positioned under control of an inducible promoter or a constitutive promoter. In some embodiments, the coronavirus (e.g., SARS-CoV and/or SARS-CoV-2 and/or PEDV) antigen or the viral, optionally coronavirus (e.g., SARS-CoV, SARS-CoV-2, and/or porcine epidemic diarrhea virus (PEDV), antigen, optionally comprising, consisting essentially of, or consisting of a SARS-CoV-2 FP and/or a PEDV FP, is expressed as a monomer, a trimer, a quadramer, a pentamer, or higher order multimer (e.g., a 6-mer or higher), including a tandemly repeated multimer, optionally a pentamer. These multimers may be homomultimers or optionally heteromultimers, in which different fusion peptide variants are expressed together as multimers. In some embodiments, the units of the multimer are separated by one or more linkers, optionally wherein the linkers are amino acid linkers. In some embodiments, the expression vector is provided in a pharmaceutically acceptable carrier. In some embodiments, the nucleotide sequence encoding the coronavirus (e.g., SARS-CoV and/or SARS-CoV-2) antigen comprises, consists essentially of, or consists of a nucleotide sequence as set forth in any of SEQ ID NOs: and 1-15 and 32-35, and/or that encodes an amino acid sequence as set forth in any of SEQ ID NOs: 16-31 and 37, or comprise, consist essentially of, or consist of any homologous and/or derivative amino acid sequences that are capable of eliciting an immune response against a coronavirus (e.g., SARS-CoV and/or SARS-CoV-2) antigen. In some embodiments, the nucleotide sequence encoding the viral, optionally coronavirus (e.g., SARS-CoV, SARS-CoV-2, and/or PEDV), antigen encoses an amino acid sequence selected from the group consisting of PSKPSKRSFIEDLLFNKVTLADAGF (SEQ ID NO: 31); PSKPSKRSFIEDLLFNVKTLADAG (SEQ ID NO: 42), SFIEDLLF (SEQ ID NO: 43), SFIEDLLFNKVTLADAGF (SEQ ID NO: 29), and GRVVQKRSFIEDLLFNKVVTNGLG (SEQ ID NO: 41).

Accordingly, it is an object of the presently disclosed subject matter to provide methods and compositions for eliciting immune responses to immunogenic viral antigens, such as immunogenic coronavirus (e.g., SARS-CoV and/or SARS-CoV-2) antigens, optionally immunogenic subsequences of the SARS-CoV-2 spike (S) protein such as but not limited to for the purpose of creating new and more effective vaccines and for antibody production. Accordingly, it is an object of the presently disclosed subject matter to provide methods and compositions for eliciting immune responses to immunogenic viral antigens, such as immunogenic coronavirus (e.g., SARS-CoV and/or SARS-CoV-2 and/or PEDV) antigens, optionally immunogenic subsequences of the SARS-CoV-2 FP and PEDV FP such as but not limited to for the purpose of creating new and more effective vaccines and for antibody production.

This and other objects are achieved in whole or in part by the presently disclosed subject matter. Further, an object of the presently disclosed subject matter having been stated above, other objects and advantages of the presently disclosed subject matter will become apparent to those skilled in the art after a study of the following Description, Figures, and EXAMPLES.

### BRIEF DESCRIPTION OF THE FIGURES

FIG. 1. Schematic showing exemplary SARS-CoV-2 stalk antigens from S protein for expression with the trimeric HA autotransporter.

FIG. 2. Schematic depicting the design of exemplary Gram-negative surface transporter expression cassette plasmid pRIAIDA (SEQ ID NO: 36). The plasmid was synthesized to include the following features: a rhamnose inducible promoter (PrhaBAD), the Gram-negative expression cassette including the N-terminal signal sequence (Signal), an HA immunotag (HA-tag), which serves both as a test antigen for the demonstration of the technology and as a “stuffer” sequence that can be removed and replaced with DNA sequence encoding an immunogen-of-interest for the production of a vaccine or immunogen to elicit useful antibodies, a trypsin cleavage site to evaluate surface expression of the HA immunotag (and any other surface expressed protein cloned into the cloning sites flanking the HA-tag coding sequence (here shown as Bbs I sites), the beta-barrel of the autotransporter (AIDA-I Autotransporter), together with the plasmid origin of replication (ori), and the kanamycin resistance gene with its promoter (PKanR, KanR)

FIG. 3. Photograph of immunoblots showing surface expression and relative amounts of a surface-expressed HA immunotag expressed by the AIDA-I autotransporter in various reduced genome E. coli. Protein extracts were made from aliquots of E. coli that had not been transformed with an HA immunotag expression cassette, wild type (WT) E. coli that had been transformed with a plasmid with an HA immunotag autotransporter surface expression cassette, and reduced genome (2.4% deleted, 15.8% deleted, and 29.7% deleted) E. coli that had been transformed with a plasmid with an HA immunotag autotransporter surface expression cassette with and without exposure to trypsin. Rhamnose was used to induce expression of the HA immunotag. The extracts were analyzed via immunoblotting using a commercial anti-HA monoclonal antibody. HA bands are seen in the extracts from the E. coli transformed with the plasmid having the HA immunotag surface expression cassette. Treatment of the bacteria with trypsin prior to production of the protein extract severely reduced or eliminated the HA band, indicating that HA was placed on the bacterial surface and that there was minimal detectable HA in the bacterial cytoplasm. DNAK (70 kDa) was used as a lane loading control.

FIG. 4. Plot showing binding and immunogenicity of a test antigen expressed on the surface of wild type and genome deleted E. coli. FIG. 4 shows binding of a commercial anti-HA mAb to wild type and genome deleted (2.4%, 15.9%, 29.7%) E. coli assessed by flow cytometry. GR E. coli harboring a pRIAIDA plasmid were grown in LB with kanamycin and induced with rhamnose. The bacteria were pelleted, washed in PBS, and fixed with formalin, then washed in PBS. Cells were incubated with anti-HA mAb (Invitrogen), with secondary anti-mouse IgG-Alexa 488 (BD Bioscience). Stained cells were measured by flow cytometry using a BD FACSCALIBUR™, and data were analyzed with FlowJo v10 to determine the percentage of GR bacteria stained with anti-HA mAb.

FIGS. 5A and 5B. Vaccine platform design and implementation. FIG. 5A. Map of synthetic plasmid pRAIDA2. Design features include a high copy origin of replication, a kanamycin resistance marker, and an AIDA-I autotransporter expression cassette under the control of a rhamnose-inducible promoter. The expression cassette has a cloning site flanked by BbsI type IIS restriction sites. In its original, parental version, pRAIDA2 expresses an influenza virus HA immunotag. FIG. 5B. A schematic diagram of the general process of candidate vaccine production using pRAIDA2 and genome-reduced bacteria.

FIGS. 6A-6C. Genes with imputed locations on the surface of the genome-reduced bacteria and increased binding of a mAb against a recombinant antigen expressed on the surfaces of genome-reduced bacteria transformed with pRAIDA2. FIG. 6A. Genes removed with imputed locations on the surface of E. coli strains ME5000, ME5110, ME5119, and ME5125 as a function of percent genome deleted. FIG. 6B. Binding of a commercial anti-HA monoclonal antibody to the surfaces of the genome-reduced bacteria as a function of percent genome deleted. FIG. 6C. Immunoblots of extracts from the wild type and genome reduced bacteria, transformed with pRAIDA2 and with expression of the HA immunotag with or without rhamanose induction (indicated at top). Also indicated is whether the bacteria were pretreated with trypsin prior to protein extraction. Rhamnose induces expression of the HA immunotag expressed via the autotransporter expression cassette. Trypsin treatment removes the HA immunotag from the surface of the bacteria. DNAK was probed as a loading control. The normalized amount of AIDA-HA differed by <16% from strain to strain, with the ME5125 29.7% deleted strain expressing the least normalized AIDA-HA.

FIGS. 7A-7D. FPs from PEDV and SARS-CoV-2 and their surface expression in genome reduced bacteria. FIG. 7A. Alignment of FPs from PEDV (GRVVQKRSFIEDLLFNKVVTNGLG; SEQ ID NO: 41) and SARS-CoV-2 (PSKPSKRSFIEDLLFNVKTLADAG; SEQ ID NO: 42) expressed in the candidate vaccines in this study. Upper case letters indicate the 13 amino acid residues that are identical between the PEDV FP and SARS-CoV-2 FP. Upper case letters in bold indicate FP sequence that is conserved among all known coronavirus sequences. FIG. 7B. Flow cytometry conducted on wild type (ME5000) and 29.7% genome-reduced E. coli (ME5125), transformed with parental pRIADA2 expressing the HA immunotag (HA), pRAIDA2-PEDV (PEDV FP), or pRAIDA2-SARS-CoV-2 (SARS-CoV-2 FP). Bacteria were treated with (+) or without (−) rhamnose to induce expression of the FP cloned into the pRAIDA2 expression cassette. Cells were stained with a commercial anti-HA monoclonal antibody, or with rabbit polyclonal anti-PEDV FP or rabbit polyclonal anti-SARS-CoV-2 FP, and then with the appropriate Alexa 486-conjugated secondary antibodies. The results revealed that the HA immunotag and the FPs are expressed on the surface of the bacteria, and binding is enhanced when the proteins are expressed on the surface of the genome reduced bacteria. FIG. 7C. Summary of additional pooled repeat flow cytometry experiments. The antigens are consistently expressed on the bacteria, and binding to the genome reduced bacteria is consistently greater than to the wild type parental bacteria. FIG. 7D. Immunoblots confirming expression of the FPs in extracts of the bacteria transformed with pRAIDA2-PEDV, or pRAIDA2-SARS-CoV-2, probed using the anti-PEDV FP or anti-SARS-CoV-2 FP rabbit polyclonal antibodies.

FIGS. 8A-8C. Pig humoral and IFN-γ responses after vaccination with killed whole cell genome reduced bacterial vaccines expressing FPs using pRAIDA2. FIGS. 8A and 8B. Pig humoral immune responses against the PEDV FP (FIG. 8A) or SARS-CoV-2 FP (FIG. 8B) following vaccination and virus challenge. Normalized OD (Sample OD−Negative control OD/Positive control OD−Negative control OD). There was a small, but statistically significant increase in anti-PEDV FP antibody in the pigs vaccinated with the SARS-CoV-2 FP vaccine (p<0.05, Wilcoxon Rank Sum Test), and a weak trend to significance in eliciting an immune response against the SARS-CoV-2 FP itself (p=0.25) in pigs vaccinated with the SARS-CoV-2 FP vaccine. There was a strong and statistically significant (p<0.05, Wilcoxon Rank Sum Test), anamnestic response against both the PEDV FP and SARS-CoV-2 FP in pigs vaccinated with PEDV and SARS-CoV-2 FP vaccines, respectively. FIG. 8C. IFN-γ responses in serum samples of vaccinated and control pigs. There were significant differences at 5 weeks post-vaccination (wpv, P<0.05) and 1 week post-challenge (wpc, P<0.05) between the vaccinated groups and control.

FIGS. 9A and 9B. Pig clinical responses after vaccination and PEDV challenge infection. FIG. 9A. Diarrhea scores following PEDV challenge. FIG. 9B. Body condition scores following PEDV challenge. Both PEDV FP and SARS-CoV-2 FP vaccines provided substantial and highly statistically significant protection against adverse clinical effects observed following the PEDV challenge infection (p<0.01 for all groups, Friedman Rank Sum Test, comparing each vaccinated group to the control for both diarrhea and body condition scores.) Diarrhea scores range from 1 to 3, where 1 is normal to pasty feces, 2 is semiliquid diarrhea with some solid content, and 3 is liquid diarrhea with no solid content. Body condition scores range from 1 to 3, where 1 is undetectable spinous processes and hook bones, 2 is spinous processes and hook bones were slightly felt, and 3 is spinous processes and hook bones were easily felt and visible.

FIGS. 10A-10F. Pig intestinal compartment viral RNA loads, and histological lesion and intestinal content clinical scoring at necropsy after vaccination and challenge with PEDV. FIGS. 10A-10D. Effects of vaccination on the viral RNA loads in the jejunum tissue (FIG. 10A), small intestine contents from necropsy (FIG. 10B), colon tissue (FIG. 10C), and cecum tissue (FIG. 10D). There was a significant difference in viral RNA loads in the comparison of control vs. PEDV vaccine in the jejunum tissue (p=0.01, Kruskal-Wallis test). There were trends to significance in the comparison of control vs. PEDV vaccine in the small intestine content collected at necropsy (p=0.158), and in the colon tissue (p=0.11). Similar to PEDV FP, the SARS-CoV-2 FP vaccinated pigs also have lower PEDV loads compared to control pigs. FIG. 10E. The Intestinal Content Score recorded at necropsy. The difference between SARS-CoV-2 FP group and control group was borderline statistically significant (p=0.055, Kruskal-Wallis test). The difference between the PEDV FP group and the control group also showed a tendency to significance (p=0.08). FIG. 10F. Histopathological scoring of intestinal villous length to crypt depth. The mean ratios of villous length to crypt depth (V:C) of jejunum tissues from both vaccine groups had higher values (more healthy jejunum) than control group, although the difference were not significant, for either the PEDV FP vaccine (p=0.20, Kruskal-Wallis) or the SARS-CoV-2 FP vaccine (p=0.27), in part due to the dispersion of the values.

### BRIEF DESCRIPTION OF THE SEQUENCE LISTING

SEQ ID NO: 1 is the pRHIA-2 insert sequence (nucleic acid).

SEQ ID NO: 2 is the pRHIA-2 complete sequence (including pUC57; nucleic acid).

SEQ ID NO: 3 is the pRHIA-2 Trnc insert sequence (nucleic acid).

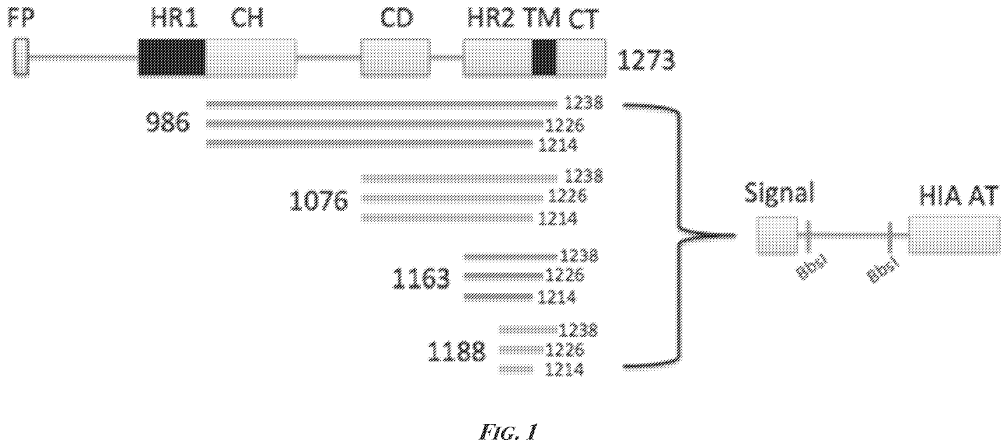

SEQ ID NOs: 4-15 are various exemplary nucleotide sequences encoding subsequences of the SARS-CoV-2 S polypeptide as set forth in FIG. 1.

SEQ ID NO: 16: is an amino acid sequence of the SARS-CoV-2 S polypeptide central helix to the end of transmembrane domain (amino acids 986-1237 of SEQ ID NO: 28).

SEQ ID NOs: 17-24 are the amino acid sequences of exemplary dominant SARS-COV T cell epitopes.

SEQ ID NOs: 25-27 are amino acid sequences of exemplary B cell, TCD4, and TCD8 epitopes, respectively.

SEQ ID NO: 28 is the amino acid sequence set forth in GENBANK® Accession No. QHD43416.1 as a full length S amino acid sequence of SARS-CoV-2. It is encoded by nucleotides 21563-25384 of GENBANK® Accession No. MN908947.3.

SEQ ID NO: 29 is the amino acid sequence of the SARS-CoV-2 Fusion Peptide, and corresponds to amino acids 816-833 of SEQ ID NO: 28. In some embodiments, other SARS-CoV-2 Fusion Peptides can comprise, consist essentially of, or consist of the amino acid sequence PSKPSKRSFIEDLLFNKVTLADAGF (amino acids 809-833 of SEQ ID NO: 28) and/or a subsequence thereof, including but not limited to the amino acid sequences SFIEDLLFNKVTLADAGF amino acids 816-833 of SEQ ID NO: 28), PSKPSKRSFIEDLLF (amino acids 809-823 of SEQ ID NO: 28), and SFIEDLLF (816-823 of SEQ ID NO: 28). In some embodiments, the presently disclosed subject matter provides biologically active fragments and/or homologs of these sequences.

SEQ ID NO: 30 is the amino acid sequence of an 5H10 hMAB epitope binding site on SARS-CoV, and corresponds to amino acids 811-823 of SEQ ID NO: 28.

SEQ ID NO: 31 is the amino acid sequence of a SARS-CoV-2 Fusion Peptide (FP) with certain added amino acids, and corresponds to amino acids 809-833 of SEQ ID NO: 28.

SEQ ID NO: 32 is an exemplary nucleic acid sequence of a SARS-CoV-2 FP monomer sequence after codon optimization with BbsI sites added to the ends.

SEQ ID NO: 33 is an exemplary nucleic acid sequence of a SARS-CoV-2 FP monomer sequence after codon optimization with BbsI sites added to the ends and cloned into pRIAIDA2.

SEQ ID NO: 34 is an exemplary nucleic acid sequence of a SARS-CoV-2 FP 5-mer sequence after codon optimization with BbsI sites added to the ends.

SEQ ID NO: 35 is an exemplary nucleic acid sequence of a SARS-CoV-2 FP 5-mer sequence after codon optimization with BbsI sites added to the ends and cloned into pRIAIDA2.

SEQ ID NO: 36 is a nucleotide Sequence of pRIAIDA2.

SEQ ID NO: 37 is an amino acid sequence of a 13 amino acid subsequence surrounding the FP core sequence of SARS-CoV-2 that is identical to the corresponding sequence found in other coronaviruses, including both human and animal viruses.

SEQ ID NO: 38 is an exemplary HIV Fusion Peptide (AVGIGAVF). There are other related HIV fusion peptides, including ALGIGAAF (SEQ ID NO: 47), AVGFGAAF (SEQ ID NO: 48), and AAGFGAMF (SEQ ID NO: 49), and these can be expressed alone or in combination, as monomers, homomultimers, or heteromultimers. See Shen et al., 2020. In some embodiments, the presently disclosed subject matter provides biologically active fragments and/or homologs of these sequences.

SEQ ID NO: 39 is the nucleotide sequence of GENBANK® Accession No. MN908947.3, which corresponds to the complete genomic nucleotide sequence of the severe acute respiratory syndrome coronavirus 2 isolate Wuhan-Hu-1.

SEQ ID NO: 40 is the amino acid sequence encoded by nucleotides 21563-25384 of GENBANK® Accession No. MN908947.3, which corresponds to the complete S polypeptide sequence of the severe acute respiratory syndrome coronavirus 2 isolate Wuhan-Hu-1.

SEQ ID NO: 41 is the amino acid sequence for a PEDV antigen.

SEQ ID NO: 42 is the amino acid sequence for a SARS-CoV-2 antigen.

SEQ ID NO: 43 is the amino acid sequence of an exemplary conserved FP subsequence.

SEQ ID NO: 44 is the amino acid sequence of an exemplary influenza virus HA tag.

SEQ ID NO: 45 is the amino acid sequence of an exemplary B-cell epitope.

SEQ ID NO: 46 is an exemplary nucleic acid sequence of a PEDV FP monomer sequence after codon optimization with BbsI sites added to the ends.

SEQ ID NOs: 47-49 are the amino acid sequences of exemplary HIV Fusion Peptides that can be expressed alone or in combination, as monomers, homomultimers, or heteromultimers. In some embodiments, the presently disclosed subject matter provides biologically active fragments and/or homologs of these sequences.

### DETAILED DESCRIPTION

The presently disclosed subject matter relates in part to the observation that proteins displayed on the surfaces of genome reduced E. coli (grEC) bind antibodies better and elicit a much better immune response than when proteins are displayed on wild type E. coli. These findings suggest that a rapid synthetic biology pathway for SARS-CoV-2 and other new epidemic vaccines would involve synthesis of DNA encoding the vaccine antigen, cloning into an AT expression cassette, transformation into grEc, followed by formalin inactivation to produce an inexpensive KWC vaccine. KWC vaccines are safe, scalable, inexpensive, and auto-adjuvanting, and can be administered orally or intranasally. There has been a growing interest in producing vaccines using similar platforms (Henderson et al., 2000; Jose & Meyer, 2007). In some embodiments, the presently disclosed subject matter provides that an immune response directed against the SARS-CoV-2 spike protein fusion peptide and/or stalk will be protective.

### I. GENERAL CONSIDERATIONS

A novel coronavirus, severe acute respiratory syndrome coronavirus 2 (SARS-CoV-2), emerged in Wuhan, China in December 2019 causing novel coronavirus disease (COVID-19), with a high mortality rate, characterized by respiratory disease, and diarrhea in some patients. A vaccine for COVID-19 is urgently needed. An ideal vaccine for global pandemic use, beyond being safe and effective, would target highly conserved neutralizing epitopes, be inexpensive to manufacture, drawing on widely-available production technology, and readily adaptable for worldwide use.

Gram-negative bacterial autotransporter (ATs), are a protein family that enable bacteria to place proteins into the outer membrane (Kilpatrick et al., 1997; Shata et al., 2000; Berry et al., 2003; Ulmer et al., 2006), have 3 domains: an N-terminal signal sequence for transport across the inner membrane, a C-terminal β-barrel that inserts a pore-like structure into the outer membrane, and a central passenger protein domain that transits through the pore to be exposed extracellularly, ‘displaying’ the passenger protein to the environment. Sequence encoding a protein of interest can replace native passenger protein sequence, yielding recombinant ATs that display ˜2×10⁵ foreign proteins on each cell (Ulmer et al., 2006). The Haemophilus influenzae Hia AT, a trimeric AT, has a structure that strongly resembles the those of class I fusion protein stalks (van Bloois et al., 2011; Nicolay et al., 2015).

One of the oldest vaccine technologies is the killed whole cell vaccine (KWCV). While some KWCVs have been replaced by newer technologies, they remain important, in part because they are inexpensive and easy to manufacture. KWCVs simply require that bacteria be grown, inactivated, concentrated, and packaged. Many developing countries produce KWCVs indigenously. KWCVs are currently licensed to prevent deadly diseases, for example cholera (Bi et al., 2017). A description of how 6 million doses of WHO-prequalified Euvichol oral cholera vaccine were produced in 1 year using a single 100 L bioreactor for <$1/dose further highlights KWCV's advantages (Odevall et al., 2018). A globally useful vaccine should be very inexpensive, and quickly and easily manufactured, particularly when vaccines are urgently needed in response to pandemics.

In accordance with aspects of the presently disclosed subject matter, placing recombinant antigens on the surfaces of bacteria lacking a large number of normally present surface proteins elicit enhanced immune responses against the foreign antigen, therefore informing a new vaccine platform to enable rapid production of very inexpensive vaccines in existing facilities. The Tokyo Metropolitan University Group (Hashimoto et al., 2005; Kato & Hashimoto, 2007) made a systematic set of deletions in the E. coli genome and showed that they can delete 29.7% of the genome, yet retain a viable, albeit slow growing organism (Hashimoto et al., 2005; Kato & Hashimoto, 2007).

A large number of different SARS-CoV-2 candidate vaccines are in development (Amanat & Krammer, 2020), but there are various concerns (Corey et al., 2020). The mRNA-based vaccines recently approved are very promising, but costly to produce and have significant logistic challenges as they require −20° C. or −70° C. cold-chain transport and storage. Most SARS-CoV-2 vaccines generally target the entire S protein, tending to elicit strong responses against the immunodominant receptor binding domain (RBD). While S-targeting vaccines are attractive, since anti-S antibodies neutralize virus, reports suggest that enhanced immune responses directed against RBD may be associated with an increased risk of rare inflammatory syndromes associated with COVID-19 (Rostad et al., 2020; Yonker et al., 2020; Zeichner & Cruz, 2020), so exploring alternative subunit vaccine targets in S is warranted.

Several virus families including coronaviruses employ trimeric type I fusion proteins to bind and enter host cells (Colman & Lawrence, 2003). The HIV-1 fusion peptide (FP) has garnered considerable interest as a vaccine target (Kong et al. (2016). Several potent broadly neutralizing monoclonal antibodies recognize the HIV-1 FP (Kong et al., 2016; van Gils et al., 2016; Xu et al., 2018). For coronaviruses, proteases cleave S into S1 and S2 to activate entry. S1 recognizes and binds to its receptor, while S2 includes an FP that mediates fusion of viral and cellular membranes. Coronavirus FPs comprise 15-25 apolar amino acids that reorder the membranes after receptor binding. For SARS-CoV-2, an 18-aa sequence SFIEDLLFNKVTLADAGF (SEQ ID NO: 29) is the FP. FPs are also attractive vaccine candidates because of their minimal sequence variation across Coronaviridae family. It would likely be difficult for a virus to evolve so that it would no longer be affected by an immune response directed against the FP (van Dorp et al. (2020). For all coronaviruses across 4 genera, the FP core IEDLLF (SEQ ID NO: 37) is identical. The SARS-CoV-2 FP is one of the sites targeted by pre-existing antibodies presumably induced by non-SARS-CoV-2 infection (Ng et al.; 2020; Shrock et al., 2020). A human SARS-CoV monoclonal antibody (mAb) was well-tolerated and provided protection in passive challenge infection in a non-human primate model (Miyoshi-Akiyama et al., 2011), suggesting that a vaccine that elicited an analogous immune response would be protective. There was no evidence of antibody-dependent enhancement or vaccine-enhanced respiratory disease, suggesting that a vaccine eliciting a response exclusively against FP would be unlikely to put a patient at an increased risk.

Much work has been done on developing coronavirus animal model systems. For SARS-CoV-2, these include mice transgenic for ACE2, the viral receptor, a hamster model, and non-human primate (NHP) models (McCray, Jr. et al., 2007; Gretebeck & Subbarao, 2015; Johansen et al., 2020; Sia et al., 2020; Sun et al., 2020). However, none of these models enable the study of a coronavirus pathogen in its native host, a model where the vaccine is tested for safety and efficacy when the native host animal is exposed to the native pathogenic virus. This may be particularly important because COVID-19 includes many baffling clinical features that involve not only pathology caused directly by the virus, but also host responses triggered by the virus including coagulopathic (Zhang et al., 2020), vasculitic (Hanafi et al. (2020), neurological, and inflammatory phenomena, for example the poorly understood Multisystem Inflammatory Syndrome of Children (MIS-C; Jiang et al., 2020; Rostad et al., 2020; Yonker et al., 2020; Zeichner & Cruz, 2020).

It would be advantageous to be able to test new vaccine concepts in an animal model in which the animal could be challenged with a virus that had evolved to infect that animal. Porcine epidemic diarrhea virus (PEDV), an alphacoronavirus, causes severe diarrhea worldwide. In 2013, PEDV emerged in the United States, killing millions of pigs and causing immense economic losses to the U.S. swine industry (Huang et al., 2013; Stevenson et al., 2013; Jung & Saif, 2015). Sequence analysis revealed that the 13 amino acid residues surrounding the core FP sequence (IEDLLF; SEQ ID NO: 37) between SARS-CoV-2 and PEDV are identical, suggesting that the efficacy of a SARS-CoV-2 FP vaccine can be tested using a PEDV challenge model in pigs. Therefore, the pig and PEDV offers a powerful surrogate model to evaluate potential SARS-CoV-2 FP vaccine concepts, while providing valuable preclinical safety and immunogenicity data helpful in the human vaccine approval process. Pigs are very similar to humans in their genetics, physiology, and anatomy, perhaps the closest model to humans next to non-human primates. They have been used as model systems for many infectious disease pathogenesis and vaccine studies (reviewed in Meurens et al., 2012). While the syndromes accompanying PEDV infection in pigs largely involve diarrhea, COVID-19 can include significant gastrointestinal (GI) tract symptoms and pathology, with significant GI viral shedding (Gu et al., 2020).

Disclosed herein is a globally appropriate SARS-CoV-2 candidate vaccine. As disclosed herein, in some embodiments the presently disclosed subject matter relates to construction of recombinant bacterial live and/or killed whole cell coronavirus (e.g., SARS-CoV and/or SARS-CoV-2) vaccines by displaying the spike protein stalk on the surfaces of genome-reduced E. coli using trimeric Gram-negative autotransporters (ATs). The coronavirus S protein is a class I viral fusion protein, similar to the HIV Env, Ebola gp, and influenza HA. The S protein stalk is a structural cognate of virion env proteins (e.g. HIV membrane-proximal external region, MPER, influenza HA stalk) targeted by broadly neutralizing mAbs. AT expression cassettes can place up to 10⁵ recombinant antigens on the surface of each cell. MPER-derived proteins on the surface of E. coli using an AT bind anti-MPER monoclonal antibodies (mAbs), and mice vaccinated with an MPER-derived surface expressed vaccine produce anti-HIV neutralizing antisera. Bacteria expressing MPER peptide as a trimer on the surface of bacteria via H. influenzae Hia trimeric ATs bind anti-MPER broadly-neutralizing mAbs. Antigens expressed on the surfaces of genome-reduced E. coli (grEc) lacking a large fraction of its genome bind mAbs much better than wild-type E. coli, making the genome-reduced E. coli a potent vaccine production platform.

As disclosed herein, displaying SARS-CoV-2 stalk on the surface of grEc results in an effective vaccine against SARS-CoV-2. Multiple DNAs encoding variants of SARS-CoV-2 stalk are described, including expressing the same on the surface of grEc using a trimeric AT expression cassette. The recombinant bacteria can be inactivated by formalin to produce a killed whole cell candidate vaccine. Expression of SARS-CoV-2 stalk proteins on the surfaces of bacteria can be characterized by flow cytometry, and the amount of viral antigen in the vaccine preparations can be quantified by western blot. Also described are immunizations of mice with the vaccines and tests of the mouse sera for anti-SARS-CoV-2 neutralizing activity and T-cell responses. Vaccines yielding the best neutralization activity can then be used to inform the design of analogous PEDV FP vaccine, and for further preclinical development.

In some embodiments, the fusion peptide region of the class I fusion proteins (e.g. coronavirus spike protein, HIV envelope, influenza HA, biologically active fragments and/or homologs thereof) are targets for vaccine developed. In some embodiments the stalk regions of the class I fusion proteins are targets for vaccine development, including the influenza virus stalk or the HIV Env membrane proximal external region (MPER), or biologically active fragments and/or homologs thereof.

It is expected that a coronavirus (e.g., SARS-CoV and/or SARS-CoV-2) stalk (the membrane proximal region of the spike protein) vaccine can induce neutralizing antibodies against several and perhaps all coronaviruses.

The fusion peptides (FPs) of coronavirus spike proteins are exemplary targets for vaccine development since they are essential for virus entry. The FP is highly conserved among all coronaviruses. As disclosed herein, displaying SARS-CoV-2 FP on the surface of genome-reduced E. coli results in an effective vaccine against SARS-CoV-2. DNAs encoding the FPs of coronavirus (e.g., SARS-CoV and/or SARS-CoV-2) can be synthesized as multiple (e.g., 3, 4, 5, 6, or more) concatemers each connected by a linker (e.g., a peptide linker, optionally a glycine linker) to express FP on surfaces of genome-reduced E. coli using an AT expression cassette. Surface expression can be confirmed, for example, by flow cytometry. The recombinant bacteria can be inactivated, for example, by formalin to produce a killed whole cell SARS-CoV-2 vaccine. Finally, immunogenicity studies can be conducted to assess vaccine immunogenicity by determining the frequency of antigen-specific T cell response, neutralizing antibody, and neutralizing responses, and T-cell proliferation responses to the antigen.

Disclosed herein is a globally appropriate SARS-CoV-2 and/or PEDV candidate vaccine. As disclosed herein, in some embodiments the presently disclosed subject matter relates to construction of recombinant bacterial live and/or killed whole cell coronavirus (e.g., SARS-CoV and/or SARS-CoV-2 and/or PEDV) vaccines by displaying a SARS-CoV-2 fusion protein (FP) and/or a PEDV FP on the surfaces of genome-reduced E. coli using trimeric Gram-negative autotransporters (ATs).

As disclosed herein, displaying SARS-CoV-2 and/or PEDV stalk on the surface of grEc results in an effective vaccine against SARS-CoV-2 and/or PEDV. Multiple DNAs encoding variants of SARS-CoV-2 and/or PEDV stalk are described, including expressing the same on the surface of grEc using a trimeric AT expression cassette. The recombinant bacteria can be inactivated by formalin to produce a killed whole cell candidate vaccine. Expression of SARS-CoV-2 and/or PEDV stalk proteins on the surfaces of bacteria can be characterized by flow cytometry, and the amount of viral antigen in the vaccine preparations can be quantified by western blot. Also described are immunizations of mice with the vaccines and tests of the mouse sera for anti-SARS-CoV-2 and/or PEDV neutralizing activity and T-cell responses.

It is expected that a coronavirus (e.g., SARS-CoV and/or SARS-CoV-2 and/or PEDV) stalk (the membrane proximal region of the spike protein) vaccine can induce neutralizing antibodies against several and perhaps all coronaviruses.

The fusion peptides (FPs) of coronavirus spike proteins are exemplary targets for vaccine development since they are essential for virus entry. The FP is highly conserved among all coronaviruses. As disclosed herein, displaying SARS-CoV-2 and/or PEDV FP on the surface of genome-reduced E. coli results in an effective vaccine against SARS-CoV-2 and/or PEDV. DNAs encoding the FPs of coronavirus (e.g., SARS-CoV and/or SARS-CoV-2 and/or PEDV) can be synthesized as multiple (e.g., 3, 4, 5, 6, or more) concatemers each connected by a linker (e.g., a peptide linker, optionally a glycine linker) to express FP on surfaces of genome-reduced E. coli using an AT expression cassette. Surface expression can be confirmed, for example, by flow cytometry. The recombinant bacteria can be inactivated, for example, by formalin to produce a killed whole cell SARS-CoV-2 and/or PEDV vaccine. Finally, immunogenicity studies can be conducted to assess vaccine immunogenicity by determining the frequency of antigen-specific T cell response, neutralizing antibody, and neutralizing responses, and T-cell proliferation responses to the antigen.

As set forth herein, the coronavirus (e.g., SARS-CoV and/or SARS-CoV-2 and/or PEDV) FP vaccines can induce potent neutralizing antibodies and antigen-specific T cell responses in animals, including but not limited to humans.

Since formalin-inactivated whole cell bacterial vaccines are approved for use in humans and have a long history of success, the results disclosed herein can be quickly translated into a safe, inexpensive, scalable, and effective vaccine appropriate for pandemic response globally.

Thus, disclosed herein are in some embodiments are synthetic biology-based killed whole cell genome-reduced E. coli recombinant SARS-COV-2 and/or PEDV vaccines and vaccines targeting other class 1 fusion protein viruses via the fusion peptide and stalk region of the fusion protein.

Also disclosed herein is a new vaccine platform to efficiently express vaccine antigens on surface of genome-reduced bacteria to enhance vaccine immunogenicity. Demonstrated herein is the effectiveness of this platform to successfully express the highly conserved fusion peptide of SARS-CoV-2 and porcine epidemic diarrhea virus on surface of E. coli to produce killed whole cell bacterial vaccines. The vaccine primes a potent anamnestic response, potentiates IFN-γ response, and protects pigs against disease following virus challenge. Since the vaccine can be produced at very low cost it offers the potential for use in developing countries and may also offer a route to a broadly protective coronavirus vaccine.

### II. GENOME REDUCED BACTERIA

The presently disclosed subject matter relates to the effects on immunogenicity of expressing immunogens, such as vaccine antigens, in bacteria have a reduced or eliminated expression of genes. Thus, in some embodiments, the bacterium with fewer expressed genes is more immunogenic. By way of example and not limitation, wholesale reduction of the bacterial genome, by means of small or large scale deletions, is one way this might be accomplished. Other approaches for decreasing expression of one or more genes are contemplated to fall within the scope of the presently disclosed subject matter, such as but not limited to specific knock outs, targeted inactivations or excisions by any one of several approaches (exemplary, but not exclusively through CRISPR/Cas9, TALENS, ZFNs), knock downs, effects on promoters, conditional mutants and/or inducible mutants (for use in better growing up the bacteria that may be growth restricted by the mutations or gene inactivations, or in live attenuated bacterial vaccines. In some representative, non-limiting embodiments, genes affecting surface structures can be affected. Expression of protein structures can be affected, as can non-protein structures.

In accordance with some embodiments of the presently disclosed subject matter, the terms “genome reduced” “genome reduction” or “GR” are used interchangeably and encompasses actual deletions but also other modifications, such as inactivation, functional inactivation, and/or mutation, that reduce expression of one or more genes. In some embodiments, reducing and/or eliminating expression of genes in the bacteria yields the enhanced immunogenicity. In some embodiments, the reduced number of expressed genes comprises a reduction of at least about 1.0%, 2.0%, 3.0%, 4.0%, 5.0%, 6.0%, 7.0%, 8.0%, 9.0%, 10, 1%, 12%, 13%, 14%, 15%, or greater than 15% of genes. In some embodiments, the reduced number of expressed genes comprises a reduction of expressed genes selected from the group consisting of at least about 2.4%, at least about 15.9%, and at least about 29.7%.

In some aspects of the presently disclosed subject matter, there is a steady increase in immunogenicity as more and more genes are deleted, without a distinct “threshold effect” or notable discontinuity, which supports that beyond the effects of deleting a specific gene, there are effects due to the overall quantitative reduction in the number of genes.

Genes may be completely or partially deleted, for example by the methods employed by Hashimoto et al., 2005 and by the lambda Red systems described by Datsenko & Wanner, 2000; by CRISPR/Cas9; and other methods to delete, inactivate, or decrease expression of bacteria genes as would be apparent to one of ordinary skill in the art upon a review of the instant disclosure.

The antigen or immunogen is any antigen against which an immune response is desired. One or more such antigens can be provided by the modified bacterium. Representative, non-limiting examples of antigens include an antigen to modulate autoimmune responses, an antigen for which it might be therapeutically useful to produce an immune response, such as fibrosis associated with atherosclerosis or the amyloid plaques of Alzheimer's disease or other degenerative diseases; an antigen used induce an immune response against specific components of the immune system to modify autoimmune or allergic diseases; and/or combinations thereof.

The presently disclosed subject matter provides the following exemplary non-limiting aspects and embodiments.

Bacteria that have a reduced expression of a set of genes and that have an immunogen of interest, such as but not limited to on their surfaces, elicit an enhanced immune response against that immunogen compared to wild type, non-gene reduced bacteria.

In some embodiments, an expression vector comprising a nucleotide sequence encoding an antigen is provided. In some embodiments, the expression vector is configured to express the antigen in a modified bacterium of the presently disclosed subject matter. The presently disclosed subject matter encompasses any suitable expression vector as would be apparent to one of ordinary skill in the art upon a review of the instant disclosure. In some embodiments, the antigen is expressed on the surface of the modified bacterium. In some embodiments, the vector comprises an autotransporter (AT) expression vector. In some embodiments, the vector comprises a codon optimized sequence encoding the antigen. In some embodiments, the AT expression vector comprises a monomeric vector or a trimeric vector. In some embodiments, the nucleotide sequence encoding the antigen is positioned under control of an inducible promoter or a constitutive promoter. In some embodiments, the antigen is expressed as a monomer or as a trimer. In some embodiments, the vector is provided in a pharmaceutically acceptable carrier. Thus, one way the immunogen can be placed on the surface of the bacteria is using an autotransporter (monomeric or trimeric), by itself, or in the context of a foreign protein or scaffold to enhance/improve formation of desired immunogen. The autotransporter expression is not the only way to express antigens. Immunogens can be placed on the surface of the bacteria using other technologies, for example by covalent or non-covalent linkage, absorption, affinity tag, and the like. Using other technologies to place immunogens on the surfaces of the reduced genome bacteria provides for the production of immunogens and/or vaccines directed against proteins or other antigens that cannot be expressed on the bacterial surface using autotransporters or against non-protein antigens (such as but not limited to polysaccharides).

There are many other ways to express antigens and/or to specifically place them on the surfaces of the bacteria, or even inside the bacteria, such as but not limited to covalent coupling of the antigen to the surface of the bacteria, association of the bacteria with antigen non-covalently using an affinity tag, non-specific adsorption, addition of a binding moiety to the antigen followed by mixing the antigen with the bacteria.

The autotransporter expression cassette approach enables a synthetic biology solution: the protein antigen need not be isolated/purified/conjugated to carrier protein. Only the identity of the protein is needed. Then the coding sequence can be rapidly synthesized and cloned into the appropriate expression vector, followed by expression in the GR bacteria.

The wild type/native protein can be used, or a component of the protein can be used, if it is desirable to produce an immune response only against a particular component of the protein. A mutated version of the protein can be used, to enhance immune responses or to bias immune responses (in a non-exclusive example, humoral vs. cellular), or direct immune responses toward a particular mutant version of the gene (for example, in a cancer application).

The antigen or immunogen, used interchangeably herein, can be used to elicit an immune response against a pathogen, as in developing a prophylactic vaccine. The immunogen can be used to elicit an immune response against a pathogen, as in developing a therapeutic vaccine, for example to treat a chronic infectious disease, including chronic viral diseases. One example would be a coronavirus, for example SARS-CoV and/or SARS-CoV-2, in a coronavirus-infected patient (such as but not limited to a patient with SARS and/or COVID-19). Another example would be HIV in an HIV-infected patient. In some embodiments, the antigen or immunogen can be an immunogenic fragment of a virus stalk (S) protein and/or of an env protein, including but not limited to a sequences derived from a fusion peptide of a virus, such as but not limited to a fusion peptide of a coronavirus, for example SARS-CoV and/or SARS-CoV-2, or of HIV (such as but not limited to HIV fusion protein 1). In some embodiments, the antigen or immunogen can be an immunogenic fragment of a virus stalk (S) protein and/or of an env protein, including but not limited to a sequences derived from a fusion peptide of a virus, such as but not limited to a fusion peptide of a coronavirus, for example SARS-CoV and/or SARS-CoV-2, or of HIV (such as but not limited to HIV fusion protein 1, which in some embodiments can comprise, consist essentially of, or consist of SEQ ID NO: 38 or an immunogenic subsequence or derivative thereof). In some embodiments, the presently disclosed subject matter provides biologically active fragments and/or homologs of SEQ ID NO. 38.

The antigen or immunogen, used interchangeably herein, can be used to elicit an immune response against a pathogen, as in developing a prophylactic vaccine. The immunogen can be used to elicit an immune response against a pathogen, as in developing a therapeutic vaccine, for example to treat a chronic infectious disease, including chronic viral diseases. One example would be a coronavirus, for example SARS-CoV and/or SARS-CoV-2 and/or PEDV, in a coronavirus-infected patient (such as but not limited to a patient with SARS and/or COVID-19). In some embodiments, the antigen or immunogen can be an immunogenic fragment of a virus stalk (S) protein and/or of an env protein, including but not limited to a sequences derived from a fusion peptide of a virus, such as but not limited to a fusion peptide of a coronavirus, for example SARS-CoV and/or SARS-CoV-2 and/or PEDV, e.g., SARS-CoV-2 FP and PEDV FP. In some embodiments, the antigen or immunogen can be an immunogenic fragment of a virus stalk (S) protein and/or of an env protein, including but not limited to a sequences derived from a fusion peptide of a virus, such as but not limited to a fusion peptide of a coronavirus, for example SARS-CoV and/or SARS-CoV-2 and/or PEDV.

The immunogen can be used to elicit an immune response some other self-protein/proteins, as a way of modifying inflammatory or autoimmune diseases, for example by targeting particular cells or subsets of cells in the patient's immune system.

All of the above prophylactic and therapeutic uses can be in humans or animals. For example, the technology can be used to make veterinary prophylactic infectious disease vaccines.

The immunogen can be used to elicit the rapid production of antibodies in animals for the purposes of producing antibodies. These can be, for example, custom polyclonal antibodies, obtained directly from various species used to make custom polyclonal antibodies, such as rabbits, goats, sheep, horses, cows, and camelidae. The antibodies can be obtained from serum or from colostrum.

The immunogen can be used to immunize animals (e.g. mice, but also other species, including rabbits) to accelerate the production of monoclonal antibodies, since the first step in making a monoclonal antibody is to immunize an animal so that it makes antibodies, so that its spleen cells can be fused with myeloma cells to make a hybridoma. Such monoclonal antibodies can be used in all the analytic, diagnostic, and therapeutic ways in which monoclonal antibodies are typically used.

The genome reduced bacterial immunogen can be a killed/inactivated bacterium or a live bacterium. The bacteria can be killed/inactivated in many different ways: formalin, glutaraldehyde, heat, radiation, other chemicals. The bacteria can be whole bacteria or derivatives of whole bacteria, for example ghost cells, blebs, vesicles. The vaccine could also be fragments of the genome reduced cells. Such derivatives are prepared in accordance with techniques recognized in the art, as would be apparent to one of ordinary skill in the art up on a review of the instant disclosure.

The bacterium can be any bacterium, including Gram-negative bacteria. E. coli are not the only genome reduced bacteria that can be used. Other Gram-negative bacteria can be used, and other genome reduced strains of other bacteria can be used, such as but not limited to genome reduced Salmonella or even Vibrio. Such genome reduced versions of other bacterial species are prepared in accordance with techniques recognized in the art, as would be apparent to one of ordinary skill in the art up on a review of the instant disclosure, and then use them to express immunogens, such as vaccine antigens. Thus, in some embodiments the bacteria are from Enterobacteriaceae, such as but not limited to Salmonella, Klebsiella, Shigella, Yersinia. In some embodiments, representative bacteria can be chosen via a systematic review of the taxonomic tree; and thus, can include all Proteobacteria.

In some embodiments, a reduced genome bacterium comprises a recombinant antigen on its surface, which in some embodiments can be a coronavirus antigen, more particularly a SARS-CoV and/or SARS-CoV-2 and/or PEDV antigen, and even more particularly an antigen derived from the SARS-CoV-2 spike (S) polypeptide, which can be used to elicit useful immune responses against such an antigen. Such modified reduced genome bacteria can be used as prophylactic and/or therapeutic vaccines against SARS-CoV-2 and/or PEDV.

Thus, in some aspects and embodiments, the presently disclosed subject matter relates to strategies for the rapid production of better immunogens for the production of new vaccines, including prophylactic vaccines for infectious diseases (e.g., diseases associated with coronavirus infections, such as but not limited to SARS-CoV and/or SARS-CoV-2 and/or PEDV infections, including but not limited to COVID-19) of humans and animals. These strategies include:
1. Direct antigen production in a bacterial cell employing a synthetic biology approach in which the antigen of interest is expressed directly in the bacteria, directed by recombinant coding sequence. In some implementations, coronavirus- (e.g., SARS-CoV and/or SARS-CoV-2 and/or PEDV) derived antigens are placed on the cell surface, and in some implementations Gram-negative autotransporter protein expression cassettes are used to place the antigen-of-interest on the bacterial cell surface. Instead of purifying the protein antigen and conjugating it to carrier protein, antigen coding sequences can be cloned into an expression cassette. In the Gram-negative autotransporter embodiment discussed herein, these autotransporters (Type 5 Secretion Systems) place the antigen on the cell surface as the vaccine immunogen. This obviates any need to isolate or synthesize the protein antigen, purify the antigen, couple the antigen to an appropriate carrier, and prepare a parental immunization, saving up to several weeks.
2. Use of genome reduced bacteria (such as, but not limited to E. coli) to express the antigen. In some embodiments, the bacteria are Gram-negative bacteria, and in some embodiments the Gram-negative bacteria are E. coli. In representative embodiments, surface expressed SARS-CoV-2-derived and/or PEDV-derived antigens would be more accessible to the immune system and elicit better immune responses by expressing the antigens, such as but not limited to vaccine antigens, in genome reduced bacteria, in some embodiments on the surfaces of genome reduced bacteria, in some embodiments Gram-negative bacteria, and in one example on the surfaces of genome reduced (GR) E. coli.
3. Mucosal immunization. As a representative, non-limiting route, intranasal immunization exposes M cells and dendritic cells directly to the immunogen, and the oropharyngeal mucosa has a large amount of lymphoid tissue, which produces enhanced immune responses to intranasally administered immunogens. Disclosed herein are preliminary data indicating that combining the above strategies can yield an unexpectedly potent induction of antibody against the test antigen. However, the presently disclosed subject matter encompasses any route of administration as would be apparent to one of ordinary skill in the art upon a review of the instant disclosure, including but not limited to topical, oral, rectally, vaginally, intravenous, intramuscular, intra-arterial, intramedullary, intrathecal, intraventricular, transdermal, subcutaneous, intraperitoneal, enteral, sublingual, or in the case of a neoplasm, intratumorally.
4. Exponential increasing (exp-inc) immunization. In a representative, non-limiting embodiment, sequential, rapid exposure to increasing amounts of immunogen can yield enhanced immune responses, thought to occur because such immunogen exposure kinetics mimic the antigen exposure a host would experience in the face of a severe, poorly controlled infection, which would trigger an enhanced immune response. The immunogens discussed in this application can be used in exp-inc immunization regimens to enhance immune responses against the antigen. Conversely, the immunogens described in this application can be used in exponential decreasing dose administration patterns, or at repeated low doses to elicit a tolerizing response.

Aspects of the presently disclosed subject matter relate at least in part to the use of genome reduced bacteria to produce an antigen capable rapidly inducing an immune response against an antigen. The antigen-expressing genome reduced bacteria enable rapid antibody production for use in making custom polyclonal antibodies and materials needed (for example plasma cells) for monoclonal antibodies. The antigen-expressing genome reduced bacteria also can serve as vaccine immunogens designed to elicit immune responses that protect against infectious agents or vaccine immunogens designed to elicit a therapeutic immune response against cancers or a therapeutic immune response designed to otherwise therapeutically modulate immune responses, for example in treatment autoimmune diseases.

As set forth herein, expressing an antigen in a genome reduced bacterium can yield substantially higher binding of an antibody directed against the antigen to the bacteria and that bacteria expressing the test antigen elicit a significantly higher immune response against the test antigen when an animal is immunized with genome reduced bacteria expressing that test antigen than when immunized with wild type bacteria, and that bacteria with progressively increasing amounts of genome deletion elicited increasingly potent immune responses.

### III. ABBREVIATIONS AND ACRONYMS

Ab: Antibody
AIDA: adhesin involved in diffuse adherence
AT: autotransporter (AT)
GALT: gut associated lymphoid tissue
KWC: killed whole cell
Mab: monoclonal antibody
OM: outer membrane
PC: phosphatidyl choline
TCIU: tissue culture infectious units
TMD: transmembrane domain

### IV. DEFINITIONS

In describing and claiming the presently disclosed subject matter, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. For example, in one aspect, the term “about” is used herein to modify a numerical value above and below the stated value by a variance of 20%.

A disease or disorder is “alleviated” if the severity of a symptom of the disease, condition, or disorder, or the frequency with which such a symptom is experienced by a subject, or both, are reduced.

The terms “additional therapeutically active compound” or “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As used herein, the term “adjuvant” refers to a substance that elicits an enhanced immune response when used in combination with a specific antigen.

As use herein, the terms “administration of” and or “administering” a compound should be understood to mean providing a compound of the presently disclosed subject matter or a prodrug of a compound of the presently disclosed subject matter to a subject in need of treatment.

As used herein, the term “aerosol” refers to suspension in the air. In particular, aerosol refers to the particlization or atomization of a formulation of the presently disclosed subject matter and its suspension in the air.

As used herein, an “analog” of a chemical compound is a compound that, by way of example, resembles another in structure but is not necessarily an isomer (e.g., 5-fluorouracil is an analog of thymine).

As used herein, “amino acids” are represented by the full name thereof, by the three letter code corresponding thereto, or by the one-letter code corresponding thereto, as indicated in Table 1:

TABLE 1
Table of the Genetic Code

| | 3-Letter | 1-Letter | Functionally |
|---|---|---|---|
| Full Name | Code | Code | Equivalent Codons |
| Aspartic Acid | Asp | D | GAC GAU |
| Glutamic Acid | Glu | E | GAA GAG |
| Lysine | Lys | K | AAA AAG |
| Arginine | Arg | R | AGA AGG CGA CGC CGG CGU |
| Histidine | His | H | CAC CAU |
| Tyrosine | Tyr | Y | UAC UAU |
| Cysteine | Cys | C | UGC UGU |
| Asparagine | Asn | N | AAC AAU |
| Glutamine | Gln | Q | CAA CAG |
| Serine | Ser | S | ACG AGU UCA UCC UCG UCU |
| Threonine | Thr | T | ACA ACC ACG ACU |
| Glycine | Gly | G | GGA GGC GGG GGU |
| Alanine | Ala | A | GCA GCC GCG GCU |
| Valine | Val | V | GUA GUC GUG GUU |
| Leucine | Leu | L | UUA UUG CUA CUC CUG CUU |
| Isoleucine | Ile | I | AUA AUC AUU |
| Methionine | Met | M | AUG |
| Proline | Pro | P | CCA CCC CCG CCU |
| Phenylalanine | Phe | F | UUC UUU |
| Tryptophan | Trp | W | UGG |

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

Amino acids may be classified into seven groups on the basis of the side chain R: (1) aliphatic side chains; (2) side chains containing a hydroxylic (OH) group; (3) side chains containing sulfur atoms; (4) side chains containing an acidic or amide group; (5) side chains containing a basic group; (6) side chains containing an aromatic ring; and (7) proline, an imino acid in which the side chain is fused to the amino group.

Synthetic or non-naturally occurring amino acids refer to amino acids which do not naturally occur in vivo but which, nevertheless, can be incorporated into the peptide structures described herein. The resulting “synthetic peptide” contain amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “conservative amino acid substitution” is defined herein as exchanges within one of the following five groups:
I. Small aliphatic, nonpolar or slightly polar residues: Ala, Ser, Thr, Pro, Gly;
II. Polar, negatively charged residues and their amides: Asp, Asn, Glu, Gln;
III. Polar, positively charged residues: His, Arg, Lys;
IV. Large, aliphatic, nonpolar residues: Met Leu, Ile, Val, Cys
V. Large, aromatic residues: Phe, Tyr, Trp

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms including, for example, polyclonal antibodies, monoclonal antibodies, Fv, Fab and F(ab)₂, as well as single chain antibodies and humanized antibodies.

An “antibody heavy chain,” as used herein, refers to the larger of the two types of polypeptide chains present in all antibody molecules.

An “antibody light chain,” as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates. The term “immunogen” is used interchangeably with “antigen” herein.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “antimicrobial agents” as used herein refers to any naturally-occurring, synthetic, or semi-synthetic compound or composition or mixture thereof, which is safe for human or animal use as practiced in the methods of this presently disclosed subject matter, and is effective in killing or substantially inhibiting the growth of microbes. “Antimicrobial” as used herein, includes antibacterial, antifungal, and antiviral agents.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the peptides encompasses natural or synthetic portions of a longer peptide or protein that are capable of specific binding to their natural ligand or of performing the desired function of the protein, for example, a fragment of a protein of larger peptide which still contains the epitope of interest and is immunogenic.

The term “biological sample,” as used herein, refers to samples obtained from a subject, including, but not limited to, skin, hair, tissue, blood, plasma, cells, sweat and urine.

As used herein, the term “carrier molecule” refers to any molecule that is chemically conjugated to the antigen of interest that enables an immune response resulting in antibodies specific to the native antigen.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions.

Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position.

Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell is a cell being examined.

A “pathoindicative” cell is a cell which, when present in a tissue, is an indication that the animal in which the tissue is located (or from which the tissue was obtained) is afflicted with a disease or disorder.

A “pathogenic” cell is a cell which, when present in a tissue, causes or contributes to a disease or disorder in the animal in which the tissue is located (or from which the tissue was obtained).

A tissue “normally comprises” a cell if one or more of the cell are present in the tissue in an animal not afflicted with a disease or disorder.

As used herein, a “derivative” of a bacterium, antigen, composition or other compound refers to a bacterium, antigen, composition or other compound that may be produced from bacterium, antigen, composition or other compound of similar structure in one or more steps.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, the term “diagnosis” refers to detecting a risk or propensity to an addictive related disease disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes.

Most antigens have many epitopes; i.e., they are multivalent. In general, an epitope is roughly at least five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

The terms “fragment” and “segment” are used interchangeably herein.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993. This algorithm is incorporated into the NBLAST and XBLAST programs of Altschul et al., 1990a, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Altschul et al., 1997) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

By the term “immunizing a subject against an antigen” is meant administering to the subject a composition, a protein complex, a DNA encoding a protein complex, an antibody or a DNA encoding an antibody, which elicits an immune response in the subject, and, for example, provides protection to the subject against a disease caused by the antigen or which prevents the function of the antigen.

The term “immunologically active fragments thereof” will generally be understood in the art to refer to a fragment of a polypeptide antigen comprising at least an epitope, which means that the fragment at least comprises 4 contiguous amino acids from the sequence of the polypeptide antigen.

As used herein, the term “inhaler” refers both to devices for nasal and pulmonary administration of a drug, e.g., in solution, powder and the like. For example, the term “inhaler” is intended to encompass a propellant driven inhaler, such as is used to administer antihistamine for acute asthma attacks, and plastic spray bottles, such as are used to administer decongestants.

The term “inhibit,” as used herein when referring to a function, refers to the ability of a compound of the presently disclosed subject matter to reduce or impede a described function. In some embodiments, inhibition is by at least 10%, in some embodiments by at least 25%, in some embodiments by at least 50%, and in some embodiments, the function is inhibited by at least 75%. When the term “inhibit” is used more generally, such as “inhibit Factor I”, it refers to inhibiting expression, levels, and activity of Factor I.

The term “inhibit a complex,” as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibit a protein,” as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting, or applying, or administering” includes administration of a compound of the presently disclosed subject matter by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, or rectal approaches.

As used herein, an “instructional material” includes a publication, a recording, a diagram, or any other medium of expression which can be used to communicate the usefulness of the peptide of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container which contains the identified compound presently disclosed subject matter or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to, through ionic or hydrogen bonds or van der Waals interactions.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels

The term “nasal administration” in all its grammatical forms refers to administration of at least one compound of the presently disclosed subject matter through the nasal mucous membrane to the bloodstream for systemic delivery of at least one compound of the presently disclosed subject matter. The advantages of nasal administration for delivery are that it does not require injection using a syringe and needle, it avoids necrosis that can accompany intramuscular administration of drugs, trans-mucosal administration of a drug is highly amenable to self administration, and intranasal administration of antigens exposes the antigen to a mucosal compartment rich in surrounding lymphoid tissues, which can promote the development of a more potent immune response, particularly more potent mucosal immune responses.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the presently disclosed subject matter.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides but when used in the context of a longer amino acid sequence can also refer to a longer polypeptide.

The term “per application” as used herein refers to administration of a drug or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

By “presensitization” is meant pre-administration of at least one innate immune system stimulator prior to challenge with an agent. This is sometimes referred to as induction of tolerance.

The term “prevent,” as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of contracting the disease and/or developing a pathology associated with the disease.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (Gerhardt et al., 1994).

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

By the term “specifically binds to”, as used herein, is meant when a compound or ligand functions in a binding reaction or assay conditions which is determinative of the presence of the compound in a sample of heterogeneous compounds.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, preferably a human.

As used herein, a “subject in need thereof” is a patient, animal, mammal, or human, who will benefit from the method of this presently disclosed subject matter.

As used herein, “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is in some embodiments at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C. with washing in 1×SSC, 0.1% SDS at 50° C.; preferably 7% SDS, 0.5 M NaPO₄, 1 mM EDTA at 50° C. with washing in 0.5×SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990b; Altschul et al., 1990a; Altschul et al., 1997). The default settings provided with these programs are suitable for determining substantial similarity of nucleic acid sequences for purposes of the presently disclosed subject matter.

The term “substantially pure” describes a compound, e.g., a protein or polypeptide which has been separated from components which naturally accompany it. Typically, a compound is substantially pure when it is in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term to “treat,” as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

By the term “vaccine,” as used herein, is meant a composition which when inoculated into a subject has the effect of stimulating an immune response in the subject, which serves to fully or partially treat and/or protect the subject against a condition, disease or its symptoms. In one aspect, the condition is HIV. TB is another application as are parasitic diseases. The term vaccine encompasses prophylactic as well as therapeutic vaccines. A combination vaccine is one which combines two or more vaccines, or two or more compounds or agents.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### V. REPRESENTATIVE COMPOSITIONS AND METHODS

In some embodiments, a pharmaceutical composition comprising one or more components of the presently disclosed subject matter is administered orally. In one aspect, it is administered intra-nasally, rectally, vaginally, parenterally, employing intradermal, subcutaneously, or intramuscularly. In some embodiments, the pharmaceutical composition is a vaccine.

The system can also be used to express other viral proteins on the surface of bacteria to be used for immunization or treatment directed against the other viral proteins.

The presently disclosed subject matter provides a series of proteins or peptides and systems to produce or express those peptides in the context of cell structures, such as a lipid bilayer and other membrane structures found to have immunogenic activity that can be used singly or in combination to elicit an immunogenic response and are useful for preventing and treating viral infections (such as infections with coronaviruses, including but not limited to SARS-CoV and/or SARS-CoV-2 and/or PEDV, e.g. SARS-CoV-2 FP and/or PEDV FP). The presently disclosed subject matter could also be used to produce immunizing antigens targeting the conserved regions of other coronavirus virion envelope proteins for, for example, a universal coronavirus vaccine.

In some embodiments, the presently disclosed subject matter provides a modified bacterium expressing a set of peptides that can be used together as a cocktail or individually as a component of a vaccine (immunogen) to prevent or to treat any condition, disease, and/or disorder as described herein. When administered, the bacterium comprising the cocktail or combination of peptides elicits an immunogenic response. The presently disclosed subject matter further encompasses the use of biologically active homologues of the peptides and wells as biologically active fragments of the peptides. The homologues can, for example, comprise one of more conservative amino acid substitutions, additions, or deletions.

In some embodiments, the presently disclosed subject matter provides an immunogenic vaccine composition for use in treating and preventing infections, such as but not limited to coronavirus infections including but not limited to SARS-CoV and/or SARS-CoV-2 infections and/or PEDV infections. Other examples include HIV infections. In some aspects, the composition comprises at least one isolated peptide selected from the group of peptides disclosed herein, or biologically active fragments or homologs thereof. In some aspects, the immunogenic vaccine composition is a system comprising a viral peptide provided by a bacterium in accordance with the presently disclosed subject matter. The vaccine composition can also include an adjuvant or a pharmaceutically acceptable carrier. In one aspect, at least two peptides are included in the composition. Any combination of the peptides can be used.

In some embodiments, an immunogenic fragment or homolog of a peptide of the presently disclosed subject matter is used. In some embodiments, the biologically active fragments or homologs of the peptide share at least about 50% sequence identity with the peptide. In some aspects, they share at least about 75% sequence identity with the peptide. In yet other aspects, they share at least about 95% sequence identity with the peptide. Exemplary peptides that can be employed include peptides that can be modified and still give rise to an anti-coronavirus (e.g., anti-SARS-CoV and/or anti-SARS-CoV-2 and/or PEDV) immune response, and such sequences are also encompassed within the presently disclosed subject matter. Exemplary peptides that can be employed include peptides that comprise, consist essentially of, or consist of and/or are encoded by any of SEQ ID NOs: 4-35 and 38 or any subsequence thereof, which in some embodiments can be encoded by any of SEQ ID NOs: 4-15 and 32-35 and/or are any of SEQ ID NOs: 16-31. It is noted that the sequences represented by SEQ ID NOs: 4-15 and 32-35 can be modified and still give rise to an anti-coronavirus (e.g., anti-SARS-CoV and/or anti-SARS-CoV-2) immune response, and such sequences are also encompassed within the presently disclosed subject matter. Exemplary peptides that can be employed include peptides that comprise, consist essentially of, or consist of

(SEQ ID NO: 31)
PSKPSKRSFIEDLLFNKVTLADAGF,
(SEQ ID NO: 42)
PSKPSKRSFIEDLLFNVKTLADAG,
(SEQ ID NO: 43)
SFIEDLLF,
(SEQ ID NO: 29)
SFIEDLLFNKVTLADAGF,
and
(SEQ ID NO: 41)
GRVVQKRSFIEDLLFNKVVINGLG.

In some embodiments, at least one of the active fragments or homologs being used comprises a serine or alanine amino acid substitution for a cysteine residue. In some embodiments, at least one of the active fragments or homologs being used comprises at least one conservative amino acid substitution. The presently disclosed subject matter encompasses the use of amino acid substitutions at any of the positions, as long as the resulting peptide maintains the desired biologic activity of being immunogenic. The presently disclosed subject matter further includes the peptides where amino acids have been deleted or inserted, as long as the resulting peptide maintains the desired biologic activity of being immunogenic.

In some embodiments, the methods of the presently disclosed subject matter provide for administering the vaccine composition to a subject at least about 2 times to about 50 times. In some embodiments, the method comprises administering the vaccine composition to a subject at least about 5 times to about 30 times. In some embodiments, the methods of the presently disclosed subject matter provide for administering the vaccine composition to a subject at least about 10 times to about 20 times. The method also provides for administering the composition daily, or weekly, or monthly. One of ordinary skill in the art can design a regimen based on the needs of a subject, taking into account the age, sex, and health of the subject.

As described herein, the peptides provided by the modified bacterium are immunogenic, so a useful composition comprising one or more of the peptides of the presently disclosed subject matter, even when using active fragments or homologs, or additionally short peptides, elicits an immunogenic response.

In some aspects, a homolog of a peptide of the presently disclosed subject matter is one with one or more amino acid substitutions, deletions, or additions, and with the sequence identities described herein. In some aspects, the substitution, deletion, or addition is conservative. In some aspects, a serine or an alanine is substituted for a cysteine residue in a peptide of the presently disclosed subject matter.

In some embodiments, the subject is a mammal. In another embodiment, the mammal is a human.

The presently disclosed subject matter encompasses the use of purified isolated, recombinant, and synthetic peptides.

The presently disclosed subject matter further provides methods for producing peptides which are not easily soluble in an aqueous solution, by immediately expressing the peptides on the surface of the bacteria.

The methods and compositions of the presently disclosed subject matter encompass multiple regimens and dosages for administering the peptides of the presently disclosed subject matter for use in preventing and treating diseases and disorders caused by infectious agents. For example, a subject can be administered a combination of peptides, such as a combination of peptides provided by a bacterium, or a combination of bacteria expressing different peptides, of the presently disclosed subject matter once or more than once. The frequency and number of doses can vary based on many parameters, including the age, sex, and health of the subject. In some embodiments, up to 50 doses are administered. In some embodiments, up to 40 doses are administered, and in another up to 30 doses are administered. In some embodiments, up to 20 doses are administered, and in another up to 10 doses are administered. In some embodiments, 5-10 doses are administered. In some embodiments, 5, 6, 7, 8, 9, or 10 doses can be administered.

In some embodiments, bacteria expressing a peptide or bacteria expressing two or more peptides are administered more than once daily, in another daily, in another on alternating days, in another weekly, and in another, monthly. Treatment periods may be for a few days, or about a week, or about several weeks, or for several months. Follow-up administration or boosters can be used as well and the timing of that can be varied.

The amount of bacteria expressing a peptide or derivative of the bacteria administered per dose can vary as well. For example, in some embodiments, the compositions and methods of the presently disclosed subject matter include a range of peptide amounts (for example as provided by bacteria expressing a peptide) between about 1 nanogram of each peptide per dose to about 10 milligrams of immunogen per dose. In some embodiments, the number of micrograms is the same for each peptide. In some embodiments, the number of micrograms is not the same for each peptide. In some embodiments, the range of amounts of each immunogen administered per dose is from about 1 nanogram to about 10 milligrams.

Subjects can be monitored before and after bacteria administration for antibody levels against the immunogens being administered (for example as provided by bacteria expressing a peptide) and by monitoring T cell responses, including CD4+ and CD8+. Methods for these tests are routinely used in the art and are either described herein or, for example, in publications cited herein.

Although a vaccine composition construct, bacteria, mixture of bacteria, derivatives thereof, or cocktail of peptides or a combination therefor is described herein, when more than one bacterial construct or peptide is administered, each different bacterial construct or peptide can be administered separately. When a vaccine composition is administered more than once to a subject, the dose of each bacterial construct or peptide may vary per administration.

To increase the immunological response, various adjuvants may be used depending on the host species, including but not limited to cholera toxin B subunit, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as cholera toxin B subunit, alum, saponins, nucleic acids, LPS, BCG (bacille Calmette-Guerin) and Corynebacterium parvum.

If peptides are to be placed on the genome reduced bacteria following exogenous production and not by protein synthesis by the bacteria themselves, those peptides for use in the presently disclosed subject matter may be readily prepared by standard, well-established techniques, such as solid-phase peptide synthesis (SPPS) as described by Stewart et al., 1984 and as described by Bodanszky & Bodanszky, 1984. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl-blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high-resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide. Prior to its use, the peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄-, C₈-, or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

It will be appreciated, of course, that the peptides or antibodies, derivatives, or fragments thereof may incorporate amino acid residues which are modified without affecting activity. For example, the termini may be derivatized to include blocking groups, i.e. chemical substituents suitable to protect and/or stabilize the N- and C-termini from “undesirable degradation,” a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the presently disclosed subject matter are also contemplated, for example, inverted peptides in which all amino acids are substituted with D-amino acid forms.

Acid addition salts of the presently disclosed subject matter are also contemplated as functional equivalents. Thus, a peptide in accordance with the presently disclosed subject matter treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the presently disclosed subject matter, for example a GR bacteria with attached additional immunogens.

The presently disclosed subject matter also provides for homologs of proteins and peptides for use in accordance with the presently disclosed subject matter. Homologs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. To that end, 10 or more conservative amino acid changes typically have no effect on protein function.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides or antibody fragments which have been modified using ordinary molecular biological techniques so as to improve their resistance to proteolytic degradation or to optimize solubility properties or to render them more suitable as a therapeutic agent. Homologs of such polypeptides include those containing residues other than naturally occurring L-amino acids, e.g., D-amino acids or non-naturally occurring synthetic amino acids. The peptides of the presently disclosed subject matter are not limited to products of any of the specific exemplary processes listed herein.

Substantially pure protein or peptide obtained as described herein may be purified by following known procedures for protein purification, wherein an immunological, enzymatic, or other assay is used to monitor purification at each stage in the procedure. Protein purification methods are well known in the art, and are described, for example in Deutscher et al., 1990.

One of ordinary skill in the art will appreciate that when more than one peptide is used (for example as provided by a bacterium expressing two or more peptides or by different bacteria expressing different peptides or derivative of the bacterium) that they do not necessarily have to be administered in the same pharmaceutical composition at the same time, and that multiple administrations can also be used. When multiple injections are used they can be administered, for example, in a short sequence such as one right after the other or they can be spaced out over predetermined periods of time, such as every 5 minutes, every 10 minutes, every 30 minutes, etc. Of course, administration can also be performed by administering a pharmaceutical comprising all components to be administered, such as a cocktail comprising bacteria expressing a peptide or derivative of the bacteria of the presently disclosed subject matter. It can also be appreciated that a treatment regimen may include more than one round of injections, spaced over time such as weeks or months, and can be altered according to the effectiveness of the treatment on the particular subject being treated.

The presently disclosed subject matter provides multiple methods of using specifically prepared bacteria expressing a peptide or derivative of the bacteria, for example, in fresh or lyophilized liposome, proper routes of administration of the bacteria or derivative thereof, proper doses of the bacteria or derivative thereof, and specific combinations of heterologous immunization including priming in one administration route followed by liposome-mediated antigen boost in a different route to tailor the immune responses in respects of enhancing cell mediated immune response, cytokine secretion, humoral immune response, especially skewing T helper responses to be Th1 or a balanced Th1 and Th2 type. For more detail, see U.S. patent application Ser. No. 11/572,453 (published as U.S. Patent Application Publication No. 2008/0193469, which is now U.S. Pat. No. 8,012,932 and each of which is incorporated herein by reference in its entirety), which claims priority to PCT International Patent Application Serial No. PCT/US2005/026102 (published as PCT International Patent Application Publication No. WO 2006/012539 and incorporated herein by reference in its entirety).

A homolog herein is understood to comprise an immunogenic peptide having in some embodiments at least 70%, in some embodiments at least 80%, in some embodiments at least 90%, in some embodiments at least 95%, in some embodiments at least 98%, and in some embodiments at least 99% amino acid sequence identity with the peptides mentioned above and is still capable of eliciting at least the immune response obtainable thereby. A homolog or analog may herein comprise substitutions, insertions, deletions, additional N- or C-terminal amino acids, and/or additional chemical moieties, such as carbohydrates, to increase stability, solubility, and immunogenicity.

In one embodiment of the presently disclosed subject matter, the present immunogenic polypeptides as defined herein, are glycosylated. Without wishing to be bound by any particular theory, it is hypothesized herein that by glycosylation of these polypeptides the immunogenicity thereof may be increased. Therefore, in one embodiment, the aforementioned immunogenic polypeptide as defined herein before, is glycosylated, having a carbohydrate content varying from 10-80 wt %, based on the total weight of the glycoprotein or glycosylated polypeptide. Said carbohydrate content ranges can be from 15-70 wt %, or from 20-60 wt %. In another embodiment, said glycosylated immunogenic polypeptide comprises a glycosylation pattern that is similar to that of the peptides of the human that is treated. It is hypothesized that this even further increases the immunogenicity of said polypeptide. Thus, in one embodiment, the immunogenic polypeptide comprises a glycosylation pattern that is similar to that of the corresponding glycoprotein.

In one embodiment, the source of a peptide comprises an effective amount of at least one immunogenic peptide selected from the peptides described herein, and immunologically active homologs thereof and fragments thereof, or a nucleic acid sequence encoding said immunogenic peptide.

In one embodiment, the present method of immunization comprises the administration of a source of immunogenically active peptide fragments, said peptide fragments being selected from the peptide fragments and/or homologs thereof as defined herein before.

Peptides may advantageously be chemically synthesized and may optionally be (partially) overlapping and/or may also be ligated to other molecules, peptides, or proteins. Peptides may also be fused to form synthetic proteins, as in Welters et al., 2004. It may also be advantageous to add to the amino- or carboxy-terminus of the peptide chemical moieties or additional (modified or D-) amino acids in order to increase the stability and/or decrease the biodegradability of the peptide. To improve immunogenicity, immuno-stimulating moieties may be attached, e.g. by lipidation or glycosylation. To enhance the solubility of the peptide, addition of charged or polar amino acids may be used, in order to enhance solubility and increase stability in vivo.

For immunization purposes, the aforementioned immunogenic peptides for use with the presently disclosed subject matter may also be fused with proteins, such as, but not limited to, tetanus toxin/toxoid, diphtheria toxin/toxoid or other carrier molecules. The polypeptides according to the presently disclosed subject matter may also be advantageously fused to heatshock proteins, such as recombinant endogenous (murine) gp96 (GRP94) as a carrier for immunodominant peptides as described in (see e.g., Rapp & Kaufmann, 2004; Zugel, 2001), or fusion proteins with Hsp70 (PCT International Patent Application Publication No. WO 1999/54464).

The individual amino acid residues of the present immunogenic (poly)peptides for use with the presently disclosed subject matter can be incorporated in the peptide by a peptide bond or peptide bond mimetic. A peptide bond mimetic of the presently disclosed subject matter includes peptide backbone modifications well known to those skilled in the art. Such modifications include modifications of the amide nitrogen, the alpha carbon, amide carbonyl, complete replacement of the amide bond, extensions, deletions, or backbone cross-links. See generally, Spatola, 1983. Several peptide backbone modifications are known and can be used in the practice of the presently disclosed subject matter.

Amino acid mimetics may also be incorporated in the polypeptides. An “amino acid mimetic” as used here is a moiety other than a naturally occurring amino acid that conformationally and functionally serves as a substitute for an amino acid in a polypeptide of the presently disclosed subject matter. Such a moiety serves as a substitute for an amino acid residue if it does not interfere with the ability of the peptide to elicit an immune response. Amino acid mimetics may include non-protein amino acids. A number of suitable amino acid mimetics are known to the skilled artisan, they include cyclohexylalanine, 3-cyclohexylpropionic acid, L-adamantyl alanine, adamantylacetic acid and the like. Peptide mimetics suitable for peptides of the presently disclosed subject matter are discussed by Morgan & Gainor, 1989.

In some embodiments, the present method comprises the administration of a composition (e.g, bacteria or derivative thereof) comprising one or more of the present immunogenic peptides as defined herein above, and at least one excipient. Excipients are well known in the art of pharmacy and may for instance be found in textbooks such as Remington's Pharmaceutical Sciences, 18th ed. (1990).

The present method for immunization may further comprise the administration, and in one aspect, the co-administration, of at least one adjuvant. Adjuvants may comprise any adjuvant known in the art of vaccination or composition for eliciting an immune response and may be selected using textbooks like Colligan et al., 1994-2004.

Adjuvants are herein intended to include any substance or compound that, when used, in combination with an antigen, to immunize a human or an animal, stimulates the immune system, thereby provoking, enhancing or facilitating the immune response against the antigen, preferably without generating a specific immune response to the adjuvant itself. In one aspect, adjuvants can enhance the immune response against a given antigen by at least a factor of 1.5, 2, 2.5, 5, 10, or 20, as compared to the immune response generated against the antigen under the same conditions but in the absence of the adjuvant. Tests for determining the statistical average enhancement of the immune response against a given antigen as produced by an adjuvant in a group of animals or humans over a corresponding control group are available in the art. The adjuvant preferably is capable of enhancing the immune response against at least two different antigens. The adjuvant of the presently disclosed subject matter will usually be a compound that is foreign to a human, thereby excluding immunostimulatory compounds that are endogenous to humans, such as e.g. interleukins, interferons, and other hormones.

A number of adjuvants are well known to one of ordinary skill in the art. Suitable adjuvants include, e.g., incomplete Freund's adjuvant, alum, aluminum phosphate, aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxy-phosphoryloxy)-ethylamine (CGP 19835A, referred to as MTP-PE), DDA (2 dimethyldioctadecylammonium bromide), polyIC, Poly-A-poly-U, RIB™, GERBU™, PAM3™, CARBOPOL™, SPECOL™, TITERMAX™, tetanus toxoid, diphtheria toxoid, meningococcal outer membrane proteins, cholera toxin B subunit, diphtheria protein CRM197. Preferred adjuvants comprise a ligand that is recognized by a Toll-like-receptor (TLR) present on antigen presenting cells. Various ligands recognized by TLR's are known in the art and include e.g. lipopeptides (see e.g., PCT International Patent Application Publication No. WO 2004/110486), lipopolysaccharides, peptidoglycans, liopteichoic acids, lipoarabinomannans, lipoproteins (from Mycoplasma or spirochetes), double-stranded RNA (poly I:C), unmethylated DNA, flagellin, CpG-containing DNA, and imidazoquinolines, as well derivatives of these ligands having chemical modifications.

In some embodiments of the present methods, one or more bacteria expressing a peptide or derivative of the bacteria are typically administered at a dosage of about 1 ug/kg patient body weight or more at least once. Often dosages are greater than 10 μg/kg. According to the presently disclosed subject matter, the dosages range in some embodiments from 1 μg/kg to 1 mg/kg.

In some embodiments typical dosage regimens comprise administering a dosage of in some embodiments 1-1000 ug/kg, in some embodiments 10-500 μg/kg, in some embodiments 10-150 μg/kg, once, twice, or three times a week for a period of one, two, three, four or five weeks. According to some embodiments, 10-100 μg/kg is administered once a week for a period of one or two weeks.

The presently disclosed methods, in some aspects, comprise administration of bacteria expressing a peptide or derivative of the bacteria and compositions comprising them via the injection, transdermal, intranasal, or oral route. In some aspects of the presently disclosed subject matter, the present method comprises vaginal or rectal administration of the present bacteria expressing a peptide or derivative of the bacteria and compositions comprising them.

Another aspect of the presently disclosed subject matter relates to a pharmaceutical preparation comprising as the active ingredient the present source of a polypeptide as defined herein before. More particularly pharmaceutical preparation comprises as the active ingredient one or more of the aforementioned immunogenic peptides, homologs thereof and fragments of said peptides and homologs thereof, as provided by a bacteria expressing a peptide or derivative of the bacteria as defined herein above.

The presently disclosed subject matter further provides a pharmaceutical preparation comprising one or more bacteria expressing a peptide or derivative of the bacteria of the presently disclosed subject matter. The concentration of said peptides in the pharmaceutical composition can vary widely, i.e., from less than about 0.1% by weight, usually being at least about 1% by weight to as much as 20% by weight or more.

The composition may comprise a pharmaceutically acceptable carrier in addition to the active ingredient. The pharmaceutical carrier can be any compatible, non-toxic substance suitable to deliver the immunogenic peptide or bacteria expressing a peptide or derivative of the bacteria to the patient. For polypeptides, sterile water, alcohol, fats, waxes, and inert solids may be used as the carrier. Pharmaceutically acceptable adjuvants, buffering agents, dispersing agents, and the like, may also be incorporated into the pharmaceutical compositions.

In some embodiments, the present bacteria expressing a peptide or derivative of the bacteria are administered by injection. The parenteral route for administration is in accordance with known methods, e.g., injection or infusion by intravenous, intraperitoneal, intramuscular, intra-arterial, subcutaneous, rectal, vaginal, or intralesional routes. The bacteria expressing a peptide or derivative of the bacteria may be administered continuously by infusion or by bolus injection. In some embodiments, a composition for intravenous infusion could be made up to contain 10 to 50 ml of sterile 0.9% NaCl or 5% glucose optionally supplemented with a 20% albumin solution and in some embodiments between 10 μg and 50 mg, in some embodiments between 50 μg and 10 mg, of the bacteria expressing a peptide or derivative of the bacteria. A typical pharmaceutical composition for intramuscular injection would be made up to contain, for example, 1-10 ml of sterile buffered water and in some embodiments between 10 μg and 50 mg, in some embodiments between 50 ug and 10 mg, of the bacteria expressing a peptide or derivative of the bacteria of the presently disclosed subject matter. Methods for preparing parenterally administrable compositions are well known in the art and described in more detail in various sources, including, for example, Remington's Pharmaceutical Sciences, 18th ed., 1990, incorporated by reference in its entirety for all purposes).

For convenience, immune responses are often described in the presently disclosed subject matter as being either “primary” or “secondary” immune responses. A primary immune response, which is also described as a “protective” immune response, refers to an immune response produced in an individual as a result of some initial exposure (e.g., the initial “immunization”) to a particular antigen. Such an immunization can occur, for example, as the result of some natural exposure to the antigen (for example, from initial infection by some pathogen that exhibits or presents the antigen). Alternatively, the immunization can occur because of vaccinating the individual with a vaccine containing the antigen. For example, the vaccine can be a vaccine comprising one or more antigenic epitopes or fragments of the peptides of the presently disclosed subject matter.

In certain embodiments, the disclosed methods and compositions may involve preparing peptides with one or more substituted amino acid residues. In various embodiments, the structural, physical and/or therapeutic characteristics of peptide sequences may be optimized by replacing one or more amino acid residues.

In some embodiments, the presently disclosed subject matter encompasses the substitution of a serine or an alanine residue for a cysteine residue in a peptide of the presently disclosed subject matter. Support for this includes what is known in the art. For example, see the following citation for justification of such a serine or alanine substitution: Kittlesen et al., 1998.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the presently disclosed subject matter are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art. For example, one can make the following isosteric and/or conservative amino acid changes in the parent polypeptide sequence with the expectation that the resulting polypeptides would have a similar or improved profile of the properties described above:
Substitution of alkyl-substituted hydrophobic amino acids: including alanine, leucine, isoleucine, valine, norleucine, S-2-aminobutyric acid, S-cyclohexylalanine or other simple alpha-amino acids substituted by an aliphatic side chain from C₁-C₁₀ carbons including branched, cyclic and straight chain alkyl, alkenyl or alkynyl substitutions.
Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-, 3- or 4-aminophenylalanine, 2-, 3- or 4-chlorophenylalanine, 2-, 3- or 4-methylphenylalanine, 2-, 3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2, 3, or 4-biphenylalanine, 2′,-3′,- or 4′-methyl-2, 3 or 4-biphenylalanine, and 2- or 3-pyridylalanine.
Substitution of amino acids containing basic functions: including arginine, lysine, histidine, ornithine, 2,3-diaminopropionic acid, homoarginine, alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.
Substitution of acidic amino acids: including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.
Substitution of side chain amide residues: including asparagine, glutamine, and alkyl or aromatic substituted derivatives of asparagine or glutamine.
Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 is preferred, within +/−1 are more preferred, and within +/−0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see e.g., Chou & Fasman, 1974; Chou & Fasman, 1978; Chou & Fasman, 1979).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, val; Arg (R) gln, asn, lys; Asn (N) his, asp, lys, arg, gln; Asp (D) asn, glu; Cys (C) ala, ser; Gln (Q) glu, asn; Glu (E) gln, asp; Gly (G) ala; His (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y) trp, phe, thr, ser; Val (V) ile, leu, met, phe, ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp (see e.g., the PROWL Rockefeller University website). For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr (see e.g., the PROWL Rockefeller University website). Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (see e.g., the PROWL Rockefeller University website).

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

The presently disclosed subject matter is also directed to methods of administering the compounds of the presently disclosed subject matter to a subject.

Pharmaceutical compositions comprising the present compositions are administered to an individual in need thereof by any number of routes including, but not limited to, topical, oral, rectally, vaginally, intravenous, intramuscular, intra-arterial, intramedullary, intrathecal, intraventricular, transdermal, subcutaneous, intraperitoneal, intranasal, enteral, topical, sublingual, or rectal means.

The presently disclosed subject matter is also directed to pharmaceutical compositions comprising the bacteria of the presently disclosed subject matter. More particularly, such compounds can be formulated as pharmaceutical compositions using standard pharmaceutically acceptable carriers, fillers, solubilizing agents and stabilizers known to those skilled in the art.

The presently disclosed subject matter also encompasses the use pharmaceutical compositions of an appropriate compound, homolog, fragment, analog, or derivative thereof to practice the methods of the presently disclosed subject matter, the composition comprising at least one appropriate compound, homolog, fragment, analog, or derivative thereof and a pharmaceutically-acceptable carrier.

The pharmaceutical compositions useful for practicing the presently disclosed subject matter may be administered to deliver a dose of between 1 ng/kg/day and 100 mg/kg/day. Pharmaceutical compositions that are useful in the methods of the presently disclosed subject matter may be administered systemically in oral solid formulations, ophthalmic, suppository, aerosol, topical or other similar formulations. In addition to the appropriate compound, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate compound according to the methods of the presently disclosed subject matter.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

Pharmaceutical compositions that are useful in the methods of the presently disclosed subject matter may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the presently disclosed subject matter may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology. A formulation of a pharmaceutical composition of the presently disclosed subject matter suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the presently disclosed subject matter which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively).

Known emulsifying agents include, but are not limited to, lecithin and acacia. Known preservatives include, but are not limited to, methyl, ethyl, or n-propyl para hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the presently disclosed subject matter may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the presently disclosed subject matter may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the presently disclosed subject matter may also be prepared, packaged, or sold in the form of oil in water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the presently disclosed subject matter may also be prepared, packaged, or sold in a formulation suitable for rectal administration, vaginal administration, parenteral administration

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example.

Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi liquid preparations such as liniments, lotions, oil in water or water in oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the presently disclosed subject matter may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder dispensing container such as a device comprising the active ingredient dissolved or suspended in a low-boiling propellant in a sealed container.

In some embodiments, such powders comprise particles wherein at least 98% of the particles by weight have a diameter greater than 0.5 nanometers and at least 95% of the particles by number have a diameter less than 7 nanometers. In some embodiments, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the presently disclosed subject matter formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the presently disclosed subject matter.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the presently disclosed subject matter may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Remington's Pharmaceutical Sciences, 18th ed., 1990, which is incorporated herein by reference.

Typically, dosages of the composition of the presently disclosed subject matter which may be administered to an animal, preferably a human, range in amount from 1 μg to about 100 g per kilogram of body weight of the subject. While the precise dosage administered will vary depending upon any number of factors, including but not limited to, the type of animal and type of disease state being treated, the age of the animal and the route of administration. In one embodiment, the dosage of the compound will vary from about 10 μg to about 10 g per kilogram of body weight of the animal. In another embodiment, the dosage will vary from about 10 mg to about 1 g per kilogram of body weight of the subject.

The composition may be administered to a subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the sex and age of the subject, etc.

The presently disclosed subject matter further provides kits comprising bacteria expressing a peptide or derivative of the bacteria of the presently disclosed subject matter useful for eliciting an immunogenic response, and further includes an applicator and an instructional material for the use thereof.

### V. OTHER EMBODIMENTS

In some embodiments, the presently disclosed subject matter also provides other systems by which antigens and/or immunogens of interest can be expressed in, on the surface of, or otherwise by bacteria. Thus, it is understood that the autotransporter expression system described herein is not the only way to express antigens. There are many other ways to express antigens and to specifically place them on the surfaces of the bacteria, or even inside the bacteria.

Similarly, the presently disclosed subject matter also provides modified bacteria other than modified E. coli. By way of example and not limitation, other Gram-negative bacteria can also be employed, and other genome reduced strains of other bacteria could also be used. Examples of other bacteria that could be employed include genome reduced Salmonella or Vibrio. As such, one of ordinary skill in the art could employ the present disclosure as a guide to construct genome reduced versions of other bacterial species for use to express vaccine antigens.

In some embodiments of the presently disclosed subject matter, the modified bacteria can be inactivated. Various methods and approaches for inactivating bacteria for use in immunizations are known to those of skill in the art, and include without limitation use of formalin and/or glutaraldehyde.

Additionally, after review of the instant disclosure one of ordinary skill in the art would also recognize that the purpose of the modified bacterium is primarily to provide a structure in which to provide the immunogen of interest to the immune system of the subject to be immunized. As such, the modified bacterium need not be a fully functional bacterium capable of living, reproducing, etc. As such, in addition to modifications that reduce the genomes of the bacteria, other bacterial derivatives can also be employed. Such derivatives include, but are not limited to ghost cells, bacterial fragments of cells, including but not limited to isolated outer membrane fragments, blebs, etc.

Furthermore, whereas in some embodiments the presently disclosed subject matter relates to the rapid production of antibodies, the presently disclosed subject matter also relates in some embodiments to the production of prophylactic vaccines for infectious diseases and/or therapeutic vaccines for infectious diseases (such as but not limited to chronic infectious diseases like HIV, other chronic viral diseases, TB, and/or parasitic diseases), therapeutic vaccines for cancer (e.g., off the shelf vaccines directed at know cancer antigens) and custom vaccines designed based on the analysis of the cancer neoantigens for a given patient's cancer (i.e., a personalized anti-cancer vaccine), and therapeutic vaccines for other diseases, particularly diseases involving inflammatory processes, like autoimmune diseases, fibrosis, atherosclerosis, etc.

### Examples

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Example 1

### Construction of Genome Reduced Bacteria

Strategies for producing Genome Reduced (GR) bacteria and for incorporating constructs encoding an antigen of interest, such those described in the EXAMPLES below, including EXAMPLE 5.

The production and characterization of custom antibodies made against a protein of interest is a slow process that can take several months from the time a purified protein antigen linked to a carrier protein becomes available to produce a custom polyclonal custom antibody. It can take a further several weeks or more to prepare the immunogen. The development of a new, faster method to produce custom antibodies against a protein of interest would have great benefit for essentially all biomedical research.

Representative Approach: Immunization with killed whole cell GR E. coli presented intranasally via exp-inc immunization schedules will dramatically decrease the time needed to produced effective custom antibodies. This EXAMPLE relates at least in part (1) to optimizing genome reduced bacterial immunization for rapid antibody production in mice and (2) to demonstrating rapid production of polyclonal antibodies in rabbits using optimized immunization procedures.

The contributions of the modifications to immunization described above on immune responses directed against a test antigen are systematically tested. With an optimized new immunization protocol in hand, the ability of that protocol to elicit production of immune responses against a test antigen in rabbits is confirmed, comparing the new procedures to conventional immunization procedures. A significant reduction in the time to produce custom antibodies would greatly aid progress in essentially all areas of biomedical research.

Gram-negative autotransporters. Gram-Autotransporter (AT) (also termed Autodisplay or Type 5 Secretion System) proteins are a protein family that mediates protein placement into Gram-bacterial outer membranes, with one region anchored in the membrane lipid bilayer and another exposed to the extracellular environment (Henderson et al., 2000; Jose & Meyer, 2007; van Bloois et al., 2011; Nicolay et al., 2015). AT proteins have 3 key domains: An N-terminal signal sequence that directs protein across the inner membrane via a secA mechanism, a C-terminal β-barrel that inserts into the Gram-OM, yielding a pore-like structure, and a central passenger protein domain that transits through the 0-barrel pore to be exposed extracellularly, attached to the 0-barrel, which remains anchored in the OM. Native passenger protein coding sequence can be replaced with sequence encoding another protein, yielding a recombinant AT protein. The AT thus ‘displays’ recombinant passenger protein to the extracellular environment, anchored in and closely adjacent to the OM lipid bilayer. About 2×10⁵ recombinant proteins can be placed on each cell's surface (Jose & Meyer, 2007). This non-limiting representative approach can be employed in some aspects of the presently disclosed subject matfer.

Genome reduced E. coli To better understand how the number of gene products on the surface of the E. coli strains changed in the TMUG GR strains, the names of the genes in each deletion (LD5510, LD5119, and LD5125) from the National BioResource Project Ecoh Strain website created by the National Institute of Genetics, Japan (https://shigen.nig.ac.jp/ecoli/strain/resource/longDeletion/lddTablelnfo) were compiled, with additional gene information from EcoCyc (https://ecocyc.org/), and UniProt (https://www.uniprot.org/), including gene name, protein name, location, function, gene ontology, and other notes about the gene. The information was organized into tables and sorted by deletion. A number of bacterial gene products with an imputed location on the exterior of the cell were eliminated in the GR E. coli. A large number of surface gene products are eliminated in the GR E. coli strains.

An exemplary list of genes that can be deleted from E. coli is presented in Table 2.

TABLE 2
Genes That Were Deleted In The Three Genome Reduced E. Coli Strains*

| | Deleted in | Deleted in | Deleted in | | | | | |
|---|---|---|---|---|---|---|---|---|
| Gene | LD5510 | LD5119 | LD5125 | Outer | Integral to | | | Cell |
| Name | (2.4%) | (15.9%) | (29.7%) | Memb¹ | Memb¹ | Memb¹ | Pil² | Surf³ |
| acrZ | 0 | 0 | 1 | 1 | 0 | 0 | 0 | 0 |
| ActP | 0 | 1 | 1 | 0 | 1 | 0 | 0 | 0 |
| AdiC | 0 | 1 | 1 | 0 | 1 | 0 | 0 | 0 |
| aer | 0 | 1 | 1 | 0 | 1 | 0 | 0 | 0 |
| AlsC | 0 | 1 | 1 | 0 | 1 | 0 | 0 | 0 |
| alx | 0 | 1 | 1 | 0 | 1 | 1 | 0 | 0 |
| amiD | 0 | 0 | 1 | 1 | 0 | 0 | 0 | 0 |
| arsB | 0 | 1 | 1 | 0 | 1 | 0 | 0 | 0 |
| artI | 0 | 0 | 1 | 0 | 0 | 1 | 0 | 0 |
| artJ | 0 | 0 | 1 | 0 | 0 | 1 | 0 | 0 |
| basS | 0 | 1 | 1 | 0 | 1 | 0 | 0 | 0 |
| bcsB | 0 | 1 | 1 | 0 | 1 | 1 | 0 | 0 |
| bcsC | 0 | 1 | 1 | 1 | 0 | 0 | 0 | 0 |
| cadB | 0 | 1 | 1 | 0 | 1 | 0 | 0 | 0 |
| clsC | 0 | 1 | 1 | 0 | 0 | 1 | 0 | 0 |
| copA | 0 | 1 | 1 | 0 | 1 | 0 | 0 | 0 |
| csgA | 0 | 1 | 1 | 0 | 0 | 0 | 1 | 0 |
| csgB | 0 | 1 | 1 | 1 | 0 | 0 | 1 | 0 |
| csgE | 0 | 1 | 1 | 1 | 0 | 0 | 0 | 0 |
| csgF | 0 | 1 | 1 | 1 | 0 | 0 | 0 | 0 |
| csgG | 0 | 1 | 1 | 1 | 0 | 0 | 0 | 0 |
| dctA | 0 | 1 | 1 | 0 | 1 | 0 | 0 | 0 |
| dcuB | 0 | 1 | 1 | 0 | 1 | 0 | 0 | 0 |
| dcuS | 0 | 1 | 1 | 0 | 1 | 1 | 0 | 0 |
| dinQ | 0 | 1 | 1 | 0 | 1 | 1 | 0 | 0 |
| dppB | 0 | 1 | 1 | 0 | 1 | 0 | 0 | 0 |
| dppC | 0 | 1 | 1 | 0 | 1 | 0 | 0 | 0 |
| dtpB | 0 | 1 | 1 | 0 | 1 | 0 | 0 | 0 |
| ecpA | 0 | 0 | 1 | 0 | 0 | 0 | 1 | 0 |
| ecpC | 0 | 0 | 1 | 1 | 0 | 0 | 0 | 0 |
| ecpD | 0 | 0 | 1 | 0 | 0 | 0 | 1 | 0 |
| eptA | 0 | 1 | 1 | 0 | 1 | 1 | 0 | 0 |
| eptB | 0 | 1 | 1 | 0 | 1 | 1 | 0 | 0 |
| exuT | 0 | 1 | 1 | 0 | 1 | 1 | 0 | 0 |
| fdnG | 0 | 0 | 1 | 0 | 0 | 1 | 0 | 0 |
| fdrA | 0 | 1 | 1 | 0 | 1 | 0 | 0 | 0 |
| fecA | 1 | 1 | 1 | 0 | 0 | 1 | 0 | 0 |
| fecE | 1 | 1 | 1 | 0 | 0 | 1 | 0 | 0 |
| fimA | 1 | 1 | 1 | 1 | 0 | 0 | 0 | 0 |
| fimD | 1 | 1 | 1 | 1 | 0 | 0 | 0 | 0 |
| fimF | 1 | 1 | 1 | 1 | 0 | 0 | 0 | 0 |
| fimG | 1 | 1 | 1 | 1 | 0 | 0 | 0 | 0 |
| fimH | 1 | 1 | 1 | 1 | 0 | 0 | 0 | 0 |
| fimI | 1 | 1 | 1 | 0 | 0 | 0 | 1 | 0 |
| fiu | 0 | 0 | 1 | 1 | 0 | 0 | 0 | 0 |
| Fsr | 0 | 1 | 1 | 0 | 1 | 0 | 0 | 0 |
| gadB | 0 | 0 | 1 | 0 | 0 | 1 | 0 | 0 |
| gfcD | 0 | 1 | 1 | 1 | 0 | 0 | 0 | 0 |
| gfcE | 0 | 1 | 1 | 1 | 0 | 0 | 0 | 0 |
| glnH | 0 | 0 | 1 | 0 | 0 | 1 | 0 | 0 |
| gltP | 0 | 1 | 1 | 0 | 1 | 0 | 0 | 0 |
| gltP | 0 | 1 | 1 | 0 | 1 | 0 | 0 | 0 |
| gntP | 1 | 1 | 1 | 0 | 1 | 0 | 0 | 0 |
| gutQ | 0 | 0 | 1 | 0 | 0 | 1 | 0 | 0 |
| hdeD | 0 | 1 | 1 | 0 | 1 | 1 | 0 | 0 |
| hslJ | 0 | 0 | 1 | 0 | 0 | 1 | 0 | 0 |
| insH1 | 0 | 1 | 1 | 1 | 0 | 0 | 0 | 0 |
| mdtF | 0 | 1 | 1 | 1 | 0 | 0 | 0 | 0 |
| mdtM | 1 | 1 | 1 | 0 | 1 | 0 | 0 | 0 |
| mdtN | 0 | 1 | 1 | 0 | 1 | 0 | 0 | 0 |
| mdtN | 0 | 1 | 1 | 0 | 1 | 0 | 0 | 0 |
| mdtO | 0 | 1 | 1 | 0 | 1 | 1 | 0 | 0 |
| mdtO | 0 | 1 | 1 | 0 | 1 | 1 | 0 | 0 |
| mdtP | 0 | 1 | 1 | 1 | 0 | 0 | 0 | 0 |
| mdtQ | 0 | 1 | 1 | 1 | 0 | 0 | 0 | 0 |
| melB | 0 | 1 | 1 | 0 | 1 | 0 | 0 | 0 |
| mltB | 0 | 0 | 1 | 1 | 0 | 0 | 0 | 0 |
| nanC | 1 | 1 | 1 | 1 | 1 | 0 | 0 | 0 |
| nrfD | 0 | 1 | 1 | 0 | 1 | 0 | 0 | 0 |
| nrfE | 0 | 1 | 1 | 0 | 1 | 0 | 0 | 0 |
| nrfE | 0 | 1 | 1 | 0 | 1 | 0 | 0 | 0 |
| ompC | 0 | 0 | 1 | 1 | 0 | 0 | 0 | 0 |
| ompG | 0 | 0 | 1 | 1 | 0 | 0 | 0 | 0 |
| ompN | 0 | 0 | 1 | 1 | 0 | 0 | 0 | 0 |
| ompX | 0 | 0 | 1 | 1 | 0 | 0 | 0 | 0 |
| opgB | 1 | 1 | 1 | 0 | 0 | 1 | 0 | 0 |
| pgaA | 0 | 1 | 1 | 1 | 0 | 0 | 0 | 0 |
| pgaB | 0 | 1 | 1 | 1 | 0 | 0 | 0 | 0 |
| phnC | 0 | 1 | 1 | 0 | 0 | 1 | 0 | 0 |
| phnE | 0 | 1 | 1 | 0 | 1 | 1 | 0 | 0 |
| pitA | 0 | 1 | 1 | 0 | 1 | 0 | 0 | 0 |
| prop | 0 | 1 | 1 | 0 | 1 | 0 | 0 | 0 |
| qmcA | 0 | 1 | 1 | 0 | 1 | 1 | 0 | 0 |
| rhsA | 0 | 1 | 1 | 0 | 1 | 1 | 0 | 0 |
| rhsB | 0 | 1 | 1 | 0 | 1 | 1 | 0 | 0 |
| rhsD | 0 | 1 | 1 | 0 | 1 | 1 | 0 | 0 |
| rzoR | 0 | 0 | 1 | 1 | 0 | 0 | 0 | 0 |
| slp | 0 | 1 | 1 | 1 | 0 | 0 | 0 | 0 |
| sstT | 0 | 1 | 1 | 0 | 1 | 1 | 0 | 0 |
| tdcC | 0 | 1 | 1 | 0 | 1 | 0 | 0 | 0 |
| Tsr | 1 | 1 | 1 | 0 | 0 | 1 | 0 | 0 |
| uspB | 0 | 1 | 1 | 0 | 1 | 0 | 0 | 0 |
| wecH | 0 | 1 | 1 | 0 | 1 | 1 | 0 | 0 |
| wza | 0 | 1 | 1 | 1 | 0 | 0 | 0 | 0 |
| xylH | 0 | 1 | 1 | 0 | 1 | 0 | 0 | 0 |
| yaiO | 0 | 0 | 1 | 1 | 0 | 0 | 0 | 0 |
| ybaL | 0 | 1 | 1 | 0 | 1 | 0 | 0 | 0 |
| ybaT | 0 | 1 | 1 | 0 | 1 | 0 | 0 | 0 |
| ybbD | 0 | 1 | 1 | 0 | 1 | 1 | 0 | 0 |
| ybbJ | 0 | 1 | 1 | 0 | 1 | 0 | 0 | 0 |
| ybbM | 0 | 1 | 1 | 0 | 1 | 0 | 0 | 0 |
| ybbP | 0 | 1 | 1 | 0 | 1 | 1 | 0 | 0 |
| ybbW | 0 | 1 | 1 | 0 | 1 | 0 | 0 | 0 |
| ybbY | 0 | 1 | 1 | 0 | 1 | 1 | 0 | 0 |
| ybhC | 0 | 0 | 1 | 1 | 0 | 0 | 0 | 0 |
| ybjC | 0 | 0 | 1 | 0 | 0 | 1 | 0 | 0 |
| yceK | 0 | 1 | 1 | 1 | 0 | 0 | 0 | 0 |
| yddB | 0 | 0 | 1 | 1 | 0 | 0 | 0 | 0 |
| yddL | 0 | 0 | 1 | 1 | 0 | 0 | 0 | 0 |
| ydeQ | 0 | 0 | 1 | 0 | 0 | 0 | 1 | 0 |
| ydeR | 0 | 0 | 1 | 0 | 0 | 0 | 1 | 0 |
| ydeS | 0 | 0 | 1 | 0 | 0 | 0 | 1 | 0 |
| ydeT | 0 | 0 | 1 | 1 | 0 | 0 | 0 | 0 |
| yehA | 0 | 1 | 1 | 0 | 0 | 0 | 1 | 0 |
| yehB | 0 | 1 | 1 | 1 | 0 | 0 | 0 | 0 |
| yehD | 0 | 1 | 1 | 0 | 0 | 0 | 1 | 0 |
| yehR | 0 | 1 | 1 | 0 | 0 | 1 | 0 | 0 |
| yehU | 0 | 1 | 1 | 0 | 1 | 1 | 0 | 0 |
| yehW | 0 | 1 | 1 | 0 | 1 | 0 | 0 | 0 |
| yehY | 0 | 1 | 1 | 0 | 1 | 0 | 0 | 0 |
| yejO | 0 | 0 | 1 | 1 | 0 | 0 | 0 | 0 |
| yfaZ | 0 | 0 | 1 | 1 | 0 | 0 | 0 | 0 |
| yfjV | 0 | 1 | 1 | 0 | 1 | 0 | 0 | 0 |
| ygjI | 0 | 1 | 1 | 0 | 1 | 1 | 0 | 0 |
| ygjQ | 0 | 1 | 1 | 0 | 1 | 1 | 0 | 0 |
| ygjR | 1 | 1 | 1 | 0 | 0 | 0 | 0 | 1 |
| ygjV | 0 | 1 | 1 | 0 | 1 | 1 | 0 | 0 |
| yhaH | 0 | 1 | 1 | 0 | 1 | 1 | 0 | 0 |
| yhaI | 0 | 1 | 1 | 0 | 1 | 1 | 0 | 0 |
| yhaO | 0 | 1 | 1 | 0 | 1 | 0 | 0 | 0 |
| yhhJ | 0 | 1 | 1 | 0 | 1 | 0 | 0 | 0 |
| yhiD | 0 | 1 | 1 | 0 | 1 | 1 | 0 | 0 |
| yhiM | 0 | 1 | 1 | 0 | 1 | 1 | 0 | 0 |
| yhjD | 0 | 1 | 1 | 0 | 1 | 1 | 0 | 0 |
| yhjE | 0 | 1 | 1 | 0 | 1 | 0 | 0 | 0 |
| yhjG | 0 | 1 | 1 | 0 | 1 | 1 | 0 | 0 |
| yhjK | 0 | 1 | 1 | 0 | 0 | 1 | 0 | 0 |
| yhjV | 0 | 1 | 1 | 0 | 1 | 0 | 0 | 0 |
| yhjX | 0 | 1 | 1 | 0 | 1 | 0 | 0 | 0 |
| yhjY | 0 | 1 | 1 | 0 | 0 | 1 | 0 | 0 |
| yiaA | 0 | 1 | 1 | 0 | 1 | 1 | 0 | 0 |
| yiaB | 0 | 1 | 1 | 0 | 1 | 1 | 0 | 0 |
| yiaD | 0 | 1 | 1 | 0 | 1 | 0 | 0 | 0 |
| yiaM | 0 | 1 | 1 | 0 | 1 | 1 | 0 | 0 |
| yiaN | 0 | 1 | 1 | 0 | 1 | 0 | 0 | 0 |
| yiaT | 0 | 1 | 1 | 1 | 0 | 0 | 0 | 0 |
| yiaV | 0 | 1 | 1 | 0 | 1 | 0 | 0 | 0 |
| yiaW | 0 | 1 | 1 | 0 | 1 | 0 | 0 | 0 |
| yibH | 0 | 1 | 1 | 0 | 1 | 0 | 0 | 0 |
| yibI | 0 | 1 | 1 | 0 | 0 | 1 | 0 | 0 |
| yjcE | 0 | 1 | 1 | 0 | 1 | 0 | 0 | 0 |
| yjcH | 0 | 1 | 1 | 0 | 1 | 1 | 0 | 0 |
| yjdF | 0 | 1 | 1 | 0 | 1 | 1 | 0 | 0 |
| yjdL | 0 | 1 | 1 | 0 | 1 | 0 | 0 | 0 |
| yjdO | 0 | 1 | 1 | 0 | 1 | 1 | 0 | 0 |
| yjhE | 1 | 1 | 1 | 0 | 0 | 1 | 0 | 0 |
| yjhP | 1 | 1 | 1 | 0 | 1 | 0 | 0 | 0 |
| yjiG | 1 | 1 | 1 | 0 | 1 | 0 | 0 | 0 |
| yjiH | 1 | 1 | 1 | 0 | 1 | 0 | 0 | 0 |
| yjiJ | 1 | 1 | 1 | 0 | 1 | 0 | 0 | 0 |
| yjiK | 1 | 1 | 1 | 1 | 1 | 0 | 0 | 0 |
| yjiN | 1 | 1 | 1 | 0 | 1 | 0 | 0 | 0 |
| yjiN | 1 | 1 | 1 | 0 | 0 | 1 | 0 | 0 |
| yjiY | 1 | 1 | 1 | 0 | 0 | 1 | 0 | 0 |
| yjjL | 1 | 1 | 1 | 0 | 0 | 1 | 0 | 0 |
| yliI | 0 | 0 | 1 | 1 | 0 | 0 | 0 | 0 |
| ynbC | 0 | 0 | 1 | 0 | 0 | 1 | 0 | 0 |
| ynbC | 0 | 0 | 1 | 0 | 0 | 1 | 0 | 0 |
| ynbC | 0 | 0 | 1 | 0 | 0 | 1 | 0 | 0 |
| yncD | 0 | 0 | 1 | 1 | 0 | 0 | 0 | 0 |
| yohC | 0 | 1 | 1 | 0 | 1 | 0 | 0 | 0 |
| yohO | 0 | 1 | 1 | 0 | 1 | 1 | 0 | 0 |
| ypjA | 0 | 1 | 1 | 1 | 0 | 0 | 0 | 0 |
| yqjA | 0 | 1 | 1 | 0 | 1 | 0 | 0 | 0 |
| yqjE | 0 | 1 | 1 | 0 | 1 | 1 | 0 | 0 |
| yqjF | 0 | 1 | 1 | 0 | 1 | 1 | 0 | 0 |
| yqjI | 0 | 1 | 1 | 0 | 0 | 0 | 1 | 0 |
| ytcA | 0 | 1 | 1 | 0 | 1 | 1 | 0 | 0 |
| ytcA | 0 | 1 | 1 | 0 | 1 | 1 | 0 | 0 |
| ythA | 1 | 1 | 1 | 0 | 0 | 1 | 0 | 0 |

*Genes are listed by locus name, with the presence (=1) or absence (=0) for each deleted mutant strain and for each location in the bacteria.
¹membrane;
²pilus;
³cell surface

Production of Gram-AT recombinant expression systems for rapid Ab production immunizations. In preliminary experiments, plasmid pRIAIDA, which has a rhamnose inducible AIDA-I Gram-AT expression cassette for expression optimization, with a cloning site, flanked by a trypsin site to evaluate surface expression was constructed. In initial experiments, a nucleic acid sequence encoding a widely-used influenza virus HA tag (YPYDVPDYA, SEQ ID NO: 44) was inserted into the surface expression cassette to make pRIAIDA-HA. A trypsination experiment confirmed that that HA immunotag resided on the exterior of the bacteria when expressed in an AIDA-I expression cassette that includes a trypsin site in the coding sequence.

Intranasal immunization. Intranasal immunization has a number of significant advantages (Davis, 2001; Jabbal-Gill, 2010; Zaman et al., 2013; Riese et al., 2014; Nizard et al., 2017; Yusuf & Kent, 2017). Intranasal immunization leads to the direct exposure in the nasal mucosa of M cells and dendritic cells to the immunogen. In addition, there are abundant nasopharyngeal lymphoid tissues with large numbers of other antigen presenting cells, like macrophages, and many T cells and B cells. Intranasal immunization can induce potent tissue-resident effector and effector memory CD8+ T cell immunity (Morabito et al., 2017). Intranasal immunization may also be particularly effective in producing antibodies in other body compartments if, for example, secretory mucosal antibodies may be useful (Russell, 2002). In addition, since most pathogens enter the host across mucosal surfaces, if the hypothesis that the best way to rapidly elicit potent antibody response is to mimic a significant pathogen threat, mucosal immunization can elicit better immune responses than a more traditional parenteral route.

Evaluation of antibody binding to the GR E. coli expressing a test immunogen on their surfaces and the ability of the GR E. coli expressing a test immunogen on their surfaces to elicit immune responses. As an initial step for a new method employing a combination of strategies to enhance immunogenicity for the production of custom Abs, pRIAIDA-HA was transformed into wt parental E. coli and three GR E. coli from the TMUG collection. Binding of a commercial anti-HA mAb to the bacteria was confirmed via flow cytometry. It was determined that the ability of the bacteria to bind the anti-HA mAb increased significantly as the fraction of the genome deleted increased. The ability of the GR E. coli to elicit an immune response was tested. Mice were immunized intranasally with 10⁸ formalin fixed bacteria. After 2 weeks, blood was collected from the immunized mice and the sera tested using an ELISA, with commercial anti-HA mAb as a standard. It was found that intranasal immunization with GR E. coli expressing the test immunogen on their surfaces could elicit the production of large amounts of Abs in only 2 weeks, and that the ability of the bacteria expressing the test antigen to elicit production of the Ab increased substantially in bacteria with more genes deleted. Preliminary data using immuno-dot blots indicated that the same amount of HA was produced per cell regardless of extent of the genome reduction, which supported the hypothesis that the increased binding and immunogenicity was due to either increased antigen accessibility in the GR strains, or an absence of immunoinhibitory surface structures or both.

Exponentially increasing (exp-inc) immunization schedules. Recent studies comparing alternative immunization schedules have shown that repeated immunizations with exp-inc amounts of immunogens can yield a dramatically improved immune response, with a >10-fold increase in Ab concentrations (Tam et al., 2016). The hypothesized reason for the enhanced response is that the exp-inc antigen dosing schedule creates signals to the host immune system similar to those initiated by serious infections that threaten the host, with the enhanced response promoted by prolonged, greater amounts of antigen present in lymph nodes acting to improve antibody maturation. Exp-inc immunization has also been observed to induce more Tfh cells and germinal center B-cells. This non-limiting representative approach can be employed in some aspects of the presently disclosed subject matter.

Scientific Premise. The immune system responds to threatening pathogens by rapidly producing a potent humoral immune response. The presently disclosed subject matter provides new methods that enable the rapid production of useful polyclonal Abs that can be employed in wide range of biomedical research projects. It has been demonstrated that a substantial antibody response against a test antigen can be produced in only two weeks. EXAMPLE 1 is aimed at extending and optimizing the work, and demonstrating that these new procedures can produce a rapid, useful polyclonal Ab response in rabbits, the typical species employed in producing useful amounts of polyclonal Abs for biomedical research. Table 3 summarizes the innovations that are employed in the work, along with the different rationales for their use.

The preliminary data showed that the production of antigen via gene synthesis in a Gram-AT expression vector in GR E. coli with intranasal immunization can elicit a strong Ab response in 2 weeks.

TABLE 3
Summary Of Features Employed And Proposed
In The New Rapid Ab Production Method

| Feature | Comments |
|---|---|
| Antigen production | Rapid immunogen production; Increased immunogenicity |
| using synthetic biology | through production of tandem multimeric antigens; |
| | Increased immunogenicity through amino acid |
| | sequence modifications, Inherent fusion to carrier |
| | protein |
| Surface expression | Direct interaction with B-Cell receptors plus |
| via autotransporter | processing/presentation by APCs; Auto-adjuvanting |
| (Type 5 secretion system) | through presence of multiple PAMPs on bacteria |
| expression cassette | |
| Genome reduced E. coli | Fewer bacterial proteins interfering with vaccine antigen- |
| | immune system interactions; enhanced exposure of |
| | the immunizing antigen; loss of components masking |
| | bacterial surface antigens from the immune system |
| Intranasal immunization | Direct access to mucosal compartment rich in dendritic |
| | cells and APCs; Needle-free immunization: |
| | Induction of mucosal immunity |
| Exponential increasing | Mimics signals to the immune system that the immunogen |
| immunogen dose | comes from a serious pathogen threat |

### Research Methods

Overview. The presently disclosed procedure rapidly produces custom polyclonal Abs. It also indicates the key areas of additional optimization that are pursued to further enhance the ability of the new procedure to rapidly produce custom polyclonal Abs. Please also note that even though preliminary work strongly supports that intranasal immunization with GR E. coli expresses an antigen-of-interest on its surfaces using a Gram-AT expression cassette, additional research can be performed to enable effective, rapid production of custom Abs. This work includes defining the kinetics of the Ab response beyond 2 weeks, a careful analysis of the Ab subtypes to maximize the utility of the custom Abs produced using the new procedure, and work to determine whether induction of the Ab response could be further enhanced. To make the new procedure broadly useful for biomedical research, that the procedure can elicit production of custom Abs in a species large enough to make useful quantities of custom Abs, such as rabbit, is also demonstrated.

### 1. Optimize Genome Reduced Bacterial Immunization for Rapid Antibody Production in Mice.

A series of methods are tested to further optimize rapid production of Abs in mice, then test the most effective of those methods in rabbits, confirming the utility of the new technology to rapidly produce custom Abs useful for research.

Design of expression cassettes, surface expression and bacterial toxicity testing. An E. coli codon-optimized version of the HA immunotag coding sequence was cloned into pRIAIDA, transforming the derivative, into wt non-deleted and GR (2.4%, 15.9%, 29.7%) E. coli (Hashimoto et al., 2005; Kato & Hashimoto, 2007), verifying expression by flow cytometry and surface expression by immunoblot with and without trypsin treatment. Since HA is short, highly immunogenic immunotag, a second, larger immunotag is also used to verify that the proposed new Ab production procedure is effective. The ability of the procedure to elicit production of Abs against another model immunogen is also tested. For this second immunogen GFP (MW 27 kDa) is chosen, which is useful for confirming surface expression on the bacteria with IF and flow cytometry. GFP has also been used as a test immunogen in many studies, including studies that showed that E. coli-derived outer membrane vesicles engineered to contain GFP elicited better immune responses than GFP alone (Chen et al., 2010). In addition, excellent, well-tested reagents, both recombinant protein and mAbs, are commercially available for GFP (e.g Abcam, SigmaAldrich, ThermoFisher). The AIDA-I AT has been shown to transport GFP and GFP fusions to bacterial surfaces (Li et al., 2008). For the GFP (and any other alternate immunogen constructs), optimum rhamnose induction of expression is determined, following bacterial growth by OD600 to determine the maximum expression possible without compromising bacterial replication. The number of immunogen molecules on the surface of each bacteria is determined using immunoblots on bacterial extracts and immunogen protein standards. A goal is 2×10⁵ molecules/bacteria, which has been achieved with other immunogens, and which has been achieved with other antigens, including HA. GFP was also chosen as a widely used marker in biological research that has no clinical use.

Production of immunogens—whole GR E. coli. The methods will be those that yielded the preliminary data. Bacteria are grown in LB broth with optimized rhamnose induction, monitoring growth by OD600. Bacteria are collected by centrifugation, washed in HBSS, without Ca²⁺/Mg²⁺, 10% formalin is added to a final 0.2% concentration, and incubated at 37 C for 1 hour with shaking. Aliquots are stored at −80 C in PBS/10% glycerol, confirming immunogen expression by flow cytometry on thawed stocks. Surface expression was confirmed using a trypsinization procedure either with immunoblots, or flow cytometry (e.g. for GFP).

Evaluating immunogenicity—humoral immunity. A sandwich ELISA was constructed by binding commercial anti-HA mAb (Invitrogen) to blocked, streptavidin-coated strips (Pierce), followed by incubation with commercially produced HA peptide, followed by commercial anti-HA mAb (Invitrogen) with HRP-conjugated goat anti-mouse secondary antibody, assayed using the Tropix CSPD luminescence system. In those experiments, it was found that anti-HA Abs could be detected to the 1 ng/ml level, below the physiologic level of antibodies against vaccine antigens following vaccination (Huang et al., 2012).

For the ELISAs on the sera from the immunized animals, a sandwich ELISA is employed. To develop assays, commercially available HA and GFP proteins were employed as standards. IgG subclasses (IgG1, IgG2a, IgG(Total)), IgA, and IgM are quantitated with HRP-conjugated anti-mouse class and subclass mAbs, with the 1-Step slow TMB-ELISA substrate, or as an alternative, the Tropix CSPD luminescence system, although it is not believed that the added sensitivity of the chemiluminescent assay will be required.

Evaluating immunogenicity—cell-mediated immunity. While the deliverable for this project is an improved method to rapidly produce custom Abs, it may be helpful in evaluating, comparing, and optimizing the different procedures to have information on the cell mediated immune responses elicited by the different immunization procedures. To characterize the cell-mediated immune responses, ELISpot assays are performed according to the kit manufacturer's instructions (Mabtech). Spleen cells (5×10⁶ cells/mL) obtained at the conclusion of the experiment will be plated and stimulated in the presence (or not) of HA (or GFP peptide mix) and incubated for 24 hours. Plates are washed and incubated with biotinylated detection antibody, then incubated with streptavidin-ALP, followed by substrate solution (BCIP/NBT-plus).

In addition to ELIspot assays, assessment of antigen specific T-cell frequency, proliferative capacity, cell surface immunophenotyping, and intracellular cytokine production profiles are determined by flow cytometry to characterize T cell responses. Cells (Maecker et al., 2005) challenged with immunogens are evaluated with a polychromatic (12 color) flow cytometric panel to determine frequencies of HA positive cells, and characterize T_{EM} or T_{CM} phenotypes, since T_{CM} cells vs. T_{EM} cells (Seder & Ahmed, 2003; Klebanoff et al., 2005; Klebanoff et al., 2006), which might imply the induction of longer-term immunity, might be helpful for additional boosting and distinguishing between different immunization strategies. For defining T_{EM} and T_{CM} cells, various markers recommended by the Human Immunology Project (Maecker et al., 2012) are employed including CD3, CD4, CD8, CCR7, CD45RA. To assess functionality of the response, intracellular cytokine production and proliferative capacity are measured, and intracellular cytokine staining for IL2, IFN-γ, and TNF-α is performed. Proliferation is evaluated by intracellular staining for Ki-67, which is expressed in cells in S, G2, and M phases, but not G₀ or G₁ (Gerdes et al., 1984). Fixable amine reactive viability dye is used to eliminate evaluation of dead cells. Non-T cells are identified with a cocktail including anti-CD19, CD14, CD16, CD56, CD11c and CD11b Abs. At least 100,000 events are acquired on a BD 4 laser 17 color FORTESSA™ flow cytometer. Data are analyzed using FlowJo10 (Treestar) software with this gating strategy: 1) Gate for single cells using a FSC area vs. FSC height plot, 2) Gate for live T cells using a CD3 vs. Viability/dump channel (lineage cocktail) plot, 3) Gate for antigen specific CD8+ T cells using CD8 vs. Pentamer plot, 4) Phenotype T_{EM} and T_{CM} using CCR7 vs. CD45RA plot, and 5) Evaluate intracellular cytokine profiles and Ki-67 positivity within the T_{EM} and T_{CM} cell populations.

Immunizations—immunization methods. Immediately before immunization, KWC preparations are thawed on ice and wash in PBS. For the single (non exp-inc) intranasal immunizations, 6 week old mice (CB6F1/J, Jackson) mice are immunized intranasally with 10′ cells in 50 ul PBS, boosting at 2 weeks.

Later, immunizations done at a single time with 10⁸ cells are compared with an exponential dose escalation, using doses of 10⁸, 3×10⁸, and 10⁹ cells on alternating days.

For the experiments with the GR E. coli, since the aim of the project is to develop an accelerated immunization schedule, a rapid schedule with an initial prime and a boost after 2 weeks is employed. Sera is collected at baseline, before immunization, 2 weeks after immunization, before the boost, and 2 weeks after the boost, at which time the mice are euthanized and larger blood volumes are collected by terminal bleed, along with spleens for the isolation of spleen mononuclear cells for the assays for cell mediated immunity (see below).

As controls (see below for more details on the head-to-head comparisons), mice are immunized SC with the same doses of bacteria used in the intranasal experiments, and with commercially purchased recombinant immunogen protein (HA, GFP), using an initial immunization with protein immunogen emulsified in CFA, followed by boosts in IFA, a standard immunization schedule, to elicit anti-protein humoral immune responses (Greenfield, 2013). The schedule will be based on a typical schedule for the production of custom polyclonal Abs against a protein immunogen.

Mice are observed daily, recording water and food consumption, abnormal clinical observations, mortality, and weekly weights. Blood is sampled and serum stored at baseline, then before boost and 2 weeks after boost, with terminal bleed via cardiac puncture. Serum is stored and spleen mononuclear cells are harvested and cryopreserved for the ELIspot and intracellular cytokine staining procedures.

Evaluating immunogenicity—analytic considerations and experiment planning. For each immunization strategy, groups of 6 animals are employed. The power analysis, dictating 6 animals/group, is based on a two-sided, two-sample t-test with hypothesized relative effect sizes, |μ1−μ2|/s, yielding at least 90% power to detect a relative effect size, |μ1−μ2|/s, of 2.7 between any two groups with a familywise type I error of 0.05, which accounts for multiple comparisons although, given the preliminary data, the observed effects are expected to be far in excess of this threshold. Using groups of 6 animals will therefore provide better than adequate power to identify the better immunization strategy in each pair.

For each of these comparisons, the primary evaluation to advance a particular immunization procedure to the next stage comparison will be the production of IgG(Total) against the immunogen, but the ability of the new procedures to elicit production of specific IgG subtypes, IgM, and IgA, and CMI is also taken into account.

The preliminary experiments presented above are also repeated in an expanded form, with additional controls. The immune responses of mice immunized with wt E. coli (ME5000, the TMUG parental strain, as well as a commonly used E. coli strain, MG1655; Hashimoto et al., 2005; Kato & Hashimoto, 2007), transformed with the immunogen expressing plasmids and bacteria not transformed with plasmids as negative controls, are compared. GR E. coli TMUG strains, with genome reductions of 2.4%, 15.9%, and 29.7%, are compared employing the more extensive immune assays described above.

As an initial comparator for traditional immunization procedures, SC immunizations with the bacteria expressing the immunogens is employed, and SC immunizations with commercial recombinant HA and GFP. For the protein antigens the CFA prime, IFA for boosts (Greenfield, 2013) are employed. In addition, for these comparison experiments, to maximize the ability of a standard immunization procedure to elicit a good humoral immune response, the HA and GFP test immunogens are conjugated to KLH using a widely available kit (e.g. AAT Bio ReadiLink KLH Conjugation Kit or Novus/Techne Imm-Link for carboxyl conjugation since the HA immunotag does not include S or M).

Evaluating immunogenicity—pairwise comparisons of immunization procedure enhancements. The preliminary data indicated, in a type of “dose-response” study, that immunization with bacteria expressing recombinant immunogen on the surface of the most highly GR E. coli strain (29.7% deleted) elicited much better immune responses than the wt or less deleted bacteria. Assuming the repeat experiments recapitulate the preliminary results, responses elicited by the largest deletion, 29.7%, GR E. coli expressing the immunogens and administered intranasally, pairwise, are compared with: a) SC immunization with the protein immunogen using the CFA prime/IFA boost regimen, b) SC immunization with wt E. coli expressing the immunogen, and c) SC immunization with the 29.7% GR E. coli expressing the immunogens. Given the preliminary results, and the known benefits of intranasal immunization described above, it is believed that the intranasal immunization with the GR E. coli vector will yield the best immune responses.

The product of the experiments above are a vaccination regimen that is used in the experiments below to show that the new procedures can rapidly yield the production of usable quantities of custom Abs.

### 2. Demonstrate Rapid Production of Polyclonal Antibodies in Rabbits Using Optimized Immunization Procedures

Overview. Since an aspect of this EXAMPLE pertains to procedures that enable the rapid production of custom Abs broadly useful for biomedical research, the following experiments are provided to demonstrate the utility of the new procedures by showing that we can rapidly produce custom Abs in the species most commonly used to produce new custom polyclonal Abs, the rabbit. Having optimized and refined the new immunization procedure in the mouse experiments above, it is then shown that the optimized procedure is effective in the rabbit. The two immunization procedures that yielded the highest mean Ab concentration elicited 14 days after the boost immunization are determined and those procedures are used to immunize the rabbits, comparing the two best procedures with each other and with SC immunizations with killed whole bacteria vaccines, and with SC immunization using purified immunogen proteins.

In these experiments, a focus is on the intranasal immunization of rabbits, in comparison with the typical CFA/IFA SC protein prime/boost schedules, given our preliminary results. If immunization via another route with the GR E. coli proves to be more effective, then that route is used. In any case, given the preliminary results, with the evidence for greatly enhanced accessibility of the surface expressed immunogen on the GR E. coli, it is believed that expression of protein immunogens on the surfaces of GR E. coli will almost certainly elicit enhanced immune responses compared to non-GR bacteria or native protein.

New Zealand White rabbits are used, since this breed is the most commonly used breed to produce custom antibodies and is of medium-large size, enabling reasonable blood volume sampling.

Rabbit immunizations. Groups of 6 rabbits are immunized with the statistical considerations described above. For the rabbit studies, due to the larger size of the animals, 10⁹ cells are used for single immunizations. If the exp-inc immunizations in the mice yield significantly better responses, for the rabbits 10⁹, 3×10⁹, and 10¹⁰ cells per dose every other day are used.

If it is determined in the initial mouse studies that intranasal immunization yields the best immune responses, which is expected given the preliminary data, this route is also used for the rabbit confirmatory studies. Intranasal immunization has been commonly used in rabbit studies, typically in a volume of 0.5 ml, administered by dripping by pipette into the nares of the rabbit held in an inverted position (Shoemaker et al., 2005; Oliveira et al., 2007).

For the comparison, rabbit SC immunizations are used, employing CFA/IFA prime-boost methods widely described in detail in standard reference works (Greenfield, 2013), which procedures are used in conducting the SC immunizations as reference for comparing the new GR E. coli methods, using the HA and GFP (conjugated, see above) test immunogens. For the comparison conventional protein immunizations, 200 μg for the prime and 100 μg for each boost are used. Boosts are used every 2 weeks for a total of 5 times, with both the conventional procedure and new GR E. coli-based immunizations.

Rabbit blood sampling. Blood samples are obtained via marginal ear vein venipuncture. Assuming a ˜5 Kg size for the typical New Zealand White, with a blood draw volume limit of 1% of body weight every 2 weeks, it is sampled ˜5 ml at baseline, then after 2 weeks, before boosting, and 2 weeks after each boost at 2 week intervals. After the final boost it is continued to sample at 2 wk intervals for a total 6 months, to evaluate any additional maturation in the immune responses and to establish that the blood draws from the rabbits immunized according to the accelerated procedure can produce commercially and biomedically useful quantities of sera over a long time. At the end of the experiment, a terminal bleed is conducted following euthanasia to confirm that a useful maximum amount of sera from the rabbit immunized can be produced using the procedures.

Testing rabbit sera elicited by the new procedures. A comparison between the standard procedure and the new procedure is Ab concentrations elicited against the HA and GFP immunogens, comparing the kinetics of Ab development in the conventional and new GR E. coli procedures. Humoral immunity is evaluated using the sandwich ELISA methods described above, using the appropriate HRP-conjugated anti-rabbit IgG(Total), IgG1, IgG2a, and IgA and IgM antibodies (Abcam, ThermoFisher, Sigma) in place of the anti-mouse used above. A comparison between the new and conventional procedures is made at the 2-week and 4-week times after the first prime immunization, and an evaluation is made of the comparison between the sera elicited from the rabbits using the new procedure at 2 and 4 weeks with the sera elicited from the rabbits using the conventional procedure at end of the experiment, 16 weeks after the prime immunization.

Testing rabbit cellular immune responses elicited by the new procedures. While a goal of this EXAMPLE is the rapid production of biotechnologically effective antisera, the ability of the GR E. coli to stimulate cell-mediated immunity is also compared. The same techniques as described above for the study of mouse cell-mediated immunity are used, modifying the procedures to use the appropriate antibodies and reagents directed against the equivalent rabbit markers. For the rabbit experiments, given that one can obtain much larger blood volumes, peripheral lymphocytes are used, and the ELIspot assays are conducted on blood from each draw, not just the terminally obtained spleen cells.

Functional evaluation of sera elicited in rabbits using the new procedures. Since a goal of this EXAMPLE is to develop a procedure to rapidly elicit highly effective custom polyclonal antibodies for use in a wide variety of biological research projects, it is confirmed that the sera elicited against the test immunogens in the rabbit are functional for key techniques in which custom antisera are used. These include immunoblotting, flow cytometry, and immunofluorescence microscopy. For each of these applications the polyclonal Abs produced in the new procedure are compared with commercially avail able rabbit polyclonal Abs (e.g Abcam). For the immunoblotting evaluation, it is confirmed that the elicited rabbit sera can detect the HA and GFP antigens produced in the bacteria, run in parallel with commercially produced GFP and HA-immunotagged protein standards (e.g. Abcam, ThermoFisher), using commercial HRP-conjugated anti-rabbit secondary Ab. For the flow cytometry experiments the ability of the elicited rabbit antisera to bind the bacteria is compared, but since many Abs in the polyclonal sera will likely be directed against bacterial antigens, an important test will be for the ability of the sera to stain mammalian cells expressing the test antigens. For the flow cytometry and IF experiments commercially available stable cell lines expressing GFP and HA-fusion proteins (e.g. GenTarget, R&D/Biotechne, ATCC) are employed, using standard protocols recommended by the vendor of the commercial polyclonal rabbit Abs (e.g. https://www.abcam.com/protocols/indirect-flow-cytometry-protocol; https://www.abcam.com/protocols/immunocytochemistry-immunofluorescence-protocol) with commercially available anti-rabbit (e.g. ThermoFisher Invitrogen Goat anti-rabbit-Alexa 594). In the case of the flow cytometry studies using the GFP cell lines, it can be compared directly signal from GFP gating with signal gating on the fluor labeling the anti-rabbit secondary Ab.

In summary, an aspect of this project is that surface expression of immunogen on the GR E. coli are particularly immunogenic. It is believed that the preliminary data indicate it is possible to rapidly elicit effective polyclonal Abs. An additional approach is taken to enhancing immune response: exp-inc immunization schedule. There are additional methods to increase immunogenicity, which can be tested, if needed. Additionally, the bacteria can be administered together with additional adjuvant, such as cholera toxin B subunit, AS03, AS04, and/or MF59, although care should be taken in using lipophilic adjuvants that they do not excessively damage the bacterial outer membrane that holds the immunogen.

In summary, the first experiments are devoted to optimizing the immunization protocols in mice. The immunogen-expressing plasmids are constructed and tested, and a series of head-to-head comparisons of routes, immunogens, and schedules (single dose vs. exp-inc), comparing the induction primarily of humoral immune responses, are performed. Thereafter, how the best, optimized immunization methods developed in the mouse models work to rapidly produce custom polyclonal Abs in the rabbit, the principle source for custom polyclonal Abs for research purposes, are determined.

An immediate future downstream biotechnological application is the accelerated production of mouse mAbs. Antigen production, purification, conjugation, immunization, and boosting for the production of mouse mAbs also occupy considerable time prior to fusion and hybridoma production. Shortening this time from months to weeks would significantly accelerate production of mouse mAbs to a similar extent that will occur with the production of custom polyclonal Abs.

A very rapid synthetic biology recombinant bacterial vaccine platform provides utility for many clinical purposes, from the rapid development of prophylactic vaccines for infectious diseases to custom tumor antigen-directed cancer immunotherapy. From a basic biological research perspective, understanding why the GR E. coli are such highly effective immunogens could yield insights helpful in many areas, including whether the enhanced immunogenicity is the result of increased immunogen accessibility, or whether some of the gene products removed from the surfaces of the GR E. coli blunt the host response against the immunogens expressed on the bacterial surface, and/or whether if some of the gene products do blunt the host immune response, what are the responsible mechanisms. While it is not desired to be bound by any particular theory of operation, understanding such mechanisms offers insights into the processes governing the assembly and maintenance of a wide range of host microbial communities.

### Example 2

### Construction of an Exemplary Antigen Expression Plasmid

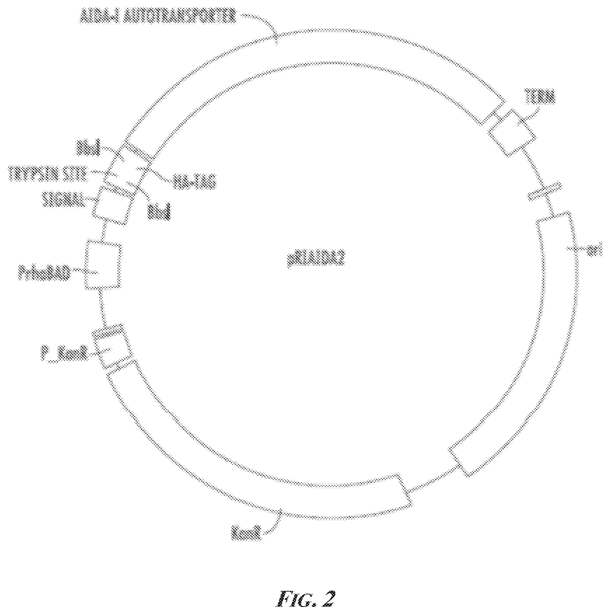

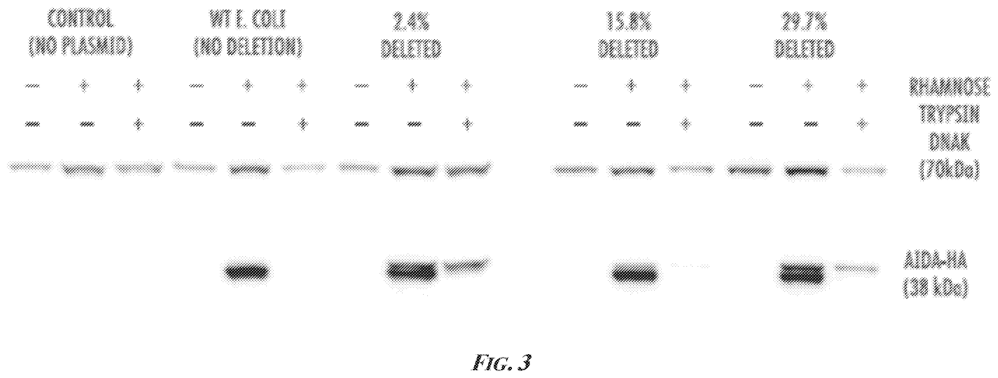

As an exemplary, non-limiting implementation, a Gram-negative AT recombinant expression system for rapid Ab production and immunization was constructed. In preliminary experiments, as an exemplary, non-limiting implementation that can used for the expression of antigen in bacteria, plasmid pRIAIDA, which has a rhamnose inducible AIDA-I Gram-negative AT expression cassette for expression optimization, with a cloning site that enables DNA encoding an antigen of interest to be expressed using the inducible AT expression cassette so that bacteria express the encoded protein on their surfaces, and flanked by a trypsin site was placed in the coding sequence to evaluate surface expression of antigens. In initial experiments, a sequence encoding a widely-used influenza virus HA immunotag (YPYDVPDYA; SEQ ID NO: 44) was inserted into the surface expression cassette to produce plasmid pRIAIDA-HA. Plasmid sequences are disclosed herein. FIG. 2 shows the map of the pRIAIDA-HA plasmid and plasmid sequences are disclosed herein. A trypsination experiment confirming that that HA immunotag was present on the exterior of bacteria when expressed in an AIDA-I expression cassette that includes a trypsin site in the coding sequence was performed, and the results are shown in FIG. 3.

### Example 3

Evaluation of Antibody Binding to GR E. coli Expressing a Test Immunogen and the Ability of GR E. coli Expressing a Test Immunogen on Its Surface to Elicit an Immune Response

While only a representative approach, intranasal immunization has a number of advantages as a route of administration. Intranasal immunization leads to the direct exposure in the nasal mucosa of M cells and dendritic cells to the immunogen. In addition, there are abundant nasopharyngeal lymphoid tissues with large numbers of other antigen presenting cells, like macrophages, and many T cells and B cells. Intranasal immunization can also induce potent tissue-resident effector and effector memory CD8+ T cell immunity, and can also be particularly effective in producing antibodies in other body compartments if, for example, secretory mucosal antibodies are useful. In addition, since most pathogens enter the host across mucosal surfaces, if a productive way to rapidly elicit potent antibody response is to mimic a significant pathogen threat, mucosal immunization might be expected to elicit better immune responses than a more traditional parenteral route.

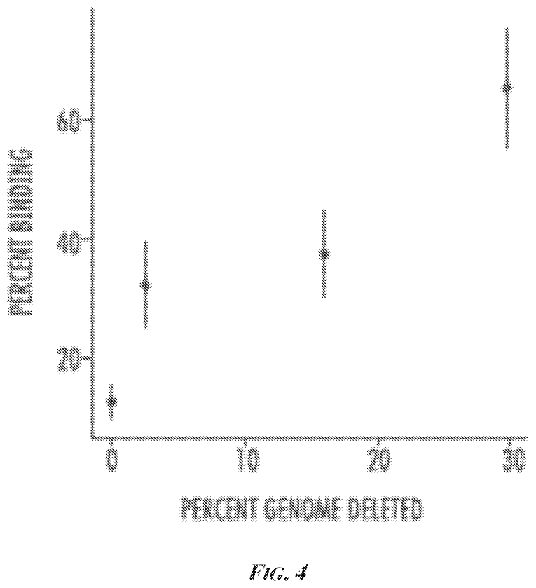

As a test of the new method for rapidly producing highly immunogenic vaccines, made by expressing an antigen in genome reduced bacteria, pRIAIDA-HA was transformed into wild type parental E. coli and also into three GR E. coli. Binding of a commercial anti-HA mAb to the bacteria was evaluated via flow cytometry (FIG. 4). The ability of the bacteria to bind the anti-HA mAb was found to be increased significantly as the fraction of the genome deleted increased.

This data shows that when an antigen is expressed in a GR bacteria, and more particularly in a Gram-negative bacteria, and more particularly on the surface of a GR bacteria that it is much more accessible to antibody binding, which is a useful surrogate for the immune system as whole. The greater ability to bind antibody and interact with the immune system should translate into a better ability elicit a potent immune response directed against that antigen. This is particularly true since many other components of the bacteria, such as LPS, pili, and fimbrae, are pathogen-associated molecular patterns, recognized by Toll-like receptors, which shoud enhance immune responses to recombinant immunogens expressed in the bacteria. The data also indicate that in the wild type bacteria, antigens expressed on the bacterial surface are in some way hidden or “cloaked” from the immune system, and so less able to elicit an immune response. This also suggests that removing large numbers of proteins from the surfaces of bacteria should enhance immune responses against all non-removed proteins present on the bacterial surface, including native bacterial proteins, which is useful in making vaccines against bacterial pathogens.

### Example 4

Evaluation of the Ability of GR E. coli Expressing a Test Immunogen on its Surface to Elicit an Immune Response

As disclosed herein, expressing an immunogen in a genome reduced bacterium greatly increased the recognition of that antigen by the immune system and elicited anti-immunogen antibodies. That finding suggested that immunogens expressed in genome reduced bacteria would be more accessible to and better recognized by the immune system in general, which implied that expressing an immunogen in a genome reduced bacteria could yield a substantially enhanced immune response against that immunogen. This substantially enhanced immune response against an antigen of interest can then be exploited in this system to: 1) Make new and better prophylactic and therapeutic vaccines for infectious diseases, by expressing pathogen antigens capable of being targeting by an inactivating or neutralizing immune response, 2) Make new and better therapeutic vaccines for cancer targeting tumor specific antigens, 3) Modulate the immune system to clear and/or attack or inactive molecules or structures mediating the pathogenesis of disease, including autoimmune or inflammatory diseases or diseases mediated by the overproduction or overexpression of particular molecules, and 4) Rapid production of custom polyclonal and/or monoclonal antibodies useful for analytic, therapeutic, and industrial purposes. To establish whether expressing an antigen of interest in a genome reduced bacteria would yield an enhanced immune response, we prepared vaccines from wild type and genome reduced bacteria having different degrees of genome reduction. The bacteria were transformed with the pRIADA plasmid that expresses the HA immunotag as a test vaccine antigen. Expression of the HA immunotag was induced with rhamnose and expression of the HA immunotag on the surface of the bacteria was verified by flow cytometry (FIG. 4).

Mice were immunized intranasally with 10⁸ formalin-fixed wild type and genome reduced bacteria expressing the HA immunotag. After 2 weeks, blood was collected from the immunized mice and the sera tested using an ELISA with a commercial anti-HA mAb as a standard. The quantity of anti-HA antibodies in the mouse sera was determined using the ELISA in pre-immune sera and after immunization. It was determined that intranasal immunization with GR E. coli expressing the test immunogen on their surfaces elicited the production of large amounts of Abs in only 2 weeks, and that the ability of the bacteria expressing the test antigen to elicit production of the Ab increased substantially in bacteria with more genes deleted. These data were confirmed using immuno-dot blots that indicated that the same amount of HA was produced per cell regardless of extent of the genome reduction, which would tend to support the hypothesis that the increased binding and immunogenicity was due to increased antigen accessibility in the GR strains and/or an absence of immunoinhibitory surface structures in the GR strains. While in this representative instance the immunoinhibitory components are on the surface of the bacterium, in other instances the immunoinhibitory components can be elsewhere on the bacteria. The examples then show that expressing a well-established test antigen on the surface of genome reduced bacteria leads to substantially increased binding to those bacteria of a monoclonal antibody that recognizes the test antigen and that immunizing an animal with genome reduced bacteria expressing a well-established test antigen on its surface elicits a much more potent immune response than immunizing animals with wild type bacteria expressing the same antigen.

### Example 5

### Representative Sequences and Constructs

pRHIA-2 Construct Design:
Construct Name: pRHIA-2

The rhamnose inducible promoter, strong ribosome binding subunit, termination phage T7 were taken from pD861 construct (ATUM), and inserted the sequence into pUC57 HiA AT to make pRHIA-2. pRHIA-2 has the full native HIA AT coding sequence (underlined below). There are also BsaI and XmaI restriction sites on the ends, and BbsI sites internally for cloning inserts. The insert was synthesized by GeneWiz in pUC57.

pRHIA-2 Insert Sequence (SEQ ID NO: 1):
GGTCTCACACACCACAATTCAGCAAATTGTGAACATCATCACGTTCATCTTTCCCTGGTTGCCAA
TGGCCCATTTTCCTGTCAGTAACGAGAAGGTCGCGAATTCAGGCGCTTTTTAGACTGGTCGTAAT
GAAATTCTTTTTAAGAAGGAGATATACATATGATTAAATTAAAATTTGGTGTTTTTTTTACAGTT
TTACTATCTTCAGCATATGCACATGGAACACCTCAAAATATTACTCACGGTGTGTTAACCCTGTC
TTCTCGTGGTCTGGACTATCCATACGATGTACCGGATTACGCGCGTTATGGAAGACGAGTGTTGA
ACAAGGGATGGTATTTAACCGTGTTAACAATGGAGATCTTCGAAAATGTGGCTAATGGCGATATT
TCTGCCACTTCCACCGATGCGATTAACGGAAGTCAGTTGTATGCTGTGGCAAAAGGGGTAACAAA
CCTTGCTGGACAAGTGAATAATCTTGAGGGCAAAGTGAATAAAGTGGGCAAACGTGCAGATGCAG
GTACAGCAAGTGCATTAGCGGCTTCACAGTTACCACAAGCCACTATGCCAGGTAAATCAATGGTT
GCTATTGCGGGAAGTAGTTATCAAGGTCAAAATGGTTTAGCTATCGGGGTATCAAGAATTTCCGA
TAATGGCAAAGTGATTATTCGCTTGTCAGGCACAACCAATAGTCAAGGTAAAACAGGCGTTGCAG
CAGGTGTTGGTTACCAGTGGTAAGTTAGAGCGGCCGCCACCGCTGAGCAATAACTAGCATAACCC
CTTGGGGCCTCTAAACGGGTCTTGAGGGGTTTTTTGCTGAAAGGAGGAACTATATCCGGGTAACC
CGGG
pRHIA-2 complete sequence (including pUC57) (SEQ ID NO: 2):
TCGCGCGTTTCGGTGATGACGGTGAAAACCTCTGACACATGCAGCTCCCGGAGACGGTCACAGCT
TGTCTGTAAGCGGATGCCGGGAGCAGACAAGCCCGTCAGGGCGCGTCAGCGGGTGTTGGCGGGTG
TCGGGGCTGGCTTAACTATGCGGCATCAGAGCAGATTGTACTGAGAGTGCACCATATGCGGTGTG
AAATACCGCACAGATGCGTAAGGAGAAAATACCGCATCAGGCGCCATTCGCCATTCAGGCTGCGC
AACTGTTGGGAAGGGCGATCGGTGCGGGCCTCTTCGCTATTACGCCAGCTGGCGAAAGGGGGATG
TGCTGCAAGGCGATTAAGTTGGGTAACGCCAGGGTTTTCCCAGTCACGACGTTGTAAAACGACGG
CCAGTGAATTGACGCGTATTGGGATGGTCTCACACACCACAATTCAGCAAATTGTGAACATCATC
ACGTTCATCTTTCCCTGGTTGCCAATGGCCCATTTTCCTGTCAGTAACGAGAAGGTCGCGAATTC
AGGCGCTTTTTAGACTGGTCGTAATGAAATTCTTTTTAAGAAGGAGATATACATATGATTAAATT
AAAATTTGGTGTTTTTTTTACAGTTTTACTATCTTCAGCATATGCACATGGAACACCTCAAAATA
TTACTCACGGTGTGTTAACCCTGTCTTCTCGTGGTCTGGACTATCCATACGATGTACCGGATTAC
GCGCGTTATGGAAGACGAGTGTTGAACAAGGGATGGTATTTAACCGTGTTAACAATGGAGATCTT
CGAAAATGTGGCTAATGGCGATATTTCTGCCACTTCCACCGATGCGATTAACGGAAGTCAGTTGT
ATGCTGTGGCAAAAGGGGTAACAAACCTTGCTGGACAAGTGAATAATCTTGAGGGCAAAGTGAAT
AAAGTGGGCAAACGTGCAGATGCAGGTACAGCAAGTGCATTAGCGGCTTCACAGTTACCACAAGC
CACTATGCCAGGTAAATCAATGGTTGCTATTGCGGGAAGTAGTTATCAAGGTCAAAATGGTTTAG
CTATCGGGGTATCAAGAATTTCCGATAATGGCAAAGTGATTATTCGCTTGTCAGGCACAACCAAT
AGTCAAGGTAAAACAGGCGTTGCAGCAGGTGTTGGTTACCAGTGGTAAGTTAGAGCGGCCGCCAC
CGCTGAGCAATAACTAGCATAACCCCTTGGGGCCTCTAAACGGGTCTTGAGGGGTTTTTTGCTGA
AAGGAGGAACTATATCCGGGTAACCCGGGATCCCAATGGCGCGCCGAGCTTGGCTCGAGCATGGT
CATAGCTGTTTCCTGTGTGAAATTGTTATCCGCTCACAATTCCACACAACATACGAGCCGGAAGC
ATAAAGTGTAAAGCCTGGGGTGCCTAATGAGTGAGCTAACTCACATTAATTGCGTTGCGCTCACT
GCCCGCTTTCCAGTCGGGAAACCTGTCGTGCCAGCTGCATTAATGAATCGGCCAACGCGCGGGGA
GAGGCGGTTTGCGTATTGGGCGCTCTTCCGCTTCCTCGCTCACTGACTCGCTGCGCTCGGTCGTT
CGGCTGCGGCGAGCGGTATCAGCTCACTCAAAGGCGGTAATACGGTTATCCACAGAATCAGGGGA
TAACGCAGGAAAGAACATGTGAGCAAAAGGCCAGCAAAAGGCCAGGAACCGTAAAAAGGCCGCGT
TGCTGGCGTTTTTCCATAGGCTCCGCCCCCCTGACGAGCATCACAAAAATCGACGCTCAAGTCAG
AGGTGGCGAAACCCGACAGGACTATAAAGATACCAGGCGTTTCCCCCTGGAAGCTCCCTCGTGCG
CTCTCCTGTTCCGACCCTGCCGCTTACCGGATACCTGTCCGCCTTTCTCCCTTCGGGAAGCGTGG
CGCTTTCTCATAGCTCACGCTGTAGGTATCTCAGTTCGGTGTAGGTCGTTCGCTCCAAGCTGGGC
TGTGTGCACGAACCCCCCGTTCAGCCCGACCGCTGCGCCTTATCCGGTAACTATCGTCTTGAGTC
CAACCCGGTAAGACACGACTTATCGCCACTGGCAGCAGCCACTGGTAACAGGATTAGCAGAGCGA
GGTATGTAGGCGGTGCTACAGAGTTCTTGAAGTGGTGGCCTAACTACGGCTACACTAGAAGAACA
GTATTTGGTATCTGCGCTCTGCTGAAGCCAGTTACCTTCGGAAAAAGAGTTGGTAGCTCTTGATC
CGGCAAACAAACCACCGCTGGTAGCGGTGGTTTTTTTGTTTGCAAGCAGCAGATTACGCGCAGAA
AAAAAGGATCTCAAGAAGATCCTTTGATCTTTTCTACGGGGTCTGACGCTCAGTGGAACGAAAAC
TCACGTTAAGGGATTTTGGTCATGAGATTATCAAAAAGGATCTTCACCTAGATCCTTTTAAATTA
AAAATGAAGTTTTAAATCAATCTAAAGTATATATGAGTAAACTTGGTCTGACAGTTAGAAAAACT
CATCGAGCATCAAATGAAACTGCAATTTATTCATATCAGGATTATCAATACCATATTTTTGAAAA
AGCCGTTTCTGTAATGAAGGAGAAAACTCACCGAGGCAGTTCCATAGGATGGCAAGATCCTGGTA
TCGGTCTGCGATTCCGACTCGTCCAACATCAATACAACCTATTAATTTCCCCTCGTCAAAAATAA
GGTTATCAAGTGAGAAATCACCATGAGTGACGACTGAATCCGGTGAGAATGGCAAAAGTTTATGC
ATTTCTTTCCAGACTTGTTCAACAGGCCAGCCATTACGCTCGTCATCAAAATCACTCGCATCAAC
CAAACCGTTATTCATTCGTGATTGCGCCTGAGCGAGACGAAATACGCGATCGCTGTTAAAAGGAC
AATTACAAACAGGAATCGAATGCAACCGGCGCAGGAACACTGCCAGCGCATCAACAATATTTTCA
CCTGAATCAGGATATTCTTCTAATACCTGGAATGCTGTTTTCCCAGGGATCGCAGTGGTGAGTAA
CCATGCATCATCAGGAGTACGGATAAAATGCTTGATGGTCGGAAGAGGCATAAATTCCGTCAGCC
AGTTTAGTCTGACCATCTCATCTGTAACATCATTGGCAACGCTACCTTTGCCATGTTTCAGAAAC
AACTCTGGCGCATCGGGCTTCCCATACAATCGATAGATTGTCGCACCTGATTGCCCGACATTATC
GCGAGCCCATTTATACCCATATAAATCAGCATCCATGTTGGAATTTAATCGCGGCCTAGAGCAAG
ACGTTTCCCGTTGAATATGGCTCATACTCTTCCTTTTTCAATATTATTGAAGCATTTATCAGGGT
TATTGTCTCATGAGCGGATACATATTTGAATGTATTTAGAAAAATAAACAAATAGGGGTTCCGCG
CACATTTCCCCGAAAAGTGCCACCTGACGTCTAAGAAACCATTATTATCATGACATTAACCTATA
AAAATAGGCGTATCACGAGGCCCTTTCGTC

pRHIA-2 Trne Construct Design:
Construct Name: pRHIA2_Trnc_Insert

The same insert as pRHIA-2 was taken and the part of the HIA AT was deleted so that the expression of the COVID19 stalk region occurs closer to the surface. This was because all of the HIV constructs being used uses a truncated HIA AT, so the same length of HIA AT as the other constructs was used and it was synthesized into pUCKan (Blue Heron's cloning vector similar to pUC57). pRHIA-2 and pRHIA-2 Trnc are used as expression vectors for COVID19 Stalk constructs. pRHIA-2 Trnc was likely codon optimized.

pRHIA-2 Trnc Insert Sequence (SEQ ID NO: 3):
GGTCTCACACACCACAATTCAGCAAATTGTGAACATCATCACGTTCATCTTTCCCTGGTTGCCAA
TGGCCCATTTTCCTGTCAGTAACGAGAAGGTCGCGAATTCAGGCGCTTTTTAGACTGGTCGTAAT
GAAATTCTTTTTAAGAAGGAGATATACATATGATTAAATTAAAATTTGGTGTTTTTTTTACAGTT
TTACTATCTTCAGCATATGCACATGGAACACCTCAAAATATTACTCACGGTGTGTTAACCCTGTC
TTCTCGTGGTCTGGACTATCCATACGATGTACCGGATTACGCGCGTTATGGAAGACGAGTGTTGA
ACAAGGGATGGTATTTAACCGTGTTAACAATGGAGATCTTCGAAAGCCAGCTGCCGCAGGCGACC
ATGCCGGGCAAAAGCATGGTGGCGATTGCGGGCAGCAGCTATCAGGGCCAGAACGGCCTGGCGAT
TGGCGTGAGCCGCATTAGCGATAACGGCAAAGTGATTATTCGCCTGAGCGGCACCACCAACAGCC
AGGGCAAAACCGGCGTGGCGGCGGGCGTGGGCTATCAGTGGTAAGTTAGAGCGGCCGCCACCGCT
GAGCAATAACTAGCATAACCCCTTGGGGCCTCTAAACGGGTCTTGAGGGGTTTTTTGCTGAAAGG
AGGAACTATATCCGGGTAACCCGGG

Referring to FIG. 1, shown are exemplary SARS-CoV-2 stalk antigens from S protein for expression with the trimeric HIA autotransporter. FIG. 1 shows the stalk immunogen designs for expression as trimers via an Hia trimeric autotransporter expression cassette. S protein domains shown include the Fusion Peptide (FP), Heptad Repeat 1 (HR1), Central Helix (CH), Connector Domain (CD), Heptad Repeat 2 (HR2), Transmembrane Domain (TM), and cytoplasmic tail (CT), with amino acid numbering following that shown in Wrapp et al., 2020, which is incorporated by reference herein in its entirety. The right of the Figure schematically shows the relevant portions of the Hia trimeric autotransporter expression cassette in an exemplary plasmid. Each group extends the sequence to different regions of the S protein; within each group constructs are fused to the autotransporter beta barrel at different points to test effects on immunogenicity of placing the expressed protein at different distances from the outer membrane and within a different register in the transmembrane region of the beta barrel.

### COVID19 Stalk Construct Design:

The stalk region of the S protein of COVID19 was considered and different regions were taken from this area to clone into pRHIA-2 and pRHIA-2 Trnc because the HIA AT resembles the trimer of coronavirus (like HIV). The construct regions (12) are shown in FIG. 1. The first group (begins at position 986) extends from the CH domain to either full transmembrane, half transmembrane, or no transmembrane. The next groups extend from the CD domain (begins at position 1076), HR2 (begins at position 1163), and half HR2 (begins at position 1188) with the same 3 TM amounts each. TM were taken, because with HIV, different amounts of TM were added for MPER.

The sequences were then codon optimized for E. coli with GeneWiz's codon optimizer tool. Compatible BbsI overhangs were also added to ends of each of the inserts to match with pRHIA-2 and pRHIA-2 Trnc for cloning. The 12 constructs were then synthesized by Blue Heron Bio.

### Construct Names and Sequence:

CH_Stalk_Full_TM (SEQ ID NO: 4; 986-1238 of FIG. 1):
AACCACGTCTTCAAAAGTGGAAGCCGAAGTTCAAATCGACCGTCTGATCACCGGTCGTCTGCAGA
GTCTGCAGACCTATGTGACCCAGCAGCTCATCCGCGCCGCGGAAATTCGTGCCAGCGCGAATCTG
GCCGCGACGAAAATGAGCGAGTGCGTGCTGGGCCAGAGCAAACGCGTGGATTTCTGCGGCAAGGG
CTACCATCTGATGAGCTTCCCACAGAGCGCGCCGCACGGTGTGGTTTTTCTGCATGTTACCTACG
TGCCAGCCCAAGAAAAAAACTTCACGACCGCGCCAGCGATCTGTCACGACGGCAAGGCCCACTTC
CCACGCGAAGGTGTGTTCGTGAGCAATGGTACCCACTGGTTTGTGACGCAGCGCAACTTCTACGA
ACCGCAGATCATCACCACGGACAACACGTTCGTGAGCGGTAACTGCGACGTGGTGATTGGCATCG
TGAACAACACGGTGTACGATCCGCTCCAGCCAGAACTGGACAGCTTCAAGGAGGAGCTGGACAAG
TACTTCAAAAACCACACCAGCCCGGACGTTGATCTGGGCGACATCAGCGGCATCAACGCGAGCGT
GGTGAACATCCAGAAAGAGATCGATCGTCTGAACGAAGTGGCGAAAAACCTCAACGAGAGTCTGA
TCGATCTGCAAGAACTGGGCAAATACGAGCAGTACATCAAGTGGCCGTGGTACATTTGGCTGGGT
TTCATTGCCGGTCTGATCGCCATCGTGATGGTGACCATCATGCTGTGCTGCATGAGAAGACTTGT
GT
CH_Stalk_Half_TM (SEQ ID NO: 5; 986-1226 of FIG. 1):
AACCACGTCTTCAAAAGTGGAAGCCGAAGTTCAAATCGACCGTCTGATCACCGGTCGTCTGCAGA
GTCTGCAGACCTATGTGACCCAGCAGCTCATCCGCGCCGCGGAAATTCGTGCCAGCGCGAATCTG
GCCGCGACGAAAATGAGCGAGTGCGTGCTGGGCCAGAGCAAACGCGTGGATTTCTGCGGCAAGGG
CTACCATCTGATGAGCTTCCCACAGAGCGCGCCGCACGGTGTGGTTTTTCTGCATGTTACCTACG
TGCCAGCCCAAGAAAAAAACTTCACGACCGCGCCAGCGATCTGTCACGACGGCAAGGCCCACTTC
CCACGCGAAGGTGTGTTCGTGAGCAATGGTACCCACTGGTTTGTGACGCAGCGCAACTTCTACGA
ACCGCAGATCATCACCACGGACAACACGTTCGTGAGCGGTAACTGCGACGTGGTGATTGGCATCG
TGAACAACACGGTGTACGATCCGCTCCAGCCAGAACTGGACAGCTTCAAGGAGGAGCTGGACAAG
TACTTCAAAAACCACACCAGCCCGGACGTTGATCTGGGCGACATCAGCGGCATCAACGCGAGCGT
GGTGAACATCCAGAAAGAGATCGATCGTCTGAACGAAGTGGCGAAAAACCTCAACGAGAGTCTGA
TCGATCTGCAAGAACTGGGCAAATACGAGCAGTACATCAAGTGGCCGTGGTACATTTGGCTGGGT
TTCATTGCCGGTCTGATCAGAAGACTTGTGT
CH_Stalk_No_TM_C (SEQ ID NO: 6; 986-1214 of FIG. 1):
AACCACGTCTTCAAAAGTGGAAGCCGAAGTTCAAATCGACCGTCTGATCACCGGTCGTCTGCAGA
GTCTGCAGACCTATGTGACCCAGCAGCTCATCCGCGCCGCGGAAATTCGTGCCAGCGCGAATCTG
GCCGCGACGAAAATGAGCGAGTGCGTGCTGGGCCAGAGCAAACGCGTGGATTTCTGCGGCAAGGG
CTACCATCTGATGAGCTTCCCACAGAGCGCGCCGCACGGTGTGGTTTTTCTGCATGTTACCTACG
TGCCAGCCCAAGAAAAAAACTTCACGACCGCGCCAGCGATCTGTCACGACGGCAAGGCCCACTTC
CCACGCGAAGGTGTGTTCGTGAGCAATGGTACCCACTGGTTTGTGACGCAGCGCAACTTCTACGA
ACCGCAGATCATCACCACGGACAACACGTTCGTGAGCGGTAACTGCGACGTGGTGATTGGCATCG
TGAACAACACGGTGTACGATCCGCTCCAGCCAGAACTGGACAGCTTCAAGGAGGAGCTGGACAAG
TACTTCAAAAACCACACCAGCCCGGACGTTGATCTGGGCGACATCAGCGGCATCAACGCGAGCGT
GGTGAACATCCAGAAAGAGATCGATCGTCTGAACGAAGTGGCGAAAAACCTCAACGAGAGTCTGA
TCGATCTGCAAGAACTGGGCAAATACGAGCAGTACATCAAGTGGCCGAGAAGACTTGTGT
CD_Stalk_Full_TM (SEQ ID NO: 7; 1076-1238 of FIG. 1):
AACCACGTCTTCAACGACCGCGCCAGCGATCTGTCACGACGGCAAGGCCCACTTCCCACGCGAAG
GTGTGTTCGTGAGCAATGGTACCCACTGGTTTGTGACGCAGCGCAACTTCTACGAACCGCAGATC
ATCACCACGGACAACACGTTCGTGAGCGGTAACTGCGACGTGGTGATTGGCATCGTGAACAACAC
GGTGTACGATCCGCTCCAGCCAGAACTGGACAGCTTCAAGGAGGAGCTGGACAAGTACTTCAAAA
ACCACACCAGCCCGGACGTTGATCTGGGCGACATCAGCGGCATCAACGCGAGCGTGGTGAACATC
CAGAAAGAGATCGATCGTCTGAACGAAGTGGCGAAAAACCTCAACGAGAGTCTGATCGATCTGCA
AGAACTGGGCAAATACGAGCAGTACATCAAGTGGCCGTGGTACATTTGGCTGGGTTTCATTGCCG
GTCTGATCGCCATCGTGATGGTGACCATCATGCTGTGCTGCATGAGAAGACTTGTGT
CD_Stalk_Half_TM (SEQ ID NO: 8; 1076-1226 of FIG. 1):
AACCACGTCTTCAACGACCGCGCCAGCGATCTGTCACGACGGCAAGGCCCACTTCCCACGCGAAG
GTGTGTTCGTGAGCAATGGTACCCACTGGTTTGTGACGCAGCGCAACTTCTACGAACCGCAGATC
ATCACCACGGACAACACGTTCGTGAGCGGTAACTGCGACGTGGTGATTGGCATCGTGAACAACAC
GGTGTACGATCCGCTCCAGCCAGAACTGGACAGCTTCAAGGAGGAGCTGGACAAGTACTTCAAAA
ACCACACCAGCCCGGACGTTGATCTGGGCGACATCAGCGGCATCAACGCGAGCGTGGTGAACATC
CAGAAAGAGATCGATCGTCTGAACGAAGTGGCGAAAAACCTCAACGAGAGTCTGATCGATCTGCA
AGAACTGGGCAAATACGAGCAGTACATCAAGTGGCCGTGGTACATTTGGCTGGGTTTCATTGCCG
GTCTGATCAGAAGACTTGTGT
CD_Stalk_No_TM_C (SEQ ID NO: 9; 1076-1214 of FIG. 1):
AACCACGTCTTCAACGACCGCGCCAGCGATCTGTCACGACGGCAAGGCCCACTTCCCACGCGAAG
GTGTGTTCGTGAGCAATGGTACCCACTGGTTTGTGACGCAGCGCAACTTCTACGAACCGCAGATC
ATCACCACGGACAACACGTTCGTGAGCGGTAACTGCGACGTGGTGATTGGCATCGTGAACAACAC
GGTGTACGATCCGCTCCAGCCAGAACTGGACAGCTTCAAGGAGGAGCTGGACAAGTACTTCAAAA
ACCACACCAGCCCGGACGTTGATCTGGGCGACATCAGCGGCATCAACGCGAGCGTGGTGAACATC
CAGAAAGAGATCGATCGTCTGAACGAAGTGGCGAAAAACCTCAACGAGAGTCTGATCGATCTGCA
AGAACTGGGCAAATACGAGCAGTACATCAAGTGGCCGAGAAGACTTGTGT
HR2_Stalk_Full_T (SEQ ID NO: 10; 1163-1238 of FIG. 1):
AACCACGTCTTCAGACGTTGATCTGGGCGACATCAGCGGCATCAACGCGAGCGTGGTGAACATCC
AGAAAGAGATCGATCGTCTGAACGAAGTGGCGAAAAACCTCAACGAGAGTCTGATCGATCTGCAA
GAACTGGGCAAATACGAGCAGTACATCAAGTGGCCGTGGTACATTTGGCTGGGTTTCATTGCCGG
TCTGATCGCCATCGTGATGGTGACCATCATGCTGTGCTGCATGAGAAGACTTGTGT
HR2_Stalk_Half_T (SEQ ID NO: 11; 1163-1226 of FIG. 1):
AACCACGTCTTCAGACGTTGATCTGGGCGACATCAGCGGCATCAACGCGAGCGTGGTGAACATCC
AGAAAGAGATCGATCGTCTGAACGAAGTGGCGAAAAACCTCAACGAGAGTCTGATCGATCTGCAA
GAACTGGGCAAATACGAGCAGTACATCAAGTGGCCGTGGTACATTTGGCTGGGTTTCATTGCCGG
TCTGATCAGAAGACTTGTGT
HR2_Stalk_No_TM (SEQ ID NO: 12; 1163-1214 of FIG. 1):
AACCACGTCTTCAGACGTTGATCTGGGCGACATCAGCGGCATCAACGCGAGCGTGGTGAACATCC
AGAAAGAGATCGATCGTCTGAACGAAGTGGCGAAAAACCTCAACGAGAGTCTGATCGATCTGCAA
GAACTGGGCAAATACGAGCAGTACATCAAGTGGCCGAGAAGACTTGTGT
Half_HR2_Stalk_F (SEQ ID NO: 13; 1188-1238 of FIG. 1):
AACCACGTCTTCAGAAGTGGCGAAAAACCTCAACGAGAGTCTGATCGATCTGCAAGAACTGGGCA
AATACGAGCAGTACATCAAGTGGCCGTGGTACATTTGGCTGGGTTTCATTGCCGGTCTGATCGCC
ATCGTGATGGTGACCATCATGCTGTGCTGCATGAGAAGACTTGTGT
Half_HR2_Stalk_H (SEQ ID NO: 14; 1188-1226 of FIG. 1):
AACCACGTCTTCAGAAGTGGCGAAAAACCTCAACGAGAGTCTGATCGATCTGCAAGAACTGGGCA
AATACGAGCAGTACATCAAGTGGCCGTGGTACATTTGGCTGGGTTTCATTGCCGGTCTGATCAGA
AGACTTGTGT
Half_HR2_Stalk_N (SEQ ID NO: 15; 1188-1214 of FIG. 1):
AACCACGTCTTCAGAAGTGGCGAAAAACCTCAACGAGAGTCTGATCGATCTGCAAGAACTGGGCA
AATACGAGCAGTACATCAAGTGGCCGAGAAGACTTGTGT
Central Helix to end of transmembrane domain of S protein (SEQ ID
NO: 16; amino acids 986-1237 of SEQ ID NO: 28):
KVEAEVQIDRLITGRLQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGTHWFVTORNFYEPQII
TTDNTFVSGNCDVVIGIVNNTVYDPLQPELDSFKEELDKYFKNHTSPDVDLGDISGINASVVNIQ
KEIDRLNEVAKNLNESLIDLQELGKYEQYIKWPWYIWLGFIAGLIAIVMVTIMLCCM

TABLE 4
Dominant SARS-COV T Cell Epitopes: 6 Sequences
TCD8 Epitopes Sequences

| | | SARS/ | |
|---|---|---|---|
| | | SARS- | |
| | mapped | CoV-2 | References |
| | star- | identify | (each of which is incorporated |
| Epitopes | end* | (%) | by reference in its entirety) |
| LITGRLQSL | 996-1004 | 100 | Wang et al., 2004b |
| SEQ ID NO: 17 | | | Chen et al., 2005 |
| | | | Tsao et al., 2006 |
| | | | Kohyama et al., 2009 |
| QLIRAAEIRASA | 1011-1028 | 100 | Yang et al., 2009 |
| NLAATK | | | |
| SEQ ID NO: 18 | | | |
| VVFLHVTYV | 1060-1068 | 100 | Tsao et al., 2006 |
| SEQ ID NO: 19 | | | |
| HWFVTQRNFYE | 1101-1115 | 73 | |
| PQII | | | |
| SEQ ID NO: 20 | | | |
| NNTVYDPLQPEL | 1133-1144 | 100 | Huang et al., 2007 |
| SEQ ID NO: 21 | | | |
| RLNEVAKNL | 1185-1193 | 100 | Wang et al., 2004a |
| SEQ ID NO: 22 | | | Chen et al., 2005 |
| | | | Kohyama et al., 2009 |
| NLNESLIDL | 1192-1200 | 100 | Lv et al., 2009 |
| SEQ ID NO: 23 | | | |
| FIAGLIAIV | 1220-1228 | 100 | Wang et al., 2004b |
| SEQ ID NO: 24 | | | Chen et al., 2005 |
| | | | Zhou et al., 2006 |
| | | | Kohyama et al., 2009 |

*Amino acid positions refer to those of SEQ ID NO: 28

TABLE 5
Poli-Epitope: B Cell And TCD4 And TCD8 to SARS-CoV-2

| | | | SARS/ | |
|---|---|---|---|---|
| | | mapped | SARS-CoV-2 | |
| | | star- | identify | |
| Cells | Sequence | end* | (%) | REFERENCE |
| B cell | ...FGAGAALQIPF | 888-909 | 100 | He et al., 2004 |
| epitope | AMQMAYRENGI... | | | |
| | (SEQ ID NO: 25) | | | |
| TCD4 | ..IPF | 896-909 | 100 | |
| epitope | AMQMAYRENGI... | | | |
| | (SEQ ID NO: 26) | | | |
| TCD8 | ...IPFAMQMAY... | 896-904 | 100 | |
| epitope | (SEQ ID NO: 27) | | | |

*Amino acid positions refer to those of SEQ ID NO: 28 (Alba et al., 2020). https://www.iedb.org/result_v3.php?cookie_id=c10360

GENBANK ® Accession No. QHD43416.1: surface glycoprotein [Severe acute
respiratory syndrome coronavirus 2]
(SEQ ID NO: 28)
MFVFLVLLPLVSSQCVNLTTRTQLPPAYTNSFTRGVYYPDKVFRSSVLHSTQDLFLPFFSNVTWF
HAIHVSGTNGTKRFDNPVLPENDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQGNFKNLREFVFK
NIDGYFKIYSKHTPINLVRDLPQGFSALEPLVDLPIGINITRFQTLLALHRSYLTPGDSSSGWTA
GAAAYYVGYLQPRTFLLKYNENGTITDAVDCALDPLSETKCTLKSFTVEKGIYQTSNERVQPTES
IVRFPNITNLCPFGEVENATRFASVYAWNRKRISNCVADYSVLYNSASFSTFKCYGVSPTKLNDL
CFTNVYADSFVIRGDEVRQIAPGQTGKIADYNYKLPDDFTGCVIAWNSNNLDSKVGGNYNYLYRL
FRKSNLKPFERDISTEIYQAGSTPCNGVEGENCYFPLQSYGFQPTNGVGYQPYRVVVLSFELLHA
PATVCGPKKSTNLVKNKCVNFNFNGLTGTGVLTESNKKFLPFQQFGRDIADTTDAVRDPQTLEIL
DITPCSFGGVSVITPGTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLGAENSVAYSNNSIAIP
TNFTISVTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLFNKVTLADAGFIKQYGDCLGDIA
ARDLICAQKENGLTVLPPLLTDEMIAQYTSALLAGTITSGWTFGAGAALQIPFAMQMAYRENGIG
VTQNVLYENQKLIANQFNSAIGKIQDSLSSTASALGKLQDVVNQNAQALNTLVKQLSSNFGAISS
VLNDILSRLDKVEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVT
QRNFYEPQIITTDNTFVSGNCDVVIGIVNNTVYDPLQPELDSFKEELDKYFKNHTSPDVDLGDIS
GINASVVNIQKEIDRLNEVAKNLNESLIDLQELGKYEQYIKWPWYIWLGFIAGLIAIVMVTIMLC
CMTSCCSCLKGCCSCGSCCKFDEDDSEPVLKGVKLHYT

### COVID19 Fusion Peptide 1Mer Construct Name: COVID19_Monomer_V2

### COVID19 Fusion Peptide 5Mer Construct Name: COVID19_5Mer_V2

### COVID19 Fusion Peptide 1Mer and 5Mer Construct Designs:

The COVID19 AA Fusion Peptide, which is conserved in the original SARS-CoV outbreak, (Madu et al., 2009; Wrapp et al., 2020) was taken and a construct was designed that has a monomer and a construct that has 5 repeats with 3 glycines as spacers in between each repeat. Seven (7) more amino acids were added from the N terminal region of the COVID19 AA FP sequence because Miyoshi-Akiyama and co-workers showed development of a human monoclonal antibody called 5H10 against the S protein of SARS-CoV of this region (Miyoshi-Akiyama et al., 2011).

BbsI restriction sites were added to the ends that match with pRIAIDA2 so that the Covid19 FP 1mer and 5mer can be expressed with the AIDA autotransporter system. The sequence codon optimized for E. coli (except for the BbsI sites added) using GeneWiz's Codon Optimization tool and then ordered through Blue Heron Biology (Seattle, WA).

COVID19 Fusion Peptide AA Sequence:
SFIEDLLENKVTLADAGF (SEQ ID NO: 29; amino acids 816-833 of SEQ ID NO: 28)
5H10 hMAB epitope binding AA Sequence on SARS-CoV:
PLKPTKRSFIEDLLF (SEQ ID NO: 30; amino acids 811-823 of SEQ ID NO: 28)
COVID19 FP with the added amino acids from COVID19:
PSKPSKRSFIEDLLFNKVTLADAGF (SEQ ID NO: 31; amino acids 809-833 of SEQ ID NO: 28)
COVID19 FP Monomer sequence after codon optimization and BbsI sites on the ends:
AACCACGTCTTCACCGAGCAAACCGAGCAAACGCAGCTTCATCGAGGATCTGCTGTTCAA
CAAGGTGACGCTGGCCGATGCCGGTTTTGGTGGCGGCAGAAGACTTGTGT
(SEQ ID NO: 32)
COVID19 FP Monomer sequence after codon optimization and BbsI sites on the ends
cloned into pRIAIDA2:
GGCCGCCACCGCTGAGCAATAACTAGCATAACCCCTTGGGGCCTCTAAACGGGTCTTGAGGGGTT
TTTTGCTGAAAGGAGGAACTATATCCGGGTAACGAATTCAAGCTTGATATCATTCAGGACGAGCC
TCAGACTCCAGCGTAACTGGACTGCAATCAACTCACTGGCTCACCTTCACGGGTGGGCCTTTCTT
CGGTAGAAGTCATCTCATGACCAAAATCCCTTAACGTGAGTTACGCGCGCGTCGTTCCACTGAGC
GTCAGACCCCGTAGAAAAGATCAAAGGATCTTCTTGAGATCCTTTTTTTCTGCGCGTAATCTGCT
GCTTGCAAACAAAAAAACCACCGCTACCAGCGGTGGTTTGTTTGCCGGATCAAGAGCTACCAACT
CTTTTTCCGAAGGTAACTGGCTTCAGCAGAGCGCAGATACCAAATACTGTTCTTCTAGTGTAGCC
GTAGTTAGCCCACCACTTCAAGAACTCTGTAGCACCGCCTACATACCTCGCTCTGCTAATCCTGT
TACCAGTGGCTGCTGCCAGTGGCGATAAGTCGTGTCTTACCGGGTTGGACTCAAGACGATAGTTA
CCGGATAAGGCGCAGCGGTCGGGCTGAACGGGGGGTTCGTGCACACAGCCCAGCTTGGAGCGAAC
GACCTACACCGAACTGAGATACCTACAGCGTGAGCTATGAGAAAGCGCCACGCTTCCCGAAGGGA
GAAAGGCGGACAGGTATCCGGTAAGCGGCAGGGTCGGAACAGGAGAGCGCACGAGGGAGCTTCCA
GGGGGAAACGCCTGGTATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACGCGGCCTTTTTACGGT
TCCTGGCCTTTTGCTGGCCTTTTGCTCACATGTTCTTTCCTGCGTTATCCCCTGATTCTGTGGAT
AACCGTATTACCGCCTTTGAGTGAGCTGATACCGCTCGCGCAGAAAGGCCCACCCGAAGGTGAGC
CAGGTGATTACATTTGGGCCCTCATTAGAAAAACTCATCGAGCATCAAATGAAATTGCAATTTAT
TCATATCAGGATTATCAATACCATATTTTTGAAAAAGCCGTTTCTGTAATGAAGGAGAAAACTCA
CCGAGGCAGTTCCATAGGATGGCAAGATCCTGGTATCGGTCTGCGATTCCGACTCGTCCAACATC
AATACAACCTATTAATTTCCCCTCGTCAAAAATAAGGTTATCAAGTGAGAAATCACCATGAGTGA
CGACTGAATCCGGTGAGAATGGCAAAAGTTTATGCATTTCTTTCCAGACTTGTTCAACAGGCCAG
CCATTACGCTCGTCATCAAAATCACTCGCATCAACCAAACCGTTATTCATTCGTGATTGCGCCTG
AGCGAGGCGAAATACGCGATCGCTGTTAAAAGGACAATTACAAACAGGAATCGAGTGCAACCGGC
GCAGGAACACTGCCAGCGCATCAACAATATTTTCACCTGAATCAGGATATTCTTCTAATACCTGG
AACGCTGTTTTTCCGGGGATCGCAGTGGTGAGTAACCATGCATCATCAGGAGTACGGATAAAATG
CTTGATGGTCGGAAGTGGCATAAATTCCGTCAGCCAGTTTAGTCTGACCATCTCATCTGTAACAT
CATTGGCAACGCTACCTTTGCCATGTTTCAGAAACAACTCTGGCGCATCGGGCTTCCCATACAAG
CGATAGATTGTCGCACCTGATTGCCCGACATTATCGCGAGCCCATTTATACCCATATAAATCAGC
ATCCATGTTGGAATTTAATCGCGGCCTCGACGTTTCCCGTTGAATATGGCTCATAGCTCCTGAAA
ATCTCGATAACTCAAAAAATACGCCCGGTAGTGATCTTATTTCATTATGGTGAAAGTTGGAACCT
CTTACGTGCCGATCAAAAAGACGGTCAAAAGCCTCCGGTCGGAGGCTTTTGACTTTCTGCTATGG
AGGTCAGGTATGATTTAAATGGTCAGTATTGAGCGATATCTAGAGAATTCGTCCACCACAATTCA
GCAAATTGTGAACATCATCACGTTCATCTTTCCCTGGTTGCCAATGGCCCATTTTCCTGTCAGTA
ACGAGAAGGTCGCGAATTCAGGCGCTTTTTAGACTGGTCGTAATGAAATTCTTTTTAAGAGGAGA
TATACATAGGTCTCACATGATTAAATTAAAATTTGGTGTTTTTTTTACAGTTTTACTATCTTCAG
CATATGCACATGGAACACCTCAAAATATTACTCACGGTGTGTTAACCACGTCTTCACCGAGCAAA
CCGAGCAAACGCAGCTTCATCGAGGATCTGCTGTTCAACAAGGTGACGCTGGCCGATGCCGGTTT
TGGTGGCGGCAGAAGACTTGTGTTAACAATGGAGATCTTCGAACAATACAGACCGGAGAACGGAA
GTTATGCTACCAATATGGCACTGGCTAACTCACTGTTCCTCATGGATTTGAATGAGCGTAAGCAA
TTCAGGGCCATGAGTGATAATACACAGCCTGAGTCTGCATCCGTGTGGATGAAGATCACTGGAGG
AATAAGCTCTGGTAAGCTTAATGACGGGCAAAATAAAACAACAACCAATCAGTTTATCAATCAGC
TCGGGGGGGATATTTATAAATTCCATGCTGAACAACTGGGTGATTTTACCTTAGGGATTATGGGA
GGATACGCGAATGCAAAAGGTAAAACGATAAATTACACGAGCAACAAAGCTGCCAGAAACACACT
GGATGGTTATTCTGTCGGGGTATACGGTACGTGGTATCAGAATGGGGAAAATGCAACAGGGCTCT
TTGCTGAAACTTGGATGCAATATAACTGGTTTAATGCATCAGTGAAAGGTGACGGACTGGAAGAA
GAAAAATATAATCTGAATGGTTTAACCGCTTCTGCAGGTGGGGGATATAACCTGAATGTGCACAC
ATGGACATCACCTGAAGGAATAACAGGTGAATTCTGGTTACAGCCTCATTTGCAGGCTGTCTGGA
TGGGGGTTACACCGGATACACATCAGGAGGATAACGGAACGGTGGTGCAGGGAGCAGGGAAAAAT
AATATTCAGACAAAAGCAGGTATTCGTGCATCCTGGAAGGTGAAAAGCACCCTGGATAAGGATAC
CGGGCGGAGGTTCCGTCCGTATATAGAGGCAAACTGGATCCATAACACTCATGAATTTGGTGTTA
AAATGAGTGATGACAGCCAGTTGTTGTCAGGTAGCCGAAATCAGGGAGAGATAAAGACAGGTATT
GAAGGGGTGATTACTCAAAACTTGTCAGTGAATGGCGGAGTCGCATATCAGGCAGGAGGTCACGG
GAGCAATGCCATCTCCGGAGCACTGGGGATAAAATACAGCTTCGGTTAGAGC (SEQ ID NO:
33)
COVID19 FP 5 mer sequence after codon optimization and BbsI sites on the ends:
AACCACGTCTTCACCGAGTAAACCGAGTAAACGCAGCTTTATTGAGGATCTGCTCTTCAACAAGG
TGACGCTGGCCGACGCCGGTTTTGGCGGCGGCCCGAGCAAGCCGAGCAAACGTAGCTTTATCGAG
GATCTGCTCTTTAATAAAGTGACGCTGGCGGATGCGGGTTTCGGCGGTGGCCCGAGCAAACCAAG
CAAACGTAGTTTCATCGAGGATCTGCTGTTCAATAAAGTTACGCTGGCCGATGCGGGTTTTGGTG
GTGGTCCAAGCAAACCAAGTAAGCGTAGTTTTATTGAGGATCTGCTGTTTAACAAGGTTACCCTC
GCCGATGCCGGTTTTGGTGGCGGTCCGAGTAAGCCGAGTAAGCGTAGCTTCATTGAAGATCTGCT
GTTCAACAAAGTGACGCTGGCCGATGCCGGCTTTGGCGGTGGTAGAAGACTTGTGT
(SEQ ID NO: 34)
COVID19 FP 5 mer sequence after codon optimization and BbsI sites on the ends
cloned into pRIAIDA2:
GGCCGCCACCGCTGAGCAATAACTAGCATAACCCCTTGGGGCCTCTAAACGGGTCTTGAGGGGTT
TTTTGCTGAAAGGAGGAACTATATCCGGGTAACGAATTCAAGCTTGATATCATTCAGGACGAGCC
TCAGACTCCAGCGTAACTGGACTGCAATCAACTCACTGGCTCACCTTCACGGGTGGGCCTTTCTT
CGGTAGAAGTCATCTCATGACCAAAATCCCTTAACGTGAGTTACGCGCGCGTCGTTCCACTGAGC
GTCAGACCCCGTAGAAAAGATCAAAGGATCTTCTTGAGATCCTTTTTTTCTGCGCGTAATCTGCT
GCTTGCAAACAAAAAAACCACCGCTACCAGCGGTGGTTTGTTTGCCGGATCAAGAGCTACCAACT
CTTTTTCCGAAGGTAACTGGCTTCAGCAGAGCGCAGATACCAAATACTGTTCTTCTAGTGTAGCC
GTAGTTAGCCCACCACTTCAAGAACTCTGTAGCACCGCCTACATACCTCGCTCTGCTAATCCTGT
TACCAGTGGCTGCTGCCAGTGGCGATAAGTCGTGTCTTACCGGGTTGGACTCAAGACGATAGTTA
CCGGATAAGGCGCAGCGGTCGGGCTGAACGGGGGGTTCGTGCACACAGCCCAGCTTGGAGCGAAC
GACCTACACCGAACTGAGATACCTACAGCGTGAGCTATGAGAAAGCGCCACGCTTCCCGAAGGGA
GAAAGGCGGACAGGTATCCGGTAAGCGGCAGGGTCGGAACAGGAGAGCGCACGAGGGAGCTTCCA
GGGGGAAACGCCTGGTATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACGCGGCCTTTTTACGGT
TCCTGGCCTTTTGCTGGCCTTTTGCTCACATGTTCTTTCCTGCGTTATCCCCTGATTCTGTGGAT
AACCGTATTACCGCCTTTGAGTGAGCTGATACCGCTCGCGCAGAAAGGCCCACCCGAAGGTGAGC
CAGGTGATTACATTTGGGCCCTCATTAGAAAAACTCATCGAGCATCAAATGAAATTGCAATTTAT
TCATATCAGGATTATCAATACCATATTTTTGAAAAAGCCGTTTCTGTAATGAAGGAGAAAACTCA
CCGAGGCAGTTCCATAGGATGGCAAGATCCTGGTATCGGTCTGCGATTCCGACTCGTCCAACATC
AATACAACCTATTAATTTCCCCTCGTCAAAAATAAGGTTATCAAGTGAGAAATCACCATGAGTGA
CGACTGAATCCGGTGAGAATGGCAAAAGTTTATGCATTTCTTTCCAGACTTGTTCAACAGGCCAG
CCATTACGCTCGTCATCAAAATCACTCGCATCAACCAAACCGTTATTCATTCGTGATTGCGCCTG
AGCGAGGCGAAATACGCGATCGCTGTTAAAAGGACAATTACAAACAGGAATCGAGTGCAACCGGC
GCAGGAACACTGCCAGCGCATCAACAATATTTTCACCTGAATCAGGATATTCTTCTAATACCTGG
AACGCTGTTTTTCCGGGGATCGCAGTGGTGAGTAACCATGCATCATCAGGAGTACGGATAAAATG
CTTGATGGTCGGAAGTGGCATAAATTCCGTCAGCCAGTTTAGTCTGACCATCTCATCTGTAACAT
CATTGGCAACGCTACCTTTGCCATGTTTCAGAAACAACTCTGGCGCATCGGGCTTCCCATACAAG
CGATAGATTGTCGCACCTGATTGCCCGACATTATCGCGAGCCCATTTATACCCATATAAATCAGC
ATCCATGTTGGAATTTAATCGCGGCCTCGACGTTTCCCGTTGAATATGGCTCATAGCTCCTGAAA
ATCTCGATAACTCAAAAAATACGCCCGGTAGTGATCTTATTTCATTATGGTGAAAGTTGGAACCT
CTTACGTGCCGATCAAAAAGACGGTCAAAAGCCTCCGGTCGGAGGCTTTTGACTTTCTGCTATGG
AGGTCAGGTATGATTTAAATGGTCAGTATTGAGCGATATCTAGAGAATTCGTCCACCACAATTCA
GCAAATTGTGAACATCATCACGTTCATCTTTCCCTGGTTGCCAATGGCCCATTTTCCTGTCAGTA
ACGAGAAGGTCGCGAATTCAGGCGCTTTTTAGACTGGTCGTAATGAAATTCTTTTTAAGAGGAGA
TATACATAGGTCTCACATGATTAAATTAAAATTTGGTGTTTTTTTTACAGTTTTACTATCTTCAG
CATATGCACATGGAACACCTCAAAATATTACTCACGGTGTGTTAACCACGTCTTCACCGAGTAAA
CCGAGTAAACGCAGCTTTATTGAGGATCTGCTCTTCAACAAGGTGACGCTGGCCGACGCCGGTTT
TGGCGGCGGCCCGAGCAAGCCGAGCAAACGTAGCTTTATCGAGGATCTGCTCTTTAATAAAGTGA
CGCTGGCGGATGCGGGTTTCGGCGGTGGCCCGAGCAAACCAAGCAAACGTAGTTTCATCGAGGAT
CTGCTGTTCAATAAAGTTACGCTGGCCGATGCGGGTTTTGGTGGTGGTCCAAGCAAACCAAGTAA
GCGTAGTTTTATTGAGGATCTGCTGTTTAACAAGGTTACCCTCGCCGATGCCGGTTTTGGTGGCG
GTCCGAGTAAGCCGAGTAAGCGTAGCTTCATTGAAGATCTGCTGTTCAACAAAGTGACGCTGGCC
GATGCCGGCTTTGGCGGTGGTAGAAGACTTGTGTTAACAATGGAGATCTTCGAACAATACAGACC
GGAGAACGGAAGTTATGCTACCAATATGGCACTGGCTAACTCACTGTTCCTCATGGATTTGAATG
AGCGTAAGCAATTCAGGGCCATGAGTGATAATACACAGCCTGAGTCTGCATCCGTGTGGATGAAG
ATCACTGGAGGAATAAGCTCTGGTAAGCTTAATGACGGGCAAAATAAAACAACAACCAATCAGTT
TATCAATCAGCTCGGGGGGGATATTTATAAATTCCATGCTGAACAACTGGGTGATTTTACCTTAG
GGATTATGGGAGGATACGCGAATGCAAAAGGTAAAACGATAAATTACACGAGCAACAAAGCTGCC
AGAAACACACTGGATGGTTATTCTGTCGGGGTATACGGTACGTGGTATCAGAATGGGGAAAATGC
AACAGGGCTCTTTGCTGAAACTTGGATGCAATATAACTGGTTTAATGCATCAGTGAAAGGTGACG
GACTGGAAGAAGAAAAATATAATCTGAATGGTTTAACCGCTTCTGCAGGTGGGGGATATAACCTG
AATGTGCACACATGGACATCACCTGAAGGAATAACAGGTGAATTCTGGTTACAGCCTCATTTGCA
GGCTGTCTGGATGGGGGTTACACCGGATACACATCAGGAGGATAACGGAACGGTGGTGCAGGGAG
CAGGGAAAAATAATATTCAGACAAAAGCAGGTATTCGTGCATCCTGGAAGGTGAAAAGCACCCTG
GATAAGGATACCGGGCGGAGGTTCCGTCCGTATATAGAGGCAAACTGGATCCATAACACTCATGA
ATTTGGTGTTAAAATGAGTGATGACAGCCAGTTGTTGTCAGGTAGCCGAAATCAGGGAGAGATAA
AGACAGGTATTGAAGGGGTGATTACTCAAAACTTGTCAGTGAATGGCGGAGTCGCATATCAGGCA
GGAGGTCACGGGAGCAATGCCATCTCCGGAGCACTGGGGATAAAATACAGCTTCGGTTAGAGC (S
EQ ID NO: 35)
Nucleotide Sequence of pRIAIDA2 (SEQ ID NO: 36):

| | |
|---|---|
| 1 | ggccgccaccgctgagcaataactagcataaccccttggggcctctaaacgggtcttgag |
| 61 | gggttttttgctgaaaggaggaactatatccgggtaacgaattcaagcttgatatcattc |
| 121 | aggacgagcctcagactccagcgtaactggactgcaatcaactcactggctcaccttcac |
| 181 | gggtgggcctttcttcggtagaagtcatctcatgaccaaaatcccttaacgtgagttacg |
| 241 | cgcgcgtcgttccactgagcgtcagaccccgtagaaaagatcaaaggatcttcttgagat |
| 301 | cctttttttctgcgcgtaatctgctgcttgcaaacaaaaaaaccaccgctaccagcggtg |
| 361 | gtttgtttgccggatcaagagctaccaactctttttccgaaggtaactggcttcagcaga |
| 421 | gcgcagataccaaatactgttcttctagtgtagccgtagttagcccaccacttcaagaac |
| 481 | tctgtagcaccgcctacatacctcgctctgctaatcctgttaccagtggctgctgccagt |
| 541 | ggcgataagtcgtgtcttaccgggttggactcaagacgatagttaccggataaggcgcag |
| 601 | cggtcgggctgaacggggggttcgtgcacacagcccagcttggagcgaacgacctacacc |
| 661 | gaactgagatacctacagcgtgagctatgagaaagcgccacgcttcccgaagggagaaag |
| 721 | gcggacaggtatccggtaagcggcagggtcggaacaggagagcgcacgagggagcttcca |
| 781 | gggggaaacgcctggtatctttatagtcctgtcgggtttcgccacctctgacttgagcgt |
| 841 | cgatttttgtgatgctcgtcaggggggcggagcctatggaaaaacgccagcaacgcggcc |
| 901 | tttttacggttcctggccttttgctggccttttgctcacatgttctttcctgcgttatcc |
| 961 | cctgattctgtggataaccgtattaccgcctttgagtgagctgataccgctcgcgcagaa |
| 1021 | aggcccacccgaaggtgagccaggtgattacatttgggccctcattagaaaaactcatcg |
| 1081 | agcatcaaatgaaattgcaatttattcatatcaggattatcaataccatatttttgaaaa |
| 1141 | agccgtttctgtaatgaaggagaaaactcaccgaggcagttccataggatggcaagatcc |
| 1201 | tggtatcggtctgcgattccgactcgtccaacatcaatacaacctattaatttcccctcg |
| 1261 | tcaaaaataaggttatcaagtgagaaatcaccatgagtgacgactgaatccggtgagaat |
| 1321 | ggcaaaagtttatgcatttctttccagacttgttcaacaggccagccattacgctcgtca |
| 1381 | tcaaaatcactcgcatcaaccaaaccgttattcattcgtgattgcgcctgagcgaggcga |
| 1441 | aatacgcgatcgctgttaaaaggacaattacaaacaggaatcgagtgcaaccggcgcagg |
| 1501 | aacactgccagcgcatcaacaatattttcacctgaatcaggatattcttctaatacctgg |
| 1561 | aacgctgtttttccggggatcgcagtggtgagtaaccatgcatcatcaggagtacggata |
| 1621 | aaatgcttgatggtcggaagtggcataaattccgtcagccagtttagtctgaccatctca |
| 1681 | tctgtaacatcattggcaacgctacctttgccatgtttcagaaacaactctggcgcatcg |
| 1741 | ggcttcccatacaagcgatagattgtcgcacctgattgcccgacattatcgcgagcccat |
| 1801 | ttatacccatataaatcagcatccatgttggaatttaatcgcggcctcgacgtttcccgt |
| 1861 | tgaatatggctcatagctcctgaaaatctcgataactcaaaaaatacgcccggtagtgat |
| 1921 | cttatttcattatggtgaaagttggaacctcttacgtgccgatcaaaaagacggtcaaaa |
| 1981 | gcctccggtcggaggcttttgactttctgctatggaggtcaggtatgatttaaatggtca |
| 2041 | gtattgagcgatatctagagaattcgtccaccacaattcagcaaattgtgaacatcatca |
| 2101 | cgttcatctttccctggttgccaatggcccattttcctgtcagtaacgagaaggtcgcga |
| 2161 | attcaggcgctttttagactggtcgtaatgaaattctttttaagaggagatatacatagg |
| 2221 | tctcacatgattaaattaaaatttggtgttttttttacagttttactatcttcagcatat |
| 2281 | gcacatggaacacctcaaaatattactcacggtgtgttaaccctgtcttctcgtggtctg |
| 2341 | gactatccatacgatgtaccggattacgcgcgttatggaagacgagtgttaacaatggag |
| 2401 | atcttcgaacaatacagaccggagaacggaagttatgctaccaatatggcactggctaac |
| 2461 | tcactgttcctcatggatttgaatgagcgtaagcaattcagggccatgagtgataataca |
| 2521 | cagcctgagtctgcatccgtgtggatgaagatcactggaggaataagctctggtaagctt |
| 2581 | aatgacgggcaaaataaaacaacaaccaatcagtttatcaatcagctcgggggggatatt |
| 2641 | tataaattccatgctgaacaactgggtgattttaccttagggattatgggaggatacgcg |
| 2701 | aatgcaaaaggtaaaacgataaattacacgagcaacaaagctgccagaaacacactggat |
| 2761 | ggttattctgtcggggtatacggtacgtggtatcagaatggggaaaatgcaacagggctc |
| 2821 | tttgctgaaacttggatgcaatataactggtttaatgcatcagtgaaaggtgacggactg |
| 2881 | gaagaagaaaaatataatctgaatggtttaaccgcttctgcaggtgggggatataacctg |
| 2941 | aatgtgcacacatggacatcacctgaaggaataacaggtgaattctggttacagcctcat |
| 3001 | ttgcaggctgtctggatgggggttacaccggatacacatcaggaggataacggaacggtg |
| 3061 | gtgcagggagcagggaaaaataatattcagacaaaagcaggtattcgtgcatcctggaag |
| 3121 | gtgaaaagcaccctggataaggataccgggcggaggttccgtccgtatatagaggcaaac |
| 3181 | tggatccataacactcatgaatttggtgttaaaatgagtgatgacagccagttgttgtca |
| 3241 | ggtagccgaaatcagggagagataaagacaggtattgaaggggtgattactcaaaacttg |
| 3301 | tcagtgaatggcggagtcgcatatcaggcaggaggtcacgggagcaatgccatctccgga |
| 3361 | gcactggggataaaatacagcttcggttagagc |

### Example 6

### SARS-CoV-2 and Analogous PEDV FP Vaccine

It has been shown that monomeric MPER-derived scaffold proteins (designed by and gift of Peter Kwong, VRC, NIH) can be expressed on the surfaces of Gram-bacteria using a monomeric AT and that native MPER sequence can be displayed using the Hia trimeric AT, and that these are bound by anti-HIV BN mAbs. It has also shown that immunizing mice with bacteria expressing the monomeric construct can elicit anti-HIV neutralizing sera in mice. It has also been shown that expressing a test antigen on the surface of grEC, produced by the Tokyo Metropolitan University Group (Hashimoto et al., 2005; Kato & Hashimoto, 2007), with up to 29.7% of the genome deleted yields dramatically better binding by flow cytometry of a mAb directed against the test Ag, and that immunizing mice with grEc expressing the test Ag yields a dramatically improved immune response.

The origin, evolution, and genotypes of emergent PEDV strains in the United States has been determined (Huang et al., 2013). A VLP-based PEDV vaccine containing B-cell epitope ⁷⁴⁸YSNIGVCK⁷⁵⁵ (SEQ ID NO: 45) from the PEDV S protein incorporated into the hepatitis B virus core capsid VLP was developed. The vaccine showed evidence of protection and reduced viral shedding in a challenge model (Subramaniam et al., 2017; Subramaniam et al., 2018).

This EXAMPLE relates to the construction of recombinant bacterial killed whole cell SARS-CoV-2 vaccines by displaying the spike protein stalk on the surfaces of genome-reduced E. coli using trimeric Gram-negative autotransporters (ATs).

Surface displaying SARS-CoV-2 stalk on grEc should yield an effective vaccine against SARS-CoV-2 and so it is proposed to synthesize SARS-CoV-2 and later analogous PEDV stalk coding sequence. Several constructs are synthesized, incorporating different amounts of stalk, fused in different configurations to the trimeric Hia AT expression cassette, to place stalk at different distances from the beta barrel and outer membrane lipid bilayer, in all cases working to maintain correct phasing of the heptad repeats (Wrapp et al., 2020). See EXAMPLE 5. Several candidate stalk vaccines are synthesized and evaluated by immunizing mice, followed by neutralization assay for anti-SARS-CoV-2 neutralizing activity. FIG. 1 shows the initial collection of stalk region variants.

Surface expression is verified by flow cytometry and immunoblot with/without trypsin treatment. (A trypsin site lies between the beta barrel region and expression cloning site.)

Constructs are selected exhibiting the best antibody binding by flow cytometry and prepare KWC vaccines, verifying expression prior to immunizations. Groups of 5 mice are immunized intranasally or intramuscularly with the vaccines, collecting sera pre-immune and at weekly intervals, with boosts at 2 and 4 weeks, followed by terminal bleed. Sera are assayed for anti-SARS-CoV-2 neutralizing Abs with a VSV SARS-CoV-2 pseudovirus neutralization assay and anti-SARS-CoV-2 cellular immune responses by ELISpot. It is established which SARS-CoV-2 stalk constructs elicits the best neutralizing Abs, then DNAs for the PEDV stalk analogue of the two most potent constructs are synthesized and cloned into the expression vector. Surface expression is verified, KWC are prepared, mice are immunized, and an assay for anti-PEDV neutralization activity (Subramaniam et al., 2017) is performed. Vaccine preparations for pig vaccine efficacy studies are made.

This EXAMPLE also relates to the assessment of protective efficacy of SARS-CoV-2 analogous candidate vaccines using surrogate pig and porcine coronavirus PEDV model system. Vaccination of pigs with a recombinant KWC PEDV stalk vaccine, analogous to a SARS-CoV-2 stalk vaccine, should protect pigs against infectious PEDV challenge.

Specific-pathogen-free (SPF) pigs in 7 groups of 5 each are used, and are immunized orally, intranasally (IN), or intramuscularly (IM) with the most potent SARS-CoV-2 analogous PEDV-stalk vaccine as determined above. A negative control group is immunized with whole cell prep without vaccine antigen. Boosts are performed at 21 days post-vaccination (DPV) and necropsy is performed at 35 DPV. Blood samples are collected before vaccination then weekly thereafter for serum and PBMCs. Anti-PEDV-stalk immunity is assessed by virus neutralization assay (Subramaniam et al., 2017; Subramaniam et al., 2018), and T cells responses by ELIspot and flow cytometry for Ag-specific T cell responses. Based on the results, the best vaccination route and most potent vaccine are selected.

For a PEDV protection study groups of 8 pigs are used and are vaccinated with the most potent candidate via the optimal route as determined above. Serum is collected prior to vaccination and weekly thereafter. KWC bacterial preparation without viral antigen are used as a negative control. A commercial PEDV vaccine “Zoetis-PED” is used as a positive control. Boosts are performed at 21 DPV. At 35 DPV, challenges are performed with a 10^{4.5} TCID₅₀ of PEDV CO/13 strain (Subramaniam et al., 2017; Subramaniam et al., 2018). Serum samples are tested for neutralizing titers. Fecal samples are collected after challenge, plus small intestine tissue samples at necropsy for viral RNA loads. Daily clinical scores, gross and histological lesion scores, viral RNA loads in feces, serum and tissues, and T cell immune responses are recorded. Animals are monitored daily for clinical signs as described (Subramaniam et al., 2018).

Statistical considerations/Rigor/Reproducibility: Both female and castrated male SPF pigs are used post-weaning. The estimated sample size in the protection study to obtain >0.9 power is n≥7 based on a power analysis using an ANOVA or ANCOVA model indicates that 8 pigs per group are sufficient. For the immunogenicity study, 5 pigs per group are sufficient, and an equivalent analysis applies to the mouse immunization studies described above. Significance is assessed at P<0.05.

It is expected that we will make a SARS-CoV-2 trimeric stalk grEc KWC vaccine that elicits a neutralizing response, with pig safety and PEDV protection data, suitable for advancement to early phase clinical trials, an important step in producing a globally appropriate SARS-CoV-2 vaccine.

### Materials and Methods for Examples 7-13

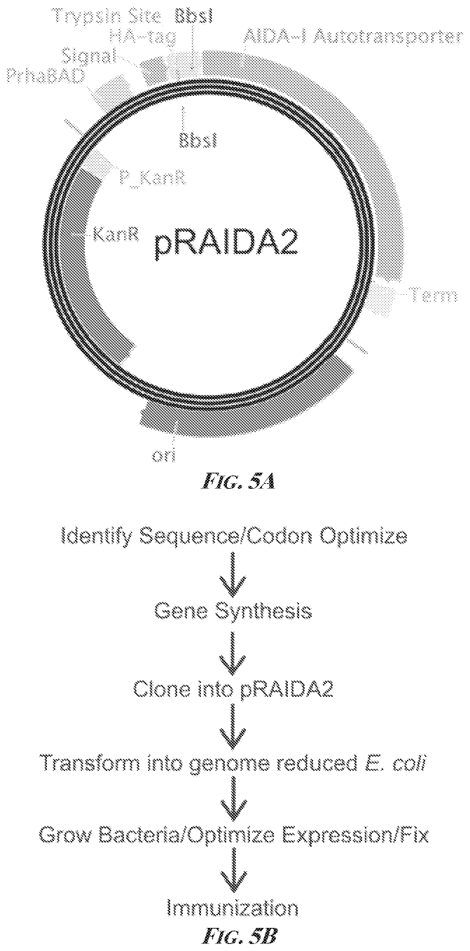

Plasmid synthesis. The plasmid pRAIDA2, which contains a high copy origin of replication, a kanamycin resistance gene, and a slightly modified AIDA-I autotransporter surface expression cassette under the control of a rhamnose inducible promoter, was synthesized by GeneWiz (FIG. 5A). The expression cassette has a cloning site with type IIS BbsI restriction sites to enable “scarless” cloning. The stuffer in the parental version of the plasmid encodes an influenza HA immunotag to enable verification of expression. The sequence of pRAIDA2 is presented as SEQ ID NO: 36 (see also GENBANK® Accession No. MW383928). Plasmids were prepared using Qiagen Plasmid Mini Prep kit, quantitated and assessed for quality spectrophotometrically. The construction of vaccines employing a synthetic gene cloned into the pRAIDA2 expression cassette and then expressed on genome-reduced bacteria is schematically illustrated (FIG. 5B).

Bacteria. E. coli strains, including the parental strain and highly genome-deleted bacterial strains, with varying amounts of bacterial genome deletion were the kind gift of J. Kato (Hashimoto et al., 2005; Kato & Hashimoto, 2007), obtained through the National Bioresource Project, E. coli Strain Office, National Institute of Genetics, Japan. The E. coli strains used in this study, MG1655 derivatives, include ME 5000 (wild type, with 0% of the genome deleted), ME 5010 (2.4% deleted), ME 5119 (15.8% deleted), and ME 5125 (29.7% deleted). E. coli strains were grown in LB media and on LB agar plates with appropriate antibiotics.

For molecular cloning work, chemically competent E. coli DH5a was obtained from ThermoFisher and transformations were performed per manufacturer's instructions.

To prepare electrocompetent cells, bacteria were grown in a shaker overnight at 37° C., in LB broth, inoculated from overnight culture and grown to log phase. Cells were collected by centrifugation and washed with ice cold phosphate-buffered saline (PBS)-10% glycerol, resuspended in ice-cold sterile water-10% glycerol, and transformed via electroporation with the pRAIDA2-derived plasmids expressing the SARS-CoV-2 FP or PEDV FP, respectively. Electroporation was conducted in 0.1 cm electroporation cuvettes with the Gene Pulser Xcell electroporation system (Bio-Rad) and pulsed at settings: 1800 V, 25 μF, and 200 K. Electroporated cells were transferred to 1.5 mL microcentrifuge tubes with 1 mL of SOC media (Life Technologies), and grown in an orbital shaker (80 rpm) at 37° C. for 1 h before plating on LB agar plates containing the appropriate antibiotic.

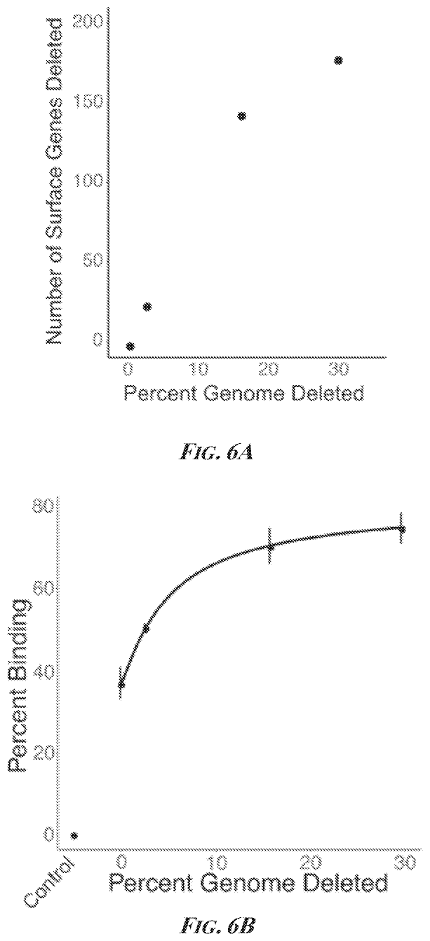

Analysis of genes with imputed expression on the bacterial surface. We examined the lists of genes included in the genome-reduced E. coli strains used in this study (Meurens et al., 2012; Gu et al., 2020). The names of the genes in each deletion were gathered from the Japan National Institute of Genetics National BioResource Project E. coli Strain website (https://shigen.nig.ac.jp/ecoli/strain). Most of the information, including the gene name, protein name, its product, location, function, gene ontology, and other notes about the genes, were retrieved from the National BioResource Project of Japan (https://shigen.nig.ac.jp/ecoli/strain/resource/longDeletion/lddTablelnfo). We also queried the Uniprot and Ecocyc databases (https://www.uniprot.org/; https://ecocyc.org/). This information is listed in Table 6, in which the genes are listed, along with the mutants they have been deleted from and their imputed location in the bacterial cell. The data were analyzed in R and plotted. The Number of Surface Genes Deleted vs Percent Genome Deleted is shown in FIG. 6A.

Design, synthesis, and cloning of FP coding sequences from SARS-CoV-2 and PEDV into pRAIDA2. We synthesized E. coli codon-optimized DNAs (Blue Heron) encoding the SARS-CoV-2 FP: AACCACGTCTTCACCGAGCAAACCGAGCAAACGCAGCTTCATCGAGGATCTGC TGTTCAACAAGGTGACGCTGGCCGATGCCGGTTTTGGTGGCGGCAGAAGACTTGTGT (SEQ ID NO: 32) and PEDV FP: AACCACGTCTTCAGGCCGCGTTGTTCAGAAACG CAGCTTCATCGAGGATCTGCTGTTCAACAAGGTGGTGACCAATGGTCTGGGCACCGG TGGCGGCAGAAGACTTGTGT (SEQ ID NO: 46).

The FP-encoding DNAs were digested with BbS I (New England Biolabs), gel purified, and ligated into Bbs I-digested pRAIDA2, transformed into chemically competent DH5a, and plated on LB agar containing kanamycin.

Production of killed whole cell vaccines. We used overnight cultures to start 50 mL cultures in LB broth with the appropriate antibiotics, incubated in a shaker at 210 rpm, at 37° C., overnight. The next day, the 50 mL overnight cultures were diluted 1:10 in LB broth with antibiotic, and cells were grown to mid-log-phase growth (OD₆₀₀˜0.5-0.6). We induced recombinant protein expression with L-rhamnose (SigmaAldrich), added to a 5 mM final concentration, and incubated the bacteria for an additional 2 h at 37° C., in a shaker at 210 rpm. The bacteria were collected by centrifugation at 5,000×g for 20 min at 4° C. For flow cytometry and vaccine production, the pellet was resuspended in 10 mL of Hank's Balanced Salt Solutions (HBSS) with 0.2% formalin (SigmaAldrich), the cells were incubated at 37° C. for 1 h, shaking at 180 rpm. The bacteria were resuspended in 1×PBS with 20% glycerol, to achieve a final OD600=1.0, ˜ 8×10⁸ cells/mL. Cells were aliquoted in 1 ml aliquots, and stored at −80° C.

Flow cytometry analysis of bacteria expressing vaccine antigens. Approximately 5×10⁷ cells/mL were added to each well of a 96-well V-bottom no-binding plate. Samples were blocked with phosphate-buffered saline (PBS) supplemented with 10% fetal bovine serum (FBS) for 30 min on ice. The plate was washed twice with PBS buffer supplemented with 2% FBS and incubated with appropriate dilution of primary antibody anti-HA (Invitrogen #26183), custom-made anti-FP peptide antibodies (Pacific Immunology; rabbit anti-SARS-CoV-2 FP (1:5000) or rabbit anti-PEDV FP (1:2000)) for 30 min on ice. After washing twice, samples were subsequently stained with 1:600 dilution of AlexaFluor 488 rat anti-mouse or anti-rabbit antibody (BD Biosciences) for 30 min on ice. Samples were examined using a FACSCalibur (BD Biosciences) flow cytometer. Data was analyzed with FlowJo V software (TreeStar). Gating was set using Alexa-488 negative sample for the bacterial population by forward-scatter (FSC) and side-scatter (SSC) and to determine background fluorescence. The binding data is presented in FIG. 6B.

Immunoblots of bacteria expressing vaccine antigens. Normalized quantities of bacterial vaccines and serial dilutions of a recombinant protein, DNAK protein quantity standard (Abcam Ab51121) were resuspended in 4× Laemmli sample buffer (Biorad) and incubated at 100° C. for 5 min. Samples were separated using Novex NuPage 4-12% Bis-Tris Gel (ThermoFisher Scientific) and electrophoretically transferred onto 0.2 m nitrocellulose membranes (BioRad). Membranes were blocked overnight in 3% non-fat dry milk in 0.05% Tween-20 in PBS (PBS-T). After the membranes were washed three times, they were incubated with a primary antibody (for the protein standard, mouse monoclonal anti-DnaK, Abcam (8E2/2) Ab69617) at dilution of 1:2000; or for detection of FP-AIDA-I recombinant proteins, polyclonal rabbit anti-SARS-CoV-2 FP antiserum, at a dilution of 1:4000; or polyclonal rabbit anti-PEDV FP antiserum, at a dilution of 1:2000) in blocking buffer for 1.5 h at room temperature. Membranes were then washed three times in PBS-T and incubated with 1:3000 dilution of goat anti-mouse HRP or goat anti-rabbit-RP (Sigma Aldrich) in blocking buffer for 1 hour at room temperature. After being washed again three times, the membranes were processed for enzyme-linked chemiluminescence using a Western Blot Signal Enhancer kit (ThermoFisher Scientific). The immunoblots signals were captured by ChemiDoc MP (Biorad) and the data was analyzed in Image Lab software (Biorad) and quantitated using ImageJ (https://imagej.nih.gov/ij/).

Propagation and titration of challenge virus. Vero cells (African green monkey kidney cell line) cultured in DMEM medium (Gibco, Waltham, MA) supplemented with 10% FBS (Gibco) were used to propagate the PEDV 2013 Colorado strain (National Veterinary Services Laboratories, Ames, IA). After one hour inoculation with virus, cells were maintained in MEM (Gibco) supplemented with 0.02% yeast extraction, 0.3% tryptose phosphate broth, 2 μg/ml trypsin at 37° C. with 5% CO₂. Five days later, the cell lysate and culture supernatant were collected via 3 rounds of freeze and thaw. After centrifugation (3,000×g, 10 min, 4° C.), the supernatant was collected and stored at −80° C. as virus stock. To determine the infectious titer, 100 uL serially-diluted virus stock (10⁻¹ to 10⁻⁵) was inoculated with each well of Vero cells in a 96-well plate at 37° C. with 5% CO₂. After three days incubation, the inoculated cells were analyzed via immunofluorescence assay (IFA) with a PEDV-specific antibody as previously described (43). Infectious viral titer was calculated using the Reed-Muench method and expressed as TCID₅₀/mL.

Experimental design for the pig vaccination and challenge study. A total of 21 PEDV-negative piglets at 5 weeks of age were randomly divided into 3 groups of 7 piglets per group housed in separate rooms in a BSL-2 swine facility. Piglets in each group were intramuscularly injected into neck muscles with killed whole genome-reduced bacterial cell vaccines expressing SARS-CoV-2 or PEDV FPs on the cell surface, or killed whole bacterial cells as control (Table 5). Pigs received a booster dose at 21 days post-vaccination (dpv). Serum samples were collected from each pig prior to vaccination and at 1, 3, 5, 6 weeks post-vaccination (wpv). At 35 dpv, the pigs were challenged with PEDV 2013 Colorado strain via oral route of inoculation with 3.0×10⁵⁰ TCID₅₀/pig (Subramaniam et al., 2018). After virus challenge, samples of fecal swab materials and scores of clinical signs were collected daily. At 7 days post-challenge (dpc), all pigs were euthanized and necropsied. Samples of intestine tissues and intestinal contents were collected for pathological evaluation and quantification of PEDV RNA loads, respectively. This study was approved by Virginia Tech Institutional Animal Care and Use Committee (approval number IACUC 20-070).

TABLE 5
Experimental design for SARS-CoV-2 FP and PEDV FP
vaccine efficacy study in a PEDV challenge pig model

| | | | | Challenge | No. of |
|---|---|---|---|---|---|
| | No. | Vaccination | Booster at | at 35 | pigs at |
| | of | at 0 | 21 dpv^{a} | dpv^{b} | necropsy |
| Group | pigs | dpv^{a} with | with | with | (7 dpc^{b}) |
| 1 | 7 | ME5125^{c} | ME5125 | PEDV | 7 |
| 2 | 7 | SARS-CoV-2 FP | SARS-CoV-2 FP | PEDV | 7 |
| 3 | 7 | PEDV FP | PEDV FP | PEDV | 7 |

^{a}dpv, days post-vaccination; Vaccination dose: SARS-CoV-2 FP 110 μg/pig, PEDV FP and ME5125: 250 μg/pig. Booster dose, 250 μg/pig for all groups.
^{b}dpc, days post-challenge; Challeneg virus: PEDV 2013 Colorado strain, 3.0 × 10^{5.0} TCID₅₀/pig.
^{c}ME5125: killed genome-reduced E. coli cells not expressing any vaccine antigen

Evaluation of clinical signs, gross and histological lesions. After virus challenge, pigs were monitored daily for clinical signs of diarrhea and body condition. Diarrhea scores range from 1 to 3: 1, normal to pasty feces; 2, semi-liquid diarrhea with some solid content; 3, liquid diarrhea with no solid content. Body condition scores range from 1 to 3: 1, undetectable spinous processes and hook bones; 2, spinous processes and hook bones were slightly felt; 3, spinous processes and hook bones were easily felt and visible (Lu et al., 2020b).

Gross and histopathological lesions were evaluated by a board-certified veterinary pathologist (TL) who was blind to the treatment groups. At necropsy, small intestines were subdivided into three sections (duodenum, jejunum, and ileum), while large intestines were subdivided into two sections (cecum and colon). Gross lesions in intestine tissues were scored 1 to 3, with 1 being normal; 2, either thin-walled or gas-distended intestine; and 3, both thin-walled and gas-distended intestine. Intestinal contents were also scored 1 to 3 with 1 being solid or pasty feces; 2, semi-watery feces; and 3, watery feces with no solid contents. Jejunum tissues were also collected at necropsy and fixed in formalin for histological examination. Hematoxylin & eosin (H&E) stained tissue slides were subsequently prepared from formalin-fixed tissues. Villous length (V) and crypt depth (C) were measured at 10 different sites on each sample slide. The average V to C ratio (V:C) was calculated. A lower V:C indicates more severe intestinal lesion.

Quantification of PEDV RNA by RT-gPCR. Total RNAs were isolated from 10% suspension of fecal swab materials, intestine contents, or samples of homogenized intestine tissues, respectively, by using Trizol LS reagent (Thermo Fisher Scientific). The PEDV RNA loads in samples were quantitated by one step RT-qPCR kit (Bioline Sensifast Probe No Rox One Step Kit) according to the manufacturer's instruction. The primer pair, probe and standard used in the assay were previously described (Opriessnig et al., 2014; Subramaniam et al., 2018). The detection limit was 10 genomic copies per reaction.

Peptide-based ELISA for detecting anti-PEDV FP and anti-SARS-CoV-2 FP antibodies. Custom-made BSA-conjugated peptides of SARS-CoV-2 FP and PEDV FP were commercially synthesized (GenScript, Piscataway, NJ). 96-well ELISA plates were coated with 0.2 μg/mL each of the BSA-conjugated peptides in 0.05M carbonate-bicarbonate buffer (pH 9.6) at 4° C. for 12 h. After extensive wash by Tris-buffered saline buffer with 0.05% Tween 20 (TBST), plates were blocked by blocking buffer (1.5% BSA in TBST) at 37° C. for 2 h. The plates were washed, and then added with diluted serum sample (1:200 in blocking buffer). PEDV hyper-immune pig serum against S1 was used as positive control, while PEDV negative pig serum was used as negative control (Subramaniam et al., 2018). After incubation at 37° C. for 1 h, plates were extensively washed and then incubated with peroxidase-conjugated rabbit anti-pig IgG (1:20,000 dilution) (MilliporeSigma) at 37° C. for 1 h. After extensive washing, plates were developed by adding One-step Ultra TMB solution (Thermo Fisher Scientific) according to the manufacturer's instruction. The reaction was stopped by 2 N sulphuric acid, and the absorbance at 450 nm (OD450) was read. The normalized OD (S/P value) was calculated as S/P=(Sample OD-Negative OD)/(Positive OD-Negative OD).

Generation of a lentiviral-based SARS-CoV-2 S pseudovirus for detecting anti-SARS-CoV-2 neutralizing antibody (NA). The full-length SARS-CoV-2 S protein coding sequence (human codon optimized) was cloned into mammalian expression vector pcDNA under the control of a CMV-promoter with a BGH-polyA terminator. The resulting construct, pcDNA-SARS-CoV2-S, was used as a packing vector to generate pseudovirus particles containing SARS-CoV2 S protein. Briefly, 293T cells were transfected with Firefly-Luciferase-containing reporter lentivirus vector pLJM1-FFLuc, pMDLg/pRRE, pRSV-Rev (Addgene, USA), and pcDNA-SARS-CoV2-S. The transfected cells were maintained in DMEM with 10% FBS and 20 mM HEPES at 37° C. and 5% CO₂. At 48 h post-transfection, cell-culture supernatant containing pseudovirus-SARS-CoV2-S particles was collected and clarified using low-speed centrifugation (2,000×g, 10 min). The clarified pseudovirus preparation was then concentrated using Amicon 100 kDa filter (MilliporeSigma, USA), and the concentrated pseudovirus (SARS-CoV2-FFLuc) was aliquoted and stored at −80° C. until use. The SARS-CoV2-FFLuc pseudovirus was titrated by serially diluting two-fold in medium containing DMEM with 2% FBS and polybrene (8 μg/mL). 100 μL of the serially diluted pseudovirus was overlaid onto hACE2-overexpressing 293T (hACE2-293T) cell monolayer in a 96-well plate, and incubated at 37° C. and 5% CO₂. After 48 h of incubation, the luciferase expression level was estimated using Luciferase kit (Promega, USA) per the manufacturer's protocols.

To detect anti-SARS-CoV-2 NA, the heat inactivated serum samples (56° C., 30 min) from the vaccination and challenge pig study were 2-fold serially diluted (starting from 1:10) and mixed with equal volume of SARS-CoV2-FFLuc lenti-pseudovirus. After one hour incubation at 37° C., 100 μL of the mixtures were added to the hACE2-293T cells in 96-well plate at 90% confluence. The plate was then incubated at 37° C. with 5% CO₂ for 48 h. The luminescence was detected by using Luciferase kit (Promega, USA) according to the manufacturer's instructions. After subtraction of background (medium only), samples with >50% luminescence unit reduction relative to the control (SARS-CoV2-FFLuc only) was considered as positive for neutralizing antibody.

High-throughput neutralization test (HTNT) for detecting anti-PEDVNA. To detect the NA against PEDV, pig sera were tested using a HTNT assay at the Iowa State University Veterinary Diagnostic Laboratory (Sarmento et al., 2020). Briefly, 1:20 diluted heat inactivated serum samples were mixed with a fixed amount of PEDV at 1:1 volume ratio (final serum dilution 1:40). The serum-virus mixtures were inoculated onto Vero cells in 96-well plate for 1.5 to 2 h at 37° C. After adding fresh culture medium, cells were incubated for 24 h, then fixed and stained with a conjugated PEDV mAb, followed by reading on image cytometry. The 1:40 diluted serum samples with a ≥85% total fluorescence reduction (% FR) relative to the control were classified as positive for NA.

Detection of IFN-γ in pig sera. The level of IFN-γ in pig serum samples was evaluated by using a commercial Swine IFN-γ ELISA Kit (MyBioSource, San Diego, CA) according to the manufacturer's instruction. Briefly, 2-fold serially-diluted IFN-γ standard (500 μg/mL to 7.8 μg/mL) and undiluted pig serum samples were added into a 96-well microplate pre-coated with IFN-gamma specific antibody. After 1 h incubation at 37° C., the plate was aspirated and added with a Biotin-conjugated antibody. The plate was incubated at 37° C. for 1 h and subsequently washed 3 times. Streptavidin-RP was added into plate and incubated for 30 min at 37° C. After 5 washes, the plate was developed by adding of TMB substrate at 37° C. for 15 min prior to the addition of stop solution. The OD450 was read using a microplate reader. All the reagents used in this assay are included in this kit.

Statistical analysis. Statistical analysis was done using R (Version 1.3.1093) with the Rstudio environment with included packages and the tidyverse and stats packages, with visualizations using ggplot2.

### Example 7

Establishment of a Novel Vaccine Platform to Express Foreign Antigen on the Surface of Genome-Reduced E. coli

To create a platform for the rapid production of new vaccines, employing killed whole cell genome-reduced E. coli expressing vaccine antigens on their surfaces, a plasmid, pRAIDA2, was designed and synthesized (FIG. 5A) that contains a high copy origin of replication, a kanamycin resistance gene, and an AIDA-I-derived autotransporter (AT) surface expression cassette with a rhamnose-inducible promoter. After the AT amino terminal signal sequence, pRAIDA2 has a cloning site flanked by type IIS BbsI restriction sites, enabling “scarless” cloning into the expression cassette. The parental version of the plasmid includes sequence encoding an influenzavirus HA immunotag as stuffer, flanked by a trypsin cleavage site, to enable confirmation and evaluation of surface expression (FIG. 5A). FIG. 5B illustrates the rapid production of synthetic biology-mediated candidate vaccines using pRAIDA2, or similar systems, and genome-reduced bacteria.

To determine if the vaccine antigens expressed on the surface of the bacteria would be much more visible to the immune system if expressed on bacteria with a large number of the genes encoding surface proteins deleted, we first examined the collection of systematically deleted E. coli strains produced by the Tokyo Metropolitan University Group (Hashimoto et al., 2005; Kato & Hashimoto, 2007) and identified genes encoding proteins with an imputed location on the surface of the cell (FIG. 6A). The strain with the largest amount of genome deleted in the collection included deletions in almost 200 genes encoding proteins imputed to be on the cell surface.

### Example 8

Enhanced Antibody Binding to an Antigen Expressed on the Surface of Genome-Reduced E. coli

The ability of bacteria with different amounts of genome deletions that had been transformed with pRAIDA2 which, in its parental versions, expresses an HA immunotag via the AIDA-I autotransporter on the bacterial surface, to bind an anti-HA monoclonal antibody (FIG. 6B) was tested. It was found that binding of the monoclonal antibody to the bacteria increased as a function of genome reduction (FIG. 6B).

To confirm that the recombinant HA immunotag was properly expressed on the surface of the bacterial strains and to eliminate the possibility that the increased binding observed in the flow cytometry experiments was due to quantitative differences in the amount of HA expressed in the bacteria, trypsinization-immunoblot experiments were conducted in which we induced expression of the HA immunotag was induced with rhamnose, then either did or did not subject the bacteria to trypsin treatment prior to making protein extracts for immunoblotting (FIG. 6C). It was found that the HA immunotag was expressed on all the tested mutants. The amount of HA protein expressed on the different strains was approximately equal, suggesting that the increased binding seen with the highly deleted strains was not the result of quantitative differences in protein expression. The HA immunotag was accessible to trypsinization in all the strains, providing further evidence that the HA immunotag expressed via pRAIDA2 was located on the surface of the bacteria.

### Example 9

Successful Surface Expression of SARS-CoV-2 FP and PEDV FP on Genome-Reduced E. coli, Production and Characterization of Candidate Vaccines

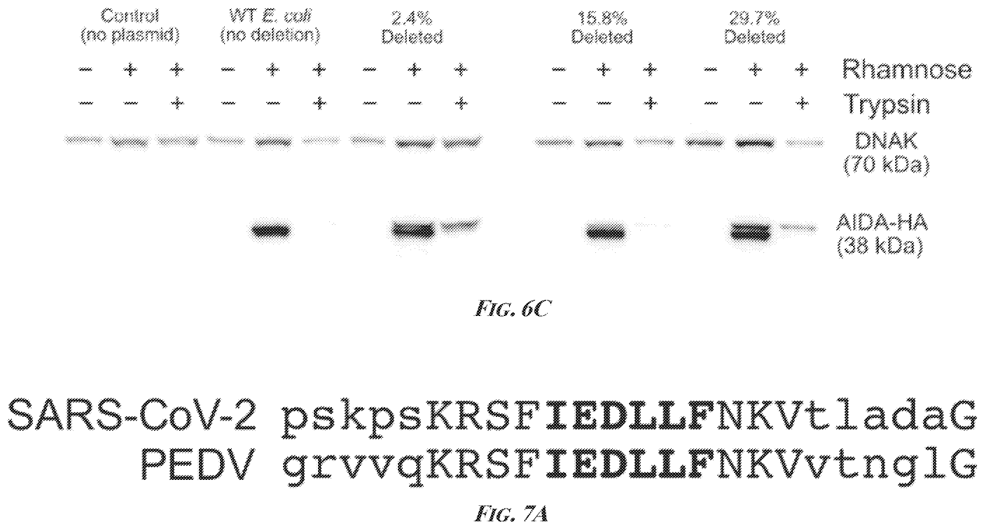

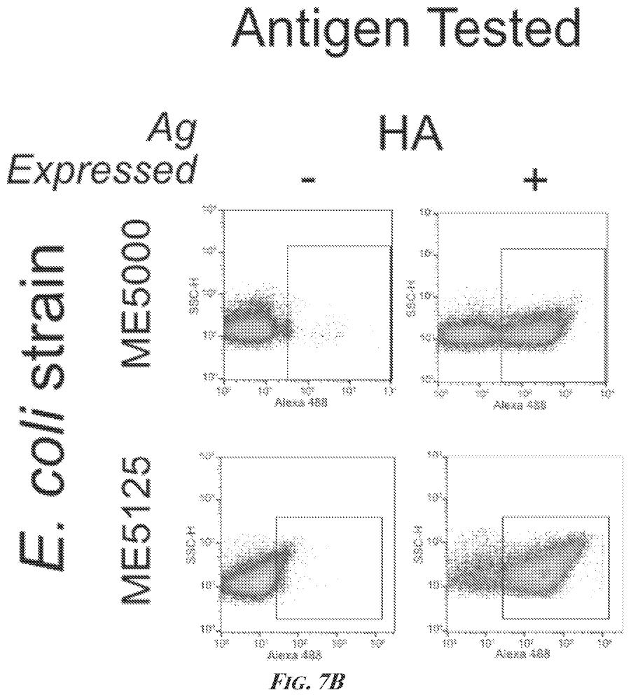

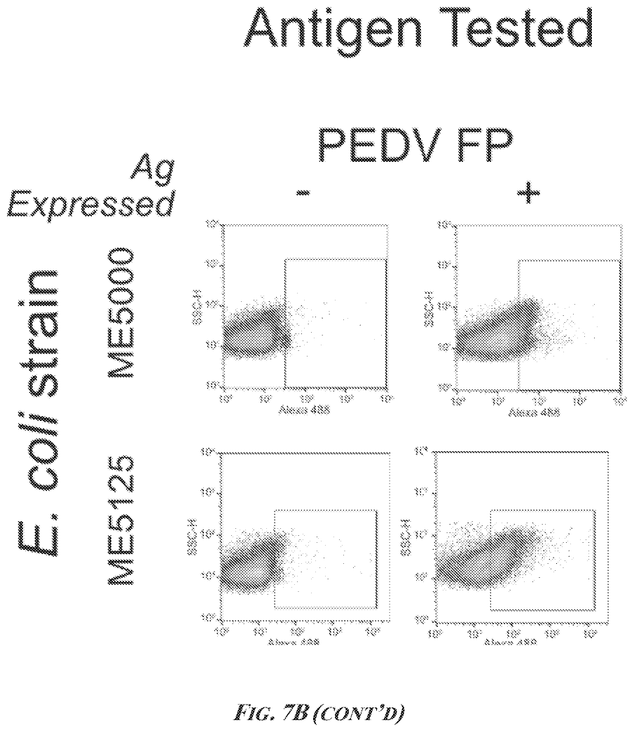

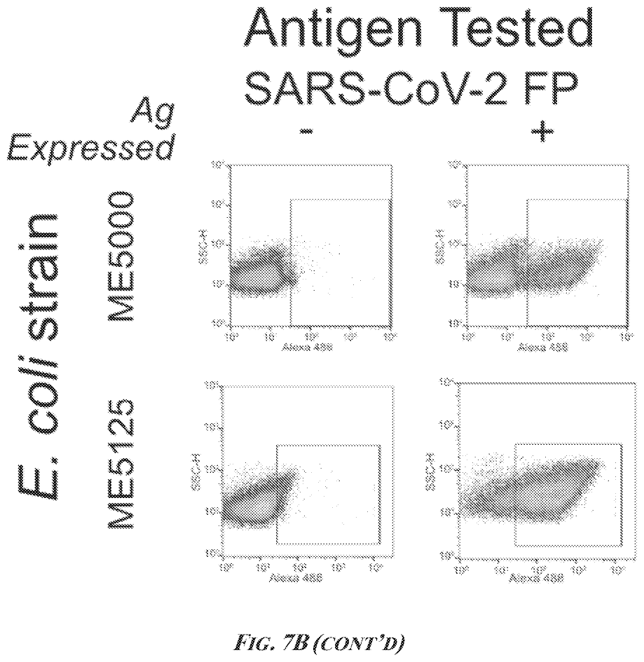

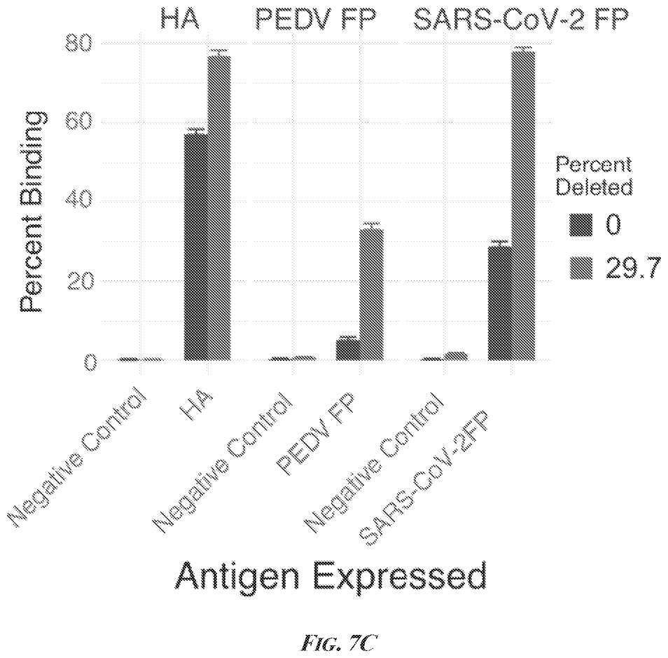

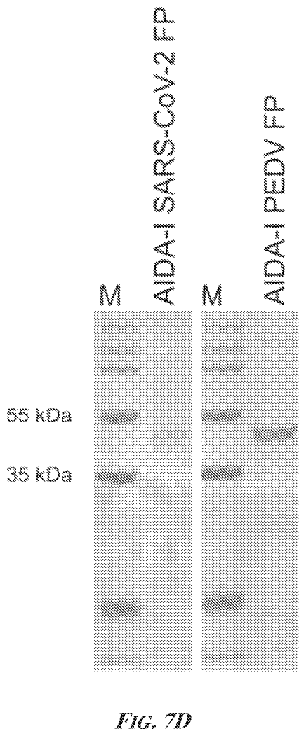

To test the ability of the genome-reduced E. coli surface expression to yield a useful vaccine, the FP region of the coronavirus S protein was selected as the target (FIG. 7A) because the FP is extremely well conserved across coronaviruses (van Dorp et al., 2020). Therefore, a FP-targeting vaccine would be relatively resistant to viral evolution or mutation. The FPs of other viruses, such as HIV-1, with type I fusion proteins, have been the target of active vaccine development efforts. Further, monoclonal antibodies directed against the SARS-CoV FP were neutralizing and protective in passive challenge experiments (Miyoshi-Akiyama et al., 2011). In addition to the SARS-CoV-2 FP, in parallel a PEDV FP vaccine was produced, and both were tested in a PEDV native virus challenge pig model because such a model is one of the few opportunities in which a vaccine against a coronavirus can be effectively tested in a relevant model that involves a coronavirus infection in its native host. For the test vaccine antigens, the basic 18-aa FP was included, plus flanking sequence that had been mapped as being included in the binding sites for neutralizing and disease-modifying-associated sera and neutralizing monoclonal antibodies against SARS-CoV and SARS-CoV-2 (Miyoshi-Akiyama et al., 2011; Ng et al., 2020; Poh et al., 2020; Shrock et al., 2020), for the SARS-CoV-2 construct, and the corresponding amino acids for PEDV, to include a total of 23 amino acids (see FIG. 7A).

It was initially conceived of the SARS-CoV-2 FP vaccine as a negative control to assess protection elicited by the PEDV FP vaccine, given that PEDV is an alphacoronavirus and SARS-CoV-2 is a betacoronavirus. However, sequence alignment of the SARS-CoV-2 and PEDV FP sequences used in this study (FIG. 7A) revealed that the 13 amino acid residues surrounding the core FP sequence are identical between SARS-CoV-2 FP and PEDV FP, suggesting that the efficacy of a SARS-CoV-2 FP vaccine may be tested by using the PEDV challenge pig model as well. Rabbit polyclonal antibodies directed against the PEDV and SARS-CoV-2 FPs, respectively were also produced. pRAIDA2-SARS-CoV-2 FP and pRAIDA2-PEDV FP were transformed into the wild type E. coli strain ME5000 (0% genome deleted) and strain ME5125 (29.7% genome deleted) and conducted flow cytometry experiments using the rabbit anti-FP antibodies to demonstrate that the FP antigens were successfully expressed on the bacteria. It was also shown that, for both these FP antigens, expression on the highly deleted ME5125 strain yielded substantially increased binding (FIGS. 7B and 7C). Expression of the FP-autotransporter fusion protein was further verified by immunoblot (FIG. 7D).

To produce and characterize the SARS-CoV-2 FP and PEDV FP candidate vaccines for the animal study, the highly deleted (29.7%) ME5125 E. coli strain transformed with pRAIDA2-SARS-CoV-2 and pRAIDA2-PEDV were grown, respectively, expression was induced with rhamnose, and then the bacteria were inactivated with formalin. Expression of FP antigens on the bacterial cell vaccines was verified by flow cytometry, and the amount of FP expression was quantitated by immunoblot, using serial dilutions of DNase K as the quantitation standard.

### Example 10

SARS-CoV-2 FP and PEDV FP Vaccines Induce Low Anti-FP Humoral Response but Potent Anamnestic Responses after Virus Challenge

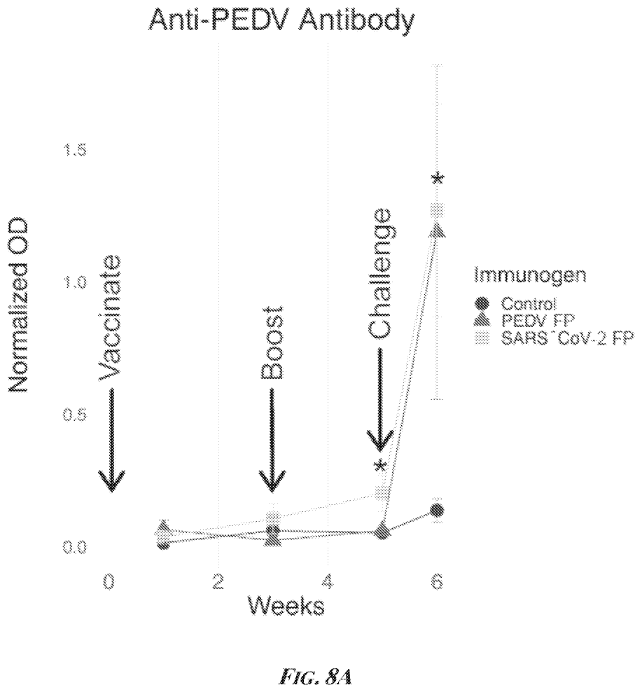

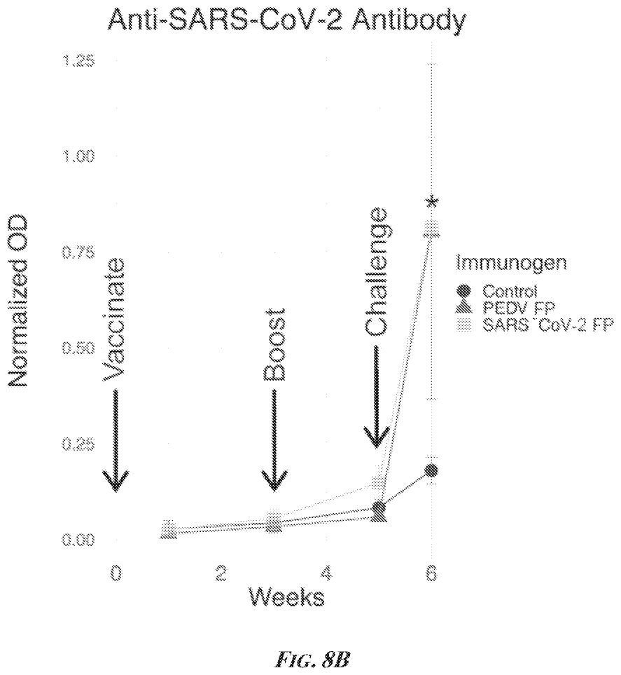

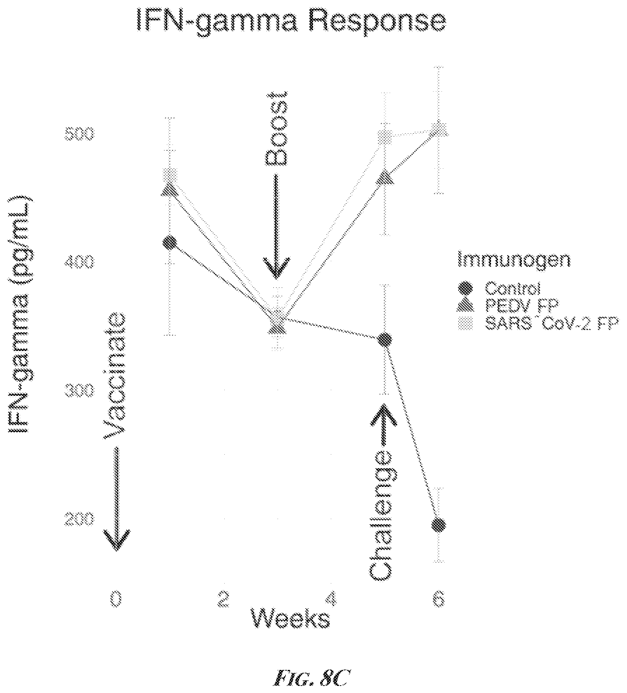

Pigs were vaccinated intramuscularly with the killed whole cells bacterial vaccines expressing the SARS-CoV-2 FP or PEDV FP or control bacteria not expressing a coronavirus FP on day 0, boosted at day 21, and then challenged with infectious PEDV orally at day 35. Fecal samples and swabs were collected daily and blood was collected weekly. The production of antibodies recognizing the FPs was examined by ELISAs (FIGS. 8A and 8B). It was found that vaccination with the SARS-CoV-2 FP or PEDV FP vaccines did not elicit a strong anti-FP response by week 5 of the study, 2 weeks after boosting, although it was noticed that the SARS-CoV-2 FP vaccine elicited a low, but statistically significant response against the PEDV FP (p<0.05, Wilcoxon Rank Sum Test), while showing a weak trend to significance in eliciting an immune response against the SARS-CoV-2 FP itself (p=0.25). Importantly, both vaccines primed the pigs for a potent anamnestic response against either FP after the pigs were infected with PEDV. At the end of the challenge experiment, anti-FP ELISA values for the pigs vaccinated with the FP vaccines were all significantly (p<0.05, Wilcoxon Rank Sum Test) higher than values for the pigs vaccinated with the control vaccine. However, no detectable neutralizing antibody against PEDV nor SARS-CoV2 S pseudovirus was detected in vaccinated or control group of pigs.

### Example 11

### Vaccines Potentiate IFN-γ Response in Vaccinated Pigs

The level of IFN-γ in pig serum samples were tested and compared between the vaccinated groups and control group at each time point (FIG. 8C). There were significant differences at 5 weeks post-vaccination (wpv, P<0.05) and 1 week post-challenge (wpc, P<0.05). The results showed that the serum IFN-γ levels significantly increased 2 weeks after the vaccine booster dose (5 wpv) and 1 week post-challenge (1 wpc or 6 wpv) in the vaccinated groups as compared to the control group. The IFN-γ levels at 1 wpv and 3 wpv are similar but the IFN-γ level at 5 wpv (i.e., 2 weeks after booster) increases in vaccinated groups, suggesting that the vaccine prime dose likely has activated T cells, and the booster dose further amplifies it. The results suggested that the FP vaccines potentiate IFN-γ response in vaccinated animals.

### Example 12

Vaccines Reduced Clinical Signs and Pathological Lesions in Pigs after PEDV Challenge

The efficacy of the two genome-reduced bacteria-vectored surface expression vaccine candidates (SARS-CoV-2 FP and PEDV FP) was evaluated in a pig vaccination and challenge study against PEDV (strain 2013 Colorado). Since severe disease is usually found in younger PEDV-infected piglets while the pigs used in this study were approximately 10-weeks-old at the time of virus challenge, a higher dose of PEDV (3.0×10⁵⁰ TCID₅₀ per pig) was used to challenge the pigs. Clinical observations were conducted for the immediate 2-4 hours after vaccination and daily thereafter, and included an assessment of the pig's body condition and stool/diarrhea output. A few vaccinated pigs exhibited lethargy, labored breathing and vomiting immediately after vaccination, which resolved shortly after intramuscular administration of diphenhydramine.

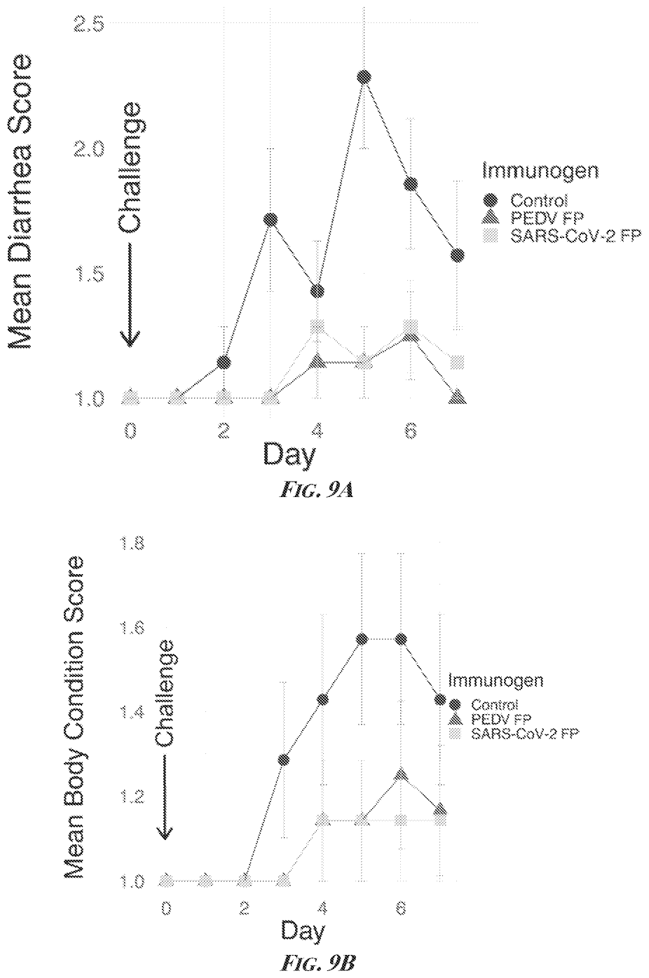

The PEDV-related clinical signs of diarrhea and body condition were scored (FIGS. 9A and 9B). At 2 dpc, one pig in the unvaccinated control group began to show clinical signs of diarrhea, and in total, 6 of the 7 pigs developed diarrhea during the course of study. Most of the pigs developed marasmus along with diarrhea. In both vaccinated groups, most of pigs remained healthy and only 1 to 2 pigs showed mild diarrhea and marasmus during the study. Friedman Rank Sum Tests comparing the vaccinated groups after day 3 to the control for both diarrhea and body condition scores were highly significant (p<0.01 for all groups), suggesting that the vaccines significantly reduced the clinical signs after virus challenge.

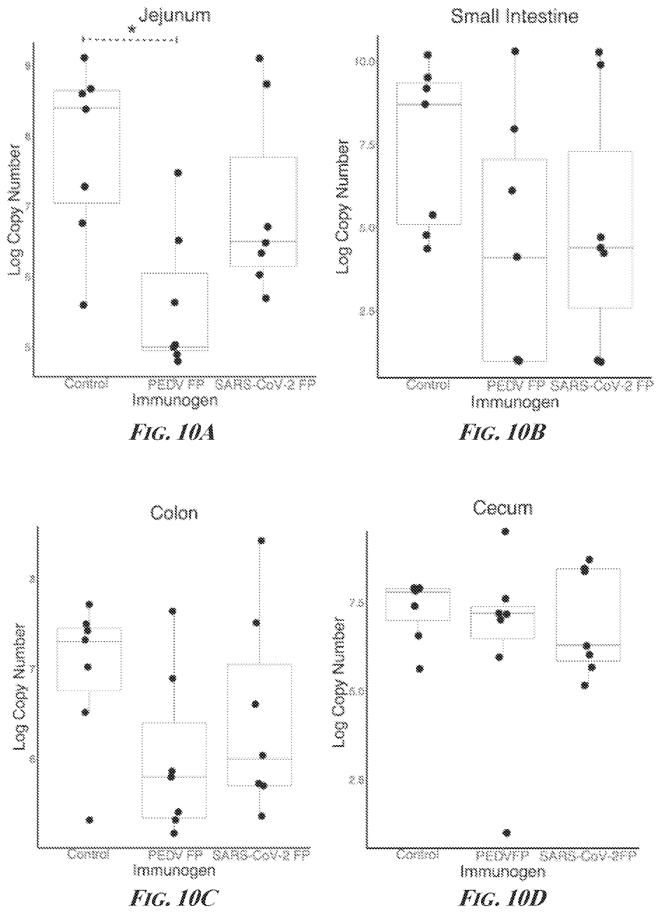

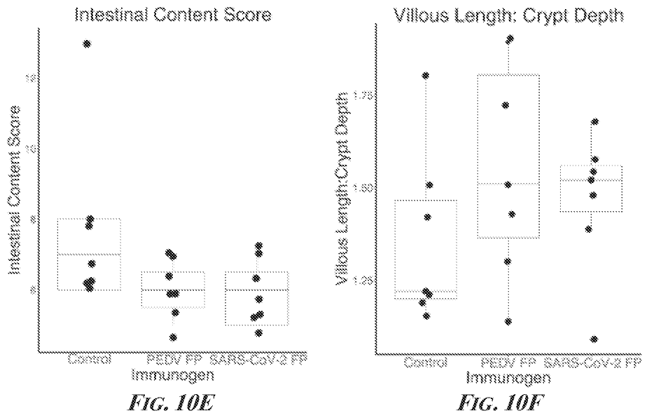

For the intestine contents score at necropsy (FIG. 10E), both vaccinated groups showed lower scores than the control group, and the difference between SARS-CoV-2 FP group and control group was borderline statistically significant (p=0.055, Kruskal-Wallis test). The difference between the PEDV FP group and the control group also showed a tendency to significance (p=0.08). For the histopathological lesion (FIG. 10F), the mean ratios of villous length to crypt depth (V:C) of jejunum tissues from both vaccine groups had higher values (more healthy jejunum) than control group, although the difference were not significant, for either the PEDV FP vaccine (p=0.20, Kruskal-Wallis) or the SARS-CoV-2 FP vaccine (p=0.27), in part due to the dispersion of the values. In all groups of pigs, only two pigs in the unvaccinated control group developed either thin walled or gas-distended jejunum, while other pigs did not show any gross lesion in intestines. The pathological data suggested that the vaccinated pigs had less severe lesions of PEDV infection. The overall gross and histological lesions of pigs in this study were mild because the 10-week-old pigs were less vulnerable to PEDV-induced disease.

### Example 13

Vaccines Decreased Viral RNA Loads in Pigs after PEDV Challenge

After challenge, daily virus shedding in fecal swab materials (0-7 dpc) was monitored. PEDV RNA was detected in pigs starting at 2 dpc, however, there was no significant difference in viral RNA loads in fecal swab materials. It should be pointed out here that quantification of viral RNA from fecal swab materials can be highly variable due to the inconsistency of collecting the amount of fecal swab materials from pig to pig.

Intestine tissues (jejunum, colon, cecum) and small intestinal contents were also collected during necropsy (7 dpc) to more accurately quantify the viral RNA loads in each pig. There was a significant difference in viral RNA loads in the comparison of control vs. PEDV vaccine in the jejunum tissue (FIG. 10A) (p=0.01, Kruskal-Wallis test). There were trends to significance in the comparison of control vs. PEDV vaccine in the small intestine content (FIG. 10B) (p=0.158), and in the colon tissue (FIG. 10C) (p=0.11). Similar to PEDV FP, the SARS-CoV-2 FP vaccinated pigs also have lower PEDV loads compared to control pigs. The data suggested that the two vaccines reduced PEDV loads in pig intestinal compartments after virus challenge.

### Discussion of Examples 7-13

There is accumulating, encouraging evidence that vaccines against SARS-CoV-2 comprising the entire S, or the receptor binding domain (RBD), can provide excellent protection against infection (Cohen, 2020). There are also some reports that patients who recover from mild COVID-19 can be re-infected by a different SARS-CoV-2 strain (Lee et al., 2020; Van Elslande et al., 2020), and that this can happen in patients who have antibodies against the S1 region of S and the RBD. It may therefore be helpful to design the next generations of SARS-CoV-2 vaccines to elicit immune responses against additional discrete targets in S. Also, it is important to further explore additional COVID-19 vaccine platforms that can be quickly and inexpensively produced, and can be stored and transported more easily for globally appropriate use.

EXAMPLES 7-13 explore whether expressing foreign antigens on the surface of genome-reduced Gram-negative bacteria using Gram-negative autotransporter expression systems could allow antigens to interact more effectively with the immune system and therefore yield a synthetic biology-based new vaccine platform to rapidly produce very inexpensive vaccines. While the candidate vaccines produced in EXAMPLES 7-13 did not elicit strong neutralizing humoral immune responses using the arbitrarily chosen dose, immunization route and vaccination schedule, protection against disease in the surrogate porcine coronavirus challenge model in pigs was observed. A strong anamnestic response upon virus challenge was demonstrated, as was evidence of differences in clinical correlates of disease and virus production. The SARS-CoV-2 FP vaccine as used in the PEDV challenge pig model was also encouraging in that it provided protection against viral pathologic effects, since a previously produced dendritic cell-targeting PEDV S protein vaccine given to sows, reduced viral shedding in their experimentally infected piglets, but was associated with enhanced gross pathologic lesions (Subramaniam et al., 2018).

It is reasonable to consider that with optimization of the dose, immunization schedule, and route, notably the use of vaccination via oral or intranasal mucosal compartments, additional reduced genome Gram-negative bacterial systems, such as Salmonella or Vibrio, or incorporation of appropriate adjuvants in the future, vaccines made using this new vaccine platform may elicit better immune responses and will be valuable for rapid responses to future pandemic viral diseases. For the specific use in coronavirus vaccines, other highly conserved antigens could also be expressed using the platform, potentially yielding better immune responses, or vaccines targeting multiple antigens could yield a better immune response. The presently disclosed vaccine platform employed E. coli with large, but essentially arbitrary mutations. Therefore, more targeted mutations limited to surface-expressed genes, with fewer effects on bacterial growth characteristics may also improve industrial production or delete even more, non-essential surface-expressed proteins, enabling more immunogenic bacteria. Additional derivatives of the genome-reduced bacteria (minicells, outer membrane vesicles) could also enhance immune responses.

Employing the presently disclosed vaccine platform, it was found that the FP may be a useful target for coronavirus vaccine development, including as a potential target for a universal coronavirus vaccine. The FP was selected because it is extremely well-conserved among coronaviruses, and because the FP of other viruses with type 1 viral fusion proteins have also been the target of considerable vaccine development efforts. The PEDV FP vaccine was produced to evaluate the new vaccine platform, and the SARS-CoV-2 FP vaccine was produced to obtain preliminary safety and immunogenicity in a non-rodent species, with an eye toward development of a SARS-CoV-2 FP vaccine for humans. The findings that the SARS-CoV-2 FP and PEDV FP vaccines exhibited essentially similar protective effect in the PEDV challenge pig model system are not surprising, since 13 amino acid residues surrounding the core FP sequence between SARS-CoV-2 FP and PEDV FP are identical. Therefore, it is expected that the SARS-CoV-2 FP vaccine will induce cross-protection against PEDV challenge. The finding that the FP vaccines from different genera of coronaviruses (alpha and beta coronaviruses) were essentially similar in protective effect suggests that the FP may be a useful target for development of a broad coronavirus vaccine, and that it may be helpful to include an FP-specific antigen in future, next-generation of SARS-CoV-2 vaccines, and in efforts to develop a universal coronavirus vaccine. The data also suggest that the PEDV challenge pig model can be useful in assessing coronavirus vaccine candidates that can elicit broadly-protective responses across different coronaviruses.

Finally, while it was not found that the two candidate vaccines, used at the dose, vaccination route and schedule described herein, elicited sterilizing immunity, they did elicit a potent anamnestic response, a significantly higher IFN-γ response, and protection against clinical disease. It remains to be seen if the current SARS-CoV-2 vaccines in phase 3 clinical trials induce sterilizing immunity, since some licensed animal coronavirus vaccines protect against clinical diseases but not against infection (Cohen, 2020). For example, canine coronavirus vaccines protect dogs from disease but not from infection (Lee et al., 2020). A recent passive immunization study in non-human primate suggested that high level neutralizing humoral immunity may not be essential for protection against SARS-CoV-2 disease (Van Elslande et al., 2020). A very inexpensive, easy to manufacture vaccine with low supply chain requirements and logistical challenges that does not elicit sterilizing immunity, but still helps protect against clinically significant disease, and is highly resistant to viral evolution or mutation may yet be helpful in a global context, particularly in regions where highly sophisticated medical care is scarce. Given estimates that sufficient courses of the current COVID-19 vaccines may not be available to vaccinate much of the global population in developing countries until substantially later than the industrialized countries (Tizard, 2020), and that the cost of many of these current vaccines and their requirement for very cold storage and transport may present a challenge for the poorer countries in the world, additional, globally appropriate SARS-CoV-2 vaccines may prove helpful.

Summarily, disclosed herein is a new synthetic biology-based, killed whole cell bacterial vaccine platform that utilizes ATs to display vaccine antigens on the surfaces of genome-reduced E. coli (grEc), enabling rapid production of a testable vaccine. As a proof of principle for the new vaccine platform, a killed whole cell vaccines targeting the FPs of two coronaviruses, SARS-CoV-2 and PEDV was produced, and it was demonstrated that these vaccines induced potent anamnestic responses upon virus challenge and elicited protection against disease in a PEDV challenge pig model, validating the novel vaccine platform technology and the use of the coronavirus FP target.

Disclosed herein is the generation and use of a new vaccine platform to express SARS-CoV-2 and porcine epidemic diarrhea virus (PEDV) fusion peptide (FP) on the surface of E. coli, and the use of the same as a killed whole cell vaccine. The FP sequence is highly conserved across coronaviruses; the 13 amino acid residues surrounding the core FP sequence of SARS-CoV-2 and PEDV are identical. Therefore, the efficacy of the SARS-CoV-2 FP vaccine was tested in parallel with a PEDV FP vaccine, using a surrogate PEDV challenge pig model. It was demonstrated that both vaccines induced potent anamnestic responses upon virus challenge, potentiated IFN-γ responses, reduced viral RNA loads in the intestine, and protected animals from clinical disease. However, neither vaccines elicited sterilizing immunity. Nevertheless, since SARS-CoV-2 FP and PEDV FP vaccines provided similar protection against disease in the PEDV pig model, the coronavirus FP can be a target for a broadly-protective vaccine. Importantly, the genome-reduced E. coli surface expressed vaccine platform has utility as an inexpensive and rapid vaccine platform for other pathogens.

TABLE 6
Summary of Bacterial Genes

| Gene | ME | ME | ME | Subcellular Localization (see key at end of Table) | | | | | | | | | | | | | |
|---|---|---|---|---|---|---|---|---|---|---|---|---|---|---|---|---|---|
| Name | 5110 | 5119 | 5125 | A | B | C | D | E | F | G | H | I | J | K | L | M | N |
| stpA | 0 | 1 | 1 | 1 | 0 | 1 | 0 | 0 | 0 | 0 | 0 | 0 | 0 | 0 | 0 | 0 | 0 |
| dps | 0 | 0 | 1 | 1 | 0 | 1 | 0 | 0 | 0 | 0 | 0 | 0 | 0 | 0 | 0 | 0 | 0 |
| yjgX | 1 | 1 | 1 | 0 | 0 | 0 | 1 | 0 | 0 | 0 | 0 | 0 | 0 | 0 | 0 | 0 | 0 |
| idnT | 1 | 1 | 1 | 0 | 1 | 0 | 1 | 0 | 0 | 0 | 0 | 0 | 0 | 0 | 0 | 0 | 0 |
| ybhC | 0 | 0 | 1 | 0 | 1 | 0 | 0 | 1 | 0 | 0 | 0 | 0 | 0 | 0 | 0 | 0 | 0 |
| torC | 0 | 1 | 1 | 0 | 1 | 0 | 0 | 0 | 1 | 0 | 0 | 0 | 0 | 0 | 0 | 0 | 0 |
| ada | 0 | 0 | 1 | 0 | 0 | 1 | 0 | 0 | 0 | 0 | 0 | 0 | 0 | 0 | 0 | 0 | 0 |
| rcnB | 0 | 1 | 1 | 0 | 0 | 1 | 0 | 0 | 1 | 0 | 0 | 0 | 0 | 0 | 0 | 0 | 0 |
| mcrB | 1 | 1 | 1 | 0 | 0 | 1 | 0 | 0 | 0 | 0 | 0 | 0 | 0 | 0 | 0 | 0 | 0 |
| tdcE | 0 | 1 | 1 | 0 | 0 | 1 | 0 | 0 | 0 | 0 | 0 | 0 | 0 | 0 | 0 | 0 | 0 |
| tdcA | 0 | 1 | 1 | 0 | 0 | 1 | 0 | 0 | 0 | 0 | 0 | 0 | 0 | 0 | 0 | 0 | 0 |
| tdcR | 0 | 1 | 1 | 0 | 0 | 1 | 0 | 0 | 0 | 0 | 0 | 0 | 0 | 0 | 0 | 0 | 0 |
| glsA | 0 | 1 | 1 | 0 | 0 | 1 | 0 | 0 | 0 | 0 | 0 | 0 | 0 | 0 | 0 | 0 | 0 |
| cueR | 0 | 1 | 1 | 0 | 0 | 1 | 0 | 0 | 0 | 0 | 0 | 0 | 0 | 0 | 0 | 0 | 0 |
| ybbL | 0 | 1 | 1 | 0 | 0 | 1 | 0 | 0 | 0 | 0 | 0 | 0 | 0 | 0 | 0 | 0 | 0 |
| ybbN | 0 | 1 | 1 | 0 | 0 | 1 | 0 | 0 | 0 | 0 | 0 | 0 | 0 | 0 | 0 | 0 | 0 |
| ybbA | 0 | 1 | 1 | 0 | 0 | 1 | 0 | 0 | 0 | 0 | 0 | 0 | 0 | 0 | 0 | 0 | 0 |
| hyi | 0 | 1 | 1 | 0 | 0 | 1 | 0 | 0 | 0 | 0 | 0 | 0 | 0 | 0 | 0 | 0 | 0 |
| allD | 0 | 1 | 1 | 0 | 0 | 1 | 0 | 0 | 0 | 0 | 0 | 0 | 0 | 0 | 0 | 0 | 0 |
| alsA | 0 | 1 | 1 | 0 | 0 | 1 | 0 | 0 | 0 | 0 | 0 | 0 | 0 | 0 | 0 | 0 | 0 |
| rpiR | 0 | 1 | 1 | 0 | 0 | 1 | 0 | 0 | 0 | 0 | 0 | 0 | 0 | 0 | 0 | 0 | 0 |
| phnN | 0 | 1 | 1 | 0 | 0 | 1 | 0 | 0 | 0 | 0 | 0 | 0 | 0 | 0 | 0 | 0 | 0 |
| phnL | 0 | 1 | 1 | 0 | 0 | 1 | 0 | 0 | 0 | 0 | 0 | 0 | 0 | 0 | 0 | 0 | 0 |
| phnK | 0 | 1 | 1 | 0 | 0 | 1 | 0 | 0 | 0 | 0 | 0 | 0 | 0 | 0 | 0 | 0 | 0 |
| basR | 0 | 1 | 1 | 0 | 0 | 1 | 0 | 0 | 0 | 0 | 0 | 0 | 0 | 0 | 0 | 0 | 0 |
| adiA | 0 | 1 | 1 | 0 | 0 | 1 | 0 | 0 | 0 | 0 | 0 | 0 | 0 | 0 | 0 | 0 | 0 |
| melR | 0 | 1 | 1 | 0 | 0 | 1 | 0 | 0 | 0 | 0 | 0 | 0 | 0 | 0 | 0 | 0 | 0 |
| dcuR | 0 | 1 | 1 | 0 | 0 | 1 | 0 | 0 | 0 | 0 | 0 | 0 | 0 | 0 | 0 | 0 | 0 |
| lysU | 0 | 1 | 1 | 0 | 0 | 1 | 0 | 0 | 0 | 0 | 0 | 0 | 0 | 0 | 0 | 0 | 0 |
| cadA | 0 | 1 | 1 | 0 | 0 | 1 | 0 | 0 | 0 | 0 | 0 | 0 | 0 | 0 | 0 | 0 | 0 |
| xylA | 0 | 1 | 1 | 0 | 0 | 1 | 0 | 0 | 0 | 0 | 0 | 0 | 0 | 0 | 0 | 0 | 0 |
| xylG | 0 | 1 | 1 | 0 | 0 | 1 | 0 | 0 | 0 | 0 | 0 | 0 | 0 | 0 | 0 | 0 | 0 |
| xylR | 0 | 1 | 1 | 0 | 0 | 1 | 0 | 0 | 0 | 0 | 0 | 0 | 0 | 0 | 0 | 0 | 0 |
| avtA | 0 | 1 | 1 | 0 | 0 | 1 | 0 | 0 | 0 | 0 | 0 | 0 | 0 | 0 | 0 | 0 | 0 |
| selB | 0 | 1 | 1 | 0 | 0 | 1 | 0 | 0 | 0 | 0 | 0 | 0 | 0 | 0 | 0 | 0 | 0 |
| selA | 0 | 1 | 1 | 0 | 0 | 1 | 0 | 0 | 0 | 0 | 0 | 0 | 0 | 0 | 0 | 0 | 0 |
| rbbA | 0 | 1 | 1 | 0 | 0 | 1 | 0 | 0 | 0 | 0 | 0 | 0 | 0 | 0 | 0 | 0 | 0 |
| uspA | 0 | 1 | 1 | 0 | 0 | 1 | 0 | 0 | 0 | 0 | 0 | 0 | 0 | 0 | 0 | 0 | 0 |
| gor | 0 | 1 | 1 | 0 | 0 | 1 | 0 | 0 | 0 | 0 | 0 | 0 | 0 | 0 | 0 | 0 | 0 |
| arsR | 0 | 1 | 1 | 0 | 0 | 1 | 0 | 0 | 0 | 0 | 0 | 0 | 0 | 0 | 0 | 0 | 0 |
| gadA | 0 | 1 | 1 | 0 | 0 | 1 | 0 | 0 | 0 | 0 | 0 | 0 | 0 | 0 | 0 | 0 | 0 |
| treF | 0 | 1 | 1 | 0 | 0 | 1 | 0 | 0 | 0 | 0 | 0 | 0 | 0 | 0 | 0 | 0 | 0 |
| kdgK | 0 | 1 | 1 | 0 | 0 | 1 | 0 | 0 | 0 | 0 | 0 | 0 | 0 | 0 | 0 | 0 | 0 |
| dppF | 0 | 1 | 1 | 0 | 0 | 1 | 0 | 0 | 0 | 0 | 0 | 0 | 0 | 0 | 0 | 0 | 0 |
| dppD | 0 | 1 | 1 | 0 | 0 | 1 | 0 | 0 | 0 | 0 | 0 | 0 | 0 | 0 | 0 | 0 | 0 |
| tag | 0 | 1 | 1 | 0 | 0 | 1 | 0 | 0 | 0 | 0 | 0 | 0 | 0 | 0 | 0 | 0 | 0 |
| ghrB | 0 | 1 | 1 | 0 | 0 | 1 | 0 | 0 | 0 | 0 | 0 | 0 | 0 | 0 | 0 | 0 | 0 |
| yehK | 0 | 1 | 1 | 0 | 0 | 1 | 0 | 0 | 0 | 0 | 0 | 0 | 0 | 0 | 0 | 0 | 0 |
| yehX | 0 | 1 | 1 | 0 | 0 | 1 | 0 | 0 | 0 | 0 | 0 | 0 | 0 | 0 | 0 | 0 | 0 |
| dld | 0 | 1 | 1 | 0 | 0 | 1 | 0 | 0 | 0 | 0 | 0 | 0 | 0 | 0 | 0 | 0 | 0 |
| preT | 0 | 1 | 1 | 0 | 0 | 1 | 0 | 0 | 0 | 0 | 0 | 0 | 0 | 0 | 0 | 0 | 0 |
| mglA | 0 | 1 | 1 | 0 | 0 | 1 | 0 | 0 | 0 | 0 | 0 | 0 | 0 | 0 | 0 | 0 | 0 |
| galS | 0 | 1 | 1 | 0 | 0 | 1 | 0 | 0 | 0 | 0 | 0 | 0 | 0 | 0 | 0 | 0 | 0 |
| hisG | 0 | 1 | 1 | 0 | 0 | 1 | 0 | 0 | 0 | 0 | 0 | 0 | 0 | 0 | 0 | 0 | 0 |
| hisA | 0 | 1 | 1 | 0 | 0 | 1 | 0 | 0 | 0 | 0 | 0 | 0 | 0 | 0 | 0 | 0 | 0 |
| hisF | 0 | 1 | 1 | 0 | 0 | 1 | 0 | 0 | 0 | 0 | 0 | 0 | 0 | 0 | 0 | 0 | 0 |
| hisI | 0 | 1 | 1 | 0 | 0 | 1 | 0 | 0 | 0 | 0 | 0 | 0 | 0 | 0 | 0 | 0 | 0 |
| ypjJ | 0 | 1 | 1 | 0 | 0 | 1 | 0 | 0 | 0 | 0 | 0 | 0 | 0 | 0 | 0 | 0 | 0 |
| pphB | 0 | 1 | 1 | 0 | 0 | 1 | 0 | 0 | 0 | 0 | 0 | 0 | 0 | 0 | 0 | 0 | 0 |
| yjhG | 0 | 1 | 1 | 0 | 0 | 1 | 0 | 0 | 0 | 0 | 0 | 0 | 0 | 0 | 0 | 0 | 0 |
| yjhH | 0 | 1 | 1 | 0 | 0 | 1 | 0 | 0 | 0 | 0 | 0 | 0 | 0 | 0 | 0 | 0 | 0 |
| sgcE | 0 | 1 | 1 | 0 | 0 | 1 | 0 | 0 | 0 | 0 | 0 | 0 | 0 | 0 | 0 | 0 | 0 |
| sgcQ | 0 | 1 | 1 | 0 | 0 | 1 | 0 | 0 | 0 | 0 | 0 | 0 | 0 | 0 | 0 | 0 | 0 |
| uxuB | 0 | 1 | 1 | 0 | 0 | 1 | 0 | 0 | 0 | 0 | 0 | 0 | 0 | 0 | 0 | 0 | 0 |
| insC1 | 0 | 1 | 1 | 0 | 0 | 1 | 0 | 0 | 0 | 0 | 0 | 0 | 0 | 0 | 0 | 0 | 0 |
| insB1 | 0 | 1 | 1 | 0 | 0 | 1 | 0 | 0 | 0 | 0 | 0 | 0 | 0 | 0 | 0 | 0 | 0 |
| yjhU | 0 | 1 | 1 | 0 | 0 | 1 | 0 | 0 | 0 | 0 | 0 | 0 | 0 | 0 | 0 | 0 | 0 |
| yjhI | 0 | 1 | 1 | 0 | 0 | 1 | 0 | 0 | 0 | 0 | 0 | 0 | 0 | 0 | 0 | 0 | 0 |
| yjiE | 0 | 1 | 1 | 0 | 0 | 1 | 0 | 0 | 0 | 0 | 0 | 0 | 0 | 0 | 0 | 0 | 0 |
| yjiR | 0 | 1 | 1 | 0 | 0 | 1 | 0 | 0 | 0 | 0 | 0 | 0 | 0 | 0 | 0 | 0 | 0 |
| insD1 | 0 | 1 | 1 | 0 | 0 | 1 | 0 | 0 | 0 | 0 | 0 | 0 | 0 | 0 | 0 | 0 | 0 |
| insG | 0 | 1 | 1 | 0 | 0 | 1 | 0 | 0 | 0 | 0 | 0 | 0 | 0 | 0 | 0 | 0 | 0 |
| yjgZ | 0 | 1 | 1 | 0 | 0 | 1 | 0 | 0 | 0 | 0 | 0 | 0 | 0 | 0 | 0 | 0 | 0 |
| sgcX | 0 | 1 | 1 | 0 | 0 | 1 | 0 | 0 | 0 | 0 | 0 | 0 | 0 | 0 | 0 | 0 | 0 |
| yjhQ | 0 | 1 | 1 | 0 | 0 | 1 | 0 | 0 | 0 | 0 | 0 | 0 | 0 | 0 | 0 | 0 | 0 |
| uxuA | 0 | 1 | 1 | 0 | 0 | 1 | 0 | 0 | 0 | 0 | 0 | 0 | 0 | 0 | 0 | 0 | 0 |
| yjiL | 0 | 1 | 1 | 0 | 0 | 1 | 0 | 0 | 0 | 0 | 0 | 0 | 0 | 0 | 0 | 0 | 0 |
| yjiA | 0 | 1 | 1 | 0 | 0 | 1 | 0 | 0 | 0 | 0 | 0 | 0 | 0 | 0 | 0 | 0 | 0 |
| patA3/4 | 0 | 1 | 1 | 0 | 0 | 1 | 0 | 0 | 0 | 0 | 0 | 0 | 0 | 0 | 0 | 0 | 0 |
| ygjH | 0 | 1 | 1 | 0 | 0 | 1 | 0 | 0 | 0 | 0 | 0 | 0 | 0 | 0 | 0 | 0 | 0 |
| ebgR3/4 | 0 | 1 | 1 | 0 | 0 | 1 | 0 | 0 | 0 | 0 | 0 | 0 | 0 | 0 | 0 | 0 | 0 |
| ebgC | 0 | 1 | 1 | 0 | 0 | 1 | 0 | 0 | 0 | 0 | 0 | 0 | 0 | 0 | 0 | 0 | 0 |
| fadH3/4 | 0 | 1 | 1 | 0 | 0 | 1 | 0 | 0 | 0 | 0 | 0 | 0 | 0 | 0 | 0 | 0 | 0 |
| higA | 0 | 1 | 1 | 0 | 0 | 1 | 0 | 0 | 0 | 0 | 0 | 0 | 0 | 0 | 0 | 0 | 0 |
| higB | 0 | 1 | 1 | 0 | 0 | 1 | 0 | 0 | 0 | 0 | 0 | 0 | 0 | 0 | 0 | 0 | 0 |
| rlmG | 0 | 1 | 1 | 0 | 0 | 1 | 0 | 0 | 0 | 0 | 0 | 0 | 0 | 0 | 0 | 0 | 0 |
| ygjP | 0 | 1 | 1 | 0 | 0 | 1 | 0 | 0 | 0 | 0 | 0 | 0 | 0 | 0 | 0 | 0 | 0 |
| uxaA | 0 | 1 | 1 | 0 | 0 | 1 | 0 | 0 | 0 | 0 | 0 | 0 | 0 | 0 | 0 | 0 | 0 |
| uxaC | 0 | 1 | 1 | 0 | 0 | 1 | 0 | 0 | 0 | 0 | 0 | 0 | 0 | 0 | 0 | 0 | 0 |
| exuR | 0 | 1 | 1 | 0 | 0 | 1 | 0 | 0 | 0 | 0 | 0 | 0 | 0 | 0 | 0 | 0 | 0 |
| yqjC | 0 | 1 | 1 | 0 | 0 | 1 | 0 | 0 | 0 | 0 | 0 | 0 | 0 | 0 | 0 | 0 | 0 |
| yqjG | 0 | 1 | 1 | 0 | 0 | 1 | 0 | 0 | 0 | 0 | 0 | 0 | 0 | 0 | 0 | 0 | 0 |
| yhaJ | 0 | 1 | 1 | 0 | 0 | 1 | 0 | 0 | 0 | 0 | 0 | 0 | 0 | 0 | 0 | 0 | 0 |
| yhaK | 0 | 1 | 1 | 0 | 0 | 1 | 0 | 0 | 0 | 0 | 0 | 0 | 0 | 0 | 0 | 0 | 0 |
| tdcF | 0 | 1 | 1 | 0 | 0 | 1 | 0 | 0 | 0 | 0 | 0 | 0 | 0 | 0 | 0 | 0 | 0 |
| tdcB | 0 | 1 | 1 | 0 | 0 | 1 | 0 | 0 | 0 | 0 | 0 | 0 | 0 | 0 | 0 | 0 | 0 |
| yhaB | 0 | 1 | 1 | 0 | 0 | 1 | 0 | 0 | 0 | 0 | 0 | 0 | 0 | 0 | 0 | 0 | 0 |
| gsk | 0 | 1 | 1 | 0 | 0 | 1 | 0 | 0 | 0 | 0 | 0 | 0 | 0 | 0 | 0 | 0 | 0 |
| ybaQ | 0 | 1 | 1 | 0 | 0 | 1 | 0 | 0 | 0 | 0 | 0 | 0 | 0 | 0 | 0 | 0 | 0 |
| selU | 0 | 1 | 1 | 0 | 0 | 1 | 0 | 0 | 0 | 0 | 0 | 0 | 0 | 0 | 0 | 0 | 0 |
| allA | 0 | 1 | 1 | 0 | 0 | 1 | 0 | 0 | 0 | 0 | 0 | 0 | 0 | 0 | 0 | 0 | 0 |
| allR | 0 | 1 | 1 | 0 | 0 | 1 | 0 | 0 | 0 | 0 | 0 | 0 | 0 | 0 | 0 | 0 | 0 |
| glxR | 0 | 1 | 1 | 0 | 0 | 1 | 0 | 0 | 0 | 0 | 0 | 0 | 0 | 0 | 0 | 0 | 0 |
| allB | 0 | 1 | 1 | 0 | 0 | 1 | 0 | 0 | 0 | 0 | 0 | 0 | 0 | 0 | 0 | 0 | 0 |
| glxK | 0 | 1 | 1 | 0 | 0 | 1 | 0 | 0 | 0 | 0 | 0 | 0 | 0 | 0 | 0 | 0 | 0 |
| ylbA | 0 | 1 | 1 | 0 | 0 | 1 | 0 | 0 | 0 | 0 | 0 | 0 | 0 | 0 | 0 | 0 | 0 |
| allC | 0 | 1 | 1 | 0 | 0 | 1 | 0 | 0 | 0 | 0 | 0 | 0 | 0 | 0 | 0 | 0 | 0 |
| acs | 0 | 1 | 1 | 0 | 0 | 1 | 0 | 0 | 0 | 0 | 0 | 0 | 0 | 0 | 0 | 0 | 0 |
| alsK | 0 | 1 | 1 | 0 | 0 | 1 | 0 | 0 | 0 | 0 | 0 | 0 | 0 | 0 | 0 | 0 | 0 |
| alsE | 0 | 1 | 1 | 0 | 0 | 1 | 0 | 0 | 0 | 0 | 0 | 0 | 0 | 0 | 0 | 0 | 0 |
| phnP | 0 | 1 | 1 | 0 | 0 | 1 | 0 | 0 | 0 | 0 | 0 | 0 | 0 | 0 | 0 | 0 | 0 |
| phnO | 0 | 1 | 1 | 0 | 0 | 1 | 0 | 0 | 0 | 0 | 0 | 0 | 0 | 0 | 0 | 0 | 0 |
| phnJ | 0 | 1 | 1 | 0 | 0 | 1 | 0 | 0 | 0 | 0 | 0 | 0 | 0 | 0 | 0 | 0 | 0 |
| phnI | 0 | 1 | 1 | 0 | 0 | 1 | 0 | 0 | 0 | 0 | 0 | 0 | 0 | 0 | 0 | 0 | 0 |
| phnH | 0 | 1 | 1 | 0 | 0 | 1 | 0 | 0 | 0 | 0 | 0 | 0 | 0 | 0 | 0 | 0 | 0 |
| phnG | 0 | 1 | 1 | 0 | 0 | 1 | 0 | 0 | 0 | 0 | 0 | 0 | 0 | 0 | 0 | 0 | 0 |
| yjdM | 0 | 1 | 1 | 0 | 0 | 1 | 0 | 0 | 0 | 0 | 0 | 0 | 0 | 0 | 0 | 0 | 0 |
| yjdA | 0 | 1 | 1 | 0 | 0 | 1 | 0 | 0 | 0 | 0 | 0 | 0 | 0 | 0 | 0 | 0 | 0 |
| adiY | 0 | 1 | 1 | 0 | 0 | 1 | 0 | 0 | 0 | 0 | 0 | 0 | 0 | 0 | 0 | 0 | 0 |
| melA | 0 | 1 | 1 | 0 | 0 | 1 | 0 | 0 | 0 | 0 | 0 | 0 | 0 | 0 | 0 | 0 | 0 |
| yjdJ | 0 | 1 | 1 | 0 | 0 | 1 | 0 | 0 | 0 | 0 | 0 | 0 | 0 | 0 | 0 | 0 | 0 |
| xylB | 0 | 1 | 1 | 0 | 0 | 1 | 0 | 0 | 0 | 0 | 0 | 0 | 0 | 0 | 0 | 0 | 0 |
| ysaA | 0 | 1 | 1 | 0 | 0 | 1 | 0 | 0 | 0 | 0 | 0 | 0 | 0 | 0 | 0 | 0 | 0 |
| yiaJ | 0 | 1 | 1 | 0 | 0 | 1 | 0 | 0 | 0 | 0 | 0 | 0 | 0 | 0 | 0 | 0 | 0 |
| yiaK | 0 | 1 | 1 | 0 | 0 | 1 | 0 | 0 | 0 | 0 | 0 | 0 | 0 | 0 | 0 | 0 | 0 |
| yiaL | 0 | 1 | 1 | 0 | 0 | 1 | 0 | 0 | 0 | 0 | 0 | 0 | 0 | 0 | 0 | 0 | 0 |
| lyxK | 0 | 1 | 1 | 0 | 0 | 1 | 0 | 0 | 0 | 0 | 0 | 0 | 0 | 0 | 0 | 0 | 0 |
| yiaQ | 0 | 1 | 1 | 0 | 0 | 1 | 0 | 0 | 0 | 0 | 0 | 0 | 0 | 0 | 0 | 0 | 0 |
| yiaR | 0 | 1 | 1 | 0 | 0 | 1 | 0 | 0 | 0 | 0 | 0 | 0 | 0 | 0 | 0 | 0 | 0 |
| yiaS | 0 | 1 | 1 | 0 | 0 | 1 | 0 | 0 | 0 | 0 | 0 | 0 | 0 | 0 | 0 | 0 | 0 |
| yiaU | 0 | 1 | 1 | 0 | 0 | 1 | 0 | 0 | 0 | 0 | 0 | 0 | 0 | 0 | 0 | 0 | 0 |
| aldB | 0 | 1 | 1 | 0 | 0 | 1 | 0 | 0 | 0 | 0 | 0 | 0 | 0 | 0 | 0 | 0 | 0 |
| yiaY | 0 | 1 | 1 | 0 | 0 | 1 | 0 | 0 | 0 | 0 | 0 | 0 | 0 | 0 | 0 | 0 | 0 |
| yibF | 0 | 1 | 1 | 0 | 0 | 1 | 0 | 0 | 0 | 0 | 0 | 0 | 0 | 0 | 0 | 0 | 0 |
| yibA | 0 | 1 | 1 | 0 | 0 | 1 | 0 | 0 | 0 | 0 | 0 | 0 | 0 | 0 | 0 | 0 | 0 |
| yhjC | 0 | 1 | 1 | 0 | 0 | 1 | 0 | 0 | 0 | 0 | 0 | 0 | 0 | 0 | 0 | 0 | 0 |
| yhjH | 0 | 1 | 1 | 0 | 0 | 1 | 0 | 0 | 0 | 0 | 0 | 0 | 0 | 0 | 0 | 0 | 0 |
| yehL | 0 | 1 | 1 | 0 | 0 | 1 | 0 | 0 | 0 | 0 | 0 | 0 | 0 | 0 | 0 | 0 | 0 |
| yehP | 0 | 1 | 1 | 0 | 0 | 1 | 0 | 0 | 0 | 0 | 0 | 0 | 0 | 0 | 0 | 0 | 0 |
| yehS | 0 | 1 | 1 | 0 | 0 | 1 | 0 | 0 | 0 | 0 | 0 | 0 | 0 | 0 | 0 | 0 | 0 |
| mlrA | 0 | 1 | 1 | 0 | 0 | 1 | 0 | 0 | 0 | 0 | 0 | 0 | 0 | 0 | 0 | 0 | 0 |
| yohF | 0 | 1 | 1 | 0 | 0 | 1 | 0 | 0 | 0 | 0 | 0 | 0 | 0 | 0 | 0 | 0 | 0 |
| dusC | 0 | 1 | 1 | 0 | 0 | 1 | 0 | 0 | 0 | 0 | 0 | 0 | 0 | 0 | 0 | 0 | 0 |
| cdd | 0 | 1 | 1 | 0 | 0 | 1 | 0 | 0 | 0 | 0 | 0 | 0 | 0 | 0 | 0 | 0 | 0 |
| preA | 0 | 1 | 1 | 0 | 0 | 1 | 0 | 0 | 0 | 0 | 0 | 0 | 0 | 0 | 0 | 0 | 0 |
| yeeY | 0 | 1 | 1 | 0 | 0 | 1 | 0 | 0 | 0 | 0 | 0 | 0 | 0 | 0 | 0 | 0 | 0 |
| yeeZ | 0 | 1 | 1 | 0 | 0 | 1 | 0 | 0 | 0 | 0 | 0 | 0 | 0 | 0 | 0 | 0 | 0 |
| yoeB | 0 | 1 | 1 | 0 | 0 | 1 | 0 | 0 | 0 | 0 | 0 | 0 | 0 | 0 | 0 | 0 | 0 |
| hisL | 0 | 1 | 1 | 0 | 0 | 1 | 0 | 0 | 0 | 0 | 0 | 0 | 0 | 0 | 0 | 0 | 0 |
| hisD | 0 | 1 | 1 | 0 | 0 | 1 | 0 | 0 | 0 | 0 | 0 | 0 | 0 | 0 | 0 | 0 | 0 |
| hisC | 0 | 1 | 1 | 0 | 0 | 1 | 0 | 0 | 0 | 0 | 0 | 0 | 0 | 0 | 0 | 0 | 0 |
| hisB | 0 | 1 | 1 | 0 | 0 | 1 | 0 | 0 | 0 | 0 | 0 | 0 | 0 | 0 | 0 | 0 | 0 |
| hisH | 0 | 1 | 1 | 0 | 0 | 1 | 0 | 0 | 0 | 0 | 0 | 0 | 0 | 0 | 0 | 0 | 0 |
| ugd | 0 | 1 | 1 | 0 | 0 | 1 | 0 | 0 | 0 | 0 | 0 | 0 | 0 | 0 | 0 | 0 | 0 |
| gnd | 0 | 1 | 1 | 0 | 0 | 1 | 0 | 0 | 0 | 0 | 0 | 0 | 0 | 0 | 0 | 0 | 0 |
| wbbJ | 0 | 1 | 1 | 0 | 0 | 1 | 0 | 0 | 0 | 0 | 0 | 0 | 0 | 0 | 0 | 0 | 0 |
| wbbI | 0 | 1 | 1 | 0 | 0 | 1 | 0 | 0 | 0 | 0 | 0 | 0 | 0 | 0 | 0 | 0 | 0 |
| glf | 0 | 1 | 1 | 0 | 0 | 1 | 0 | 0 | 0 | 0 | 0 | 0 | 0 | 0 | 0 | 0 | 0 |
| rfbC | 0 | 1 | 1 | 0 | 0 | 1 | 0 | 0 | 0 | 0 | 0 | 0 | 0 | 0 | 0 | 0 | 0 |
| rfbA | 0 | 1 | 1 | 0 | 0 | 1 | 0 | 0 | 0 | 0 | 0 | 0 | 0 | 0 | 0 | 0 | 0 |
| rfbD | 0 | 1 | 1 | 0 | 0 | 1 | 0 | 0 | 0 | 0 | 0 | 0 | 0 | 0 | 0 | 0 | 0 |
| rfbB | 0 | 1 | 1 | 0 | 0 | 1 | 0 | 0 | 0 | 0 | 0 | 0 | 0 | 0 | 0 | 0 | 0 |
| wcaN | 0 | 1 | 1 | 0 | 0 | 1 | 0 | 0 | 0 | 0 | 0 | 0 | 0 | 0 | 0 | 0 | 0 |
| wcaM | 0 | 1 | 1 | 0 | 0 | 1 | 0 | 0 | 0 | 0 | 0 | 0 | 0 | 0 | 0 | 0 | 0 |
| wcaL | 0 | 1 | 1 | 0 | 0 | 1 | 0 | 0 | 0 | 0 | 0 | 0 | 0 | 0 | 0 | 0 | 0 |
| wcaK | 0 | 1 | 1 | 0 | 0 | 1 | 0 | 0 | 0 | 0 | 0 | 0 | 0 | 0 | 0 | 0 | 0 |
| cpsG | 0 | 1 | 1 | 0 | 0 | 1 | 0 | 0 | 0 | 0 | 0 | 0 | 0 | 0 | 0 | 0 | 0 |
| cpsB | 0 | 1 | 1 | 0 | 0 | 1 | 0 | 0 | 0 | 0 | 0 | 0 | 0 | 0 | 0 | 0 | 0 |
| wcaI | 0 | 1 | 1 | 0 | 0 | 1 | 0 | 0 | 0 | 0 | 0 | 0 | 0 | 0 | 0 | 0 | 0 |
| wcaH | 0 | 1 | 1 | 0 | 0 | 1 | 0 | 0 | 0 | 0 | 0 | 0 | 0 | 0 | 0 | 0 | 0 |
| wcaG | 0 | 1 | 1 | 0 | 0 | 1 | 0 | 0 | 0 | 0 | 0 | 0 | 0 | 0 | 0 | 0 | 0 |
| gmd | 0 | 1 | 1 | 0 | 0 | 1 | 0 | 0 | 0 | 0 | 0 | 0 | 0 | 0 | 0 | 0 | 0 |
| wcaF | 0 | 1 | 1 | 0 | 0 | 1 | 0 | 0 | 0 | 0 | 0 | 0 | 0 | 0 | 0 | 0 | 0 |
| wcaE | 0 | 1 | 1 | 0 | 0 | 1 | 0 | 0 | 0 | 0 | 0 | 0 | 0 | 0 | 0 | 0 | 0 |
| wcaC | 0 | 1 | 1 | 0 | 0 | 1 | 0 | 0 | 0 | 0 | 0 | 0 | 0 | 0 | 0 | 0 | 0 |
| wcaB | 0 | 1 | 1 | 0 | 0 | 1 | 0 | 0 | 0 | 0 | 0 | 0 | 0 | 0 | 0 | 0 | 0 |
| wcaA | 0 | 1 | 1 | 0 | 0 | 1 | 0 | 0 | 0 | 0 | 0 | 0 | 0 | 0 | 0 | 0 | 0 |
| wzb | 0 | 1 | 1 | 0 | 0 | 1 | 0 | 0 | 0 | 0 | 0 | 0 | 0 | 0 | 0 | 0 | 0 |
| dcd | 0 | 1 | 1 | 0 | 0 | 1 | 0 | 0 | 0 | 0 | 0 | 0 | 0 | 0 | 0 | 0 | 0 |
| udk | 0 | 1 | 1 | 0 | 0 | 1 | 0 | 0 | 0 | 0 | 0 | 0 | 0 | 0 | 0 | 0 | 0 |
| alkA | 0 | 1 | 1 | 0 | 0 | 1 | 0 | 0 | 0 | 0 | 0 | 0 | 0 | 0 | 0 | 0 | 0 |
| yegD | 0 | 1 | 1 | 0 | 0 | 1 | 0 | 0 | 0 | 0 | 0 | 0 | 0 | 0 | 0 | 0 | 0 |
| baeR | 0 | 1 | 1 | 0 | 0 | 1 | 0 | 0 | 0 | 0 | 0 | 0 | 0 | 0 | 0 | 0 | 0 |
| yegQ | 0 | 1 | 1 | 0 | 0 | 1 | 0 | 0 | 0 | 0 | 0 | 0 | 0 | 0 | 0 | 0 | 0 |
| ogrK | 0 | 1 | 1 | 0 | 0 | 1 | 0 | 0 | 0 | 0 | 0 | 0 | 0 | 0 | 0 | 0 | 0 |
| yegS | 0 | 1 | 1 | 0 | 0 | 1 | 0 | 0 | 0 | 0 | 0 | 0 | 0 | 0 | 0 | 0 | 0 |
| insE1 | 0 | 1 | 1 | 0 | 0 | 1 | 0 | 0 | 0 | 0 | 0 | 0 | 0 | 0 | 0 | 0 | 0 |
| insF1 | 0 | 1 | 1 | 0 | 0 | 1 | 0 | 0 | 0 | 0 | 0 | 0 | 0 | 0 | 0 | 0 | 0 |
| gatD | 0 | 1 | 1 | 0 | 0 | 1 | 0 | 0 | 0 | 0 | 0 | 0 | 0 | 0 | 0 | 0 | 0 |
| gatB | 0 | 1 | 1 | 0 | 0 | 1 | 0 | 0 | 0 | 0 | 0 | 0 | 0 | 0 | 0 | 0 | 0 |
| gatA | 0 | 1 | 1 | 0 | 0 | 1 | 0 | 0 | 0 | 0 | 0 | 0 | 0 | 0 | 0 | 0 | 0 |
| gatY | 0 | 1 | 1 | 0 | 0 | 1 | 0 | 0 | 0 | 0 | 0 | 0 | 0 | 0 | 0 | 0 | 0 |
| fbaB | 0 | 1 | 1 | 0 | 0 | 1 | 0 | 0 | 0 | 0 | 0 | 0 | 0 | 0 | 0 | 0 | 0 |
| yegU | 0 | 1 | 1 | 0 | 0 | 1 | 0 | 0 | 0 | 0 | 0 | 0 | 0 | 0 | 0 | 0 | 0 |
| yegV | 0 | 1 | 1 | 0 | 0 | 1 | 0 | 0 | 0 | 0 | 0 | 0 | 0 | 0 | 0 | 0 | 0 |
| yegW | 0 | 1 | 1 | 0 | 0 | 1 | 0 | 0 | 0 | 0 | 0 | 0 | 0 | 0 | 0 | 0 | 0 |
| thiD | 0 | 1 | 1 | 0 | 0 | 1 | 0 | 0 | 0 | 0 | 0 | 0 | 0 | 0 | 0 | 0 | 0 |
| thiM | 0 | 1 | 1 | 0 | 0 | 1 | 0 | 0 | 0 | 0 | 0 | 0 | 0 | 0 | 0 | 0 | 0 |
| rcnR | 0 | 1 | 1 | 0 | 0 | 1 | 0 | 0 | 0 | 0 | 0 | 0 | 0 | 0 | 0 | 0 | 0 |
| mrp | 0 | 1 | 1 | 0 | 0 | 1 | 0 | 0 | 0 | 0 | 0 | 0 | 0 | 0 | 0 | 0 | 0 |
| etp | 0 | 1 | 1 | 0 | 0 | 1 | 0 | 0 | 0 | 0 | 0 | 0 | 0 | 0 | 0 | 0 | 0 |
| cspH | 0 | 1 | 1 | 0 | 0 | 1 | 0 | 0 | 0 | 0 | 0 | 0 | 0 | 0 | 0 | 0 | 0 |
| cspG | 0 | 1 | 1 | 0 | 0 | 1 | 0 | 0 | 0 | 0 | 0 | 0 | 0 | 0 | 0 | 0 | 0 |
| torR | 0 | 1 | 1 | 0 | 0 | 1 | 0 | 0 | 0 | 0 | 0 | 0 | 0 | 0 | 0 | 0 | 0 |
| torD | 0 | 1 | 1 | 0 | 0 | 1 | 0 | 0 | 0 | 0 | 0 | 0 | 0 | 0 | 0 | 0 | 0 |
| cbpM | 0 | 1 | 1 | 0 | 0 | 1 | 0 | 0 | 0 | 0 | 0 | 0 | 0 | 0 | 0 | 0 | 0 |
| yccJ | 0 | 1 | 1 | 0 | 0 | 1 | 0 | 0 | 0 | 0 | 0 | 0 | 0 | 0 | 0 | 0 | 0 |
| wrbA | 0 | 1 | 1 | 0 | 0 | 1 | 0 | 0 | 0 | 0 | 0 | 0 | 0 | 0 | 0 | 0 | 0 |
| ymdF | 0 | 1 | 1 | 0 | 0 | 1 | 0 | 0 | 0 | 0 | 0 | 0 | 0 | 0 | 0 | 0 | 0 |
| rutF | 0 | 1 | 1 | 0 | 0 | 1 | 0 | 0 | 0 | 0 | 0 | 0 | 0 | 0 | 0 | 0 | 0 |
| rutC | 0 | 1 | 1 | 0 | 0 | 1 | 0 | 0 | 0 | 0 | 0 | 0 | 0 | 0 | 0 | 0 | 0 |
| rutB | 0 | 1 | 1 | 0 | 0 | 1 | 0 | 0 | 0 | 0 | 0 | 0 | 0 | 0 | 0 | 0 | 0 |
| rutR | 0 | 1 | 1 | 0 | 0 | 1 | 0 | 0 | 0 | 0 | 0 | 0 | 0 | 0 | 0 | 0 | 0 |
| phoH | 0 | 1 | 1 | 0 | 0 | 1 | 0 | 0 | 0 | 0 | 0 | 0 | 0 | 0 | 0 | 0 | 0 |
| ycdX | 0 | 1 | 1 | 0 | 0 | 1 | 0 | 0 | 0 | 0 | 0 | 0 | 0 | 0 | 0 | 0 | 0 |
| ycdY | 0 | 1 | 1 | 0 | 0 | 1 | 0 | 0 | 0 | 0 | 0 | 0 | 0 | 0 | 0 | 0 | 0 |
| ymdB | 0 | 1 | 1 | 0 | 0 | 1 | 0 | 0 | 0 | 0 | 0 | 0 | 0 | 0 | 0 | 0 | 0 |
| msyB | 0 | 1 | 1 | 0 | 0 | 1 | 0 | 0 | 0 | 0 | 0 | 0 | 0 | 0 | 0 | 0 | 0 |
| yfjH | 0 | 1 | 1 | 0 | 0 | 1 | 0 | 0 | 0 | 0 | 0 | 0 | 0 | 0 | 0 | 0 | 0 |
| alpA | 0 | 1 | 1 | 0 | 0 | 1 | 0 | 0 | 0 | 0 | 0 | 0 | 0 | 0 | 0 | 0 | 0 |
| yfjI | 0 | 1 | 1 | 0 | 0 | 1 | 0 | 0 | 0 | 0 | 0 | 0 | 0 | 0 | 0 | 0 | 0 |
| yfjK | 0 | 1 | 1 | 0 | 0 | 1 | 0 | 0 | 0 | 0 | 0 | 0 | 0 | 0 | 0 | 0 | 0 |
| yfjL | 0 | 1 | 1 | 0 | 0 | 1 | 0 | 0 | 0 | 0 | 0 | 0 | 0 | 0 | 0 | 0 | 0 |
| rnlA | 0 | 1 | 1 | 0 | 0 | 1 | 0 | 0 | 0 | 0 | 0 | 0 | 0 | 0 | 0 | 0 | 0 |
| rnlB | 0 | 1 | 1 | 0 | 0 | 1 | 0 | 0 | 0 | 0 | 0 | 0 | 0 | 0 | 0 | 0 | 0 |
| yfjP | 0 | 1 | 1 | 0 | 0 | 1 | 0 | 0 | 0 | 0 | 0 | 0 | 0 | 0 | 0 | 0 | 0 |
| yfjQ | 0 | 1 | 1 | 0 | 0 | 1 | 0 | 0 | 0 | 0 | 0 | 0 | 0 | 0 | 0 | 0 | 0 |
| yfjR | 0 | 1 | 1 | 0 | 0 | 1 | 0 | 0 | 0 | 0 | 0 | 0 | 0 | 0 | 0 | 0 | 0 |
| ypjK | 0 | 1 | 1 | 0 | 0 | 1 | 0 | 0 | 0 | 0 | 0 | 0 | 0 | 0 | 0 | 0 | 0 |
| yfjU | 0 | 1 | 1 | 0 | 0 | 1 | 0 | 0 | 0 | 0 | 0 | 0 | 0 | 0 | 0 | 0 | 0 |
| yfjX | 0 | 1 | 1 | 0 | 0 | 1 | 0 | 0 | 0 | 0 | 0 | 0 | 0 | 0 | 0 | 0 | 0 |
| yfjY | 0 | 1 | 1 | 0 | 0 | 1 | 0 | 0 | 0 | 0 | 0 | 0 | 0 | 0 | 0 | 0 | 0 |
| yfjZ | 0 | 1 | 1 | 0 | 0 | 1 | 0 | 0 | 0 | 0 | 0 | 0 | 0 | 0 | 0 | 0 | 0 |
| ypjF | 0 | 1 | 1 | 0 | 0 | 1 | 0 | 0 | 0 | 0 | 0 | 0 | 0 | 0 | 0 | 0 | 0 |
| csiD | 0 | 1 | 1 | 0 | 0 | 1 | 0 | 0 | 0 | 0 | 0 | 0 | 0 | 0 | 0 | 0 | 0 |
| gabD | 0 | 1 | 1 | 0 | 0 | 1 | 0 | 0 | 0 | 0 | 0 | 0 | 0 | 0 | 0 | 0 | 0 |
| gabT | 0 | 1 | 1 | 0 | 0 | 1 | 0 | 0 | 0 | 0 | 0 | 0 | 0 | 0 | 0 | 0 | 0 |
| csiR | 0 | 1 | 1 | 0 | 0 | 1 | 0 | 0 | 0 | 0 | 0 | 0 | 0 | 0 | 0 | 0 | 0 |
| ygaU | 0 | 1 | 1 | 0 | 0 | 1 | 0 | 0 | 0 | 0 | 0 | 0 | 0 | 0 | 0 | 0 | 0 |
| ygaV | 0 | 1 | 1 | 0 | 0 | 1 | 0 | 0 | 0 | 0 | 0 | 0 | 0 | 0 | 0 | 0 | 0 |
| ygaC | 0 | 1 | 1 | 0 | 0 | 1 | 0 | 0 | 0 | 0 | 0 | 0 | 0 | 0 | 0 | 0 | 0 |
| nrdH | 0 | 1 | 1 | 0 | 0 | 1 | 0 | 0 | 0 | 0 | 0 | 0 | 0 | 0 | 0 | 0 | 0 |
| nrdI | 0 | 1 | 1 | 0 | 0 | 1 | 0 | 0 | 0 | 0 | 0 | 0 | 0 | 0 | 0 | 0 | 0 |
| nrdE | 0 | 1 | 1 | 0 | 0 | 1 | 0 | 0 | 0 | 0 | 0 | 0 | 0 | 0 | 0 | 0 | 0 |
| nrdF | 0 | 1 | 1 | 0 | 0 | 1 | 0 | 0 | 0 | 0 | 0 | 0 | 0 | 0 | 0 | 0 | 0 |
| mprA | 0 | 1 | 1 | 0 | 0 | 1 | 0 | 0 | 0 | 0 | 0 | 0 | 0 | 0 | 0 | 0 | 0 |
| srlB | 0 | 0 | 1 | 0 | 0 | 1 | 0 | 0 | 0 | 0 | 0 | 0 | 0 | 0 | 0 | 0 | 0 |
| srlD | 0 | 0 | 1 | 0 | 0 | 1 | 0 | 0 | 0 | 0 | 0 | 0 | 0 | 0 | 0 | 0 | 0 |
| srlR | 0 | 0 | 1 | 0 | 0 | 1 | 0 | 0 | 0 | 0 | 0 | 0 | 0 | 0 | 0 | 0 | 0 |
| norR | 0 | 0 | 1 | 0 | 0 | 1 | 0 | 0 | 0 | 0 | 0 | 0 | 0 | 0 | 0 | 0 | 0 |
| norV | 0 | 0 | 1 | 0 | 0 | 1 | 0 | 0 | 0 | 0 | 0 | 0 | 0 | 0 | 0 | 0 | 0 |
| norW | 0 | 0 | 1 | 0 | 0 | 1 | 0 | 0 | 0 | 0 | 0 | 0 | 0 | 0 | 0 | 0 | 0 |
| hypF | 0 | 0 | 1 | 0 | 0 | 1 | 0 | 0 | 0 | 0 | 0 | 0 | 0 | 0 | 0 | 0 | 0 |
| hydN | 0 | 0 | 1 | 0 | 0 | 1 | 0 | 0 | 0 | 0 | 0 | 0 | 0 | 0 | 0 | 0 | 0 |
| ascG | 0 | 0 | 1 | 0 | 0 | 1 | 0 | 0 | 0 | 0 | 0 | 0 | 0 | 0 | 0 | 0 | 0 |
| ascB | 0 | 0 | 1 | 0 | 0 | 1 | 0 | 0 | 0 | 0 | 0 | 0 | 0 | 0 | 0 | 0 | 0 |
| hycI | 0 | 0 | 1 | 0 | 0 | 1 | 0 | 0 | 0 | 0 | 0 | 0 | 0 | 0 | 0 | 0 | 0 |
| hycH | 0 | 0 | 1 | 0 | 0 | 1 | 0 | 0 | 0 | 0 | 0 | 0 | 0 | 0 | 0 | 0 | 0 |
| hycG | 0 | 0 | 1 | 0 | 0 | 1 | 0 | 0 | 0 | 0 | 0 | 0 | 0 | 0 | 0 | 0 | 0 |
| hycE | 0 | 0 | 1 | 0 | 0 | 1 | 0 | 0 | 0 | 0 | 0 | 0 | 0 | 0 | 0 | 0 | 0 |
| hycB | 0 | 0 | 1 | 0 | 0 | 1 | 0 | 0 | 0 | 0 | 0 | 0 | 0 | 0 | 0 | 0 | 0 |
| hypE | 0 | 0 | 1 | 0 | 0 | 1 | 0 | 0 | 0 | 0 | 0 | 0 | 0 | 0 | 0 | 0 | 0 |
| fhlA | 0 | 0 | 1 | 0 | 0 | 1 | 0 | 0 | 0 | 0 | 0 | 0 | 0 | 0 | 0 | 0 | 0 |
| mutS | 0 | 0 | 1 | 0 | 0 | 1 | 0 | 0 | 0 | 0 | 0 | 0 | 0 | 0 | 0 | 0 | 0 |
| ygbI | 0 | 0 | 1 | 0 | 0 | 1 | 0 | 0 | 0 | 0 | 0 | 0 | 0 | 0 | 0 | 0 | 0 |
| ygbJ | 0 | 0 | 1 | 0 | 0 | 1 | 0 | 0 | 0 | 0 | 0 | 0 | 0 | 0 | 0 | 0 | 0 |
| ygbL | 0 | 0 | 1 | 0 | 0 | 1 | 0 | 0 | 0 | 0 | 0 | 0 | 0 | 0 | 0 | 0 | 0 |
| ygbM | 0 | 0 | 1 | 0 | 0 | 1 | 0 | 0 | 0 | 0 | 0 | 0 | 0 | 0 | 0 | 0 | 0 |
| narP | 0 | 0 | 1 | 0 | 0 | 1 | 0 | 0 | 0 | 0 | 0 | 0 | 0 | 0 | 0 | 0 | 0 |
| napD | 0 | 0 | 1 | 0 | 0 | 1 | 0 | 0 | 0 | 0 | 0 | 0 | 0 | 0 | 0 | 0 | 0 |
| rcsB | 0 | 0 | 1 | 0 | 0 | 1 | 0 | 0 | 0 | 0 | 0 | 0 | 0 | 0 | 0 | 0 | 0 |
| atoC | 0 | 0 | 1 | 0 | 0 | 1 | 0 | 0 | 0 | 0 | 0 | 0 | 0 | 0 | 0 | 0 | 0 |
| atoA | 0 | 0 | 1 | 0 | 0 | 1 | 0 | 0 | 0 | 0 | 0 | 0 | 0 | 0 | 0 | 0 | 0 |
| atoB | 0 | 0 | 1 | 0 | 0 | 1 | 0 | 0 | 0 | 0 | 0 | 0 | 0 | 0 | 0 | 0 | 0 |
| yfaA | 0 | 0 | 1 | 0 | 0 | 1 | 0 | 0 | 0 | 0 | 0 | 0 | 0 | 0 | 0 | 0 | 0 |
| inaA | 0 | 0 | 1 | 0 | 0 | 1 | 0 | 0 | 0 | 0 | 0 | 0 | 0 | 0 | 0 | 0 | 0 |
| yfaD | 0 | 0 | 1 | 0 | 0 | 1 | 0 | 0 | 0 | 0 | 0 | 0 | 0 | 0 | 0 | 0 | 0 |
| rhmD | 0 | 0 | 1 | 0 | 0 | 1 | 0 | 0 | 0 | 0 | 0 | 0 | 0 | 0 | 0 | 0 | 0 |
| rhmR | 0 | 0 | 1 | 0 | 0 | 1 | 0 | 0 | 0 | 0 | 0 | 0 | 0 | 0 | 0 | 0 | 0 |
| yfaY | 0 | 0 | 1 | 0 | 0 | 1 | 0 | 0 | 0 | 0 | 0 | 0 | 0 | 0 | 0 | 0 | 0 |
| nudI | 0 | 0 | 1 | 0 | 0 | 1 | 0 | 0 | 0 | 0 | 0 | 0 | 0 | 0 | 0 | 0 | 0 |
| arnB | 0 | 0 | 1 | 0 | 0 | 1 | 0 | 0 | 0 | 0 | 0 | 0 | 0 | 0 | 0 | 0 | 0 |
| arnA | 0 | 0 | 1 | 0 | 0 | 1 | 0 | 0 | 0 | 0 | 0 | 0 | 0 | 0 | 0 | 0 | 0 |
| arnD | 0 | 0 | 1 | 0 | 0 | 1 | 0 | 0 | 0 | 0 | 0 | 0 | 0 | 0 | 0 | 0 | 0 |
| pmrD | 0 | 0 | 1 | 0 | 0 | 1 | 0 | 0 | 0 | 0 | 0 | 0 | 0 | 0 | 0 | 0 | 0 |
| menE | 0 | 0 | 1 | 0 | 0 | 1 | 0 | 0 | 0 | 0 | 0 | 0 | 0 | 0 | 0 | 0 | 0 |
| menC | 0 | 0 | 1 | 0 | 0 | 1 | 0 | 0 | 0 | 0 | 0 | 0 | 0 | 0 | 0 | 0 | 0 |
| menB | 0 | 0 | 1 | 0 | 0 | 1 | 0 | 0 | 0 | 0 | 0 | 0 | 0 | 0 | 0 | 0 | 0 |
| menH | 0 | 0 | 1 | 0 | 0 | 1 | 0 | 0 | 0 | 0 | 0 | 0 | 0 | 0 | 0 | 0 | 0 |
| menF | 0 | 0 | 1 | 0 | 0 | 1 | 0 | 0 | 0 | 0 | 0 | 0 | 0 | 0 | 0 | 0 | 0 |
| rbn | 0 | 0 | 1 | 0 | 0 | 1 | 0 | 0 | 0 | 0 | 0 | 0 | 0 | 0 | 0 | 0 | 0 |
| yfbM | 0 | 0 | 1 | 0 | 0 | 1 | 0 | 0 | 0 | 0 | 0 | 0 | 0 | 0 | 0 | 0 | 0 |
| yfbN | 0 | 0 | 1 | 0 | 0 | 1 | 0 | 0 | 0 | 0 | 0 | 0 | 0 | 0 | 0 | 0 | 0 |
| yfbO | 0 | 0 | 1 | 0 | 0 | 1 | 0 | 0 | 0 | 0 | 0 | 0 | 0 | 0 | 0 | 0 | 0 |
| ymjA | 0 | 0 | 1 | 0 | 0 | 1 | 0 | 0 | 0 | 0 | 0 | 0 | 0 | 0 | 0 | 0 | 0 |
| puuA | 0 | 0 | 1 | 0 | 0 | 1 | 0 | 0 | 0 | 0 | 0 | 0 | 0 | 0 | 0 | 0 | 0 |
| puuD | 0 | 0 | 1 | 0 | 0 | 1 | 0 | 0 | 0 | 0 | 0 | 0 | 0 | 0 | 0 | 0 | 0 |
| puuR | 0 | 0 | 1 | 0 | 0 | 1 | 0 | 0 | 0 | 0 | 0 | 0 | 0 | 0 | 0 | 0 | 0 |
| puuC | 0 | 0 | 1 | 0 | 0 | 1 | 0 | 0 | 0 | 0 | 0 | 0 | 0 | 0 | 0 | 0 | 0 |
| puuB | 0 | 0 | 1 | 0 | 0 | 1 | 0 | 0 | 0 | 0 | 0 | 0 | 0 | 0 | 0 | 0 | 0 |
| puuE | 0 | 0 | 1 | 0 | 0 | 1 | 0 | 0 | 0 | 0 | 0 | 0 | 0 | 0 | 0 | 0 | 0 |
| pspF | 0 | 0 | 1 | 0 | 0 | 1 | 0 | 0 | 0 | 0 | 0 | 0 | 0 | 0 | 0 | 0 | 0 |
| ycjM | 0 | 0 | 1 | 0 | 0 | 1 | 0 | 0 | 0 | 0 | 0 | 0 | 0 | 0 | 0 | 0 | 0 |
| ycjQ | 0 | 0 | 1 | 0 | 0 | 1 | 0 | 0 | 0 | 0 | 0 | 0 | 0 | 0 | 0 | 0 | 0 |
| ycjT | 0 | 0 | 1 | 0 | 0 | 1 | 0 | 0 | 0 | 0 | 0 | 0 | 0 | 0 | 0 | 0 | 0 |
| ycjU | 0 | 0 | 1 | 0 | 0 | 1 | 0 | 0 | 0 | 0 | 0 | 0 | 0 | 0 | 0 | 0 | 0 |
| ycjW | 0 | 0 | 1 | 0 | 0 | 1 | 0 | 0 | 0 | 0 | 0 | 0 | 0 | 0 | 0 | 0 | 0 |
| ycjX | 0 | 0 | 1 | 0 | 0 | 1 | 0 | 0 | 0 | 0 | 0 | 0 | 0 | 0 | 0 | 0 | 0 |
| tyrR | 0 | 0 | 1 | 0 | 0 | 1 | 0 | 0 | 0 | 0 | 0 | 0 | 0 | 0 | 0 | 0 | 0 |
| ycjG | 0 | 0 | 1 | 0 | 0 | 1 | 0 | 0 | 0 | 0 | 0 | 0 | 0 | 0 | 0 | 0 | 0 |
| mpaA | 0 | 0 | 1 | 0 | 0 | 1 | 0 | 0 | 0 | 0 | 0 | 0 | 0 | 0 | 0 | 0 | 0 |
| ycjZ | 0 | 0 | 1 | 0 | 0 | 1 | 0 | 0 | 0 | 0 | 0 | 0 | 0 | 0 | 0 | 0 | 0 |
| uspE | 0 | 0 | 1 | 0 | 0 | 1 | 0 | 0 | 0 | 0 | 0 | 0 | 0 | 0 | 0 | 0 | 0 |
| fnr | 0 | 0 | 1 | 0 | 0 | 1 | 0 | 0 | 0 | 0 | 0 | 0 | 0 | 0 | 0 | 0 | 0 |
| ogt | 0 | 0 | 1 | 0 | 0 | 1 | 0 | 0 | 0 | 0 | 0 | 0 | 0 | 0 | 0 | 0 | 0 |
| abgB | 0 | 0 | 1 | 0 | 0 | 1 | 0 | 0 | 0 | 0 | 0 | 0 | 0 | 0 | 0 | 0 | 0 |
| abgA | 0 | 0 | 1 | 0 | 0 | 1 | 0 | 0 | 0 | 0 | 0 | 0 | 0 | 0 | 0 | 0 | 0 |
| abgR | 0 | 0 | 1 | 0 | 0 | 1 | 0 | 0 | 0 | 0 | 0 | 0 | 0 | 0 | 0 | 0 | 0 |
| smrA | 0 | 0 | 1 | 0 | 0 | 1 | 0 | 0 | 0 | 0 | 0 | 0 | 0 | 0 | 0 | 0 | 0 |
| dbpA | 0 | 0 | 1 | 0 | 0 | 1 | 0 | 0 | 0 | 0 | 0 | 0 | 0 | 0 | 0 | 0 | 0 |
| ttcA | 0 | 0 | 1 | 0 | 0 | 1 | 0 | 0 | 0 | 0 | 0 | 0 | 0 | 0 | 0 | 0 | 0 |
| intR | 0 | 0 | 1 | 0 | 0 | 1 | 0 | 0 | 0 | 0 | 0 | 0 | 0 | 0 | 0 | 0 | 0 |
| recT | 0 | 0 | 1 | 0 | 0 | 1 | 0 | 0 | 0 | 0 | 0 | 0 | 0 | 0 | 0 | 0 | 0 |
| racC | 0 | 0 | 1 | 0 | 0 | 1 | 0 | 0 | 0 | 0 | 0 | 0 | 0 | 0 | 0 | 0 | 0 |
| kilR | 0 | 0 | 1 | 0 | 0 | 1 | 0 | 0 | 0 | 0 | 0 | 0 | 0 | 0 | 0 | 0 | 0 |
| ydaG | 0 | 0 | 1 | 0 | 0 | 1 | 0 | 0 | 0 | 0 | 0 | 0 | 0 | 0 | 0 | 0 | 0 |
| racR | 0 | 0 | 1 | 0 | 0 | 1 | 0 | 0 | 0 | 0 | 0 | 0 | 0 | 0 | 0 | 0 | 0 |
| ydaU | 0 | 0 | 1 | 0 | 0 | 1 | 0 | 0 | 0 | 0 | 0 | 0 | 0 | 0 | 0 | 0 | 0 |
| ydaV | 0 | 0 | 1 | 0 | 0 | 1 | 0 | 0 | 0 | 0 | 0 | 0 | 0 | 0 | 0 | 0 | 0 |
| ydaW | 0 | 0 | 1 | 0 | 0 | 1 | 0 | 0 | 0 | 0 | 0 | 0 | 0 | 0 | 0 | 0 | 0 |
| ynaK | 0 | 0 | 1 | 0 | 0 | 1 | 0 | 0 | 0 | 0 | 0 | 0 | 0 | 0 | 0 | 0 | 0 |
| tfaR | 0 | 0 | 1 | 0 | 0 | 1 | 0 | 0 | 0 | 0 | 0 | 0 | 0 | 0 | 0 | 0 | 0 |
| pinR | 0 | 0 | 1 | 0 | 0 | 1 | 0 | 0 | 0 | 0 | 0 | 0 | 0 | 0 | 0 | 0 | 0 |
| ldhA | 0 | 0 | 1 | 0 | 0 | 1 | 0 | 0 | 0 | 0 | 0 | 0 | 0 | 0 | 0 | 0 | 0 |
| ydbH | 0 | 0 | 1 | 0 | 0 | 1 | 0 | 0 | 0 | 0 | 0 | 0 | 0 | 0 | 0 | 0 | 0 |
| ynbE | 0 | 0 | 1 | 0 | 0 | 1 | 0 | 0 | 0 | 0 | 0 | 0 | 0 | 0 | 0 | 0 | 0 |
| feaR | 0 | 0 | 1 | 0 | 0 | 1 | 0 | 0 | 0 | 0 | 0 | 0 | 0 | 0 | 0 | 0 | 0 |
| feaB | 0 | 0 | 1 | 0 | 0 | 1 | 0 | 0 | 0 | 0 | 0 | 0 | 0 | 0 | 0 | 0 | 0 |
| paaA | 0 | 0 | 1 | 0 | 0 | 1 | 0 | 0 | 0 | 0 | 0 | 0 | 0 | 0 | 0 | 0 | 0 |
| paaC | 0 | 0 | 1 | 0 | 0 | 1 | 0 | 0 | 0 | 0 | 0 | 0 | 0 | 0 | 0 | 0 | 0 |
| paaE | 0 | 0 | 1 | 0 | 0 | 1 | 0 | 0 | 0 | 0 | 0 | 0 | 0 | 0 | 0 | 0 | 0 |
| paaG | 0 | 0 | 1 | 0 | 0 | 1 | 0 | 0 | 0 | 0 | 0 | 0 | 0 | 0 | 0 | 0 | 0 |
| paaI | 0 | 0 | 1 | 0 | 0 | 1 | 0 | 0 | 0 | 0 | 0 | 0 | 0 | 0 | 0 | 0 | 0 |
| paaJ | 0 | 0 | 1 | 0 | 0 | 1 | 0 | 0 | 0 | 0 | 0 | 0 | 0 | 0 | 0 | 0 | 0 |
| paaK | 0 | 0 | 1 | 0 | 0 | 1 | 0 | 0 | 0 | 0 | 0 | 0 | 0 | 0 | 0 | 0 | 0 |
| paaX | 0 | 0 | 1 | 0 | 0 | 1 | 0 | 0 | 0 | 0 | 0 | 0 | 0 | 0 | 0 | 0 | 0 |
| paaY | 0 | 0 | 1 | 0 | 0 | 1 | 0 | 0 | 0 | 0 | 0 | 0 | 0 | 0 | 0 | 0 | 0 |
| azoR | 0 | 0 | 1 | 0 | 0 | 1 | 0 | 0 | 0 | 0 | 0 | 0 | 0 | 0 | 0 | 0 | 0 |
| aldA | 0 | 0 | 1 | 0 | 0 | 1 | 0 | 0 | 0 | 0 | 0 | 0 | 0 | 0 | 0 | 0 | 0 |
| ydcI | 0 | 0 | 1 | 0 | 0 | 1 | 0 | 0 | 0 | 0 | 0 | 0 | 0 | 0 | 0 | 0 | 0 |
| ydcJ | 0 | 0 | 1 | 0 | 0 | 1 | 0 | 0 | 0 | 0 | 0 | 0 | 0 | 0 | 0 | 0 | 0 |
| rimL | 0 | 0 | 1 | 0 | 0 | 1 | 0 | 0 | 0 | 0 | 0 | 0 | 0 | 0 | 0 | 0 | 0 |
| ydcK | 0 | 0 | 1 | 0 | 0 | 1 | 0 | 0 | 0 | 0 | 0 | 0 | 0 | 0 | 0 | 0 | 0 |
| tehB | 0 | 0 | 1 | 0 | 0 | 1 | 0 | 0 | 0 | 0 | 0 | 0 | 0 | 0 | 0 | 0 | 0 |
| ydcN | 0 | 0 | 1 | 0 | 0 | 1 | 0 | 0 | 0 | 0 | 0 | 0 | 0 | 0 | 0 | 0 | 0 |
| ydcP | 0 | 0 | 1 | 0 | 0 | 1 | 0 | 0 | 0 | 0 | 0 | 0 | 0 | 0 | 0 | 0 | 0 |
| hicB | 0 | 0 | 1 | 0 | 0 | 1 | 0 | 0 | 0 | 0 | 0 | 0 | 0 | 0 | 0 | 0 | 0 |
| ydcR | 0 | 0 | 1 | 0 | 0 | 1 | 0 | 0 | 0 | 0 | 0 | 0 | 0 | 0 | 0 | 0 | 0 |
| patD | 0 | 0 | 1 | 0 | 0 | 1 | 0 | 0 | 0 | 0 | 0 | 0 | 0 | 0 | 0 | 0 | 0 |
| mnaT | 0 | 0 | 1 | 0 | 0 | 1 | 0 | 0 | 0 | 0 | 0 | 0 | 0 | 0 | 0 | 0 | 0 |
| curA | 0 | 0 | 1 | 0 | 0 | 1 | 0 | 0 | 0 | 0 | 0 | 0 | 0 | 0 | 0 | 0 | 0 |
| mcbR | 0 | 0 | 1 | 0 | 0 | 1 | 0 | 0 | 0 | 0 | 0 | 0 | 0 | 0 | 0 | 0 | 0 |
| yncG | 0 | 0 | 1 | 0 | 0 | 1 | 0 | 0 | 0 | 0 | 0 | 0 | 0 | 0 | 0 | 0 | 0 |
| ydcC | 0 | 0 | 1 | 0 | 0 | 1 | 0 | 0 | 0 | 0 | 0 | 0 | 0 | 0 | 0 | 0 | 0 |
| pptA | 0 | 0 | 1 | 0 | 0 | 1 | 0 | 0 | 0 | 0 | 0 | 0 | 0 | 0 | 0 | 0 | 0 |
| yddH | 0 | 0 | 1 | 0 | 0 | 1 | 0 | 0 | 0 | 0 | 0 | 0 | 0 | 0 | 0 | 0 | 0 |
| nhoA | 0 | 0 | 1 | 0 | 0 | 1 | 0 | 0 | 0 | 0 | 0 | 0 | 0 | 0 | 0 | 0 | 0 |
| yddE | 0 | 0 | 1 | 0 | 0 | 1 | 0 | 0 | 0 | 0 | 0 | 0 | 0 | 0 | 0 | 0 | 0 |
| narW | 0 | 0 | 1 | 0 | 0 | 1 | 0 | 0 | 0 | 0 | 0 | 0 | 0 | 0 | 0 | 0 | 0 |
| yddM | 0 | 0 | 1 | 0 | 0 | 1 | 0 | 0 | 0 | 0 | 0 | 0 | 0 | 0 | 0 | 0 | 0 |
| adhP | 0 | 0 | 1 | 0 | 0 | 1 | 0 | 0 | 0 | 0 | 0 | 0 | 0 | 0 | 0 | 0 | 0 |
| maeA | 0 | 0 | 1 | 0 | 0 | 1 | 0 | 0 | 0 | 0 | 0 | 0 | 0 | 0 | 0 | 0 | 0 |
| osmC | 0 | 0 | 1 | 0 | 0 | 1 | 0 | 0 | 0 | 0 | 0 | 0 | 0 | 0 | 0 | 0 | 0 |
| pqqL | 0 | 0 | 1 | 0 | 0 | 1 | 0 | 0 | 0 | 0 | 0 | 0 | 0 | 0 | 0 | 0 | 0 |
| ydeM | 0 | 0 | 1 | 0 | 0 | 1 | 0 | 0 | 0 | 0 | 0 | 0 | 0 | 0 | 0 | 0 | 0 |
| ydeO | 0 | 0 | 1 | 0 | 0 | 1 | 0 | 0 | 0 | 0 | 0 | 0 | 0 | 0 | 0 | 0 | 0 |
| hipA | 0 | 0 | 1 | 0 | 0 | 1 | 0 | 0 | 0 | 0 | 0 | 0 | 0 | 0 | 0 | 0 | 0 |
| hipB | 0 | 0 | 1 | 0 | 0 | 1 | 0 | 0 | 0 | 0 | 0 | 0 | 0 | 0 | 0 | 0 | 0 |
| lsrK | 0 | 0 | 1 | 0 | 0 | 1 | 0 | 0 | 0 | 0 | 0 | 0 | 0 | 0 | 0 | 0 | 0 |
| lsrR | 0 | 0 | 1 | 0 | 0 | 1 | 0 | 0 | 0 | 0 | 0 | 0 | 0 | 0 | 0 | 0 | 0 |
| lsrF | 0 | 0 | 1 | 0 | 0 | 1 | 0 | 0 | 0 | 0 | 0 | 0 | 0 | 0 | 0 | 0 | 0 |
| lsrG | 0 | 0 | 1 | 0 | 0 | 1 | 0 | 0 | 0 | 0 | 0 | 0 | 0 | 0 | 0 | 0 | 0 |
| tam | 0 | 0 | 1 | 0 | 0 | 1 | 0 | 0 | 0 | 0 | 0 | 0 | 0 | 0 | 0 | 0 | 0 |
| uxaB | 0 | 0 | 1 | 0 | 0 | 1 | 0 | 0 | 0 | 0 | 0 | 0 | 0 | 0 | 0 | 0 | 0 |
| sad | 0 | 0 | 1 | 0 | 0 | 1 | 0 | 0 | 0 | 0 | 0 | 0 | 0 | 0 | 0 | 0 | 0 |
| yneJ | 0 | 0 | 1 | 0 | 0 | 1 | 0 | 0 | 0 | 0 | 0 | 0 | 0 | 0 | 0 | 0 | 0 |
| marR | 0 | 0 | 1 | 0 | 0 | 1 | 0 | 0 | 0 | 0 | 0 | 0 | 0 | 0 | 0 | 0 | 0 |
| marA | 0 | 0 | 1 | 0 | 0 | 1 | 0 | 0 | 0 | 0 | 0 | 0 | 0 | 0 | 0 | 0 | 0 |
| ydeJ | 0 | 0 | 1 | 0 | 0 | 1 | 0 | 0 | 0 | 0 | 0 | 0 | 0 | 0 | 0 | 0 | 0 |
| ydfG | 0 | 0 | 1 | 0 | 0 | 1 | 0 | 0 | 0 | 0 | 0 | 0 | 0 | 0 | 0 | 0 | 0 |
| ydfH | 0 | 0 | 1 | 0 | 0 | 1 | 0 | 0 | 0 | 0 | 0 | 0 | 0 | 0 | 0 | 0 | 0 |
| ydfZ | 0 | 0 | 1 | 0 | 0 | 1 | 0 | 0 | 0 | 0 | 0 | 0 | 0 | 0 | 0 | 0 | 0 |
| ydfI | 0 | 0 | 1 | 0 | 0 | 1 | 0 | 0 | 0 | 0 | 0 | 0 | 0 | 0 | 0 | 0 | 0 |
| pinQ | 0 | 0 | 1 | 0 | 0 | 1 | 0 | 0 | 0 | 0 | 0 | 0 | 0 | 0 | 0 | 0 | 0 |
| tfaQ | 0 | 0 | 1 | 0 | 0 | 1 | 0 | 0 | 0 | 0 | 0 | 0 | 0 | 0 | 0 | 0 | 0 |
| nohQ | 0 | 0 | 1 | 0 | 0 | 1 | 0 | 0 | 0 | 0 | 0 | 0 | 0 | 0 | 0 | 0 | 0 |
| gnsB | 0 | 0 | 1 | 0 | 0 | 1 | 0 | 0 | 0 | 0 | 0 | 0 | 0 | 0 | 0 | 0 | 0 |
| ynfN | 0 | 0 | 1 | 0 | 0 | 1 | 0 | 0 | 0 | 0 | 0 | 0 | 0 | 0 | 0 | 0 | 0 |
| cspI | 0 | 0 | 1 | 0 | 0 | 1 | 0 | 0 | 0 | 0 | 0 | 0 | 0 | 0 | 0 | 0 | 0 |
| rzpQ | 0 | 0 | 1 | 0 | 0 | 1 | 0 | 0 | 0 | 0 | 0 | 0 | 0 | 0 | 0 | 0 | 0 |
| ydfR | 0 | 0 | 1 | 0 | 0 | 1 | 0 | 0 | 0 | 0 | 0 | 0 | 0 | 0 | 0 | 0 | 0 |
| cspB | 0 | 0 | 1 | 0 | 0 | 1 | 0 | 0 | 0 | 0 | 0 | 0 | 0 | 0 | 0 | 0 | 0 |
| cspF | 0 | 0 | 1 | 0 | 0 | 1 | 0 | 0 | 0 | 0 | 0 | 0 | 0 | 0 | 0 | 0 | 0 |
| quuQ | 0 | 0 | 1 | 0 | 0 | 1 | 0 | 0 | 0 | 0 | 0 | 0 | 0 | 0 | 0 | 0 | 0 |
| ydfU | 0 | 0 | 1 | 0 | 0 | 1 | 0 | 0 | 0 | 0 | 0 | 0 | 0 | 0 | 0 | 0 | 0 |
| rem | 0 | 0 | 1 | 0 | 0 | 1 | 0 | 0 | 0 | 0 | 0 | 0 | 0 | 0 | 0 | 0 | 0 |
| relE | 0 | 0 | 1 | 0 | 0 | 1 | 0 | 0 | 0 | 0 | 0 | 0 | 0 | 0 | 0 | 0 | 0 |
| ykfI | 0 | 0 | 1 | 0 | 0 | 1 | 0 | 0 | 0 | 0 | 0 | 0 | 0 | 0 | 0 | 0 | 0 |
| yafW | 0 | 0 | 1 | 0 | 0 | 1 | 0 | 0 | 0 | 0 | 0 | 0 | 0 | 0 | 0 | 0 | 0 |
| ykfG | 0 | 0 | 1 | 0 | 0 | 1 | 0 | 0 | 0 | 0 | 0 | 0 | 0 | 0 | 0 | 0 | 0 |
| yafX | 0 | 0 | 1 | 0 | 0 | 1 | 0 | 0 | 0 | 0 | 0 | 0 | 0 | 0 | 0 | 0 | 0 |
| yafZ | 0 | 0 | 1 | 0 | 0 | 1 | 0 | 0 | 0 | 0 | 0 | 0 | 0 | 0 | 0 | 0 | 0 |
| ykfA | 0 | 0 | 1 | 0 | 0 | 1 | 0 | 0 | 0 | 0 | 0 | 0 | 0 | 0 | 0 | 0 | 0 |
| perR | 0 | 0 | 1 | 0 | 0 | 1 | 0 | 0 | 0 | 0 | 0 | 0 | 0 | 0 | 0 | 0 | 0 |
| ykfC | 0 | 0 | 1 | 0 | 0 | 1 | 0 | 0 | 0 | 0 | 0 | 0 | 0 | 0 | 0 | 0 | 0 |
| mmuM | 0 | 0 | 1 | 0 | 0 | 1 | 0 | 0 | 0 | 0 | 0 | 0 | 0 | 0 | 0 | 0 | 0 |
| yagB | 0 | 0 | 1 | 0 | 0 | 1 | 0 | 0 | 0 | 0 | 0 | 0 | 0 | 0 | 0 | 0 | 0 |
| yagA | 0 | 0 | 1 | 0 | 0 | 1 | 0 | 0 | 0 | 0 | 0 | 0 | 0 | 0 | 0 | 0 | 0 |
| yagE | 0 | 0 | 1 | 0 | 0 | 1 | 0 | 0 | 0 | 0 | 0 | 0 | 0 | 0 | 0 | 0 | 0 |
| yagF | 0 | 0 | 1 | 0 | 0 | 1 | 0 | 0 | 0 | 0 | 0 | 0 | 0 | 0 | 0 | 0 | 0 |
| yagI | 0 | 0 | 1 | 0 | 0 | 1 | 0 | 0 | 0 | 0 | 0 | 0 | 0 | 0 | 0 | 0 | 0 |
| argF | 0 | 0 | 1 | 0 | 0 | 1 | 0 | 0 | 0 | 0 | 0 | 0 | 0 | 0 | 0 | 0 | 0 |
| yagK | 0 | 0 | 1 | 0 | 0 | 1 | 0 | 0 | 0 | 0 | 0 | 0 | 0 | 0 | 0 | 0 | 0 |
| yagL | 0 | 0 | 1 | 0 | 0 | 1 | 0 | 0 | 0 | 0 | 0 | 0 | 0 | 0 | 0 | 0 | 0 |
| yagM | 0 | 0 | 1 | 0 | 0 | 1 | 0 | 0 | 0 | 0 | 0 | 0 | 0 | 0 | 0 | 0 | 0 |
| yagP | 0 | 0 | 1 | 0 | 0 | 1 | 0 | 0 | 0 | 0 | 0 | 0 | 0 | 0 | 0 | 0 | 0 |
| paoD | 0 | 0 | 1 | 0 | 0 | 1 | 0 | 0 | 0 | 0 | 0 | 0 | 0 | 0 | 0 | 0 | 0 |
| ecpR | 0 | 0 | 1 | 0 | 0 | 1 | 0 | 0 | 0 | 0 | 0 | 0 | 0 | 0 | 0 | 0 | 0 |
| ykgC | 0 | 0 | 1 | 0 | 0 | 1 | 0 | 0 | 0 | 0 | 0 | 0 | 0 | 0 | 0 | 0 | 0 |
| ykgD | 0 | 0 | 1 | 0 | 0 | 1 | 0 | 0 | 0 | 0 | 0 | 0 | 0 | 0 | 0 | 0 | 0 |
| ykgE | 0 | 0 | 1 | 0 | 0 | 1 | 0 | 0 | 0 | 0 | 0 | 0 | 0 | 0 | 0 | 0 | 0 |
| ykgF | 0 | 0 | 1 | 0 | 0 | 1 | 0 | 0 | 0 | 0 | 0 | 0 | 0 | 0 | 0 | 0 | 0 |
| ykgG | 0 | 0 | 1 | 0 | 0 | 1 | 0 | 0 | 0 | 0 | 0 | 0 | 0 | 0 | 0 | 0 | 0 |
| betB | 0 | 0 | 1 | 0 | 0 | 1 | 0 | 0 | 0 | 0 | 0 | 0 | 0 | 0 | 0 | 0 | 0 |
| betI | 0 | 0 | 1 | 0 | 0 | 1 | 0 | 0 | 0 | 0 | 0 | 0 | 0 | 0 | 0 | 0 | 0 |
| yahB | 0 | 0 | 1 | 0 | 0 | 1 | 0 | 0 | 0 | 0 | 0 | 0 | 0 | 0 | 0 | 0 | 0 |
| yahD | 0 | 0 | 1 | 0 | 0 | 1 | 0 | 0 | 0 | 0 | 0 | 0 | 0 | 0 | 0 | 0 | 0 |
| yahE | 0 | 0 | 1 | 0 | 0 | 1 | 0 | 0 | 0 | 0 | 0 | 0 | 0 | 0 | 0 | 0 | 0 |
| yahF | 0 | 0 | 1 | 0 | 0 | 1 | 0 | 0 | 0 | 0 | 0 | 0 | 0 | 0 | 0 | 0 | 0 |
| yahI | 0 | 0 | 1 | 0 | 0 | 1 | 0 | 0 | 0 | 0 | 0 | 0 | 0 | 0 | 0 | 0 | 0 |
| prpR | 0 | 0 | 1 | 0 | 0 | 1 | 0 | 0 | 0 | 0 | 0 | 0 | 0 | 0 | 0 | 0 | 0 |
| prpB | 0 | 0 | 1 | 0 | 0 | 1 | 0 | 0 | 0 | 0 | 0 | 0 | 0 | 0 | 0 | 0 | 0 |
| prpC | 0 | 0 | 1 | 0 | 0 | 1 | 0 | 0 | 0 | 0 | 0 | 0 | 0 | 0 | 0 | 0 | 0 |
| prpD | 0 | 0 | 1 | 0 | 0 | 1 | 0 | 0 | 0 | 0 | 0 | 0 | 0 | 0 | 0 | 0 | 0 |
| prpE | 0 | 0 | 1 | 0 | 0 | 1 | 0 | 0 | 0 | 0 | 0 | 0 | 0 | 0 | 0 | 0 | 0 |
| codA | 0 | 0 | 1 | 0 | 0 | 1 | 0 | 0 | 0 | 0 | 0 | 0 | 0 | 0 | 0 | 0 | 0 |
| cynR | 0 | 0 | 1 | 0 | 0 | 1 | 0 | 0 | 0 | 0 | 0 | 0 | 0 | 0 | 0 | 0 | 0 |
| cynT | 0 | 0 | 1 | 0 | 0 | 1 | 0 | 0 | 0 | 0 | 0 | 0 | 0 | 0 | 0 | 0 | 0 |
| cynS | 0 | 0 | 1 | 0 | 0 | 1 | 0 | 0 | 0 | 0 | 0 | 0 | 0 | 0 | 0 | 0 | 0 |
| lacA | 0 | 0 | 1 | 0 | 0 | 1 | 0 | 0 | 0 | 0 | 0 | 0 | 0 | 0 | 0 | 0 | 0 |
| lacZ | 0 | 0 | 1 | 0 | 0 | 1 | 0 | 0 | 0 | 0 | 0 | 0 | 0 | 0 | 0 | 0 | 0 |
| lacI | 0 | 0 | 1 | 0 | 0 | 1 | 0 | 0 | 0 | 0 | 0 | 0 | 0 | 0 | 0 | 0 | 0 |
| mhpR | 0 | 0 | 1 | 0 | 0 | 1 | 0 | 0 | 0 | 0 | 0 | 0 | 0 | 0 | 0 | 0 | 0 |
| mhpA | 0 | 0 | 1 | 0 | 0 | 1 | 0 | 0 | 0 | 0 | 0 | 0 | 0 | 0 | 0 | 0 | 0 |
| mhpC | 0 | 0 | 1 | 0 | 0 | 1 | 0 | 0 | 0 | 0 | 0 | 0 | 0 | 0 | 0 | 0 | 0 |
| mhpD | 0 | 0 | 1 | 0 | 0 | 1 | 0 | 0 | 0 | 0 | 0 | 0 | 0 | 0 | 0 | 0 | 0 |
| mhpF | 0 | 0 | 1 | 0 | 0 | 1 | 0 | 0 | 0 | 0 | 0 | 0 | 0 | 0 | 0 | 0 | 0 |
| mhpE | 0 | 0 | 1 | 0 | 0 | 1 | 0 | 0 | 0 | 0 | 0 | 0 | 0 | 0 | 0 | 0 | 0 |
| yaiL | 0 | 0 | 1 | 0 | 0 | 1 | 0 | 0 | 0 | 0 | 0 | 0 | 0 | 0 | 0 | 0 | 0 |
| frmB | 0 | 0 | 1 | 0 | 0 | 1 | 0 | 0 | 0 | 0 | 0 | 0 | 0 | 0 | 0 | 0 | 0 |
| frmA | 0 | 0 | 1 | 0 | 0 | 1 | 0 | 0 | 0 | 0 | 0 | 0 | 0 | 0 | 0 | 0 | 0 |
| galM | 0 | 0 | 1 | 0 | 0 | 1 | 0 | 0 | 0 | 0 | 0 | 0 | 0 | 0 | 0 | 0 | 0 |
| galK | 0 | 0 | 1 | 0 | 0 | 1 | 0 | 0 | 0 | 0 | 0 | 0 | 0 | 0 | 0 | 0 | 0 |
| galT | 0 | 0 | 1 | 0 | 0 | 1 | 0 | 0 | 0 | 0 | 0 | 0 | 0 | 0 | 0 | 0 | 0 |
| galE | 0 | 0 | 1 | 0 | 0 | 1 | 0 | 0 | 0 | 0 | 0 | 0 | 0 | 0 | 0 | 0 | 0 |
| modE | 0 | 0 | 1 | 0 | 0 | 1 | 0 | 0 | 0 | 0 | 0 | 0 | 0 | 0 | 0 | 0 | 0 |
| ybhA | 0 | 0 | 1 | 0 | 0 | 1 | 0 | 0 | 0 | 0 | 0 | 0 | 0 | 0 | 0 | 0 | 0 |
| pgl | 0 | 0 | 1 | 0 | 0 | 1 | 0 | 0 | 0 | 0 | 0 | 0 | 0 | 0 | 0 | 0 | 0 |
| ybhD | 0 | 0 | 1 | 0 | 0 | 1 | 0 | 0 | 0 | 0 | 0 | 0 | 0 | 0 | 0 | 0 | 0 |
| ybhJ | 0 | 0 | 1 | 0 | 0 | 1 | 0 | 0 | 0 | 0 | 0 | 0 | 0 | 0 | 0 | 0 | 0 |
| ybhB | 0 | 0 | 1 | 0 | 0 | 1 | 0 | 0 | 0 | 0 | 0 | 0 | 0 | 0 | 0 | 0 | 0 |
| bioA | 0 | 0 | 1 | 0 | 0 | 1 | 0 | 0 | 0 | 0 | 0 | 0 | 0 | 0 | 0 | 0 | 0 |
| bioF | 0 | 0 | 1 | 0 | 0 | 1 | 0 | 0 | 0 | 0 | 0 | 0 | 0 | 0 | 0 | 0 | 0 |
| bioC | 0 | 0 | 1 | 0 | 0 | 1 | 0 | 0 | 0 | 0 | 0 | 0 | 0 | 0 | 0 | 0 | 0 |
| bioD | 0 | 0 | 1 | 0 | 0 | 1 | 0 | 0 | 0 | 0 | 0 | 0 | 0 | 0 | 0 | 0 | 0 |
| uvrB | 0 | 0 | 1 | 0 | 0 | 1 | 0 | 0 | 0 | 0 | 0 | 0 | 0 | 0 | 0 | 0 | 0 |
| ybhK | 0 | 0 | 1 | 0 | 0 | 1 | 0 | 0 | 0 | 0 | 0 | 0 | 0 | 0 | 0 | 0 | 0 |
| moaA | 0 | 0 | 1 | 0 | 0 | 1 | 0 | 0 | 0 | 0 | 0 | 0 | 0 | 0 | 0 | 0 | 0 |
| moaB | 0 | 0 | 1 | 0 | 0 | 1 | 0 | 0 | 0 | 0 | 0 | 0 | 0 | 0 | 0 | 0 | 0 |
| moaC | 0 | 0 | 1 | 0 | 0 | 1 | 0 | 0 | 0 | 0 | 0 | 0 | 0 | 0 | 0 | 0 | 0 |
| moaD | 0 | 0 | 1 | 0 | 0 | 1 | 0 | 0 | 0 | 0 | 0 | 0 | 0 | 0 | 0 | 0 | 0 |
| moaE | 0 | 0 | 1 | 0 | 0 | 1 | 0 | 0 | 0 | 0 | 0 | 0 | 0 | 0 | 0 | 0 | 0 |
| ybhP | 0 | 0 | 1 | 0 | 0 | 1 | 0 | 0 | 0 | 0 | 0 | 0 | 0 | 0 | 0 | 0 | 0 |
| ybiH | 0 | 0 | 1 | 0 | 0 | 1 | 0 | 0 | 0 | 0 | 0 | 0 | 0 | 0 | 0 | 0 | 0 |
| rhlE | 0 | 0 | 1 | 0 | 0 | 1 | 0 | 0 | 0 | 0 | 0 | 0 | 0 | 0 | 0 | 0 | 0 |
| dinG | 0 | 0 | 1 | 0 | 0 | 1 | 0 | 0 | 0 | 0 | 0 | 0 | 0 | 0 | 0 | 0 | 0 |
| ybiB | 0 | 0 | 1 | 0 | 0 | 1 | 0 | 0 | 0 | 0 | 0 | 0 | 0 | 0 | 0 | 0 | 0 |
| ybiC | 0 | 0 | 1 | 0 | 0 | 1 | 0 | 0 | 0 | 0 | 0 | 0 | 0 | 0 | 0 | 0 | 0 |
| ybiI | 0 | 0 | 1 | 0 | 0 | 1 | 0 | 0 | 0 | 0 | 0 | 0 | 0 | 0 | 0 | 0 | 0 |
| ybiX | 0 | 0 | 1 | 0 | 0 | 1 | 0 | 0 | 0 | 0 | 0 | 0 | 0 | 0 | 0 | 0 | 0 |
| rlmF | 0 | 0 | 1 | 0 | 0 | 1 | 0 | 0 | 0 | 0 | 0 | 0 | 0 | 0 | 0 | 0 | 0 |
| mntR | 0 | 0 | 1 | 0 | 0 | 1 | 0 | 0 | 0 | 0 | 0 | 0 | 0 | 0 | 0 | 0 | 0 |
| ybiU | 0 | 0 | 1 | 0 | 0 | 1 | 0 | 0 | 0 | 0 | 0 | 0 | 0 | 0 | 0 | 0 | 0 |
| ybiV | 0 | 0 | 1 | 0 | 0 | 1 | 0 | 0 | 0 | 0 | 0 | 0 | 0 | 0 | 0 | 0 | 0 |
| ybiW | 0 | 0 | 1 | 0 | 0 | 1 | 0 | 0 | 0 | 0 | 0 | 0 | 0 | 0 | 0 | 0 | 0 |
| ybiY | 0 | 0 | 1 | 0 | 0 | 1 | 0 | 0 | 0 | 0 | 0 | 0 | 0 | 0 | 0 | 0 | 0 |
| fsaA | 0 | 0 | 1 | 0 | 0 | 1 | 0 | 0 | 0 | 0 | 0 | 0 | 0 | 0 | 0 | 0 | 0 |
| moeB | 0 | 0 | 1 | 0 | 0 | 1 | 0 | 0 | 0 | 0 | 0 | 0 | 0 | 0 | 0 | 0 | 0 |
| moeA | 0 | 0 | 1 | 0 | 0 | 1 | 0 | 0 | 0 | 0 | 0 | 0 | 0 | 0 | 0 | 0 | 0 |
| rimO | 0 | 0 | 1 | 0 | 0 | 1 | 0 | 0 | 0 | 0 | 0 | 0 | 0 | 0 | 0 | 0 | 0 |
| bssR | 0 | 0 | 1 | 0 | 0 | 1 | 0 | 0 | 0 | 0 | 0 | 0 | 0 | 0 | 0 | 0 | 0 |
| gstB | 0 | 0 | 1 | 0 | 0 | 1 | 0 | 0 | 0 | 0 | 0 | 0 | 0 | 0 | 0 | 0 | 0 |
| deoR | 0 | 0 | 1 | 0 | 0 | 1 | 0 | 0 | 0 | 0 | 0 | 0 | 0 | 0 | 0 | 0 | 0 |
| ybjI | 0 | 0 | 1 | 0 | 0 | 1 | 0 | 0 | 0 | 0 | 0 | 0 | 0 | 0 | 0 | 0 | 0 |
| ybjK | 0 | 0 | 1 | 0 | 0 | 1 | 0 | 0 | 0 | 0 | 0 | 0 | 0 | 0 | 0 | 0 | 0 |
| grxA | 0 | 0 | 1 | 0 | 0 | 1 | 0 | 0 | 0 | 0 | 0 | 0 | 0 | 0 | 0 | 0 | 0 |
| nfsA | 0 | 0 | 1 | 0 | 0 | 1 | 0 | 0 | 0 | 0 | 0 | 0 | 0 | 0 | 0 | 0 | 0 |
| rimK | 0 | 0 | 1 | 0 | 0 | 1 | 0 | 0 | 0 | 0 | 0 | 0 | 0 | 0 | 0 | 0 | 0 |
| ybjN | 0 | 0 | 1 | 0 | 0 | 1 | 0 | 0 | 0 | 0 | 0 | 0 | 0 | 0 | 0 | 0 | 0 |
| rlmC | 0 | 0 | 1 | 0 | 0 | 1 | 0 | 0 | 0 | 0 | 0 | 0 | 0 | 0 | 0 | 0 | 0 |
| ybjQ | 0 | 0 | 1 | 0 | 0 | 1 | 0 | 0 | 0 | 0 | 0 | 0 | 0 | 0 | 0 | 0 | 0 |
| ybjS | 0 | 0 | 1 | 0 | 0 | 1 | 0 | 0 | 0 | 0 | 0 | 0 | 0 | 0 | 0 | 0 | 0 |
| ltaE | 0 | 0 | 1 | 0 | 0 | 1 | 0 | 0 | 0 | 0 | 0 | 0 | 0 | 0 | 0 | 0 | 0 |
| hcp | 0 | 0 | 1 | 0 | 0 | 1 | 0 | 0 | 0 | 0 | 0 | 0 | 0 | 0 | 0 | 0 | 0 |
| ybjD | 0 | 0 | 1 | 0 | 0 | 1 | 0 | 0 | 0 | 0 | 0 | 0 | 0 | 0 | 0 | 0 | 0 |
| ybjX | 0 | 0 | 1 | 0 | 0 | 1 | 0 | 0 | 0 | 0 | 0 | 0 | 0 | 0 | 0 | 0 | 0 |
| hycA | 0 | 0 | 1 | 0 | 0 | 1 | 0 | 0 | 0 | 0 | 0 | 0 | 0 | 0 | 0 | 0 | 0 |
| hypB | 0 | 0 | 1 | 0 | 0 | 1 | 0 | 0 | 0 | 0 | 0 | 0 | 0 | 0 | 0 | 0 | 0 |
| hypC | 0 | 0 | 1 | 0 | 0 | 1 | 0 | 0 | 0 | 0 | 0 | 0 | 0 | 0 | 0 | 0 | 0 |
| hypD | 0 | 0 | 1 | 0 | 0 | 1 | 0 | 0 | 0 | 0 | 0 | 0 | 0 | 0 | 0 | 0 | 0 |
| cbpA | 0 | 1 | 1 | 1 | 0 | 1 | 0 | 0 | 0 | 0 | 0 | 0 | 0 | 0 | 0 | 0 | 0 |
| ddpX | 0 | 0 | 1 | 1 | 0 | 1 | 0 | 0 | 0 | 0 | 0 | 0 | 0 | 0 | 0 | 0 | 0 |
| csgD | 0 | 1 | 1 | 0 | 0 | 1 | 1 | 0 | 0 | 0 | 0 | 0 | 0 | 0 | 0 | 0 | 0 |
| ccmE | 0 | 0 | 1 | 0 | 0 | 1 | 1 | 0 | 0 | 0 | 0 | 0 | 0 | 0 | 0 | 0 | 0 |
| ccmA | 0 | 0 | 1 | 0 | 0 | 1 | 1 | 0 | 0 | 0 | 0 | 0 | 0 | 0 | 0 | 0 | 0 |
| mqo | 0 | 0 | 1 | 0 | 0 | 1 | 1 | 0 | 0 | 0 | 0 | 0 | 0 | 0 | 0 | 0 | 0 |
| glpA | 0 | 0 | 1 | 0 | 0 | 1 | 1 | 0 | 0 | 0 | 0 | 0 | 0 | 0 | 0 | 0 | 0 |
| glpB | 0 | 0 | 1 | 0 | 0 | 1 | 1 | 0 | 0 | 0 | 0 | 0 | 0 | 0 | 0 | 0 | 0 |
| glpC | 0 | 0 | 1 | 0 | 0 | 1 | 1 | 0 | 0 | 0 | 0 | 0 | 0 | 0 | 0 | 0 | 0 |
| pspC | 0 | 0 | 1 | 0 | 0 | 1 | 1 | 0 | 0 | 0 | 0 | 0 | 0 | 0 | 0 | 0 | 0 |
| pspD | 0 | 0 | 1 | 0 | 0 | 1 | 1 | 0 | 0 | 0 | 0 | 0 | 0 | 0 | 0 | 0 | 0 |
| narY | 0 | 0 | 1 | 0 | 0 | 1 | 1 | 0 | 0 | 0 | 0 | 0 | 0 | 0 | 0 | 0 | 0 |
| betA | 0 | 0 | 1 | 0 | 0 | 1 | 1 | 0 | 0 | 0 | 0 | 0 | 0 | 0 | 0 | 0 | 0 |
| tauB | 0 | 0 | 1 | 0 | 0 | 1 | 1 | 0 | 0 | 0 | 0 | 0 | 0 | 0 | 0 | 0 | 0 |
| clsB | 0 | 0 | 1 | 0 | 0 | 1 | 1 | 0 | 0 | 0 | 0 | 0 | 0 | 0 | 0 | 0 | 0 |
| potG | 0 | 0 | 1 | 0 | 0 | 1 | 1 | 0 | 0 | 0 | 0 | 0 | 0 | 0 | 0 | 0 | 0 |
| gutQ | 0 | 0 | 1 | 0 | 0 | 1 | 0 | 0 | 0 | 0 | 0 | 1 | 0 | 0 | 0 | 0 | 0 |
| gadB | 0 | 0 | 1 | 0 | 0 | 1 | 0 | 0 | 0 | 0 | 0 | 1 | 0 | 0 | 0 | 0 | 0 |
| acrZ | 0 | 0 | 1 | 0 | 0 | 1 | 1 | 1 | 0 | 0 | 0 | 0 | 0 | 0 | 0 | 0 | 0 |
| dcp | 0 | 0 | 1 | 0 | 0 | 1 | 0 | 0 | 1 | 0 | 0 | 0 | 0 | 0 | 0 | 0 | 0 |
| ykgI | 0 | 0 | 1 | 0 | 0 | 1 | 0 | 0 | 1 | 0 | 0 | 0 | 0 | 0 | 0 | 0 | 0 |
| iaaA | 0 | 0 | 1 | 0 | 0 | 1 | 0 | 0 | 1 | 0 | 0 | 0 | 0 | 0 | 0 | 0 | 0 |
| sra | 0 | 0 | 1 | 0 | 0 | 1 | 0 | 0 | 0 | 1 | 0 | 0 | 0 | 0 | 0 | 0 | 0 |
| ykgM | 0 | 0 | 1 | 0 | 0 | 1 | 0 | 0 | 0 | 1 | 0 | 0 | 0 | 0 | 0 | 0 | 0 |
| intB | 1 | 1 | 1 | 0 | 0 | 1 | 0 | 0 | 0 | 0 | 0 | 0 | 0 | 0 | 0 | 0 | 0 |
| yjjA | 1 | 1 | 1 | 0 | 0 | 1 | 0 | 0 | 0 | 0 | 0 | 0 | 0 | 0 | 0 | 0 | 0 |
| yehD | 0 | 1 | 1 | 0 | 0 | 0 | 0 | 0 | 0 | 0 | 0 | 0 | 1 | 1 | 0 | 0 | 0 |
| stfR | 0 | 0 | 1 | 0 | 0 | 0 | 0 | 0 | 0 | 0 | 0 | 0 | 0 | 1 | 0 | 0 | 0 |
| yddK | 0 | 0 | 1 | 0 | 0 | 0 | 0 | 0 | 0 | 0 | 0 | 0 | 0 | 1 | 0 | 0 | 0 |
| yhjY | 0 | 1 | 1 | 0 | 0 | 0 | 0 | 0 | 0 | 0 | 0 | 1 | 0 | 1 | 0 | 0 | 0 |
| ypjA | 0 | 1 | 1 | 0 | 0 | 0 | 0 | 1 | 0 | 0 | 0 | 0 | 0 | 1 | 0 | 0 | 0 |
| ybiS | 0 | 0 | 1 | 0 | 0 | 0 | 0 | 0 | 1 | 0 | 0 | 0 | 0 | 1 | 0 | 0 | 0 |
| fimI | 1 | 1 | 1 | 0 | 0 | 0 | 0 | 0 | 0 | 0 | 0 | 0 | 1 | 1 | 0 | 0 | 0 |
| yqjI | 0 | 1 | 1 | 0 | 0 | 0 | 0 | 0 | 0 | 0 | 0 | 0 | 1 | 1 | 0 | 0 | 0 |
| ydeQ | 0 | 0 | 1 | 0 | 0 | 0 | 0 | 0 | 0 | 0 | 0 | 0 | 1 | 1 | 0 | 0 | 0 |
| ydeR | 0 | 0 | 1 | 0 | 0 | 0 | 0 | 0 | 0 | 0 | 0 | 0 | 1 | 1 | 0 | 0 | 0 |
| ydeS | 0 | 0 | 1 | 0 | 0 | 0 | 0 | 0 | 0 | 0 | 0 | 0 | 1 | 1 | 0 | 0 | 0 |
| yjhB | 1 | 1 | 1 | 0 | 0 | 0 | 1 | 0 | 0 | 0 | 0 | 0 | 0 | 0 | 0 | 0 | 0 |
| fecD | 1 | 1 | 1 | 0 | 0 | 0 | 1 | 0 | 0 | 0 | 0 | 0 | 0 | 0 | 0 | 0 | 0 |
| fecC | 1 | 1 | 1 | 0 | 0 | 0 | 1 | 0 | 0 | 0 | 0 | 0 | 0 | 0 | 0 | 0 | 0 |
| yjhF | 1 | 1 | 1 | 0 | 0 | 0 | 1 | 0 | 0 | 0 | 0 | 0 | 0 | 0 | 0 | 0 | 0 |
| sgcC | 1 | 1 | 1 | 0 | 0 | 0 | 1 | 0 | 0 | 0 | 0 | 0 | 0 | 0 | 0 | 0 | 0 |
| nrfC | 0 | 1 | 1 | 0 | 0 | 0 | 1 | 0 | 0 | 0 | 0 | 0 | 0 | 0 | 0 | 0 | 0 |
| nrfD | 0 | 1 | 1 | 0 | 0 | 0 | 1 | 0 | 0 | 0 | 0 | 0 | 0 | 0 | 0 | 0 | 0 |
| phnM | 0 | 1 | 1 | 0 | 0 | 0 | 1 | 0 | 0 | 0 | 0 | 0 | 0 | 0 | 0 | 0 | 0 |
| pmrR | 0 | 1 | 1 | 0 | 0 | 0 | 1 | 0 | 0 | 0 | 0 | 0 | 0 | 0 | 0 | 0 | 0 |
| yhiI | 0 | 1 | 1 | 0 | 0 | 0 | 1 | 0 | 0 | 0 | 0 | 0 | 0 | 0 | 0 | 0 | 0 |
| mdtE | 0 | 1 | 1 | 0 | 0 | 0 | 1 | 0 | 0 | 0 | 0 | 0 | 0 | 0 | 0 | 0 | 0 |
| bcsA | 0 | 1 | 1 | 0 | 0 | 0 | 1 | 0 | 0 | 0 | 0 | 0 | 0 | 0 | 0 | 0 | 0 |
| bcsF | 0 | 1 | 1 | 0 | 0 | 0 | 1 | 0 | 0 | 0 | 0 | 0 | 0 | 0 | 0 | 0 | 0 |
| bcsG | 0 | 1 | 1 | 0 | 0 | 0 | 1 | 0 | 0 | 0 | 0 | 0 | 0 | 0 | 0 | 0 | 0 |
| ldrD | 0 | 1 | 1 | 0 | 0 | 0 | 1 | 0 | 0 | 0 | 0 | 0 | 0 | 0 | 0 | 0 | 0 |
| yohD | 0 | 1 | 1 | 0 | 0 | 0 | 1 | 0 | 0 | 0 | 0 | 0 | 0 | 0 | 0 | 0 | 0 |
| yohP | 0 | 1 | 1 | 0 | 0 | 0 | 1 | 0 | 0 | 0 | 0 | 0 | 0 | 0 | 0 | 0 | 0 |
| yohJ | 0 | 1 | 1 | 0 | 0 | 0 | 1 | 0 | 0 | 0 | 0 | 0 | 0 | 0 | 0 | 0 | 0 |
| yohK | 0 | 1 | 1 | 0 | 0 | 0 | 1 | 0 | 0 | 0 | 0 | 0 | 0 | 0 | 0 | 0 | 0 |
| yeiS | 0 | 1 | 1 | 0 | 0 | 0 | 1 | 0 | 0 | 0 | 0 | 0 | 0 | 0 | 0 | 0 | 0 |
| mglC | 0 | 1 | 1 | 0 | 0 | 0 | 1 | 0 | 0 | 0 | 0 | 0 | 0 | 0 | 0 | 0 | 0 |
| yeiB | 0 | 1 | 1 | 0 | 0 | 0 | 1 | 0 | 0 | 0 | 0 | 0 | 0 | 0 | 0 | 0 | 0 |
| plaP | 0 | 1 | 1 | 0 | 0 | 0 | 1 | 0 | 0 | 0 | 0 | 0 | 0 | 0 | 0 | 0 | 0 |
| wzzB | 0 | 1 | 1 | 0 | 0 | 0 | 1 | 0 | 0 | 0 | 0 | 0 | 0 | 0 | 0 | 0 | 0 |
| wbbK | 0 | 1 | 1 | 0 | 0 | 0 | 1 | 0 | 0 | 0 | 0 | 0 | 0 | 0 | 0 | 0 | 0 |
| wbbH | 0 | 1 | 1 | 0 | 0 | 0 | 1 | 0 | 0 | 0 | 0 | 0 | 0 | 0 | 0 | 0 | 0 |
| wzxB | 0 | 1 | 1 | 0 | 0 | 0 | 1 | 0 | 0 | 0 | 0 | 0 | 0 | 0 | 0 | 0 | 0 |
| wzxC | 0 | 1 | 1 | 0 | 0 | 0 | 1 | 0 | 0 | 0 | 0 | 0 | 0 | 0 | 0 | 0 | 0 |
| wcaJ | 0 | 1 | 1 | 0 | 0 | 0 | 1 | 0 | 0 | 0 | 0 | 0 | 0 | 0 | 0 | 0 | 0 |
| wcaD | 0 | 1 | 1 | 0 | 0 | 0 | 1 | 0 | 0 | 0 | 0 | 0 | 0 | 0 | 0 | 0 | 0 |
| wzc | 0 | 1 | 1 | 0 | 0 | 0 | 1 | 0 | 0 | 0 | 0 | 0 | 0 | 0 | 0 | 0 | 0 |
| yegH | 0 | 1 | 1 | 0 | 0 | 0 | 1 | 0 | 0 | 0 | 0 | 0 | 0 | 0 | 0 | 0 | 0 |
| yegE | 0 | 1 | 1 | 0 | 0 | 0 | 1 | 0 | 0 | 0 | 0 | 0 | 0 | 0 | 0 | 0 | 0 |
| ibsA | 0 | 1 | 1 | 0 | 0 | 0 | 1 | 0 | 0 | 0 | 0 | 0 | 0 | 0 | 0 | 0 | 0 |
| mdtB | 0 | 1 | 1 | 0 | 0 | 0 | 1 | 0 | 0 | 0 | 0 | 0 | 0 | 0 | 0 | 0 | 0 |
| mdtC | 0 | 1 | 1 | 0 | 0 | 0 | 1 | 0 | 0 | 0 | 0 | 0 | 0 | 0 | 0 | 0 | 0 |
| mdtD | 0 | 1 | 1 | 0 | 0 | 0 | 1 | 0 | 0 | 0 | 0 | 0 | 0 | 0 | 0 | 0 | 0 |
| baeS | 0 | 1 | 1 | 0 | 0 | 0 | 1 | 0 | 0 | 0 | 0 | 0 | 0 | 0 | 0 | 0 | 0 |
| gatZ | 0 | 1 | 1 | 0 | 0 | 0 | 1 | 0 | 0 | 0 | 0 | 0 | 0 | 0 | 0 | 0 | 0 |
| yegT | 0 | 1 | 1 | 0 | 0 | 0 | 1 | 0 | 0 | 0 | 0 | 0 | 0 | 0 | 0 | 0 | 0 |
| rcnA | 0 | 1 | 1 | 0 | 0 | 0 | 1 | 0 | 0 | 0 | 0 | 0 | 0 | 0 | 0 | 0 | 0 |
| gfcB | 0 | 1 | 1 | 0 | 0 | 0 | 1 | 0 | 0 | 0 | 0 | 0 | 0 | 0 | 0 | 0 | 0 |
| gfcA | 0 | 1 | 1 | 0 | 0 | 0 | 1 | 0 | 0 | 0 | 0 | 0 | 0 | 0 | 0 | 0 | 0 |
| ymcE | 0 | 1 | 1 | 0 | 0 | 0 | 1 | 0 | 0 | 0 | 0 | 0 | 0 | 0 | 0 | 0 | 0 |
| yccM | 0 | 1 | 1 | 0 | 0 | 0 | 1 | 0 | 0 | 0 | 0 | 0 | 0 | 0 | 0 | 0 | 0 |
| rutG | 0 | 1 | 1 | 0 | 0 | 0 | 1 | 0 | 0 | 0 | 0 | 0 | 0 | 0 | 0 | 0 | 0 |
| putP | 0 | 1 | 1 | 0 | 0 | 0 | 1 | 0 | 0 | 0 | 0 | 0 | 0 | 0 | 0 | 0 | 0 |
| pgaD | 0 | 1 | 1 | 0 | 0 | 0 | 1 | 0 | 0 | 0 | 0 | 0 | 0 | 0 | 0 | 0 | 0 |
| pgaC | 0 | 1 | 1 | 0 | 0 | 0 | 1 | 0 | 0 | 0 | 0 | 0 | 0 | 0 | 0 | 0 | 0 |
| ycdT | 0 | 1 | 1 | 0 | 0 | 0 | 1 | 0 | 0 | 0 | 0 | 0 | 0 | 0 | 0 | 0 | 0 |
| ycdU | 0 | 1 | 1 | 0 | 0 | 0 | 1 | 0 | 0 | 0 | 0 | 0 | 0 | 0 | 0 | 0 | 0 |
| ycdZ | 0 | 1 | 1 | 0 | 0 | 0 | 1 | 0 | 0 | 0 | 0 | 0 | 0 | 0 | 0 | 0 | 0 |
| opgC | 0 | 1 | 1 | 0 | 0 | 0 | 1 | 0 | 0 | 0 | 0 | 0 | 0 | 0 | 0 | 0 | 0 |
| opgH | 0 | 1 | 1 | 0 | 0 | 0 | 1 | 0 | 0 | 0 | 0 | 0 | 0 | 0 | 0 | 0 | 0 |
| mdtG | 0 | 1 | 1 | 0 | 0 | 0 | 1 | 0 | 0 | 0 | 0 | 0 | 0 | 0 | 0 | 0 | 0 |
| yfjS | 0 | 1 | 1 | 0 | 0 | 0 | 1 | 0 | 0 | 0 | 0 | 0 | 0 | 0 | 0 | 0 | 0 |
| yfjW | 0 | 1 | 1 | 0 | 0 | 0 | 1 | 0 | 0 | 0 | 0 | 0 | 0 | 0 | 0 | 0 | 0 |
| gabP | 0 | 1 | 1 | 0 | 0 | 0 | 1 | 0 | 0 | 0 | 0 | 0 | 0 | 0 | 0 | 0 | 0 |
| ygaP | 0 | 1 | 1 | 0 | 0 | 0 | 1 | 0 | 0 | 0 | 0 | 0 | 0 | 0 | 0 | 0 | 0 |
| alaE | 0 | 1 | 1 | 0 | 0 | 0 | 1 | 0 | 0 | 0 | 0 | 0 | 0 | 0 | 0 | 0 | 0 |
| proV | 0 | 1 | 1 | 0 | 0 | 0 | 1 | 0 | 0 | 0 | 0 | 0 | 0 | 0 | 0 | 0 | 0 |
| proW | 0 | 1 | 1 | 0 | 0 | 0 | 1 | 0 | 0 | 0 | 0 | 0 | 0 | 0 | 0 | 0 | 0 |
| ygaY | 0 | 1 | 1 | 0 | 0 | 0 | 1 | 0 | 0 | 0 | 0 | 0 | 0 | 0 | 0 | 0 | 0 |
| ygaZ | 0 | 1 | 1 | 0 | 0 | 0 | 1 | 0 | 0 | 0 | 0 | 0 | 0 | 0 | 0 | 0 | 0 |
| ygaH | 0 | 1 | 1 | 0 | 0 | 0 | 1 | 0 | 0 | 0 | 0 | 0 | 0 | 0 | 0 | 0 | 0 |
| emrB | 0 | 1 | 1 | 0 | 0 | 0 | 1 | 0 | 0 | 0 | 0 | 0 | 0 | 0 | 0 | 0 | 0 |
| srlA | 0 | 1 | 1 | 0 | 0 | 0 | 1 | 0 | 0 | 0 | 0 | 0 | 0 | 0 | 0 | 0 | 0 |
| srlE | 0 | 1 | 1 | 0 | 0 | 0 | 1 | 0 | 0 | 0 | 0 | 0 | 0 | 0 | 0 | 0 | 0 |
| ascF | 0 | 1 | 1 | 0 | 0 | 0 | 1 | 0 | 0 | 0 | 0 | 0 | 0 | 0 | 0 | 0 | 0 |
| hycD | 0 | 1 | 1 | 0 | 0 | 0 | 1 | 0 | 0 | 0 | 0 | 0 | 0 | 0 | 0 | 0 | 0 |
| hycC | 0 | 1 | 1 | 0 | 0 | 0 | 1 | 0 | 0 | 0 | 0 | 0 | 0 | 0 | 0 | 0 | 0 |
| ygbN | 0 | 1 | 1 | 0 | 0 | 0 | 1 | 0 | 0 | 0 | 0 | 0 | 0 | 0 | 0 | 0 | 0 |
| ccmH | 0 | 1 | 1 | 0 | 0 | 0 | 1 | 0 | 0 | 0 | 0 | 0 | 0 | 0 | 0 | 0 | 0 |
| ccmF | 0 | 1 | 1 | 0 | 0 | 0 | 1 | 0 | 0 | 0 | 0 | 0 | 0 | 0 | 0 | 0 | 0 |
| ccmD | 0 | 1 | 1 | 0 | 0 | 0 | 1 | 0 | 0 | 0 | 0 | 0 | 0 | 0 | 0 | 0 | 0 |
| ccmC | 0 | 1 | 1 | 0 | 0 | 0 | 1 | 0 | 0 | 0 | 0 | 0 | 0 | 0 | 0 | 0 | 0 |
| ccmB | 0 | 1 | 1 | 0 | 0 | 0 | 1 | 0 | 0 | 0 | 0 | 0 | 0 | 0 | 0 | 0 | 0 |
| napC | 0 | 1 | 1 | 0 | 0 | 0 | 1 | 0 | 0 | 0 | 0 | 0 | 0 | 0 | 0 | 0 | 0 |
| napH | 0 | 1 | 1 | 0 | 0 | 0 | 1 | 0 | 0 | 0 | 0 | 0 | 0 | 0 | 0 | 0 | 0 |
| yojI | 0 | 1 | 1 | 0 | 0 | 0 | 1 | 0 | 0 | 0 | 0 | 0 | 0 | 0 | 0 | 0 | 0 |
| rcsD | 0 | 1 | 1 | 0 | 0 | 0 | 1 | 0 | 0 | 0 | 0 | 0 | 0 | 0 | 0 | 0 | 0 |
| rcsC | 0 | 1 | 1 | 0 | 0 | 0 | 1 | 0 | 0 | 0 | 0 | 0 | 0 | 0 | 0 | 0 | 0 |
| atoS | 0 | 1 | 1 | 0 | 0 | 0 | 1 | 0 | 0 | 0 | 0 | 0 | 0 | 0 | 0 | 0 | 0 |
| atoE | 0 | 1 | 1 | 0 | 0 | 0 | 1 | 0 | 0 | 0 | 0 | 0 | 0 | 0 | 0 | 0 | 0 |
| glpT | 0 | 1 | 1 | 0 | 0 | 0 | 1 | 0 | 0 | 0 | 0 | 0 | 0 | 0 | 0 | 0 | 0 |
| rhmT | 0 | 1 | 1 | 0 | 0 | 0 | 1 | 0 | 0 | 0 | 0 | 0 | 0 | 0 | 0 | 0 | 0 |
| arnC | 0 | 1 | 1 | 0 | 0 | 0 | 1 | 0 | 0 | 0 | 0 | 0 | 0 | 0 | 0 | 0 | 0 |
| arnT | 0 | 1 | 1 | 0 | 0 | 0 | 1 | 0 | 0 | 0 | 0 | 0 | 0 | 0 | 0 | 0 | 0 |
| arnE | 0 | 1 | 1 | 0 | 0 | 0 | 1 | 0 | 0 | 0 | 0 | 0 | 0 | 0 | 0 | 0 | 0 |
| arnF | 0 | 1 | 1 | 0 | 0 | 0 | 1 | 0 | 0 | 0 | 0 | 0 | 0 | 0 | 0 | 0 | 0 |
| yfbL | 0 | 1 | 1 | 0 | 0 | 0 | 1 | 0 | 0 | 0 | 0 | 0 | 0 | 0 | 0 | 0 | 0 |
| sapD | 0 | 1 | 1 | 0 | 0 | 0 | 1 | 0 | 0 | 0 | 0 | 0 | 0 | 0 | 0 | 0 | 0 |
| sapC | 0 | 1 | 1 | 0 | 0 | 0 | 1 | 0 | 0 | 0 | 0 | 0 | 0 | 0 | 0 | 0 | 0 |
| sapB | 0 | 1 | 1 | 0 | 0 | 0 | 1 | 0 | 0 | 0 | 0 | 0 | 0 | 0 | 0 | 0 | 0 |
| puuP | 0 | 1 | 1 | 0 | 0 | 0 | 1 | 0 | 0 | 0 | 0 | 0 | 0 | 0 | 0 | 0 | 0 |
| pspB | 0 | 1 | 1 | 0 | 0 | 0 | 1 | 0 | 0 | 0 | 0 | 0 | 0 | 0 | 0 | 0 | 0 |
| ycjO | 0 | 1 | 1 | 0 | 0 | 0 | 1 | 0 | 0 | 0 | 0 | 0 | 0 | 0 | 0 | 0 | 0 |
| ycjP | 0 | 1 | 1 | 0 | 0 | 0 | 1 | 0 | 0 | 0 | 0 | 0 | 0 | 0 | 0 | 0 | 0 |
| ycjF | 0 | 1 | 1 | 0 | 0 | 0 | 1 | 0 | 0 | 0 | 0 | 0 | 0 | 0 | 0 | 0 | 0 |
| ynaI | 0 | 1 | 1 | 0 | 0 | 0 | 1 | 0 | 0 | 0 | 0 | 0 | 0 | 0 | 0 | 0 | 0 |
| ynaJ | 0 | 1 | 1 | 0 | 0 | 0 | 1 | 0 | 0 | 0 | 0 | 0 | 0 | 0 | 0 | 0 | 0 |
| abgT | 0 | 1 | 1 | 0 | 0 | 0 | 1 | 0 | 0 | 0 | 0 | 0 | 0 | 0 | 0 | 0 | 0 |
| ydaM | 0 | 1 | 1 | 0 | 0 | 0 | 1 | 0 | 0 | 0 | 0 | 0 | 0 | 0 | 0 | 0 | 0 |
| ydaN | 0 | 1 | 1 | 0 | 0 | 0 | 1 | 0 | 0 | 0 | 0 | 0 | 0 | 0 | 0 | 0 | 0 |
| sieB | 0 | 1 | 1 | 0 | 0 | 0 | 1 | 0 | 0 | 0 | 0 | 0 | 0 | 0 | 0 | 0 | 0 |
| trkG | 0 | 1 | 1 | 0 | 0 | 0 | 1 | 0 | 0 | 0 | 0 | 0 | 0 | 0 | 0 | 0 | 0 |
| paaF | 0 | 1 | 1 | 0 | 0 | 0 | 1 | 0 | 0 | 0 | 0 | 0 | 0 | 0 | 0 | 0 | 0 |
| ydbC | 0 | 1 | 1 | 0 | 0 | 0 | 1 | 0 | 0 | 0 | 0 | 0 | 0 | 0 | 0 | 0 | 0 |
| ynbA | 0 | 1 | 1 | 0 | 0 | 0 | 1 | 0 | 0 | 0 | 0 | 0 | 0 | 0 | 0 | 0 | 0 |
| ynbB | 0 | 1 | 1 | 0 | 0 | 0 | 1 | 0 | 0 | 0 | 0 | 0 | 0 | 0 | 0 | 0 | 0 |
| ynbD | 0 | 1 | 1 | 0 | 0 | 0 | 1 | 0 | 0 | 0 | 0 | 0 | 0 | 0 | 0 | 0 | 0 |
| ynbA | 0 | 1 | 1 | 0 | 0 | 0 | 1 | 0 | 0 | 0 | 0 | 0 | 0 | 0 | 0 | 0 | 0 |
| ynbB | 0 | 1 | 1 | 0 | 0 | 0 | 1 | 0 | 0 | 0 | 0 | 0 | 0 | 0 | 0 | 0 | 0 |
| ynbD | 0 | 1 | 1 | 0 | 0 | 0 | 1 | 0 | 0 | 0 | 0 | 0 | 0 | 0 | 0 | 0 | 0 |
| ynbA | 0 | 1 | 1 | 0 | 0 | 0 | 1 | 0 | 0 | 0 | 0 | 0 | 0 | 0 | 0 | 0 | 0 |
| ynbB | 0 | 1 | 1 | 0 | 0 | 0 | 1 | 0 | 0 | 0 | 0 | 0 | 0 | 0 | 0 | 0 | 0 |
| ynbD | 0 | 1 | 1 | 0 | 0 | 0 | 1 | 0 | 0 | 0 | 0 | 0 | 0 | 0 | 0 | 0 | 0 |
| cybB | 0 | 1 | 1 | 0 | 0 | 0 | 1 | 0 | 0 | 0 | 0 | 0 | 0 | 0 | 0 | 0 | 0 |
| trg | 0 | 1 | 1 | 0 | 0 | 0 | 1 | 0 | 0 | 0 | 0 | 0 | 0 | 0 | 0 | 0 | 0 |
| tehA | 0 | 1 | 1 | 0 | 0 | 0 | 1 | 0 | 0 | 0 | 0 | 0 | 0 | 0 | 0 | 0 | 0 |
| ydcO | 0 | 1 | 1 | 0 | 0 | 0 | 1 | 0 | 0 | 0 | 0 | 0 | 0 | 0 | 0 | 0 | 0 |
| ydcT | 0 | 1 | 1 | 0 | 0 | 0 | 1 | 0 | 0 | 0 | 0 | 0 | 0 | 0 | 0 | 0 | 0 |
| ydcU | 0 | 1 | 1 | 0 | 0 | 0 | 1 | 0 | 0 | 0 | 0 | 0 | 0 | 0 | 0 | 0 | 0 |
| ydcV | 0 | 1 | 1 | 0 | 0 | 0 | 1 | 0 | 0 | 0 | 0 | 0 | 0 | 0 | 0 | 0 | 0 |
| ydcX | 0 | 1 | 1 | 0 | 0 | 0 | 1 | 0 | 0 | 0 | 0 | 0 | 0 | 0 | 0 | 0 | 0 |
| ydcZ | 0 | 1 | 1 | 0 | 0 | 0 | 1 | 0 | 0 | 0 | 0 | 0 | 0 | 0 | 0 | 0 | 0 |
| ansP | 0 | 1 | 1 | 0 | 0 | 0 | 1 | 0 | 0 | 0 | 0 | 0 | 0 | 0 | 0 | 0 | 0 |
| narV | 0 | 1 | 1 | 0 | 0 | 0 | 1 | 0 | 0 | 0 | 0 | 0 | 0 | 0 | 0 | 0 | 0 |
| narZ | 0 | 1 | 1 | 0 | 0 | 0 | 1 | 0 | 0 | 0 | 0 | 0 | 0 | 0 | 0 | 0 | 0 |
| narU | 0 | 1 | 1 | 0 | 0 | 0 | 1 | 0 | 0 | 0 | 0 | 0 | 0 | 0 | 0 | 0 | 0 |
| fdnH | 0 | 1 | 1 | 0 | 0 | 0 | 1 | 0 | 0 | 0 | 0 | 0 | 0 | 0 | 0 | 0 | 0 |
| fdnI | 0 | 1 | 1 | 0 | 0 | 0 | 1 | 0 | 0 | 0 | 0 | 0 | 0 | 0 | 0 | 0 | 0 |
| ddpD | 0 | 1 | 1 | 0 | 0 | 0 | 1 | 0 | 0 | 0 | 0 | 0 | 0 | 0 | 0 | 0 | 0 |
| ddpC | 0 | 1 | 1 | 0 | 0 | 0 | 1 | 0 | 0 | 0 | 0 | 0 | 0 | 0 | 0 | 0 | 0 |
| ddpB | 0 | 1 | 1 | 0 | 0 | 0 | 1 | 0 | 0 | 0 | 0 | 0 | 0 | 0 | 0 | 0 | 0 |
| dosP | 0 | 1 | 1 | 0 | 0 | 0 | 1 | 0 | 0 | 0 | 0 | 0 | 0 | 0 | 0 | 0 | 0 |
| gadC | 0 | 1 | 1 | 0 | 0 | 0 | 1 | 0 | 0 | 0 | 0 | 0 | 0 | 0 | 0 | 0 | 0 |
| yddA | 0 | 1 | 1 | 0 | 0 | 0 | 1 | 0 | 0 | 0 | 0 | 0 | 0 | 0 | 0 | 0 | 0 |
| safA | 0 | 1 | 1 | 0 | 0 | 0 | 1 | 0 | 0 | 0 | 0 | 0 | 0 | 0 | 0 | 0 | 0 |
| lsrA | 0 | 1 | 1 | 0 | 0 | 0 | 1 | 0 | 0 | 0 | 0 | 0 | 0 | 0 | 0 | 0 | 0 |
| lsrC | 0 | 1 | 1 | 0 | 0 | 0 | 1 | 0 | 0 | 0 | 0 | 0 | 0 | 0 | 0 | 0 | 0 |
| lsrD | 0 | 1 | 1 | 0 | 0 | 0 | 1 | 0 | 0 | 0 | 0 | 0 | 0 | 0 | 0 | 0 | 0 |
| yneE | 0 | 1 | 1 | 0 | 0 | 0 | 1 | 0 | 0 | 0 | 0 | 0 | 0 | 0 | 0 | 0 | 0 |
| yneF | 0 | 1 | 1 | 0 | 0 | 0 | 1 | 0 | 0 | 0 | 0 | 0 | 0 | 0 | 0 | 0 | 0 |
| ydeA | 0 | 1 | 1 | 0 | 0 | 0 | 1 | 0 | 0 | 0 | 0 | 0 | 0 | 0 | 0 | 0 | 0 |
| marC | 0 | 1 | 1 | 0 | 0 | 0 | 1 | 0 | 0 | 0 | 0 | 0 | 0 | 0 | 0 | 0 | 0 |
| eamA | 0 | 1 | 1 | 0 | 0 | 0 | 1 | 0 | 0 | 0 | 0 | 0 | 0 | 0 | 0 | 0 | 0 |
| ydeE | 0 | 1 | 1 | 0 | 0 | 0 | 1 | 0 | 0 | 0 | 0 | 0 | 0 | 0 | 0 | 0 | 0 |
| ydfJ | 0 | 1 | 1 | 0 | 0 | 0 | 1 | 0 | 0 | 0 | 0 | 0 | 0 | 0 | 0 | 0 | 0 |
| hokD | 0 | 1 | 1 | 0 | 0 | 0 | 1 | 0 | 0 | 0 | 0 | 0 | 0 | 0 | 0 | 0 | 0 |
| yafY | 0 | 1 | 1 | 0 | 0 | 0 | 1 | 0 | 0 | 0 | 0 | 0 | 0 | 0 | 0 | 0 | 0 |
| mmuP | 0 | 1 | 1 | 0 | 0 | 0 | 1 | 0 | 0 | 0 | 0 | 0 | 0 | 0 | 0 | 0 | 0 |
| yagG | 0 | 1 | 1 | 0 | 0 | 0 | 1 | 0 | 0 | 0 | 0 | 0 | 0 | 0 | 0 | 0 | 0 |
| yagU | 0 | 1 | 1 | 0 | 0 | 0 | 1 | 0 | 0 | 0 | 0 | 0 | 0 | 0 | 0 | 0 | 0 |
| ykgB | 0 | 1 | 1 | 0 | 0 | 0 | 1 | 0 | 0 | 0 | 0 | 0 | 0 | 0 | 0 | 0 | 0 |
| ykgH | 0 | 1 | 1 | 0 | 0 | 0 | 1 | 0 | 0 | 0 | 0 | 0 | 0 | 0 | 0 | 0 | 0 |
| betT | 0 | 1 | 1 | 0 | 0 | 0 | 1 | 0 | 0 | 0 | 0 | 0 | 0 | 0 | 0 | 0 | 0 |
| yahC | 0 | 1 | 1 | 0 | 0 | 0 | 1 | 0 | 0 | 0 | 0 | 0 | 0 | 0 | 0 | 0 | 0 |
| yahG | 0 | 1 | 1 | 0 | 0 | 0 | 1 | 0 | 0 | 0 | 0 | 0 | 0 | 0 | 0 | 0 | 0 |
| yahN | 0 | 1 | 1 | 0 | 0 | 0 | 1 | 0 | 0 | 0 | 0 | 0 | 0 | 0 | 0 | 0 | 0 |
| codB | 0 | 1 | 1 | 0 | 0 | 0 | 1 | 0 | 0 | 0 | 0 | 0 | 0 | 0 | 0 | 0 | 0 |
| cynX | 0 | 1 | 1 | 0 | 0 | 0 | 1 | 0 | 0 | 0 | 0 | 0 | 0 | 0 | 0 | 0 | 0 |
| lacY | 0 | 1 | 1 | 0 | 0 | 0 | 1 | 0 | 0 | 0 | 0 | 0 | 0 | 0 | 0 | 0 | 0 |
| mhpT | 0 | 1 | 1 | 0 | 0 | 0 | 1 | 0 | 0 | 0 | 0 | 0 | 0 | 0 | 0 | 0 | 0 |
| yaiP | 0 | 1 | 1 | 0 | 0 | 0 | 1 | 0 | 0 | 0 | 0 | 0 | 0 | 0 | 0 | 0 | 0 |
| tauC | 0 | 1 | 1 | 0 | 0 | 0 | 1 | 0 | 0 | 0 | 0 | 0 | 0 | 0 | 0 | 0 | 0 |
| modF3/4 | 0 | 1 | 1 | 0 | 0 | 0 | 1 | 0 | 0 | 0 | 0 | 0 | 0 | 0 | 0 | 0 | 0 |
| modB | 0 | 1 | 1 | 0 | 0 | 0 | 1 | 0 | 0 | 0 | 0 | 0 | 0 | 0 | 0 | 0 | 0 |
| modC | 0 | 1 | 1 | 0 | 0 | 0 | 1 | 0 | 0 | 0 | 0 | 0 | 0 | 0 | 0 | 0 | 0 |
| ybhI | 0 | 1 | 1 | 0 | 0 | 0 | 1 | 0 | 0 | 0 | 0 | 0 | 0 | 0 | 0 | 0 | 0 |
| ybhL | 0 | 1 | 1 | 0 | 0 | 0 | 1 | 0 | 0 | 0 | 0 | 0 | 0 | 0 | 0 | 0 | 0 |
| ybhM | 0 | 1 | 1 | 0 | 0 | 0 | 1 | 0 | 0 | 0 | 0 | 0 | 0 | 0 | 0 | 0 | 0 |
| ybhN | 0 | 1 | 1 | 0 | 0 | 0 | 1 | 0 | 0 | 0 | 0 | 0 | 0 | 0 | 0 | 0 | 0 |
| ybhQ | 0 | 1 | 1 | 0 | 0 | 0 | 1 | 0 | 0 | 0 | 0 | 0 | 0 | 0 | 0 | 0 | 0 |
| ybhR | 0 | 1 | 1 | 0 | 0 | 0 | 1 | 0 | 0 | 0 | 0 | 0 | 0 | 0 | 0 | 0 | 0 |
| ybhS | 0 | 1 | 1 | 0 | 0 | 0 | 1 | 0 | 0 | 0 | 0 | 0 | 0 | 0 | 0 | 0 | 0 |
| ybhF | 0 | 1 | 1 | 0 | 0 | 0 | 1 | 0 | 0 | 0 | 0 | 0 | 0 | 0 | 0 | 0 | 0 |
| ybiO | 0 | 1 | 1 | 0 | 0 | 0 | 1 | 0 | 0 | 0 | 0 | 0 | 0 | 0 | 0 | 0 | 0 |
| glnQ | 0 | 1 | 1 | 0 | 0 | 0 | 1 | 0 | 0 | 0 | 0 | 0 | 0 | 0 | 0 | 0 | 0 |
| glnP | 0 | 1 | 1 | 0 | 0 | 0 | 1 | 0 | 0 | 0 | 0 | 0 | 0 | 0 | 0 | 0 | 0 |
| rhtA | 0 | 1 | 1 | 0 | 0 | 0 | 1 | 0 | 0 | 0 | 0 | 0 | 0 | 0 | 0 | 0 | 0 |
| ybiP | 0 | 1 | 1 | 0 | 0 | 0 | 1 | 0 | 0 | 0 | 0 | 0 | 0 | 0 | 0 | 0 | 0 |
| ybiR | 0 | 1 | 1 | 0 | 0 | 0 | 1 | 0 | 0 | 0 | 0 | 0 | 0 | 0 | 0 | 0 | 0 |
| ybiT | 0 | 1 | 1 | 0 | 0 | 0 | 1 | 0 | 0 | 0 | 0 | 0 | 0 | 0 | 0 | 0 | 0 |
| gsiA | 0 | 1 | 1 | 0 | 0 | 0 | 1 | 0 | 0 | 0 | 0 | 0 | 0 | 0 | 0 | 0 | 0 |
| gsiC | 0 | 1 | 1 | 0 | 0 | 0 | 1 | 0 | 0 | 0 | 0 | 0 | 0 | 0 | 0 | 0 | 0 |
| gsiD | 0 | 1 | 1 | 0 | 0 | 0 | 1 | 0 | 0 | 0 | 0 | 0 | 0 | 0 | 0 | 0 | 0 |
| yliE | 0 | 1 | 1 | 0 | 0 | 0 | 1 | 0 | 0 | 0 | 0 | 0 | 0 | 0 | 0 | 0 | 0 |
| yliF | 0 | 1 | 1 | 0 | 0 | 0 | 1 | 0 | 0 | 0 | 0 | 0 | 0 | 0 | 0 | 0 | 0 |
| ybjG | 0 | 1 | 1 | 0 | 0 | 0 | 1 | 0 | 0 | 0 | 0 | 0 | 0 | 0 | 0 | 0 | 0 |
| mdfA | 0 | 1 | 1 | 0 | 0 | 0 | 1 | 0 | 0 | 0 | 0 | 0 | 0 | 0 | 0 | 0 | 0 |
| ybjJ | 0 | 1 | 1 | 0 | 0 | 0 | 1 | 0 | 0 | 0 | 0 | 0 | 0 | 0 | 0 | 0 | 0 |
| ybjL | 0 | 1 | 1 | 0 | 0 | 0 | 1 | 0 | 0 | 0 | 0 | 0 | 0 | 0 | 0 | 0 | 0 |
| ybjM | 0 | 1 | 1 | 0 | 0 | 0 | 1 | 0 | 0 | 0 | 0 | 0 | 0 | 0 | 0 | 0 | 0 |
| potH | 0 | 1 | 1 | 0 | 0 | 0 | 1 | 0 | 0 | 0 | 0 | 0 | 0 | 0 | 0 | 0 | 0 |
| potI | 0 | 1 | 1 | 0 | 0 | 0 | 1 | 0 | 0 | 0 | 0 | 0 | 0 | 0 | 0 | 0 | 0 |
| ybjO | 0 | 1 | 1 | 0 | 0 | 0 | 1 | 0 | 0 | 0 | 0 | 0 | 0 | 0 | 0 | 0 | 0 |
| artM | 0 | 1 | 1 | 0 | 0 | 0 | 1 | 0 | 0 | 0 | 0 | 0 | 0 | 0 | 0 | 0 | 0 |
| artQ | 0 | 1 | 1 | 0 | 0 | 0 | 1 | 0 | 0 | 0 | 0 | 0 | 0 | 0 | 0 | 0 | 0 |
| artP | 0 | 1 | 1 | 0 | 0 | 0 | 1 | 0 | 0 | 0 | 0 | 0 | 0 | 0 | 0 | 0 | 0 |
| ybjP | 0 | 1 | 1 | 0 | 0 | 0 | 1 | 0 | 0 | 0 | 0 | 0 | 0 | 0 | 0 | 0 | 0 |
| ybjT | 0 | 1 | 1 | 0 | 0 | 0 | 1 | 0 | 0 | 0 | 0 | 0 | 0 | 0 | 0 | 0 | 0 |
| ybjE | 0 | 1 | 1 | 0 | 0 | 0 | 1 | 0 | 0 | 0 | 0 | 0 | 0 | 0 | 0 | 0 | 0 |
| aqpZ | 0 | 1 | 1 | 0 | 0 | 0 | 1 | 0 | 0 | 0 | 0 | 0 | 0 | 0 | 0 | 0 | 0 |
| macA | 0 | 1 | 1 | 0 | 0 | 0 | 1 | 0 | 0 | 0 | 0 | 0 | 0 | 0 | 0 | 0 | 0 |
| yqaE | 0 | 1 | 1 | 0 | 0 | 0 | 1 | 0 | 0 | 0 | 0 | 0 | 0 | 0 | 0 | 0 | 0 |
| yqjK | 0 | 1 | 1 | 0 | 0 | 1 | 1 | 0 | 0 | 0 | 0 | 0 | 0 | 0 | 0 | 0 | 0 |
| putA | 0 | 1 | 1 | 0 | 0 | 1 | 1 | 0 | 0 | 0 | 0 | 0 | 0 | 0 | 0 | 0 | 0 |
| ghrA | 0 | 1 | 1 | 0 | 0 | 1 | 1 | 0 | 0 | 0 | 0 | 0 | 0 | 0 | 0 | 0 | 0 |
| lhgO | 0 | 1 | 1 | 0 | 0 | 1 | 1 | 0 | 0 | 0 | 0 | 0 | 0 | 0 | 0 | 0 | 0 |
| ygaM | 0 | 1 | 1 | 0 | 0 | 1 | 1 | 0 | 0 | 0 | 0 | 0 | 0 | 0 | 0 | 0 | 0 |
| hycF | 0 | 0 | 1 | 0 | 0 | 1 | 1 | 0 | 0 | 0 | 0 | 0 | 0 | 0 | 0 | 0 | 0 |
| hypA | 0 | 0 | 1 | 0 | 0 | 1 | 1 | 0 | 0 | 0 | 0 | 0 | 0 | 0 | 0 | 0 | 0 |
| elaB | 0 | 0 | 1 | 0 | 0 | 1 | 1 | 0 | 0 | 0 | 0 | 0 | 0 | 0 | 0 | 0 | 0 |
| elaA | 0 | 0 | 1 | 0 | 0 | 1 | 1 | 0 | 0 | 0 | 0 | 0 | 0 | 0 | 0 | 0 | 0 |
| sapF | 0 | 0 | 1 | 0 | 0 | 1 | 1 | 0 | 0 | 0 | 0 | 0 | 0 | 0 | 0 | 0 | 0 |
| pspA | 0 | 0 | 1 | 0 | 0 | 1 | 1 | 0 | 0 | 0 | 0 | 0 | 0 | 0 | 0 | 0 | 0 |
| ydcF | 0 | 0 | 1 | 0 | 0 | 1 | 1 | 0 | 0 | 0 | 0 | 0 | 0 | 0 | 0 | 0 | 0 |
| ddpF | 0 | 0 | 1 | 0 | 0 | 1 | 1 | 0 | 0 | 0 | 0 | 0 | 0 | 0 | 0 | 0 | 0 |
| dosC | 0 | 0 | 1 | 0 | 0 | 1 | 1 | 0 | 0 | 0 | 0 | 0 | 0 | 0 | 0 | 0 | 0 |
| ydeH | 0 | 0 | 1 | 0 | 0 | 1 | 1 | 0 | 0 | 0 | 0 | 0 | 0 | 0 | 0 | 0 | 0 |
| afuC | 0 | 0 | 1 | 0 | 0 | 1 | 1 | 0 | 0 | 0 | 0 | 0 | 0 | 0 | 0 | 0 | 0 |
| poxB | 0 | 0 | 1 | 0 | 0 | 1 | 1 | 0 | 0 | 0 | 0 | 0 | 0 | 0 | 0 | 0 | 0 |
| gntP | 1 | 1 | 1 | 0 | 0 | 0 | 1 | 0 | 0 | 0 | 1 | 0 | 0 | 0 | 0 | 0 | 0 |
| mdtM | 1 | 1 | 1 | 0 | 0 | 0 | 1 | 0 | 0 | 0 | 1 | 0 | 0 | 0 | 0 | 0 | 0 |
| ygjA | 0 | 1 | 1 | 0 | 0 | 0 | 1 | 0 | 0 | 0 | 1 | 0 | 0 | 0 | 0 | 0 | 0 |
| yhaO | 0 | 1 | 1 | 0 | 0 | 0 | 1 | 0 | 0 | 0 | 1 | 0 | 0 | 0 | 0 | 0 | 0 |
| tdcC | 0 | 1 | 1 | 0 | 0 | 0 | 1 | 0 | 0 | 0 | 1 | 0 | 0 | 0 | 0 | 0 | 0 |
| ybaL | 0 | 1 | 1 | 0 | 0 | 0 | 1 | 0 | 0 | 0 | 1 | 0 | 0 | 0 | 0 | 0 | 0 |
| fsr | 0 | 1 | 1 | 0 | 0 | 0 | 1 | 0 | 0 | 0 | 1 | 0 | 0 | 0 | 0 | 0 | 0 |
| copA | 0 | 1 | 1 | 0 | 0 | 0 | 1 | 0 | 0 | 0 | 1 | 0 | 0 | 0 | 0 | 0 | 0 |
| ybaT | 0 | 1 | 1 | 0 | 0 | 0 | 1 | 0 | 0 | 0 | 1 | 0 | 0 | 0 | 0 | 0 | 0 |
| ybbJ | 0 | 1 | 1 | 0 | 0 | 0 | 1 | 0 | 0 | 0 | 1 | 0 | 0 | 0 | 0 | 0 | 0 |
| ybbM | 0 | 1 | 1 | 0 | 0 | 0 | 1 | 0 | 0 | 0 | 1 | 0 | 0 | 0 | 0 | 0 | 0 |
| ybbW | 0 | 1 | 1 | 0 | 0 | 0 | 1 | 0 | 0 | 0 | 1 | 0 | 0 | 0 | 0 | 0 | 0 |
| fdrA | 0 | 1 | 1 | 0 | 0 | 0 | 1 | 0 | 0 | 0 | 1 | 0 | 0 | 0 | 0 | 0 | 0 |
| yjcE | 0 | 1 | 1 | 0 | 0 | 0 | 1 | 0 | 0 | 0 | 1 | 0 | 0 | 0 | 0 | 0 | 0 |
| actP | 0 | 1 | 1 | 0 | 0 | 0 | 1 | 0 | 0 | 0 | 1 | 0 | 0 | 0 | 0 | 0 | 0 |
| nrfD | 0 | 1 | 1 | 0 | 0 | 0 | 1 | 0 | 0 | 0 | 1 | 0 | 0 | 0 | 0 | 0 | 0 |
| nrfE | 0 | 1 | 1 | 0 | 0 | 0 | 1 | 0 | 0 | 0 | 1 | 0 | 0 | 0 | 0 | 0 | 0 |
| gltP | 0 | 1 | 1 | 0 | 0 | 0 | 1 | 0 | 0 | 0 | 1 | 0 | 0 | 0 | 0 | 0 | 0 |
| mdtN | 0 | 1 | 1 | 0 | 0 | 0 | 1 | 0 | 0 | 0 | 1 | 0 | 0 | 0 | 0 | 0 | 0 |
| nrfE | 0 | 1 | 1 | 0 | 0 | 0 | 1 | 0 | 0 | 0 | 1 | 0 | 0 | 0 | 0 | 0 | 0 |
| gltP | 0 | 1 | 1 | 0 | 0 | 0 | 1 | 0 | 0 | 0 | 1 | 0 | 0 | 0 | 0 | 0 | 0 |
| mdtN | 0 | 1 | 1 | 0 | 0 | 0 | 1 | 0 | 0 | 0 | 1 | 0 | 0 | 0 | 0 | 0 | 0 |
| alsC | 0 | 1 | 1 | 0 | 0 | 0 | 1 | 0 | 0 | 0 | 1 | 0 | 0 | 0 | 0 | 0 | 0 |
| proP | 0 | 1 | 1 | 0 | 0 | 0 | 1 | 0 | 0 | 0 | 1 | 0 | 0 | 0 | 0 | 0 | 0 |
| basS | 0 | 1 | 1 | 0 | 0 | 0 | 1 | 0 | 0 | 0 | 1 | 0 | 0 | 0 | 0 | 0 | 0 |
| adiC | 0 | 1 | 1 | 0 | 0 | 0 | 1 | 0 | 0 | 0 | 1 | 0 | 0 | 0 | 0 | 0 | 0 |
| melB | 0 | 1 | 1 | 0 | 0 | 0 | 1 | 0 | 0 | 0 | 1 | 0 | 0 | 0 | 0 | 0 | 0 |
| dcuB | 0 | 1 | 1 | 0 | 0 | 0 | 1 | 0 | 0 | 0 | 1 | 0 | 0 | 0 | 0 | 0 | 0 |
| yjdL | 0 | 1 | 1 | 0 | 0 | 0 | 1 | 0 | 0 | 0 | 1 | 0 | 0 | 0 | 0 | 0 | 0 |
| cadB | 0 | 1 | 1 | 0 | 0 | 0 | 1 | 0 | 0 | 0 | 1 | 0 | 0 | 0 | 0 | 0 | 0 |
| xylH | 0 | 1 | 1 | 0 | 0 | 0 | 1 | 0 | 0 | 0 | 1 | 0 | 0 | 0 | 0 | 0 | 0 |
| yiaN | 0 | 1 | 1 | 0 | 0 | 0 | 1 | 0 | 0 | 0 | 1 | 0 | 0 | 0 | 0 | 0 | 0 |
| yiaV | 0 | 1 | 1 | 0 | 0 | 0 | 1 | 0 | 0 | 0 | 1 | 0 | 0 | 0 | 0 | 0 | 0 |
| yiaW | 0 | 1 | 1 | 0 | 0 | 0 | 1 | 0 | 0 | 0 | 1 | 0 | 0 | 0 | 0 | 0 | 0 |
| yibH | 0 | 1 | 1 | 0 | 0 | 0 | 1 | 0 | 0 | 0 | 1 | 0 | 0 | 0 | 0 | 0 | 0 |
| yhhJ | 0 | 1 | 1 | 0 | 0 | 0 | 1 | 0 | 0 | 0 | 1 | 0 | 0 | 0 | 0 | 0 | 0 |
| pitA | 0 | 1 | 1 | 0 | 0 | 0 | 1 | 0 | 0 | 0 | 1 | 0 | 0 | 0 | 0 | 0 | 0 |
| uspB | 0 | 1 | 1 | 0 | 0 | 0 | 1 | 0 | 0 | 0 | 1 | 0 | 0 | 0 | 0 | 0 | 0 |
| dtpB | 0 | 1 | 1 | 0 | 0 | 0 | 1 | 0 | 0 | 0 | 1 | 0 | 0 | 0 | 0 | 0 | 0 |
| arsB | 0 | 1 | 1 | 0 | 0 | 0 | 1 | 0 | 0 | 0 | 1 | 0 | 0 | 0 | 0 | 0 | 0 |
| yhjE | 0 | 1 | 1 | 0 | 0 | 0 | 1 | 0 | 0 | 0 | 1 | 0 | 0 | 0 | 0 | 0 | 0 |
| dctA | 0 | 1 | 1 | 0 | 0 | 0 | 1 | 0 | 0 | 0 | 1 | 0 | 0 | 0 | 0 | 0 | 0 |
| yhjV | 0 | 1 | 1 | 0 | 0 | 0 | 1 | 0 | 0 | 0 | 1 | 0 | 0 | 0 | 0 | 0 | 0 |
| dppC | 0 | 1 | 1 | 0 | 0 | 0 | 1 | 0 | 0 | 0 | 1 | 0 | 0 | 0 | 0 | 0 | 0 |
| dppB | 0 | 1 | 1 | 0 | 0 | 0 | 1 | 0 | 0 | 0 | 1 | 0 | 0 | 0 | 0 | 0 | 0 |
| yhjX | 0 | 1 | 1 | 0 | 0 | 0 | 1 | 0 | 0 | 0 | 1 | 0 | 0 | 0 | 0 | 0 | 0 |
| yiaD | 0 | 1 | 1 | 0 | 0 | 0 | 1 | 0 | 0 | 0 | 1 | 0 | 0 | 0 | 0 | 0 | 0 |
| yehW | 0 | 1 | 1 | 0 | 0 | 0 | 1 | 0 | 0 | 0 | 1 | 0 | 0 | 0 | 0 | 0 | 0 |
| yehY | 0 | 1 | 1 | 0 | 0 | 0 | 1 | 0 | 0 | 0 | 1 | 0 | 0 | 0 | 0 | 0 | 0 |
| yohC | 0 | 1 | 1 | 0 | 0 | 0 | 1 | 0 | 0 | 0 | 1 | 0 | 0 | 0 | 0 | 0 | 0 |
| aer | 0 | 1 | 1 | 0 | 0 | 0 | 1 | 0 | 0 | 0 | 1 | 0 | 0 | 0 | 0 | 0 | 0 |
| nrfG | 0 | 1 | 1 | 0 | 0 | 0 | 1 | 0 | 1 | 0 | 0 | 0 | 0 | 0 | 0 | 0 | 0 |
| yhjA | 0 | 1 | 1 | 0 | 0 | 0 | 1 | 0 | 1 | 0 | 0 | 0 | 0 | 0 | 0 | 0 | 0 |
| asmA | 0 | 1 | 1 | 0 | 0 | 0 | 1 | 0 | 1 | 0 | 0 | 0 | 0 | 0 | 0 | 0 | 0 |
| mdtA | 0 | 1 | 1 | 0 | 0 | 0 | 1 | 0 | 1 | 0 | 0 | 0 | 0 | 0 | 0 | 0 | 0 |
| proX | 0 | 1 | 1 | 0 | 0 | 0 | 1 | 0 | 1 | 0 | 0 | 0 | 0 | 0 | 0 | 0 | 0 |
| emrA | 0 | 1 | 1 | 0 | 0 | 0 | 1 | 0 | 1 | 0 | 0 | 0 | 0 | 0 | 0 | 0 | 0 |
| dsbE | 0 | 0 | 1 | 0 | 0 | 0 | 1 | 0 | 1 | 0 | 0 | 0 | 0 | 0 | 0 | 0 | 0 |
| apbE | 0 | 0 | 1 | 0 | 0 | 0 | 1 | 0 | 1 | 0 | 0 | 0 | 0 | 0 | 0 | 0 | 0 |
| sapA | 0 | 0 | 1 | 0 | 0 | 0 | 1 | 0 | 1 | 0 | 0 | 0 | 0 | 0 | 0 | 0 | 0 |
| mppA | 0 | 0 | 1 | 0 | 0 | 0 | 1 | 0 | 1 | 0 | 0 | 0 | 0 | 0 | 0 | 0 | 0 |
| ddpA | 0 | 0 | 1 | 0 | 0 | 0 | 1 | 0 | 1 | 0 | 0 | 0 | 0 | 0 | 0 | 0 | 0 |
| lsrB | 0 | 0 | 1 | 0 | 0 | 0 | 1 | 0 | 1 | 0 | 0 | 0 | 0 | 0 | 0 | 0 | 0 |
| tauA | 0 | 0 | 1 | 0 | 0 | 0 | 1 | 0 | 1 | 0 | 0 | 0 | 0 | 0 | 0 | 0 | 0 |
| ybhG | 0 | 0 | 1 | 0 | 0 | 0 | 1 | 0 | 1 | 0 | 0 | 0 | 0 | 0 | 0 | 0 | 0 |
| gsiB | 0 | 0 | 1 | 0 | 0 | 0 | 1 | 0 | 1 | 0 | 0 | 0 | 0 | 0 | 0 | 0 | 0 |
| yjhP | 1 | 1 | 1 | 0 | 0 | 0 | 0 | 0 | 0 | 0 | 1 | 0 | 0 | 0 | 0 | 0 | 0 |
| yjiG | 1 | 1 | 1 | 0 | 0 | 0 | 0 | 0 | 0 | 0 | 1 | 0 | 0 | 0 | 0 | 0 | 0 |
| yjiH | 1 | 1 | 1 | 0 | 0 | 0 | 0 | 0 | 0 | 0 | 1 | 0 | 0 | 0 | 0 | 0 | 0 |
| yjiJ | 1 | 1 | 1 | 0 | 0 | 0 | 0 | 0 | 0 | 0 | 1 | 0 | 0 | 0 | 0 | 0 | 0 |
| yjiN | 1 | 1 | 1 | 0 | 0 | 0 | 0 | 0 | 0 | 0 | 1 | 0 | 0 | 0 | 0 | 0 | 0 |
| yfjV | 0 | 1 | 1 | 0 | 0 | 0 | 0 | 0 | 0 | 0 | 1 | 0 | 0 | 0 | 0 | 0 | 0 |
| idnD | 1 | 1 | 1 | 0 | 0 | 0 | 0 | 0 | 0 | 0 | 0 | 0 | 0 | 0 | 0 | 1 | 0 |
| yjgB | 1 | 1 | 1 | 0 | 0 | 0 | 0 | 0 | 0 | 0 | 0 | 0 | 0 | 0 | 0 | 1 | 0 |
| fecI | 1 | 1 | 1 | 0 | 0 | 0 | 0 | 0 | 0 | 0 | 0 | 0 | 0 | 0 | 0 | 1 | 0 |
| sgcR | 1 | 1 | 1 | 0 | 0 | 0 | 0 | 0 | 0 | 0 | 0 | 0 | 0 | 0 | 0 | 1 | 0 |
| fimB | 1 | 1 | 1 | 0 | 0 | 0 | 0 | 0 | 0 | 0 | 0 | 0 | 0 | 0 | 0 | 1 | 0 |
| fimE | 1 | 1 | 1 | 0 | 0 | 0 | 0 | 0 | 0 | 0 | 0 | 0 | 0 | 0 | 0 | 1 | 0 |
| uxuR | 1 | 1 | 1 | 0 | 0 | 0 | 0 | 0 | 0 | 0 | 0 | 0 | 0 | 0 | 0 | 1 | 0 |
| mcrC | 1 | 1 | 1 | 0 | 0 | 0 | 0 | 0 | 0 | 0 | 0 | 0 | 0 | 0 | 0 | 1 | 0 |
| hsdS | 1 | 1 | 1 | 0 | 0 | 0 | 0 | 0 | 0 | 0 | 0 | 0 | 0 | 0 | 0 | 1 | 0 |
| mrr | 1 | 1 | 1 | 0 | 0 | 0 | 0 | 0 | 0 | 0 | 0 | 0 | 0 | 0 | 0 | 1 | 0 |
| hsdM | 1 | 1 | 1 | 0 | 0 | 0 | 0 | 0 | 0 | 0 | 0 | 0 | 0 | 0 | 0 | 1 | 0 |
| hsdR | 1 | 1 | 1 | 0 | 0 | 0 | 0 | 0 | 0 | 0 | 0 | 0 | 0 | 0 | 0 | 1 | 0 |
| fecB | 1 | 1 | 1 | 0 | 0 | 0 | 0 | 0 | 0 | 0 | 0 | 0 | 0 | 0 | 0 | 1 | 0 |
| yjjM | 1 | 1 | 1 | 0 | 0 | 0 | 0 | 0 | 0 | 0 | 0 | 0 | 0 | 0 | 0 | 1 | 0 |
| idnK | 1 | 1 | 1 | 0 | 0 | 0 | 0 | 0 | 0 | 0 | 0 | 0 | 0 | 0 | 0 | 1 | 0 |
| kptA | 1 | 1 | 1 | 0 | 0 | 0 | 0 | 0 | 0 | 0 | 0 | 0 | 0 | 0 | 0 | 1 | 0 |
| symE | 1 | 1 | 1 | 0 | 0 | 0 | 0 | 0 | 0 | 0 | 0 | 0 | 0 | 0 | 0 | 1 | 0 |
| tdcD | 0 | 1 | 1 | 0 | 0 | 0 | 0 | 0 | 0 | 0 | 0 | 0 | 0 | 0 | 0 | 1 | 0 |
| phnF | 0 | 1 | 1 | 0 | 0 | 0 | 0 | 0 | 0 | 0 | 0 | 0 | 0 | 0 | 0 | 1 | 0 |
| dctR | 0 | 1 | 1 | 0 | 0 | 0 | 0 | 0 | 0 | 0 | 0 | 0 | 0 | 0 | 0 | 1 | 0 |
| gadE | 0 | 1 | 1 | 0 | 0 | 0 | 0 | 0 | 0 | 0 | 0 | 0 | 0 | 0 | 0 | 1 | 0 |
| gadW | 0 | 1 | 1 | 0 | 0 | 0 | 0 | 0 | 0 | 0 | 0 | 0 | 0 | 0 | 0 | 1 | 0 |
| gadX | 0 | 1 | 1 | 0 | 0 | 0 | 0 | 0 | 0 | 0 | 0 | 0 | 0 | 0 | 0 | 1 | 0 |
| yhjB | 0 | 1 | 1 | 0 | 0 | 0 | 0 | 0 | 0 | 0 | 0 | 0 | 0 | 0 | 0 | 1 | 0 |
| indO | 1 | 1 | 1 | 0 | 0 | 1 | 0 | 0 | 0 | 0 | 0 | 0 | 0 | 0 | 0 | 1 | 0 |
| idnR | 1 | 1 | 1 | 0 | 0 | 1 | 0 | 0 | 0 | 0 | 0 | 0 | 0 | 0 | 0 | 1 | 1 |
| sgcB | 1 | 1 | 1 | 0 | 0 | 1 | 0 | 0 | 0 | 0 | 0 | 0 | 0 | 0 | 0 | 1 | 1 |
| iraD | 1 | 1 | 1 | 0 | 0 | 1 | 0 | 0 | 0 | 0 | 0 | 0 | 0 | 0 | 0 | 1 | 1 |
| iadA | 1 | 1 | 1 | 0 | 0 | 1 | 0 | 0 | 0 | 0 | 0 | 0 | 0 | 0 | 0 | 1 | 1 |
| yjiN | 1 | 1 | 1 | 0 | 0 | 0 | 0 | 0 | 0 | 0 | 0 | 1 | 0 | 0 | 0 | 0 | 0 |
| yjhE | 1 | 1 | 1 | 0 | 0 | 0 | 0 | 0 | 0 | 0 | 0 | 1 | 0 | 0 | 0 | 0 | 0 |
| fecE | 1 | 1 | 1 | 0 | 0 | 0 | 0 | 0 | 0 | 0 | 0 | 1 | 0 | 0 | 0 | 0 | 0 |
| fecA | 1 | 1 | 1 | 0 | 0 | 0 | 0 | 0 | 0 | 0 | 0 | 1 | 0 | 0 | 0 | 0 | 0 |
| yjjL | 1 | 1 | 1 | 0 | 0 | 0 | 0 | 0 | 0 | 0 | 0 | 1 | 0 | 0 | 0 | 0 | 0 |
| ythA | 1 | 1 | 1 | 0 | 0 | 0 | 0 | 0 | 0 | 0 | 0 | 1 | 0 | 0 | 0 | 0 | 0 |
| yjiY | 1 | 1 | 1 | 0 | 0 | 0 | 0 | 0 | 0 | 0 | 0 | 1 | 0 | 0 | 0 | 0 | 0 |
| tsr | 1 | 1 | 1 | 0 | 0 | 0 | 0 | 0 | 0 | 0 | 0 | 1 | 0 | 0 | 0 | 0 | 0 |
| opgB | 1 | 1 | 1 | 0 | 0 | 0 | 0 | 0 | 0 | 0 | 0 | 1 | 0 | 0 | 0 | 0 | 0 |
| yhjK | 0 | 1 | 1 | 0 | 0 | 0 | 0 | 0 | 0 | 0 | 0 | 1 | 0 | 0 | 0 | 0 | 0 |
| yehR | 0 | 1 | 1 | 0 | 0 | 0 | 0 | 0 | 0 | 0 | 0 | 1 | 0 | 0 | 0 | 0 | 0 |
| clsC | 0 | 1 | 1 | 0 | 0 | 0 | 0 | 0 | 0 | 0 | 0 | 1 | 0 | 0 | 0 | 0 | 0 |
| hslJ | 0 | 0 | 1 | 0 | 0 | 0 | 0 | 0 | 0 | 0 | 0 | 1 | 0 | 0 | 0 | 0 | 0 |
| ynbC | 0 | 0 | 1 | 0 | 0 | 0 | 0 | 0 | 0 | 0 | 0 | 1 | 0 | 0 | 0 | 0 | 0 |
| ynbC | 0 | 0 | 1 | 0 | 0 | 0 | 0 | 0 | 0 | 0 | 0 | 1 | 0 | 0 | 0 | 0 | 0 |
| ynbC | 0 | 0 | 1 | 0 | 0 | 0 | 0 | 0 | 0 | 0 | 0 | 1 | 0 | 0 | 0 | 0 | 0 |
| ybjC | 0 | 0 | 1 | 0 | 0 | 0 | 0 | 0 | 0 | 0 | 0 | 1 | 0 | 0 | 0 | 0 | 0 |
| phnC | 0 | 1 | 1 | 0 | 0 | 1 | 0 | 0 | 0 | 0 | 0 | 1 | 0 | 0 | 0 | 0 | 0 |
| ygjI | 0 | 1 | 1 | 0 | 0 | 0 | 0 | 0 | 0 | 0 | 1 | 1 | 0 | 0 | 0 | 0 | 0 |
| ygjQ | 0 | 1 | 1 | 0 | 0 | 0 | 0 | 0 | 0 | 0 | 1 | 1 | 0 | 0 | 0 | 0 | 0 |
| alx | 0 | 1 | 1 | 0 | 0 | 0 | 0 | 0 | 0 | 0 | 1 | 1 | 0 | 0 | 0 | 0 | 0 |
| sstT | 0 | 1 | 1 | 0 | 0 | 0 | 0 | 0 | 0 | 0 | 1 | 1 | 0 | 0 | 0 | 0 | 0 |
| ygjV | 0 | 1 | 1 | 0 | 0 | 0 | 0 | 0 | 0 | 0 | 1 | 1 | 0 | 0 | 0 | 0 | 0 |
| exuT | 0 | 1 | 1 | 0 | 0 | 0 | 0 | 0 | 0 | 0 | 1 | 1 | 0 | 0 | 0 | 0 | 0 |
| yqjE | 0 | 1 | 1 | 0 | 0 | 0 | 0 | 0 | 0 | 0 | 1 | 1 | 0 | 0 | 0 | 0 | 0 |
| yqjF3/4 | 0 | 1 | 1 | 0 | 0 | 0 | 0 | 0 | 0 | 0 | 1 | 1 | 0 | 0 | 0 | 0 | 0 |
| yhaH3/4 | 0 | 1 | 1 | 0 | 0 | 0 | 0 | 0 | 0 | 0 | 1 | 1 | 0 | 0 | 0 | 0 | 0 |
| yhaI | 0 | 1 | 1 | 0 | 0 | 0 | 0 | 0 | 0 | 0 | 1 | 1 | 0 | 0 | 0 | 0 | 0 |
| qmcA | 0 | 1 | 1 | 0 | 0 | 0 | 0 | 0 | 0 | 0 | 1 | 1 | 0 | 0 | 0 | 0 | 0 |
| ybbP | 0 | 1 | 1 | 0 | 0 | 0 | 0 | 0 | 0 | 0 | 1 | 1 | 0 | 0 | 0 | 0 | 0 |
| rhsD | 0 | 1 | 1 | 0 | 0 | 0 | 0 | 0 | 0 | 0 | 1 | 1 | 0 | 0 | 0 | 0 | 0 |
| ybbD | 0 | 1 | 1 | 0 | 0 | 0 | 0 | 0 | 0 | 0 | 1 | 1 | 0 | 0 | 0 | 0 | 0 |
| ybbY | 0 | 1 | 1 | 0 | 0 | 0 | 0 | 0 | 0 | 0 | 1 | 1 | 0 | 0 | 0 | 0 | 0 |
| yjcH | 0 | 1 | 1 | 0 | 0 | 0 | 0 | 0 | 0 | 0 | 1 | 1 | 0 | 0 | 0 | 0 | 0 |
| mdtO | 0 | 1 | 1 | 0 | 0 | 0 | 0 | 0 | 0 | 0 | 1 | 1 | 0 | 0 | 0 | 0 | 0 |
| ytcA | 0 | 1 | 1 | 0 | 0 | 0 | 0 | 0 | 0 | 0 | 1 | 1 | 0 | 0 | 0 | 0 | 0 |
| mdtO | 0 | 1 | 1 | 0 | 0 | 0 | 0 | 0 | 0 | 0 | 1 | 1 | 0 | 0 | 0 | 0 | 0 |
| ytcA | 0 | 1 | 1 | 0 | 0 | 0 | 0 | 0 | 0 | 0 | 1 | 1 | 0 | 0 | 0 | 0 | 0 |
| phnE | 0 | 1 | 1 | 0 | 0 | 0 | 0 | 0 | 0 | 0 | 1 | 1 | 0 | 0 | 0 | 0 | 0 |
| eptA | 0 | 1 | 1 | 0 | 0 | 0 | 0 | 0 | 0 | 0 | 1 | 1 | 0 | 0 | 0 | 0 | 0 |
| yjdF | 0 | 1 | 1 | 0 | 0 | 0 | 0 | 0 | 0 | 0 | 1 | 1 | 0 | 0 | 0 | 0 | 0 |
| dcuS | 0 | 1 | 1 | 0 | 0 | 0 | 0 | 0 | 0 | 0 | 1 | 1 | 0 | 0 | 0 | 0 | 0 |
| yjdO | 0 | 1 | 1 | 0 | 0 | 0 | 0 | 0 | 0 | 0 | 1 | 1 | 0 | 0 | 0 | 0 | 0 |
| wecH | 0 | 1 | 1 | 0 | 0 | 0 | 0 | 0 | 0 | 0 | 1 | 1 | 0 | 0 | 0 | 0 | 0 |
| yiaA | 0 | 1 | 1 | 0 | 0 | 0 | 0 | 0 | 0 | 0 | 1 | 1 | 0 | 0 | 0 | 0 | 0 |
| yiaB | 0 | 1 | 1 | 0 | 0 | 0 | 0 | 0 | 0 | 0 | 1 | 1 | 0 | 0 | 0 | 0 | 0 |
| yiaM | 0 | 1 | 1 | 0 | 0 | 0 | 0 | 0 | 0 | 0 | 1 | 1 | 0 | 0 | 0 | 0 | 0 |
| rhsA | 0 | 1 | 1 | 0 | 0 | 0 | 0 | 0 | 0 | 0 | 1 | 1 | 0 | 0 | 0 | 0 | 0 |
| rhsB | 0 | 1 | 1 | 0 | 0 | 0 | 0 | 0 | 0 | 0 | 1 | 1 | 0 | 0 | 0 | 0 | 0 |
| yhiM | 0 | 1 | 1 | 0 | 0 | 0 | 0 | 0 | 0 | 0 | 1 | 1 | 0 | 0 | 0 | 0 | 0 |
| dinQ | 0 | 1 | 1 | 0 | 0 | 0 | 0 | 0 | 0 | 0 | 1 | 1 | 0 | 0 | 0 | 0 | 0 |
| yhiD | 0 | 1 | 1 | 0 | 0 | 0 | 0 | 0 | 0 | 0 | 1 | 1 | 0 | 0 | 0 | 0 | 0 |
| hdeD | 0 | 1 | 1 | 0 | 0 | 0 | 0 | 0 | 0 | 0 | 1 | 1 | 0 | 0 | 0 | 0 | 0 |
| yhjD | 0 | 1 | 1 | 0 | 0 | 0 | 0 | 0 | 0 | 0 | 1 | 1 | 0 | 0 | 0 | 0 | 0 |
| yhjG | 0 | 1 | 1 | 0 | 0 | 0 | 0 | 0 | 0 | 0 | 1 | 1 | 0 | 0 | 0 | 0 | 0 |
| bcsB | 0 | 1 | 1 | 0 | 0 | 0 | 0 | 0 | 0 | 0 | 1 | 1 | 0 | 0 | 0 | 0 | 0 |
| eptB | 0 | 1 | 1 | 0 | 0 | 0 | 0 | 0 | 0 | 0 | 1 | 1 | 0 | 0 | 0 | 0 | 0 |
| yehU | 0 | 1 | 1 | 0 | 0 | 0 | 0 | 0 | 0 | 0 | 1 | 1 | 0 | 0 | 0 | 0 | 0 |
| yohO | 0 | 1 | 1 | 0 | 0 | 0 | 0 | 0 | 0 | 0 | 1 | 1 | 0 | 0 | 0 | 0 | 0 |
| fimA | 1 | 1 | 1 | 0 | 0 | 0 | 0 | 1 | 0 | 0 | 0 | 0 | 0 | 0 | 0 | 0 | 0 |
| fimD | 1 | 1 | 1 | 0 | 0 | 0 | 0 | 1 | 0 | 0 | 0 | 0 | 0 | 0 | 0 | 0 | 0 |
| fimF | 1 | 1 | 1 | 0 | 0 | 0 | 0 | 1 | 0 | 0 | 0 | 0 | 0 | 0 | 0 | 0 | 0 |
| fimG | 1 | 1 | 1 | 0 | 0 | 0 | 0 | 1 | 0 | 0 | 0 | 0 | 0 | 0 | 0 | 0 | 0 |
| fimH | 1 | 1 | 1 | 0 | 0 | 0 | 0 | 1 | 0 | 0 | 0 | 0 | 0 | 0 | 0 | 0 | 0 |
| mdtP | 0 | 1 | 1 | 0 | 0 | 0 | 0 | 1 | 0 | 0 | 0 | 0 | 0 | 0 | 0 | 0 | 0 |
| yiaT | 0 | 1 | 1 | 0 | 0 | 0 | 0 | 1 | 0 | 0 | 0 | 0 | 0 | 0 | 0 | 0 | 0 |
| insH1 | 0 | 1 | 1 | 0 | 0 | 0 | 0 | 1 | 0 | 0 | 0 | 0 | 0 | 0 | 0 | 0 | 0 |
| slp | 0 | 1 | 1 | 0 | 0 | 0 | 0 | 1 | 0 | 0 | 0 | 0 | 0 | 0 | 0 | 0 | 0 |
| mdtF | 0 | 1 | 1 | 0 | 0 | 0 | 0 | 1 | 0 | 0 | 0 | 0 | 0 | 0 | 0 | 0 | 0 |
| bcsC | 0 | 1 | 1 | 0 | 0 | 0 | 0 | 1 | 0 | 0 | 0 | 0 | 0 | 0 | 0 | 0 | 0 |
| mdtQ | 0 | 1 | 1 | 0 | 0 | 0 | 0 | 1 | 0 | 0 | 0 | 0 | 0 | 0 | 0 | 0 | 0 |
| wza | 0 | 1 | 1 | 0 | 0 | 0 | 0 | 1 | 0 | 0 | 0 | 0 | 0 | 0 | 0 | 0 | 0 |
| yehB | 0 | 1 | 1 | 0 | 0 | 0 | 0 | 1 | 0 | 0 | 0 | 0 | 0 | 0 | 0 | 0 | 0 |
| gfcE | 0 | 1 | 1 | 0 | 0 | 0 | 0 | 1 | 0 | 0 | 0 | 0 | 0 | 0 | 0 | 0 | 0 |
| pgaB | 0 | 1 | 1 | 0 | 0 | 0 | 0 | 1 | 0 | 0 | 0 | 0 | 0 | 0 | 0 | 0 | 0 |
| pgaA | 0 | 1 | 1 | 0 | 0 | 0 | 0 | 1 | 0 | 0 | 0 | 0 | 0 | 0 | 0 | 0 | 0 |
| csgF | 0 | 1 | 1 | 0 | 0 | 0 | 0 | 1 | 0 | 0 | 0 | 0 | 0 | 0 | 0 | 0 | 0 |
| yceK | 0 | 1 | 1 | 0 | 0 | 0 | 0 | 1 | 0 | 0 | 0 | 0 | 0 | 0 | 0 | 0 | 0 |
| yejO | 0 | 0 | 1 | 0 | 0 | 0 | 0 | 1 | 0 | 0 | 0 | 0 | 0 | 0 | 0 | 0 | 0 |
| ompC | 0 | 0 | 1 | 0 | 0 | 0 | 0 | 1 | 0 | 0 | 0 | 0 | 0 | 0 | 0 | 0 | 0 |
| yfaZ | 0 | 0 | 1 | 0 | 0 | 0 | 0 | 1 | 0 | 0 | 0 | 0 | 0 | 0 | 0 | 0 | 0 |
| ompG | 0 | 0 | 1 | 0 | 0 | 0 | 0 | 1 | 0 | 0 | 0 | 0 | 0 | 0 | 0 | 0 | 0 |
| rzoR | 0 | 0 | 1 | 0 | 0 | 0 | 0 | 1 | 0 | 0 | 0 | 0 | 0 | 0 | 0 | 0 | 0 |
| ompN | 0 | 0 | 1 | 0 | 0 | 0 | 0 | 1 | 0 | 0 | 0 | 0 | 0 | 0 | 0 | 0 | 0 |
| yncD | 0 | 0 | 1 | 0 | 0 | 0 | 0 | 1 | 0 | 0 | 0 | 0 | 0 | 0 | 0 | 0 | 0 |
| yddL | 0 | 0 | 1 | 0 | 0 | 0 | 0 | 1 | 0 | 0 | 0 | 0 | 0 | 0 | 0 | 0 | 0 |
| yddB | 0 | 0 | 1 | 0 | 0 | 0 | 0 | 1 | 0 | 0 | 0 | 0 | 0 | 0 | 0 | 0 | 0 |
| ydeT | 0 | 0 | 1 | 0 | 0 | 0 | 0 | 1 | 0 | 0 | 0 | 0 | 0 | 0 | 0 | 0 | 0 |
| ecpC | 0 | 0 | 1 | 0 | 0 | 0 | 0 | 1 | 0 | 0 | 0 | 0 | 0 | 0 | 0 | 0 | 0 |
| yaiO | 0 | 0 | 1 | 0 | 0 | 0 | 0 | 1 | 0 | 0 | 0 | 0 | 0 | 0 | 0 | 0 | 0 |
| fiu | 0 | 0 | 1 | 0 | 0 | 0 | 0 | 1 | 0 | 0 | 0 | 0 | 0 | 0 | 0 | 0 | 0 |
| ompX | 0 | 0 | 1 | 0 | 0 | 0 | 0 | 1 | 0 | 0 | 0 | 0 | 0 | 0 | 0 | 0 | 0 |
| amiD | 0 | 0 | 1 | 0 | 0 | 0 | 0 | 1 | 0 | 0 | 0 | 0 | 0 | 0 | 0 | 0 | 0 |
| gfcD | 0 | 1 | 1 | 0 | 0 | 0 | 1 | 1 | 0 | 0 | 0 | 0 | 0 | 0 | 0 | 0 | 0 |
| nanC | 1 | 1 | 1 | 0 | 0 | 0 | 0 | 1 | 0 | 0 | 1 | 0 | 0 | 0 | 0 | 0 | 0 |
| yjiK | 1 | 1 | 1 | 0 | 0 | 0 | 0 | 1 | 0 | 0 | 1 | 0 | 0 | 0 | 0 | 0 | 0 |
| yliI | 0 | 0 | 1 | 0 | 0 | 0 | 0 | 1 | 1 | 0 | 0 | 0 | 0 | 0 | 0 | 0 | 0 |
| xylF | 0 | 1 | 1 | 0 | 0 | 0 | 0 | 0 | 1 | 0 | 0 | 0 | 0 | 0 | 0 | 0 | 0 |
| malS | 0 | 1 | 1 | 0 | 0 | 0 | 0 | 0 | 1 | 0 | 0 | 0 | 0 | 0 | 0 | 0 | 0 |
| yiaO | 0 | 1 | 1 | 0 | 0 | 0 | 0 | 0 | 1 | 0 | 0 | 0 | 0 | 0 | 0 | 0 | 0 |
| yddW | 0 | 0 | 1 | 0 | 0 | 0 | 1 | 0 | 1 | 0 | 0 | 0 | 0 | 0 | 0 | 0 | 0 |
| fecR | 1 | 1 | 1 | 0 | 0 | 0 | 0 | 0 | 1 | 0 | 0 | 0 | 0 | 0 | 0 | 0 | 0 |
| nanS | 1 | 1 | 1 | 0 | 0 | 0 | 0 | 0 | 1 | 0 | 0 | 0 | 0 | 0 | 0 | 0 | 0 |
| nanM | 1 | 1 | 1 | 0 | 0 | 0 | 0 | 0 | 1 | 0 | 0 | 0 | 0 | 0 | 0 | 0 | 0 |
| fimC | 1 | 1 | 1 | 0 | 0 | 0 | 0 | 0 | 1 | 0 | 0 | 0 | 0 | 0 | 0 | 0 | 0 |
| ushA | 0 | 1 | 1 | 0 | 0 | 0 | 0 | 0 | 1 | 0 | 0 | 0 | 0 | 0 | 0 | 0 | 0 |
| ybaK | 0 | 1 | 1 | 0 | 0 | 0 | 0 | 0 | 1 | 0 | 0 | 0 | 0 | 0 | 0 | 0 | 0 |
| tesA | 0 | 1 | 1 | 0 | 0 | 0 | 0 | 0 | 1 | 0 | 0 | 0 | 0 | 0 | 0 | 0 | 0 |
| nrfA | 0 | 1 | 1 | 0 | 0 | 0 | 0 | 0 | 1 | 0 | 0 | 0 | 0 | 0 | 0 | 0 | 0 |
| nrfB | 0 | 1 | 1 | 0 | 0 | 0 | 0 | 0 | 1 | 0 | 0 | 0 | 0 | 0 | 0 | 0 | 0 |
| nrfF | 0 | 1 | 1 | 0 | 0 | 0 | 0 | 0 | 1 | 0 | 0 | 0 | 0 | 0 | 0 | 0 | 0 |
| nrfA | 0 | 1 | 1 | 0 | 0 | 0 | 0 | 0 | 1 | 0 | 0 | 0 | 0 | 0 | 0 | 0 | 0 |
| nrfB | 0 | 1 | 1 | 0 | 0 | 0 | 0 | 0 | 1 | 0 | 0 | 0 | 0 | 0 | 0 | 0 | 0 |
| nrfF | 0 | 1 | 1 | 0 | 0 | 0 | 0 | 0 | 1 | 0 | 0 | 0 | 0 | 0 | 0 | 0 | 0 |
| yjcS | 0 | 1 | 1 | 0 | 0 | 0 | 0 | 0 | 1 | 0 | 0 | 0 | 0 | 0 | 0 | 0 | 0 |
| alsB | 0 | 1 | 1 | 0 | 0 | 0 | 0 | 0 | 1 | 0 | 0 | 0 | 0 | 0 | 0 | 0 | 0 |
| rpiB | 0 | 1 | 1 | 0 | 0 | 0 | 0 | 0 | 1 | 0 | 0 | 0 | 0 | 0 | 0 | 0 | 0 |
| phnD | 0 | 1 | 1 | 0 | 0 | 0 | 0 | 0 | 1 | 0 | 0 | 0 | 0 | 0 | 0 | 0 | 0 |
| hdeA | 0 | 1 | 1 | 0 | 0 | 0 | 0 | 0 | 1 | 0 | 0 | 0 | 0 | 0 | 0 | 0 | 0 |
| yhjJ | 0 | 1 | 1 | 0 | 0 | 0 | 0 | 0 | 1 | 0 | 0 | 0 | 0 | 0 | 0 | 0 | 0 |
| dppA | 0 | 1 | 1 | 0 | 0 | 0 | 0 | 0 | 1 | 0 | 0 | 0 | 0 | 0 | 0 | 0 | 0 |
| osmF | 0 | 1 | 1 | 0 | 0 | 0 | 0 | 0 | 1 | 0 | 0 | 0 | 0 | 0 | 0 | 0 | 0 |
| bglX | 0 | 1 | 1 | 0 | 0 | 0 | 0 | 0 | 1 | 0 | 0 | 0 | 0 | 0 | 0 | 0 | 0 |
| pbpG | 0 | 1 | 1 | 0 | 0 | 0 | 0 | 0 | 1 | 0 | 0 | 0 | 0 | 0 | 0 | 0 | 0 |
| mglB | 0 | 1 | 1 | 0 | 0 | 0 | 0 | 0 | 1 | 0 | 0 | 0 | 0 | 0 | 0 | 0 | 0 |
| yehC | 0 | 1 | 1 | 0 | 0 | 0 | 0 | 0 | 1 | 0 | 0 | 0 | 0 | 0 | 0 | 0 | 0 |
| torT | 0 | 1 | 1 | 0 | 0 | 0 | 0 | 0 | 1 | 0 | 0 | 0 | 0 | 0 | 0 | 0 | 0 |
| torA | 0 | 1 | 1 | 0 | 0 | 0 | 0 | 0 | 1 | 0 | 0 | 0 | 0 | 0 | 0 | 0 | 0 |
| agp | 0 | 1 | 1 | 0 | 0 | 0 | 0 | 0 | 1 | 0 | 0 | 0 | 0 | 0 | 0 | 0 | 0 |
| efeO | 0 | 1 | 1 | 0 | 0 | 0 | 0 | 0 | 1 | 0 | 0 | 0 | 0 | 0 | 0 | 0 | 0 |
| csgC | 0 | 1 | 1 | 0 | 0 | 0 | 0 | 0 | 1 | 0 | 0 | 0 | 0 | 0 | 0 | 0 | 0 |
| opgG | 0 | 1 | 1 | 0 | 0 | 0 | 0 | 0 | 1 | 0 | 0 | 0 | 0 | 0 | 0 | 0 | 0 |
| napB | 0 | 0 | 1 | 0 | 0 | 0 | 0 | 0 | 1 | 0 | 0 | 0 | 0 | 0 | 0 | 0 | 0 |
| napA | 0 | 0 | 1 | 0 | 0 | 0 | 0 | 0 | 1 | 0 | 0 | 0 | 0 | 0 | 0 | 0 | 0 |
| eco | 0 | 0 | 1 | 0 | 0 | 0 | 0 | 0 | 1 | 0 | 0 | 0 | 0 | 0 | 0 | 0 | 0 |
| glpQ | 0 | 0 | 1 | 0 | 0 | 0 | 0 | 0 | 1 | 0 | 0 | 0 | 0 | 0 | 0 | 0 | 0 |
| ais | 0 | 0 | 1 | 0 | 0 | 0 | 0 | 0 | 1 | 0 | 0 | 0 | 0 | 0 | 0 | 0 | 0 |
| pspE | 0 | 0 | 1 | 0 | 0 | 0 | 0 | 0 | 1 | 0 | 0 | 0 | 0 | 0 | 0 | 0 | 0 |
| ycjN | 0 | 0 | 1 | 0 | 0 | 0 | 0 | 0 | 1 | 0 | 0 | 0 | 0 | 0 | 0 | 0 | 0 |
| tynA | 0 | 0 | 1 | 0 | 0 | 0 | 0 | 0 | 1 | 0 | 0 | 0 | 0 | 0 | 0 | 0 | 0 |
| opgD | 0 | 0 | 1 | 0 | 0 | 0 | 0 | 0 | 1 | 0 | 0 | 0 | 0 | 0 | 0 | 0 | 0 |
| ydcS | 0 | 0 | 1 | 0 | 0 | 0 | 0 | 0 | 1 | 0 | 0 | 0 | 0 | 0 | 0 | 0 | 0 |
| yncE | 0 | 0 | 1 | 0 | 0 | 0 | 0 | 0 | 1 | 0 | 0 | 0 | 0 | 0 | 0 | 0 | 0 |
| ydeN | 0 | 0 | 1 | 0 | 0 | 0 | 0 | 0 | 1 | 0 | 0 | 0 | 0 | 0 | 0 | 0 | 0 |
| ydeP | 0 | 0 | 1 | 0 | 0 | 0 | 0 | 0 | 1 | 0 | 0 | 0 | 0 | 0 | 0 | 0 | 0 |
| marB | 0 | 0 | 1 | 0 | 0 | 0 | 0 | 0 | 1 | 0 | 0 | 0 | 0 | 0 | 0 | 0 | 0 |
| ydel | 0 | 0 | 1 | 0 | 0 | 0 | 0 | 0 | 1 | 0 | 0 | 0 | 0 | 0 | 0 | 0 | 0 |
| paoC | 0 | 0 | 1 | 0 | 0 | 0 | 0 | 0 | 1 | 0 | 0 | 0 | 0 | 0 | 0 | 0 | 0 |
| paoB | 0 | 0 | 1 | 0 | 0 | 0 | 0 | 0 | 1 | 0 | 0 | 0 | 0 | 0 | 0 | 0 | 0 |
| paoA | 0 | 0 | 1 | 0 | 0 | 0 | 0 | 0 | 1 | 0 | 0 | 0 | 0 | 0 | 0 | 0 | 0 |
| ecpE | 0 | 0 | 1 | 0 | 0 | 0 | 0 | 0 | 1 | 0 | 0 | 0 | 0 | 0 | 0 | 0 | 0 |
| yahO | 0 | 0 | 1 | 0 | 0 | 0 | 0 | 0 | 1 | 0 | 0 | 0 | 0 | 0 | 0 | 0 | 0 |
| modA | 0 | 0 | 1 | 0 | 0 | 0 | 0 | 0 | 1 | 0 | 0 | 0 | 0 | 0 | 0 | 0 | 0 |
| ybiJ | 0 | 0 | 1 | 0 | 0 | 0 | 0 | 0 | 1 | 0 | 0 | 0 | 0 | 0 | 0 | 0 | 0 |
| potF | 0 | 0 | 1 | 0 | 0 | 0 | 0 | 0 | 1 | 0 | 0 | 0 | 0 | 0 | 0 | 0 | 0 |
| fdhF | 0 | 1 | 1 | 0 | 0 | 1 | 0 | 0 | 1 | 0 | 0 | 0 | 0 | 0 | 0 | 0 | 0 |
| bisC | 0 | 1 | 1 | 0 | 0 | 1 | 0 | 0 | 1 | 0 | 0 | 0 | 0 | 0 | 0 | 0 | 0 |
| efeB | 0 | 1 | 1 | 0 | 0 | 1 | 0 | 0 | 1 | 0 | 0 | 0 | 0 | 0 | 0 | 0 | 0 |
| napF | 0 | 0 | 1 | 0 | 0 | 1 | 0 | 0 | 1 | 0 | 0 | 0 | 0 | 0 | 0 | 0 | 0 |
| tpx | 0 | 0 | 1 | 0 | 0 | 1 | 0 | 0 | 1 | 0 | 0 | 0 | 0 | 0 | 0 | 0 | 0 |
| csgE | 0 | 1 | 1 | 0 | 0 | 1 | 0 | 1 | 1 | 0 | 0 | 0 | 0 | 0 | 0 | 0 | 0 |
| sanA | 0 | 1 | 1 | 0 | 0 | 0 | 1 | 0 | 1 | 0 | 0 | 0 | 0 | 0 | 0 | 0 | 0 |
| torS | 0 | 1 | 1 | 0 | 0 | 0 | 1 | 0 | 1 | 0 | 0 | 0 | 0 | 0 | 0 | 0 | 0 |
| napG | 0 | 0 | 1 | 0 | 0 | 0 | 1 | 0 | 1 | 0 | 0 | 0 | 0 | 0 | 0 | 0 | 0 |
| ycjR | 0 | 0 | 1 | 0 | 0 | 0 | 1 | 0 | 1 | 0 | 0 | 0 | 0 | 0 | 0 | 0 | 0 |
| ydcL | 0 | 0 | 1 | 0 | 0 | 0 | 1 | 0 | 1 | 0 | 0 | 0 | 0 | 0 | 0 | 0 | 0 |
| mcbA | 0 | 0 | 1 | 0 | 0 | 0 | 1 | 0 | 1 | 0 | 0 | 0 | 0 | 0 | 0 | 0 | 0 |
| dacC | 0 | 0 | 1 | 0 | 0 | 0 | 1 | 0 | 1 | 0 | 0 | 0 | 0 | 0 | 0 | 0 | 0 |
| csgG | 0 | 1 | 1 | 0 | 0 | 0 | 1 | 1 | 1 | 0 | 0 | 0 | 0 | 0 | 0 | 0 | 0 |
| fdnG | 0 | 0 | 1 | 0 | 0 | 0 | 0 | 0 | 1 | 0 | 0 | 1 | 0 | 0 | 0 | 0 | 0 |
| glnH | 0 | 0 | 1 | 0 | 0 | 0 | 0 | 0 | 1 | 0 | 0 | 1 | 0 | 0 | 0 | 0 | 0 |
| artJ | 0 | 0 | 1 | 0 | 0 | 0 | 0 | 0 | 1 | 0 | 0 | 1 | 0 | 0 | 0 | 0 | 0 |
| artI | 0 | 0 | 1 | 0 | 0 | 0 | 0 | 0 | 1 | 0 | 0 | 1 | 0 | 0 | 0 | 0 | 0 |
| mltB | 0 | 0 | 1 | 0 | 0 | 0 | 0 | 1 | 1 | 0 | 0 | 0 | 0 | 0 | 0 | 0 | 0 |
| yehA | 0 | 1 | 1 | 0 | 0 | 0 | 0 | 0 | 0 | 0 | 0 | 0 | 1 | 0 | 0 | 0 | 0 |
| csgA | 0 | 1 | 1 | 0 | 0 | 0 | 0 | 0 | 0 | 0 | 0 | 0 | 1 | 0 | 0 | 0 | 0 |
| ecpD | 0 | 0 | 1 | 0 | 0 | 0 | 0 | 0 | 0 | 0 | 0 | 0 | 1 | 0 | 0 | 0 | 0 |
| ecpA | 0 | 0 | 1 | 0 | 0 | 0 | 0 | 0 | 0 | 0 | 0 | 0 | 1 | 0 | 0 | 0 | 0 |
| csgB | 0 | 1 | 1 | 0 | 0 | 0 | 0 | 1 | 0 | 0 | 0 | 0 | 1 | 0 | 0 | 0 | 0 |
| yibI | 0 | 1 | 1 | 0 | 0 | 0 | 0 | 0 | 0 | 0 | 0 | 1 | 0 | 0 | 0 | 0 | 0 |
| ygjR | 1 | 1 | 1 | 0 | 0 | 0 | 0 | 0 | 0 | 0 | 0 | 0 | 0 | 0 | 1 | 0 | 0 |
| yjhC | 1 | 1 | 1 | 0 | 0 | 0 | 0 | 0 | 0 | 0 | 0 | 0 | 0 | 0 | 0 | 0 | 1 |
| yjhR | 1 | 1 | 1 | 0 | 0 | 0 | 0 | 0 | 0 | 0 | 0 | 0 | 0 | 0 | 0 | 0 | 1 |
| insO | 1 | 1 | 1 | 0 | 0 | 0 | 0 | 0 | 0 | 0 | 0 | 0 | 0 | 0 | 0 | 0 | 1 |
| leuX (tRNA) | 1 | 1 | 1 | 0 | 0 | 0 | 0 | 0 | 0 | 0 | 0 | 0 | 0 | 0 | 0 | 0 | 1 |
| yjhD | 1 | 1 | 1 | 0 | 0 | 0 | 0 | 0 | 0 | 0 | 0 | 0 | 0 | 0 | 0 | 0 | 1 |
| insM | 1 | 1 | 1 | 0 | 0 | 0 | 0 | 0 | 0 | 0 | 0 | 0 | 0 | 0 | 0 | 0 | 1 |
| yjhV | 1 | 1 | 1 | 0 | 0 | 0 | 0 | 0 | 0 | 0 | 0 | 0 | 0 | 0 | 0 | 0 | 1 |
| insA | 1 | 1 | 1 | 0 | 0 | 0 | 0 | 0 | 0 | 0 | 0 | 0 | 0 | 0 | 0 | 0 | 1 |
| ryjB | 1 | 1 | 1 | 0 | 0 | 0 | 0 | 0 | 0 | 0 | 0 | 0 | 0 | 0 | 0 | 0 | 1 |
| yjhY | 1 | 1 | 1 | 0 | 0 | 0 | 0 | 0 | 0 | 0 | 0 | 0 | 0 | 0 | 0 | 0 | 1 |
| yjhX | 1 | 1 | 1 | 0 | 0 | 0 | 0 | 0 | 0 | 0 | 0 | 0 | 0 | 0 | 0 | 0 | 1 |
| yjhZ | 1 | 1 | 1 | 0 | 0 | 0 | 0 | 0 | 0 | 0 | 0 | 0 | 0 | 0 | 0 | 0 | 1 |
| yjiC | 1 | 1 | 1 | 0 | 0 | 0 | 0 | 0 | 0 | 0 | 0 | 0 | 0 | 0 | 0 | 0 | 1 |
| yjiM | 1 | 1 | 1 | 0 | 0 | 0 | 0 | 0 | 0 | 0 | 0 | 0 | 0 | 0 | 0 | 0 | 1 |
| yjiP | 1 | 1 | 1 | 0 | 0 | 0 | 0 | 0 | 0 | 0 | 0 | 0 | 0 | 0 | 0 | 0 | 1 |
| yjiS | 1 | 1 | 1 | 0 | 0 | 0 | 0 | 0 | 0 | 0 | 0 | 0 | 0 | 0 | 0 | 0 | 1 |
| yjiT | 1 | 1 | 1 | 0 | 0 | 0 | 0 | 0 | 0 | 0 | 0 | 0 | 0 | 0 | 0 | 0 | 1 |
| yjiV | 1 | 1 | 1 | 0 | 0 | 0 | 0 | 0 | 0 | 0 | 0 | 0 | 0 | 0 | 0 | 0 | 1 |
| symR | 1 | 1 | 1 | 0 | 0 | 0 | 0 | 0 | 0 | 0 | 0 | 0 | 0 | 0 | 0 | 0 | 1 |
| yjiX | 1 | 1 | 1 | 0 | 0 | 0 | 0 | 0 | 0 | 0 | 0 | 0 | 0 | 0 | 0 | 0 | 1 |
| ebgA | 0 | 1 | 1 | 0 | 0 | 0 | 0 | 0 | 0 | 0 | 0 | 0 | 0 | 0 | 0 | 0 | 0 |
| ygjK3/4 | 0 | 1 | 1 | 0 | 0 | 0 | 0 | 0 | 0 | 0 | 0 | 0 | 0 | 0 | 0 | 0 | 0 |
| ygjR3/4 | 0 | 1 | 1 | 0 | 0 | 0 | 0 | 0 | 0 | 0 | 0 | 0 | 0 | 0 | 0 | 0 | 0 |
| mzrA | 0 | 1 | 1 | 0 | 0 | 0 | 0 | 0 | 0 | 0 | 0 | 0 | 0 | 0 | 0 | 0 | 0 |
| yqjD | 0 | 1 | 1 | 0 | 0 | 0 | 0 | 0 | 0 | 0 | 0 | 0 | 0 | 0 | 0 | 0 | 0 |
| yhaL | 0 | 1 | 1 | 0 | 0 | 0 | 0 | 0 | 0 | 0 | 0 | 0 | 0 | 0 | 0 | 0 | 0 |
| yhaM | 0 | 1 | 1 | 0 | 0 | 0 | 0 | 0 | 0 | 0 | 0 | 0 | 0 | 0 | 0 | 0 | 0 |
| tdcG | 0 | 1 | 1 | 0 | 0 | 0 | 0 | 0 | 0 | 0 | 0 | 0 | 0 | 0 | 0 | 0 | 0 |
| yhaC | 0 | 1 | 1 | 0 | 0 | 0 | 0 | 0 | 0 | 0 | 0 | 0 | 0 | 0 | 0 | 0 | 0 |
| chiX | 0 | 1 | 1 | 0 | 0 | 0 | 0 | 0 | 0 | 0 | 0 | 0 | 0 | 0 | 0 | 0 | 0 |
| ybaP | 0 | 1 | 1 | 0 | 0 | 0 | 0 | 0 | 0 | 0 | 0 | 0 | 0 | 0 | 0 | 0 | 0 |
| ybbO | 0 | 1 | 1 | 0 | 0 | 0 | 0 | 0 | 0 | 0 | 0 | 0 | 0 | 0 | 0 | 0 | 0 |
| ybbC | 0 | 1 | 1 | 0 | 0 | 0 | 0 | 0 | 0 | 0 | 0 | 0 | 0 | 0 | 0 | 0 | 0 |
| ylbH | 0 | 1 | 1 | 0 | 0 | 0 | 0 | 0 | 0 | 0 | 0 | 0 | 0 | 0 | 0 | 0 | 0 |
| ylbI | 0 | 1 | 1 | 0 | 0 | 0 | 0 | 0 | 0 | 0 | 0 | 0 | 0 | 0 | 0 | 0 | 0 |
| ylbG | 0 | 1 | 1 | 0 | 0 | 0 | 0 | 0 | 0 | 0 | 0 | 0 | 0 | 0 | 0 | 0 | 0 |
| allS | 0 | 1 | 1 | 0 | 0 | 0 | 0 | 0 | 0 | 0 | 0 | 0 | 0 | 0 | 0 | 0 | 0 |
| gcl | 0 | 1 | 1 | 0 | 0 | 0 | 0 | 0 | 0 | 0 | 0 | 0 | 0 | 0 | 0 | 0 | 0 |
| ylbE | 0 | 1 | 1 | 0 | 0 | 0 | 0 | 0 | 0 | 0 | 0 | 0 | 0 | 0 | 0 | 0 | 0 |
| ylbF | 0 | 1 | 1 | 0 | 0 | 0 | 0 | 0 | 0 | 0 | 0 | 0 | 0 | 0 | 0 | 0 | 0 |
| yjcF | 0 | 1 | 1 | 0 | 0 | 0 | 0 | 0 | 0 | 0 | 0 | 0 | 0 | 0 | 0 | 0 | 0 |
| yjcO | 0 | 1 | 1 | 0 | 0 | 0 | 0 | 0 | 0 | 0 | 0 | 0 | 0 | 0 | 0 | 0 | 0 |
| acs | 0 | 1 | 1 | 0 | 0 | 0 | 0 | 0 | 0 | 0 | 0 | 0 | 0 | 0 | 0 | 0 | 0 |
| yjcO | 0 | 1 | 1 | 0 | 0 | 0 | 0 | 0 | 0 | 0 | 0 | 0 | 0 | 0 | 0 | 0 | 0 |
| yjdP | 0 | 1 | 1 | 0 | 0 | 0 | 0 | 0 | 0 | 0 | 0 | 0 | 0 | 0 | 0 | 0 | 0 |
| yjdN | 0 | 1 | 1 | 0 | 0 | 0 | 0 | 0 | 0 | 0 | 0 | 0 | 0 | 0 | 0 | 0 | 0 |
| yjcZ | 0 | 1 | 1 | 0 | 0 | 0 | 0 | 0 | 0 | 0 | 0 | 0 | 0 | 0 | 0 | 0 | 0 |
| fumB | 0 | 1 | 1 | 0 | 0 | 0 | 0 | 0 | 0 | 0 | 0 | 0 | 0 | 0 | 0 | 0 | 0 |
| yjdI | 0 | 1 | 1 | 0 | 0 | 0 | 0 | 0 | 0 | 0 | 0 | 0 | 0 | 0 | 0 | 0 | 0 |
| yjdK | 0 | 1 | 1 | 0 | 0 | 0 | 0 | 0 | 0 | 0 | 0 | 0 | 0 | 0 | 0 | 0 | 0 |
| bax | 0 | 1 | 1 | 0 | 0 | 0 | 0 | 0 | 0 | 0 | 0 | 0 | 0 | 0 | 0 | 0 | 0 |
| ysaC | 0 | 1 | 1 | 0 | 0 | 0 | 0 | 0 | 0 | 0 | 0 | 0 | 0 | 0 | 0 | 0 | 0 |
| ysaD | 0 | 1 | 1 | 0 | 0 | 0 | 0 | 0 | 0 | 0 | 0 | 0 | 0 | 0 | 0 | 0 | 0 |
| yibJ | 0 | 1 | 1 | 0 | 0 | 0 | 0 | 0 | 0 | 0 | 0 | 0 | 0 | 0 | 0 | 0 | 0 |
| yibG | 0 | 1 | 1 | 0 | 0 | 0 | 0 | 0 | 0 | 0 | 0 | 0 | 0 | 0 | 0 | 0 | 0 |
| yhhH | 0 | 1 | 1 | 0 | 0 | 0 | 0 | 0 | 0 | 0 | 0 | 0 | 0 | 0 | 0 | 0 | 0 |
| yhhI | 0 | 1 | 1 | 0 | 0 | 0 | 0 | 0 | 0 | 0 | 0 | 0 | 0 | 0 | 0 | 0 | 0 |
| yhiJ | 0 | 1 | 1 | 0 | 0 | 0 | 0 | 0 | 0 | 0 | 0 | 0 | 0 | 0 | 0 | 0 | 0 |
| yhiL | 0 | 1 | 1 | 0 | 0 | 0 | 0 | 0 | 0 | 0 | 0 | 0 | 0 | 0 | 0 | 0 | 0 |
| yhiN | 0 | 1 | 1 | 0 | 0 | 0 | 0 | 0 | 0 | 0 | 0 | 0 | 0 | 0 | 0 | 0 | 0 |
| rsmJ | 0 | 1 | 1 | 0 | 0 | 0 | 0 | 0 | 0 | 0 | 0 | 0 | 0 | 0 | 0 | 0 | 0 |
| prlC | 0 | 1 | 1 | 0 | 0 | 0 | 0 | 0 | 0 | 0 | 0 | 0 | 0 | 0 | 0 | 0 | 0 |
| rlmJ | 0 | 1 | 1 | 0 | 0 | 0 | 0 | 0 | 0 | 0 | 0 | 0 | 0 | 0 | 0 | 0 | 0 |
| arsC | 0 | 1 | 1 | 0 | 0 | 0 | 0 | 0 | 0 | 0 | 0 | 0 | 0 | 0 | 0 | 0 | 0 |
| yhiS | 0 | 1 | 1 | 0 | 0 | 0 | 0 | 0 | 0 | 0 | 0 | 0 | 0 | 0 | 0 | 0 | 0 |
| hdeB | 0 | 1 | 1 | 0 | 0 | 0 | 0 | 0 | 0 | 0 | 0 | 0 | 0 | 0 | 0 | 0 | 0 |
| arrS | 0 | 1 | 1 | 0 | 0 | 0 | 0 | 0 | 0 | 0 | 0 | 0 | 0 | 0 | 0 | 0 | 0 |
| gadY | 0 | 1 | 1 | 0 | 0 | 0 | 0 | 0 | 0 | 0 | 0 | 0 | 0 | 0 | 0 | 0 | 0 |
| bcsQ | 0 | 1 | 1 | 0 | 0 | 0 | 0 | 0 | 0 | 0 | 0 | 0 | 0 | 0 | 0 | 0 | 0 |
| yhjR | 0 | 1 | 1 | 0 | 0 | 0 | 0 | 0 | 0 | 0 | 0 | 0 | 0 | 0 | 0 | 0 | 0 |
| bcsE | 0 | 1 | 1 | 0 | 0 | 0 | 0 | 0 | 0 | 0 | 0 | 0 | 0 | 0 | 0 | 0 | 0 |
| rdlD | 0 | 1 | 1 | 0 | 0 | 0 | 0 | 0 | 0 | 0 | 0 | 0 | 0 | 0 | 0 | 0 | 0 |
| yehH | 0 | 1 | 1 | 0 | 0 | 0 | 0 | 0 | 0 | 0 | 0 | 0 | 0 | 0 | 0 | 0 | 0 |
| yehI | 0 | 1 | 1 | 0 | 0 | 0 | 0 | 0 | 0 | 0 | 0 | 0 | 0 | 0 | 0 | 0 | 0 |
| yehM | 0 | 1 | 1 | 0 | 0 | 0 | 0 | 0 | 0 | 0 | 0 | 0 | 0 | 0 | 0 | 0 | 0 |
| yehQ | 0 | 1 | 1 | 0 | 0 | 0 | 0 | 0 | 0 | 0 | 0 | 0 | 0 | 0 | 0 | 0 | 0 |
| yehT | 0 | 1 | 1 | 0 | 0 | 0 | 0 | 0 | 0 | 0 | 0 | 0 | 0 | 0 | 0 | 0 | 0 |
| yoeI | 0 | 1 | 1 | 0 | 0 | 0 | 0 | 0 | 0 | 0 | 0 | 0 | 0 | 0 | 0 | 0 | 0 |
| yefM | 0 | 1 | 1 | 0 | 0 | 0 | 0 | 0 | 0 | 0 | 0 | 0 | 0 | 0 | 0 | 0 | 0 |
| wbbL | 0 | 1 | 1 | 0 | 0 | 0 | 0 | 0 | 0 | 0 | 0 | 0 | 0 | 0 | 0 | 0 | 0 |
| yegI | 0 | 1 | 1 | 0 | 0 | 0 | 0 | 0 | 0 | 0 | 0 | 0 | 0 | 0 | 0 | 0 | 0 |
| yegJ | 0 | 1 | 1 | 0 | 0 | 0 | 0 | 0 | 0 | 0 | 0 | 0 | 0 | 0 | 0 | 0 | 0 |
| yegK | 0 | 1 | 1 | 0 | 0 | 0 | 0 | 0 | 0 | 0 | 0 | 0 | 0 | 0 | 0 | 0 | 0 |
| yegL | 0 | 1 | 1 | 0 | 0 | 0 | 0 | 0 | 0 | 0 | 0 | 0 | 0 | 0 | 0 | 0 | 0 |
| sibA | 0 | 1 | 1 | 0 | 0 | 0 | 0 | 0 | 0 | 0 | 0 | 0 | 0 | 0 | 0 | 0 | 0 |
| sibB | 0 | 1 | 1 | 0 | 0 | 0 | 0 | 0 | 0 | 0 | 0 | 0 | 0 | 0 | 0 | 0 | 0 |
| yegP | 0 | 1 | 1 | 0 | 0 | 0 | 0 | 0 | 0 | 0 | 0 | 0 | 0 | 0 | 0 | 0 | 0 |
| cyaR | 0 | 1 | 1 | 0 | 0 | 0 | 0 | 0 | 0 | 0 | 0 | 0 | 0 | 0 | 0 | 0 | 0 |
| yegZ | 0 | 1 | 1 | 0 | 0 | 0 | 0 | 0 | 0 | 0 | 0 | 0 | 0 | 0 | 0 | 0 | 0 |
| yegR | 0 | 1 | 1 | 0 | 0 | 0 | 0 | 0 | 0 | 0 | 0 | 0 | 0 | 0 | 0 | 0 | 0 |
| gatR | 0 | 1 | 1 | 0 | 0 | 0 | 0 | 0 | 0 | 0 | 0 | 0 | 0 | 0 | 0 | 0 | 0 |
| gatC | 0 | 1 | 1 | 0 | 0 | 0 | 0 | 0 | 0 | 0 | 0 | 0 | 0 | 0 | 0 | 0 | 0 |
| yegX | 0 | 1 | 1 | 0 | 0 | 0 | 0 | 0 | 0 | 0 | 0 | 0 | 0 | 0 | 0 | 0 | 0 |
| yehE | 0 | 1 | 1 | 0 | 0 | 0 | 0 | 0 | 0 | 0 | 0 | 0 | 0 | 0 | 0 | 0 | 0 |
| gfcC | 0 | 1 | 1 | 0 | 0 | 0 | 0 | 0 | 0 | 0 | 0 | 0 | 0 | 0 | 0 | 0 | 0 |
| yccE | 0 | 1 | 1 | 0 | 0 | 0 | 0 | 0 | 0 | 0 | 0 | 0 | 0 | 0 | 0 | 0 | 0 |
| rutE | 0 | 1 | 1 | 0 | 0 | 0 | 0 | 0 | 0 | 0 | 0 | 0 | 0 | 0 | 0 | 0 | 0 |
| rutD | 0 | 1 | 1 | 0 | 0 | 0 | 0 | 0 | 0 | 0 | 0 | 0 | 0 | 0 | 0 | 0 | 0 |
| rutA | 0 | 1 | 1 | 0 | 0 | 0 | 0 | 0 | 0 | 0 | 0 | 0 | 0 | 0 | 0 | 0 | 0 |
| efeU | 0 | 1 | 1 | 0 | 0 | 0 | 0 | 0 | 0 | 0 | 0 | 0 | 0 | 0 | 0 | 0 | 0 |
| ymdE | 0 | 1 | 1 | 0 | 0 | 0 | 0 | 0 | 0 | 0 | 0 | 0 | 0 | 0 | 0 | 0 | 0 |
| ymdA | 0 | 1 | 1 | 0 | 0 | 0 | 0 | 0 | 0 | 0 | 0 | 0 | 0 | 0 | 0 | 0 | 0 |
| yfjJ | 0 | 1 | 1 | 0 | 0 | 0 | 0 | 0 | 0 | 0 | 0 | 0 | 0 | 0 | 0 | 0 | 0 |
| yfjM | 0 | 1 | 1 | 0 | 0 | 0 | 0 | 0 | 0 | 0 | 0 | 0 | 0 | 0 | 0 | 0 | 0 |
| yfjT | 0 | 1 | 1 | 0 | 0 | 0 | 0 | 0 | 0 | 0 | 0 | 0 | 0 | 0 | 0 | 0 | 0 |
| ypjI | 0 | 1 | 1 | 0 | 0 | 0 | 0 | 0 | 0 | 0 | 0 | 0 | 0 | 0 | 0 | 0 | 0 |
| psaA | 0 | 1 | 1 | 0 | 0 | 0 | 0 | 0 | 0 | 0 | 0 | 0 | 0 | 0 | 0 | 0 | 0 |
| pinH | 0 | 1 | 1 | 0 | 0 | 0 | 0 | 0 | 0 | 0 | 0 | 0 | 0 | 0 | 0 | 0 | 0 |
| ypjC | 0 | 1 | 1 | 0 | 0 | 0 | 0 | 0 | 0 | 0 | 0 | 0 | 0 | 0 | 0 | 0 | 0 |
| ygaQ | 0 | 1 | 1 | 0 | 0 | 0 | 0 | 0 | 0 | 0 | 0 | 0 | 0 | 0 | 0 | 0 | 0 |
| gutM | 0 | 0 | 1 | 0 | 0 | 0 | 0 | 0 | 0 | 0 | 0 | 0 | 0 | 0 | 0 | 0 | 1 |

*A: Bacterial Nucleoid; B: Cell Wall; C: Cytosol Cytoplasm; D: Inner Membrane; E: Outer Membrane; F: Periplasmic Space; G: Ribosome; H: Integral To Membrane; I Membrane; J: Pilus; K: Extracellular; L: Surface Cell; M: Intracellular; N: Unknown

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Alba et al. (2020) Cell Host & Microbe 27:1-10.
Alsaadi et al. (2019) A Fusion Peptide in the Spike Protein of MERS Coronavirus. Viruses 11(9):825. PMC6784214.
Altieri (2013) Cancer Lett. 332(2):225-8. PMC3695618
Altschul et al. (1990a) J Mol Biol 215:403-410.
Altschul et al. (1990b) Proc Natl Acad Sci USA 87:14:5509-13.
Altschul et al. (1997) Nucleic Acids Res 25:3389-3402.
Amanat & Krammer (2020) Immunity 52:583-589.
Berry et al. (2003) Hybrid Hybridomics 22(1):23-31.
Bi et al. (2017) Lancet Infect Dis 17:1080-1088.
Bodanszky & Bodanszky (1984) in The Practice of Peptide Synthesis, Springer-Verlag, New York, New York, United States of America.
Carleton et al. (2013) Nat Commun 4:1590. PMC3693737
Chen & Schifferli (2007) Vaccine 25:1626-33.
Chen et al. (2005) J Immunol 175(1):591-598.
Chen et al. (2010) Proc Natl Acad Sci USA 107(7):3099-104. PMC2840271
Chen et al. (2020) J Med Virol 92:418-423.
Chou & Fasman (1974) Biochemistry 13:222-245.
Chou & Fasman (1978) Ann Rev Biochem 47:251-276.
Chou & Fasman (1979) Biophys J 26:367-384.
Cohen (2020) Science 370:894-895.
Colligan et al. (eds.) (1994-2004) Current Protocols in Immunology, John Wiley & Sons, Inc., New York, New York, United States of America.
Colman & Lawrence (2003) Nature Reviews Molecular Cell Biology 4:309-319.
Corey et al. (2020) Science 368:948-950.
Cui et al. (2019) Nat Rev Microbiol 17:181-192.
D. G. H. I. Center (2020) Launch and Scale Speedometer. (Duke Global Health Innovation Center).
Datsenko & Wanner (2000) Proc Natl Acad Sci USA 97:6640-6645.
Davis (2001) Adv Drug Deliv Rev. 51(1-3):21-42.
de Boer et al. (1989) Cell 56(4):641-9.
Delany et al. (2014) EMBO Mol Med. 6(6):708-720.
Deutscher et al. (ed.) (1990) Guide to Protein Purification, Harcourt Brace Jovanovich, San Diego, California, United States of America.
Devereux et al. (1984) Nucl Acids Res 12:387.
Dhanak et al. (2017) Cell Chem Biol. 24(9):1148-60.
Fangusaro et al. (2006) Pediatr Blood Cancer. 2006; 47(1):4-13.
Farley et al. (2016) J Bacteriol 198(8):1186-95. PMC4859596
Finkelstein et al. (2012) Immunotherapy. 4(3):283-90. PMC4501768 Frazer & Curtiss (1975) Curr Top Microbiol Immunol. 69:1-84.
Garg et al. (2016) Cancer Cell Int. 2016; 16:49. PMC4917988 GENBANK® Accession Nos. MN908947, MW383928, QHD43416.1.
Gerdes et al. (1984) J Immunol. 133(4):1710-5.
Gerhardt et al. (eds.) (1994) Methods for General and Molecular Bacteriology, American Society for Microbiology, Washington, DC, United States of America. page 574.
Graham et al. (2018) J Am Med Assoc 319:1431-1432.
Greenfield (ed.) (2013) Antibodies: A Laboratory Manual. 2nd Ed. Cold Spring Harbor Laboratory Press, Cold Spring Harbor, New York, United States of America.
Gretebeck & Subbarao (2015) Curr Opin Virol 13:123-129.
Gross & Mienhofer (eds.) (1981) The Peptides. Vol. 3, Academic Press, New York, New York, United States of America. pages 3-88.
Gu et al. (2020) Gastroenterology 158:1518-1519.
Hanafi et al. (2020) COVID-19 Am J Neuroradiol 41:1384-1387.
Hangartner et al. (2006) Nat Rev Immuno. 6(3):231-43.
Harrison (2015) Virology 479-480:498-507.
Hasbold et al. (1998) Eur J Immunol 28(3):1040-51.
Hashimoto et al. (2005) Mol Microbiol 55:137-149.
He et al. (2004) J Immunol. 173(6):4050-7
Henderson et al. (2000) Trends Microbiol 8(12):529-32. PMID: 11115743
Hidalgo et al. (2003) Pediatr Infect Dis J 22(6):570-2.
Huang et al. (2007) Vaccine. 25(39-40):6981-91.
Huang et al. (2012) Nature 491(7424):406-12. PMC4854285
Huang et al. (2013) mBio 4(5):e00737-13. doi: 10.1128/mBio.00737-13.
Jabbal-Gill (2010) J Drug Target. 18(10):771-86.
Jain et al. (2006) J Bacteriol. 188(13):4841-50. PMC1483012
Jiang et al. (2020) Lancet Infect Dis 20(11):e276-e288.
Johansen et al. (2020) Mucosal Immunology 13(6):877-891.
Jong et al. (2012) Microb Cell Fact 11:85.
Jose & Meyer (2007) Microbiol Mol Biol Rev 71(4):600-19. PMC2168652
Jung & Saif (2015) Vet J204:134-143.
Kanekiyo et al. (2019) J Infect Dis 219:S88-S96.
Karlin & Altschul (1990) Proc Natl Acad Sci USA 87:2264-2268.
Karlin & Altschul (1993) Proc Natl Acad Sci USA 90:5873-5877.
Kato & Hashimoto (2007) Mol Syst Biol. 3:132. PMC1964801
Kavunja et al. (2017) Cancer Immunol Immunother. 66(4):451-60. PMC5470645
Kilpatrick et al. (1997) Hybridoma 16(4):381-9.
Kittlesen et al. (1998) J Immunol 60:2099-2106.
Kjorgaard et al. (2002) J Bacteriol 184:4197-4204.
Klebanoff et al. (2005) Proc Natl Acad Sci USA. 102(27):9571-6. 1172264
Klebanoff et al. (2006) Immunol Rev 211:214-24. PMC1501075
Kohyama et al. (2009) Antiviral Res. 84:168-177.
Kong et al. (2016) Science 352:828-833.
Kyte & Doolittle (1982) J Mol Biol 157:105-132.
LaBreck et al. (2019) J Bacteriol 201(4):e00374-18. doi: 10.1128/JB.00374-18.
Lee et al. (2020) Clinical Infectious Diseases November 21; ciaa1421. doi: 10.1093/cid/ciaa1421.
Li et al. (2008) Biotechnol Bioeng 99(2):485-90.
Lu et al. (2020a) Lancet 395:565-574.
Lu et al. (2020b) Vaccine 38:5212-5218.
Luria-Perez et al. (2007) Vaccine 25:5071-85.
Lv et al. (2009) BMC Immunol. 10:61.
Madu et al. (2009) J Virol 83:7411-21
Maecker et al. (2005) BMC Immunol 6:17. PMC1190174
Maecker et al. (2012) Nat Rev Immunol. 12(3):191-200. PMC3409649
Majzner et al. (2017) Cancer Cell. 2017; 31(4):476-85.
McCray Jr. et al. (2007) J Virol 81:813-821.
McMahan et al. (2020) Nature December 4. doi: 10.1038/s41586-020-03041-6.
Melief et al. (2015) J Clin Invest. 125(9):3401-12. PMC4588240
Meng et al. (2006) EMBO J 25:2297-304.
Meurens et al. (2012) Trends Microbiol 20:50-57.
Miyoshi-Akiyama et al. (2011) J Infect Dis 203(11):1574-81
Morabito et al. (2017) Mucosal Immunol. 10(2):545-54. PMC5123975 Morgan & Gainor (1989) Ann Rep Med Chem 24:243-252.
Nagaraj et al. (2007) J Immunother. 30(2):169-79.
Newman et al. (1992) J Immunol 149(10):3260-72.
Ng et al. (2020) Science 370(6522):1339-1343.
Nicolay et al. (2015) Crit Rev Microbiol 41(1):109-23.
Nizard et al. (2017) Nat Commun 8:15221. PMC5458068
Odevall et al. (2018) Vaccine 36:6606-6614.
Oliveira et al. (2007) Expert Rev Vaccines 6(3):419-29.
Onodera et al. (2008) J Immunol 180(2):907-13.
Opriessnig et al. (2014) PLoS One 9:e104766.
Ou et al. (2016) J Virol 90:5586-5600.
PCT International Patent Application Publication Nos. WO 1999/54464; WO 2004/110486; WO 2006/012539; WO 2021/076897.
PCT International Patent Application Serial No. PCT/US2005/026102.
Poh et al. (2020) Nat Commun 11:2806.
Pompa-Mera et al. (2011) Exp Parasitol. 129(4):393-401. PMID: 21907709
Pratelli et al. (2004) Vet Microbiol 99:43-49.
Qin et al. (2014) Nat Med. 20(6):676-81. PMC4048321
Rapp & Kaufmann (2004) Int Immunol 16:597-605.
Rauch et al. (2018) Frontiers in Immunology 19; 9:1963.
Rey & Lok (2018) Cell 172:1319-1334.
Riese et al. (2014) Expert Opin Drug Deliv 11(10):1619-34.
Rizos et al. (2003) Infection and Immunity 71:6320-6328.
Roe (2017) JAMA Oncol. 2017; 3(9):1169-70.
Roost et al. (1995) Proc Natl Acad Sci USA 92(5):1257-61. PMC42498
Rostad et al. (2020) Pediatrics 146(6):e2020018242.
Ruiz-Pérez et al. (2002) Infect Immun 70:3611-3620.
Russell (2002) Am J Reprod Immunol 47(5):265-8.
Sarmento et al. (2020) J Vet Diagn Invest 32:324-328.
Seder & Ahmed (2003) Nat Immunol 4(9):835-42.
Shata et al. (2000) Mol Med Today 6(2):66-71.
Shen et al. (2020) Cell Host Microbe. 27(4):531-543.e6.
Shoemaker et al. (2005) Infect Immun 73(8):5031-8. PMC1201206
Shrock et al. (2020) Science 370(6520):eabd4250.
Sia et al. (2020) Nature 583:834-838.
Sowa et al. (2009) Cell 138(2):389-403. PMC2716422
Spatola (1983) Chemistry and Biochemistry of Amino Acids, Peptides and Proteins, Vol. VII. Weinstein (ed.). Marcel Dekker, Inc., New York, New York, United States of America, pages 267-357.
Stavnezer et al. (2008) Annu Rev Immunol 26:261-92. PMC2707252
Stevenson et al. (2013) Journal of Veterinary Diagnostic Investigation 25:649-654.
Stewart et al. (1984) in Solid Phase Peptide Synthesis, 2nd Edition, Pierce Chemical Company, Rockford, Illinois, United States of America.
Sturmhoefel et al. (1999) Cancer Res. 59(19):4964-72.
Subramaniam et al. (2017) Virus Res 227:212-219.
Subramaniam et al. (2018) J Gen Virol 99:230-239.
Sun et al. (2020) Cell Host Microbe 28(1):124-133.
Surana et al. (2004) J Biol Chem 279:14679-85.
Tam et al. (2016) Proc Natl Acad Sci USA. 113(43):E6639-E48. PMC5086995
Thomsen et al. (1997) Int Immunol 9(11):1757-66.
Tizard (2020) Vaccine 38:5123-5130.
Tsao et al. (2006) Biochem Biophys Res Commun. 344(1):63-71.
U.S. Patent Application Publication No. 2008/0193469.
U.S. patent application Ser. No. 11/572,453.
U.S. Pat. Nos. 4,554,101; 8,012,932.
Ulmer et al. (2006) Nat Biotechnol 24(11):1377-83.
Unnikrishnan et al. (2012) Curr Opin Immunol. 24(3):337-42.
van Bloois et al. (2011) Trends Biotechnol. 29(2):79-86. PMID: 21146237
van Dorp et al. (2020) Infect Genet Evol 83:104351.
Van Elslande et al. (2020) Clin Infect Dis September 5; ciaa1330. doi: 10.1093/cid/ciaa1330.
van Gils et al. (2016) Nat Microbiol 2:16199.
Vincent et al. (2010) Cancer Res. 70(8):3052-61.
Walls et al. (2020) Cell 181(2):281-292. doi:10.1016/j.cell.2020.02.058.
Wang & Anderson (2019) Curr Opin Virol 34:79-89.
Wang et al. (2004a) Blood 104 (1):200-206.
Wang et al. (2004b) J Virol. 78(11):5612-5618.
Wang et al. (2019) Curr Opin Virol 34:39-49.
Welters et al. (2004) Vaccine 23:305-311.
White et al. (2008) Crit Rev Biochem Mol Biol. 43(3):189-219 (erratum in Crit Rev Biochem Mol Biol. 2008 43(4):287-288).
Wrapp et al. (2020) Science 367:1260-1263
Xia et al. (2020) Cell Mol Immunol 17(7):765-767. doi:10.1038/s41423-020-0374-2.
Xu et al. (2018) Nat Med 24:857-867.
Yang et al. (2009) Int Immunol. 21(1):63-71.
Yang et al. (2010) Vaccine. 28(15):2735-42. PMID: 20105428
Yonker et al. (2020) J Pediatr 227:45-52.
Yusuf & Kett (2017) Hum Vaccin Immunother. 13(1):34-45. PMC5287317
Zaman et al. (2013) Drug Deliv Transl Res. 2013; 3(1):100-9. PMC3539070
Zeichner & Cruz (2020) Pediatrics 146(6):e2020032888.
Zhang et al. (2020) N Engl J Med 382:e38.
Zhou et al. (2006) J Immunol. 177(4):2138-45.
Zhu et al. (2007) Vaccine. 25(46):7955-61.
Zugel (2001) Infect Immun 69:4164-4167.

It will be understood that various details of the presently disclosed subject matter can be changed without departing from the scope of the presently disclosed subject matter. Furthermore, the foregoing description is for the purpose of illustration only, and not for the purpose of limitation.

1. A modified bacterium or derivative thereof having a reduced number of expressed genes and comprising a viral antigen, optionally an antigen from a virus with a class I fusion protein, optionally wherein the viral antigen is expressed on a surface of a membrane or derivative thereof, wherein the bacterium induces an enhanced immune response against the viral antigen when administered to a subject as compared to an immune response that would have been induced in the subject by a bacterium of the same strain that has a full complement of expressed genes.

2. The modified bacterium of claim 1, wherein the antigen from a Retroviridae, optionally HIV, Orthomyxoviridae, Paramyxoviridae, Arenaviridae, Filoviridae, and/or Coronaviridae, optionally a SARS-CoV and/or SARS-CoV-2 antigen.

3. The modified bacterium of claim 1, wherein the antigen is from a Coronaviridae, optionally SARS-CoV, SARS-CoV-2, and/or porcine epidemic diarrhea virus (PEDV)) antigen.

4. (canceled)

5. The modified bacterium of claim 1, wherein the bacterium is a Gram-negative bacterium, optionally a member of the Enterobacteriaceae.

6. The modified bacterium of claim 1, wherein the bacterium is an E. coli.

7. The modified bacterium of claim 1, wherein the reduced number of expressed genes comprises a reduction of at least about 1.0%, 2.0%, 3.0%, 4.0%, 5.0%, 6.0%, 7.0%, 8.0%, 9.0%, 10%, 11%, 12%, 13%, 14%, 15%, or greater than 15% of genes.

8. (canceled)

9. The modified bacterium of claim 1, wherein the viral antigen is a coronavirus antigen, optionally a SARS-CoV and/or SARS-CoV-2 antigen, or an HIV antigen, which is put on the surface of the bacterium by an approach selected from the group consisting of expression by the cell itself, covalent or non-covalent association with the outer membrane, and combinations thereof.

10. The modified bacterium of claim 1, wherein the viral antigen is a coronavirus, optionally a SARS-CoV, SARS-CoV-2, and/or porcine epidemic diarrhea virus (PEDV)) antigen, which is put on the surface of the bacterium by an approach selected from the group consisting of expression by the cell itself, covalent or non-covalent association with the outer membrane, and combinations thereof.

11. The modified bacterium of claim 9, comprising an autotransporter (AT) expression vector encoding the antigen, wherein the expression on the surface is provided by the AT expression vector.

12. The modified bacterium of claim 11, wherein the autotransporter expression vector comprises a codon optimized sequence encoding the antigen.

13. The modified bacterium of claim 11, wherein the AT expression vector comprises a monomeric autotransporter vector or a trimeric autotransporter vector.

14. The modified bacterium of claim 1, wherein the coronavirus, optionally SARS-CoV and/or SARS-CoV-2, antigen comprises, consists essentially of, or consists of, and/or is encoded by, any of SEQ ID NOs: 4-35 and 37 or any subsequence and/or derivative thereof, optionally PSKPSKRSFIEDLLFNKVTLADAGF (SEQ ID NO: 31), SFIEDLLFNKVTLADAGF (SEQ ID NO: 29), and SFIEDLLF (SEQ ID NO: 43), and/or the HIV antigen comprises, consists essentially of, or consists of an amino acid sequence selected from the group consisting of AVGIGAVF (SEQ ID NO: 38), ALGIGAAF (SEQ ID NO: 48), AVGFGAAF (SEQ ID NO: 49), and AAGFGAMF (SEQ ID NO: 50).

15. The modified bacterium of claim 1, wherein the antigen comprises, consists essentially of, or consists of an amino acid sequence as set forth in any of SEQ ID NOs: 16-31 and 37 or an immunogenic subsequence or derivative thereof, and/or an amino acid sequence selected from the group consisting of PSKPSKRSFIEDLLFNKVTLADAGF (SEQ ID NO: 31), SFIEDLLFNKVTLADAGF (SEQ ID NO: 29), and SFIEDLLF (SEQ ID NO: 43), and/or the HIV antigen comprises, consists essentially of, or consists of an amino acid sequence selected from the group consisting of AVGIGAVF (SEQ ID NO: 38), ALGIGAAF (SEQ ID NO: 48), AVGFGAAF (SEQ ID NO: 49), and AAGFGAMF (SEQ ID NO: 50), and/or an immunogenic subsequence or derivative thereof.

16. The modified bacterium of claim 1, wherein the coronavirus, optionally SARS-CoV, SARS-CoV-2, and/or porcine epidemic diarrhea virus (PEDV), antigen comprises, consists essentially of, and/or consists of an amino acid sequence selected from the group consisting of PSKPSKRSFIEDLLFNKVTLADAGF (SEQ ID NO: 31), PSKPSKRSFIEDLLFNVKTLADAG (SEQ ID NO: 42), SFIEDLLF (SEQ ID NO: 43), SFIEDLLFNKVTLADAGF (SEQ ID NO: 29), and GRVVQKRSFIEDLLFNKVVTNGLG (SEQ ID NO: 41).

17. (canceled)

18. A method for producing an antibody in a subject, the method comprising providing a modified bacterium according to claim 1 and administering the modified bacterium to a subject in an amount and via a route sufficient to produce an antibody in the subject against the viral antigen expressed by the modified bacterium, optionally wherein the production of the antibody is enhanced in the subject as compared to an immune response produced in a subject by a bacterium of the same strain that has a full complement of expressed genes and that expresses the viral antigen on its surface.

19. The method of claim 18, comprising administering the modified bacterium to the subject intranasally, transmucosally, including but not limited to orally, rectally, and vaginally; subcutaneously, intradermially, intramuscualrly, other parenteral routes, or any combination thereof.

20. A vaccine composition comprising a modified bacterium according to claim 1 and a pharmaceutically acceptable carrier, optionally wherein the vaccine composition further comprises one or more adjuvants.

21. The vaccine composition of claim 20, wherein the modified bacterium is a live attenuated bacterium or a killed whole cell bacterium, or derivatives or fragments thereof.

22. (canceled)

23. (canceled)

24. A method for vaccinating a subject in need thereof against a viral class I fusion protein, optionally wherein the viral class I fusion protein is a fusion protein from a virus of a viral family selected from the group consisting of Retroviridae, optionally HIV, Orthomyxoviridae, Paramyxoviridae, Arenaviridae, Filoviridae, and/or Coronaviridae, the method comprising providing a vaccine composition according to claim 20 and administering the vaccine composition to the subject.

25. A method for treating an infection of a virus, optionally a virus of a viral family selected from the group consisting of Retroviridae (e.g., HIV), Orthomyxoviridae, Paramyxoviridae, Arenaviridae, Filoviridae, and/or Coronaviridae in a subject in need thereof, the method comprising providing a vaccine composition according to claim 20 and administering the vaccine to the subject.

26. A method for vaccinating a subject in need thereof against a viral class I fusion protein, optionally wherein the viral class I fusion protein is a fusion protein (FP) from a virus of a Coronaviridae, optionally SARS-CoV, SARS-CoV-2, and/or porcine epidemic diarrhea virus (PEDV), optionally a SARS-CoV-2 FP and/or a PEDV FP, the method comprising providing a vaccine composition according to claim 20 and administering the vaccine composition to the subject.

27. A method for treating an infection of a virus, optionally a virus of a Coronaviridae, optionally a SARS-CoV, SARS-CoV-2, and/or porcine epidemic diarrhea virus (PEDV), optionally a SARS-CoV-2 FP and/or a PEDV FP, in a subject in need thereof, the method comprising providing a vaccine composition according to claim 20 and administering the vaccine to the subject.

28. (canceled)

29. An expression vector comprising a nucleotide sequence encoding a viral antigen, wherein the expression vector is configured to express the viral antigen in a modified bacterium or derivative thereof having a reduced number of expressed genes, optionally on the surface of the modified bacterium or derivative thereof.

30. The expression vector of claim 29, wherein the antigen is from a Retroviridae, optionally HIV, Orthomyxoviridae, Paramyxoviridae, Arenaviridae, Filoviridae, and/or Coronaviridae, optionally a SARS-CoV and/or SARS-CoV-2 antigen.

31-39. (canceled)
